(19) United States
(12) Patent Application Publication
(10) Pub. No.: US 2026/0337681 A1
(43) Pub. Date: Oct. 29, 2026

(54) SMALL RNA-BASED PROGNOSTIC SIGNATURES AND THERAPEUTIC COMPOSITIONS FOR CORONARY MICROVASCULAR DYSFUNCTION

(71) Applicant: Gatehouse Bio, Inc., Natick, MA (US)

(72) Inventors: David W. SALZMAN, Natick, MA (US); Neal C. FOSTER, Natick, MA (US); Matthew R. LONG, Natick, MA (US); Molly SROUR, Natick, MA (US); Christian BRION, Natick, MA (US); Nathan RAY, Natick, MA (US); Sara DEFREGGER, Natick, MA (US); Sean MELVILLE, Natick, MA (US); Guangliang WANG, Natick, MA (US); Jessie ANG, Natick, MA (US); Alan P. SALZMAN, Natick, MA (US)

(21) Appl. No.: 18/994,374

(22) PCT Filed: Jul. 17, 2023
(86) PCT No.: PCT/US2023/027938

§ 371 Date: Jan. 14, 2025

Related U.S. Application Data
(60) Provisional application No. 63/389,604, filed on Jul. 15, 2022.

(51) Int. Cl. C12Q 1/6883; C12N 15/113

(52) U.S. Cl. CPC C12Q 1/6883; C12N 15/113; C12N 2310/113; C12N 2310/14; C12N 2310/312; C12N 2310/3125; C12N 2310/313; C12N 2310/315; C12N 2310/321; C12N 2310/322; C12N 2310/3231; C12N 2310/3233; C12N 2310/3341; C12Q 2600/118; C12Q 2600/158

(57) ABSTRACT

The present disclosure provides methods and kits for evaluating and risk stratifying subjects with HFpEF. Specifically, the present disclosure provides methods and kits for determining an expression profile of small, non-coding RNA biomarkers that can predict coronary flow reserve (CFR). CFR is an important clinical measure that is correlated to the presence of coronary microvascular dysfunction (CMD) and predicts morbidity in patients diagnosed with HFpEF. In other aspects, the present disclosure provides therapeutic compositions based on the small, non-coding RNA markers, for example, with the potential to correct dysregulation of several messenger RNA targets with an sRNA mimetic, or with the potential to inhibit expression or activity of the small RNA itself with an antisense oligonucleotide. The therapeutic compositions find use in subjects with CMD and/or HFpEF.

### PRIORITY

This application claims the benefit of, and priority to, U.S. Provisional Application No. 63/389,604 filed Jul. 15, 2022, which is hereby incorporated by reference in its entirety.

### BACKGROUND

The microvasculature is an integral component to all tissues. It is necessary for establishing and maintaining healthy tissue through a network arterioles, capillaries, and venules that regulates local blood perfusion and conducts blood-to-tissue exchange. The coronary microvasculature plays a fundamental role in the regulation of coronary blood flow in response to cardiac oxygen requirements. Impairment of this mechanism, defined as coronary microvascular dysfunction (CMD), carries an increased risk of adverse cardiovascular clinical outcomes such as but not limited to Heart failure with preserved ejection fraction.

Heart Failure with preserved ejection fraction (HFpEF) is heart failure with left ventricular ejection fraction (LVEF) being greater than 50%. Patients with HFpEF generally have abnormal diastolic function, specifically where there is an increase in the stiffness of the left ventricle. During diastole, the increase in the stiffness of the left ventricle will cause increased pressure and/or impaired filling of blood in the left ventricle. The etiology of and pathophysiology of HFpEF is not fully understood due to the presence of comorbidities such as obesity, diabetes, and atrial fibrillation. This disparate disease course presents a major challenge for both clinicians and drug developers.

Approximately half of all patients with heart failure (HF) have a preserved ejection fraction. A common attribute in HFpEF is coronary microvascular dysfunction (CMD), where an estimation of coronary flow reserve (CFR) is indicative of disease progression and outcome. Indeed, a higher morbidity rate is observed in patients with a CFR <2.5 compared to a normal range of ≥2.5. Although CFR is informative, measurement requires specialized techniques that impede use in longitudinal or large-scale studies. Thus, the discovery of blood-based biomarkers that predict CFR would be desirable.

Currently there is one disease modifying approved drug for HFpEF, Entresto®. While the recent approval of Entresto represents a significant advancement in the treatment of HFpEF, the drug does not represent a cure, with efficacy readouts showing 13-20% reductions in hospitalization and death, marginally above statistical significance. Given the disparate disease course of HFpEF, it is unlikely that a single therapeutic can reverse disease course across the entire population.

There is a need for therapies that utilize a precision medicine-based approach to subtype HFpEF, understand the underlying cause, and treat each subpopulation accordingly. There is further a need for therapies that treat or ameliorate CMD, including with comorbidities such as but not limited to HFpEF.

In the various aspects and embodiments, this disclosure meets these and other objectives.

### DESCRIPTION OF THE FIGURES

FIG. 1 is a graph showing the correlation between the small RNA signature score and measured CFR. Subjects were plotted according to their measured CFR and sRNA-signature Model Score calculated using Formula 1 with the log 10(unique molecular index read count+1) for each small RNAs in Feature Set 1. Subjects are coded as CMD-positive or CMD-negative. A best-fit line and 95% confidence interval were plotted; a Pearson r=0.79 with a two-tailed P value <0.0001 was calculated.

FIG. 2 is a graph showing the correlation between the small RNA signature score and measured CFR. Subjects were plotted according to their measured CFR and sRNA-signature Model Score calculated using Formula 2 with the log 10(unique molecular index read count+1) for each small RNAs in Feature Set 2. Subjects are coded as CMD-positive or CMD-negative. A best-fit line and 95% confidence interval were plotted; a Pearson r=0.77 with a two-tailed P value <0.0001 was calculated.

FIG. 3 illustrates an analysis of genes targeted by the small RNA panel.

FIGS. 4A and 4B illustrate a reporter-based assay to develop therapeutic compositions based on the small RNA signature by validating sRNA-mRNA interactions. (A) Schematic diagram of the psi-CHECK-2 dual-luciferase reporter plasmid. Renilla luciferase is transcribed in mammalian cells using the SV40 promoter, Firefly luciferase is transcribed using the HSV-TK promoter. A 75 base pair target site corresponding to a small RNA feature associated with CFR is subcloned into the 3′ UTR of the Renilla luciferase gene. (B) Cells are transfected with reporter plasmids in the presence or absence of either mimetic or antisense oligonucleotide. After an incubation period the cells are lysed and dual-luciferase activity is measured.

FIGS. 5A to 5D illustrate validation of the luciferase reporter assay using small RNA mimetics and antisense oligonucleotides. (A) Sequences of the wild type (WT) and mutant (MT) sRNA Target Site that were cloned into dual-luciferase reporter, antisense oligonucleotide, and double-stranded mimetic used in the validation experiment. (B) Schematic diagram showing the binding interaction of the endogenous hsa-let-7a-3p sRNA to the WT sRNA Target Site, as well as the binding of the antisense oligonucleotide to the endogenous hsa-let-7a-3p sRNA. (C) Schematic diagram showing the binding interaction of the endogenous hsa-let-7a-3p sRNA to the MT sRNA Target Site, as well as the binding of the mimetic to the MT sRNA Target Site. (D) Analysis of Normalized Light Units (Renilla/Firefly) of A549 cells co-transfected with a dual-luciferase reporter harboring a 75 base pair fully complementary (WT) or a mutated (MT) target site for hsa-let-7a-5p, with or without InM of sRNA mimetics or 10 nM of antisense oligonucleotides.

FIG. 6 is a table showing clinical data from the discovery cohort in the PROMIS study.

FIG. 7 is a table showing clinical data from the validation cohort in the GSE53080 study.

FIG. 8 is a graph comparing sRNA sequencing data of miR-22-3p, miR-22-Iso126, and other miR-22 isoforms in serum from subjects enrolled in PROMIS-HFpEF.

FIG. 9 is a table showing Iso126 and miR-22-3p levels in samples from patients from PROMIS-HFpEF study.

FIG. 10 is a schematic showing proposed mechanism for Iso126 biogenesis.

FIG. 11 is a table showing predicted Iso126 targets in genes associated with microvascular dysfunction.

FIG. 12 is a graph showing relative Iso126 activity against Iso126 target genes after A549 cells were co transfected with an Iso126 mimetic (Mimic126) or Iso126 antisense oligonucleotide (ASO126).

FIG. 13 is a graph showing a comparison of relative expression of ASO126 targets ITGA7, LAMA2, KYAT1, PRKCE, and PGR after ASO126 treatment of primary cardiac fibroblasts.

FIG. 14 is a graph showing Iso126 expression in cells treated with ASO126 compared to a Control, non-targeting antisense oligo.

FIG. 15 is a graph displaying a subset of significantly (FDR <0.05) differentially expressed pathways relevant to heart failure and the impact of ASO126 levels.

### DETAILED DESCRIPTION OF EMBODIMENTS

The present disclosure provides methods and kits for evaluating and risk stratifying subjects with HFpEF. Specifically, the present disclosure provides methods and kits for determining an expression profile of small, non-coding RNA biomarkers that can predict coronary flow reserve (CFR). CFR is an important clinical measure that is correlated to the presence of coronary microvascular dysfunction (CMD) and predicts morbidity in patients diagnosed with HFpEF. In other aspects, the present disclosure provides therapeutic compositions based on the small, non-coding RNA markers, for example, with the potential to correct dysregulation of several messenger RNA targets with an sRNA mimetic, or with the potential to inhibit expression or activity of the small RNA itself with an antisense oligonucleotide. The therapeutic compositions find use in subjects with CMD and/or HFpEF.

In one aspect, the present disclosure provides a method for evaluating coronary flow reserve (CFR) in a subject, comprising, providing a cardiac tissue biopsy, blood, serum, or plasma sample from the subject (or sRNA isolated therefrom), and determining an expression profile of one or more or a plurality of (e.g., at least 5) small RNAs listed in Table 1 from the sample. Based on the expression profile, a patient can be given a CFR Score and subsequently stratified into discrete groups having a CFR of less than 2.5 or a CFR of 2.5 or greater. In some embodiments, the subject has HFpEF, and may be suspected of having CMD.

CFR is the capacity of the coronary artery to dilate in response to increased myocardial metabolic demand and is calculated as the difference between the hyperemic flow (peak stress) and the resting flow curve. In healthy subjects, CFR is usually over 3, meaning their coronary circulation can triple the baseline flow when needed. Subjects with a CFR of less than 2.5 typically have CFR impairment, which correlates directly to disease severity in HFpEF (e.g., the presence of CMD). Further, a CRF less than 2.5 correlates to a higher probability of HFpEF-related morbidity. In various embodiments, a HFpEF subject with a CRF less than 2.5 is treated by surgical or pharmaceutical intervention as described elsewhere herein.

In a related aspect, the present disclosure provides a method for risk stratifying a subject diagnosed with HFpEF. The method comprises providing a blood, serum, or plasma sample from the subject (or sRNA isolated therefrom) and determining an expression profile comprising the expression level of one or more or a plurality of (e.g., at least 5) small RNAs in Table 1. Based on the expression profile, the subject is determined to be at high risk or low risk of HF progression and morbidity associated with HF.

In various aspects and embodiments, the present disclosure provides a small RNA panel whose expression correlates to CFR. Small RNA (“sRNAs”) are non-coding RNAs less than 200 nucleotides in length and include microRNAs (miRNAs) (including iso-miRs), Piwi-interacting RNAs (piRNAs), small interfering RNAs (siRNAs), vault RNAs (vtRNAs), small nucleolar RNAs (snoRNAs), transfer RNA-derived small RNAs (tsRNAs), ribosomal RNA-derived small RNA fragments (rsRNAs), small rRNA-derived RNAs (srRNA), and small nuclear RNAs (U-RNAs), as well as novel uncharacterized RNA species. Generally, “isoforms” refer to those sequences that have variations with respect to a reference sequence (e.g., the human genome GRCh38/hg38 build, miRBase, piRNAdb, etc.). In miRBase, each miRNA is associated with a miRNA precursor and with one or two mature miRNA (−5p and −3p). Deep sequencing has detected a large amount of variability in small RNA biogenesis, meaning that from the same precursor RNA many different sequences can be generated. There are three main variations of isoforms: (1) templated variants, where the 5′ and 3′ end are upstream or downstream of the reference; (2) non-templated variants, where nucleotides are added to the 5′ and 3′ end that do not align to the reference; (3) nucleotide substitutions, where internal nucleotides do not align to the reference.

In various embodiments, the expression profile comprises the expression levels of a plurality of sRNAs in Table 1. Table 1 provides 206 sRNA sequences whose expression level is correlated to CFR in HFpEF patients, and which can be used to prepare models for evaluating and risk stratifying HFpEF subjects. As shown in Table 1, the sRNAs include various types of RNA species including piRNAs, miRNAs, tRNA-derived sRNA, yRNA, and other species. The expression level of 148 of the sRNAs in Table 1 were shown to positively correlate to CFR, while the expression level of 58 sRNAs from Table 1 were shown to negatively correlate to CFR.

Table 1 shows sRNA sequences in DNA format. It is understood that where sequences described herein are intended to be RNA or comprise RNA nucleotides, thymine (T) will be replaced with uracil (U) nucleobases.

In various embodiments, the expression profile comprises the expression level of at least 10 sRNAs from Table 1. In embodiments, the expression profile comprises the expression level of at least 20 sRNAs from Table 1. In embodiments, the expression profile comprises the expression level of at least 30 sRNAs from Table 1. In embodiments, the expression profile comprises the expression level of at least 40 sRNAs from Table 1. In embodiments, the expression profile comprises, consists essentially of, or consists of the expression levels of sRNAs from Table 1. In this context, the term “consists essentially of” means that additional sRNAs can also be measured as part of the expression profile, and that such sRNAs do not significantly impact (i.e., reduce) the correlation of the expression profile with CFR. In some embodiments, the additional sRNAs can be used as expression level controls. Models can be developed using training cohorts and employing supervised, regression modeling of expression profiles determined for subjects (which can be HFpEF subjects) with CFR less than 2.5 and subjects with CFR greater than 2.5 (and optionally controls) randomized into training and test groups.

In various embodiments, the expression profile comprises the expression level of at least 10 sRNAs selected from Table 1 (SEQ ID NOS: 1-42) or Table 1 (SEQ ID NOS: 43-84). In embodiments, the expression profile comprises the expression level of at least 20 sRNAs from Table 1 (SEQ ID NOS: 1-42) or Table 1 (SEQ ID NOS: 43-84). In embodiments, the expression profile comprises the expression level of at least 30 sRNAs from Table 1 (SEQ ID NOS: 1-42) or Table 1 (SEQ ID NOS: 43-84). In embodiments, the expression profile comprises the expression level of at least 40 sRNAs from Table 1 (SEQ ID NOS: 1-42) or Table 1 (SEQ ID NOS: 43-84). In embodiments, the expression profile comprises, consists essentially of, or consists of the expression levels of sRNAs from Table 1 (SEQ ID NOS: 1-42) or Table 1 (SEQ ID NOS: 43-84). In this context, the term “consists essentially of”′ means that additional sRNAs can also be measured as part of the expression profile, and that such sRNAs do not significantly impact (i.e., reduce) the correlation of the expression profile with CFR. In some embodiments, the additional sRNAs can be used as expression level controls. Models can be developed using training cohorts and employing supervised, regression modeling of expression profiles determined for subjects (which can be HFpEF subjects) with CFR less than 2.5 and subjects with CFR greater than 2.5 (and optionally controls) randomized into training and test groups.

In some embodiments, the level of at least SEQ ID NO: 86 is determined (“Iso126”), for example, in blood, serum, or plasma.

RNA can be extracted from the sample prior to sRNA detection and quantification. RNA may be purified using a variety of standard procedures as described, for example, in RNA Methodologies, A laboratory guide for isolation and characterization, 2^{nd} edition, 1998, Robert E. Farrell, Jr., Ed., Academic Press. In addition, there are various processes as well as products commercially available for isolation of small molecular weight RNAs, including mirVANA™ Paris miRNA Isolation Kit (Ambion), miRNeasy™ kits (Qiagen), MagMAX™ kits (Life Technologies), and Pure Link™ kits (Life Technologies). For example, small molecular weight RNA may be isolated by organic extraction followed by purification on a glass fiber filter. Alternative methods for isolating sRNAs include hybridization to magnetic beads. Alternatively, sRNA processing for detection (e.g., cDNA synthesis) may be conducted in the biofluid sample, that is, without an RNA extraction step.

In various embodiments, detection of the sRNAs in the expression profile involves one of various detection platforms, which can employ reverse-transcription and amplification. In some embodiments, the detection platform involves hybridization of a probe. In some embodiments, the detection platform involves reverse transcription and quantitative PCR (e.g., RT-qPCR). In some embodiments, the sRNAs are reverse transcribed using stem-loop RT primers. Exemplary stem loop primers are shown in Table 2. In embodiments, the reverse transcripts are amplified with forward and reverse primers. Exemplary forward and reverse primers are also shown in Table 2. The reverse primer can be a universal primer, based on a constant sequence of the stem loop primer. In various embodiments, the quantitative PCR assay employs a fluorescent dye or fluorescent-labeled probe. In embodiments, the quantitative PCR assay employs a fluorescent-labeled probe further comprising a quencher moiety (e.g., TAQMAN Probe).

Generally, real-time PCR monitors the amplification of a targeted DNA molecule during the PCR, i.e. in real-time. Real-time PCR can be used quantitatively, and semi-quantitatively. Two common methods for the detection of PCR products in real-time PCR are: (1) non-specific fluorescent dyes that intercalate with any double-stranded DNA (e.g., SYBR Green (I or II), or ethidium bromide), and (2) sequence-specific DNA probes consisting of oligonucleotides that are labelled with a fluorescent reporter which permits detection only after hybridization of the probe with its complementary sequence (e.g., TAQMAN).

In some embodiments, the assay format is TAQMAN real-time PCR. TAQMAN probes are hydrolysis probes that are designed to increase the specificity of quantitative PCR. The TAQMAN probe principle relies on the 5′ to 3′ exonuclease activity of Taq polymerase to cleave a dual-labeled probe during hybridization to the complementary target sequence, with fluorophore-based detection. TAQMAN probes are dual labeled with a fluorophore and a quencher, and when the fluorophore is cleaved from the oligonucleotide probe by the Taq exonuclease activity, the fluorophore signal is detected (e.g., the signal is no longer quenched by the proximity of the labels). As in other quantitative PCR methods, the resulting fluorescence signal permits quantitative measurements of the accumulation of the product during the exponential stages of the PCR. The TAQMAN probe format provides high sensitivity and specificity of the detection.

Accordingly, in some embodiments, sRNAs in the expression profile are converted to cDNA using specific primers, e.g., a stem-loop primer. Amplification of the cDNA may then be quantified in real time, for example, by detecting the signal from a fluorescent reporting molecule, where the signal intensity correlates with the level of DNA at each amplification cycle.

In embodiments, the expression profile is determined using a hybridization assay. In some embodiments, the hybridization assay employs a hybridization array comprising sRNA-specific probes. Exemplary platforms for detecting hybridization include surface plasmon resonance (SPR) and microarray technology. Detection platforms can use microfluidics in some embodiments, for convenient sample processing and sRNA detection. In other embodiments, the expression profile is determined by nucleic acid sequencing, and sRNAs are identified in the sample by a process that comprises trimming 5′ and 3′ sequencing adaptors from sRNA sequences. See, U.S. Pat. Nos. 10,889,862 and 11,028,440 (the full contents of which are hereby incorporated by reference), which disclose a process that includes computational trimming of sequencing adapters from RNA sequencing data and sorting data according to unique sequence reads. In some embodiments, RNA from multiple samples is pooled for determining expression profiles by sRNA sequencing, with sequences from different samples containing an identifying sample tag sequence (which can be added by RT-PCR or by ligation). In embodiments, the expression profile further comprises the expression level of one or more expression normalization controls.

Generally, any method for determining the presence of sRNAs in samples can be employed. Such methods further include nucleic acid sequence based amplification (NASBA), flap endonuclease-based assays, as well as direct RNA capture with branched DNA (QuantiGene™), Hybrid Capture™ (Digene), or nCounter™ miRNA detection (nanostring). The assay format, in addition to determining the abundance of sRNAs may also provide for the control of, inter alia, intrinsic signal intensity variation. Such controls may include, for example, controls for background signal intensity and/or sample processing, and/or hybridization efficiency, as well as other desirable controls for detecting sRNAs in patient samples (e.g., collectively referred to as “normalization controls”).

In some embodiments, the assay format is a flap endonuclease-based format, such as the Invader™ assay (Third Wave Technologies). In the case of using the invader method, an invader probe containing a sequence specific to the region 3′ to a target site, and a primary probe containing a sequence specific to the region 5′ to the target site of a template and an unrelated flap sequence, are prepared. Cleavase is then allowed to act in the presence of these probes, the target molecule, as well as a FRET probe containing a sequence complementary to the flap sequence and an auto-complementary sequence that is labeled with both a fluorescent dye and a quencher. When the primary probe hybridizes with the template, the 3′ end of the invader probe penetrates the target site, and this structure is cleaved by the Cleavase resulting in dissociation of the flap. The flap binds to the FRET probe and the fluorescent dye portion is cleaved by the Cleavase resulting in emission of fluorescence.

In various embodiments, the log 10 (unique molecular index read count+1) for each small RNAs in the expression profile is scored, for example according to the equation and coefficients in Formula 1 or 2 (described herein), create a composite CFR Score with a Spearman correlation coefficient to CFR with an absolute value of at least 0.770 and P-value less than 0.001, facilitating the discrimination of patients with coronary microvascular dysfunction. In embodiments, expression levels of the sRNAs within the model are altered (up or down) and have a Spearman rho correlation coefficient to CFR with an absolute value of greater than or equal to 0.20. In various embodiments, the sRNAs have a Spearman rho correlation coefficient to CFR equal to an absolute value of at least about 0.20, or at least about 0.30, or at least about 0.40, or at least about 0.50, or at least about 0.60, or at least about 0.70, or at least about 0.80, or at least about 0.90.

In various embodiments, the small RNAs are selected from Table 1 so that: (1) map to miRNA, piRNA, tRNA, snoRNA, and/or esiRNA loci, (2) have a read count greater than 0 in greater than or equal to 70% of the study cohort and, (3) have an uncorrected Spearman rho correlation to CFR with a P-value less than 0.10, (4) have a batch corrected Spearman rho correlation to CFR with an absolute value greater than or equal to 0.097 with a P-value less than 0.21.

In various embodiments, where the subject is determined to have a CFR of less than 2.5 (or otherwise determined to be a high risk HFpEF subject), the subject is treated with surgical or pharmaceutical intervention. As used herein, the term “pharmaceutical intervention” means that the subject is prescribed (and administered) at least one additional drug (compared to any existing treatment prior to the expression profiling), or the subjects' drug regimen is altered by at least one drug (i.e., at least one active agent is replaced in an ongoing regimen with one or more other agents), based on the results of the sRNA expression profiling.

In embodiments, surgical interventions for treating HFpEF subjects or subjects determined to have CMD according to this disclosure are selected from, but not limited to, percutaneous coronary intervention, coronary artery bypass grafting, angioplasty, balloon angioplasty, laser angioplasty, rotational atherectomy, angioplasty with a stent, impella-supported percutaneous coronary intervention, coronary stent, and/or revascularization.

In embodiments where pharmaceutical interventions are desired, pharmaceuticals can be selected from, but not limited to, antiplatelets, anticoagulants, antithrombotics, fibrinolytics, antihypertensives, diuretics, antianginals, hypolipidaemic agents, angiotensin-converting-enzyme (ACE) inhibitors, cardiac glycosides, phosphodiesterase inhibitors, antiarrhythmics, calcium antagonists, statins, among others. For example, pharmaceutical interventions can include fibrates, niacin, bile acid sequestrants, ezetimibe, lomitapide, omega-3 fatty acids, PCSK9 inhibitors, choline, pycnogenol, nitroglycerin, calcium channel blockers, beta blockers, adenosine diphosphate (ADP) receptor inhibitors, phosphodiesterase inhibitors, protease-activated receptor-1 (PAR-1) antagonists, glycoprotein IIB/IIIA inhibitors, adenosine reuptake inhibitors, thromboxane inhibitors, thromboxane synthase inhibitors, thromboxane receptor antagonists, aspirin, triflusal, cangrelor, clopidogrel, prasugrel, ticagrelor, ticlopidine, cilostazol, vorapaxar, abciximab, eptifibatide, tirofiban, dipyridamole, terutroban, vasodilators, angiotensin II receptor blockers, methyldopa, clonidine hydrochloride, guanabenz acetate, guanfacine hydrochloride, hydralazine, minoxidil, adenosine antagonist, alpha blockers, amyl nitrite, atrial natriuretic peptide, nitric oxide inducers, glyceryl trinitrate, isosorbide mononitrate, isosorbide dinitrate, pentaerythritol tetranitrate (PETN), sodium nitroprusside, PDE5 inhibitors, sildenafil, tadalafil, vardenafil, tetrahydrocannabinol (THC), theobromine, papaverine, warfarin, heparins, dalteparin, enoxaparin, tinzaparin, heparinoid, coumarins, indandiones, vitamin K antagonists, factor Xa inhibitors, rivaroxaban, dabigatran, apixaban, edoxaban, fondaparinux, thrombin inhibitors, argatroban, bivalirudin, dabigatran, desirudin, and lepirudin. In some embodiments, the pharmaceutical intervention is Entresto® (sacubitril/valsartan)

In various embodiments, the pharmaceutical intervention comprises administering a regimen comprising one or more of a statin, angiotensin-converting enzyme (ACE) inhibitor, low dose aspirin, beta blocker, calcium channel blocker, nitrate, and ranolazine.

In embodiments, pharmaceutical interventions for HFpEF include sodium-glucose cotransport-2 inhibitors (SGLT2i) such as empagliflozin or dapagliflozin. SGLT2i treatments have been shown to reduce risk of cardiovascular death or hospitalization in patients with HFpEF. See, Wagdy K. and Nagy S., EMPEROR-Preserved: SGLT2 inhibitors breakthrough in the management of heart failure with preserved ejection fraction. Global cardiology science & practice vol. e202117 (2021).

In embodiments, pharmaceutical interventions for HFpEF include Mineralocorticoid Receptor Agonists (MRA) such as spironolactone or eplerenone. MRAs reduced the risk of heart failure hospitalizations in patients with HFpEF. See, Pitt B, et al., Spironolactone for heart failure with preserved ejection fraction. N Engl J Med. 2014 Apr. 10; 370(15): 1383-92.

In another aspect, the present disclosure provides a kit for evaluating samples for CFR or risk stratifying HFpEF, e.g., in accordance with the methods described herein. In some embodiments, the kit comprises sRNA-specific probes and/or primers configured for detecting one or a plurality of sRNAs listed in Table 1 (SEQ ID NOS: 1-206). In embodiments, the kit comprises sRNA-specific probes and/or primers configured for detecting at least 10 sRNAs listed in Table 1 (SEQ ID NOS: 1-206). In embodiments, the kit comprises sRNA-specific probes and/or primers configured for detecting at least 20 sRNAs listed in Table 1 (SEQ ID NOS: 1-206). In embodiments, the kit comprises sRNA-specific probes and/or primers configured for detecting at least 30 sRNAs listed in Table 1 (SEQ ID NOS: 1-206). In embodiments, the kit comprises sRNA-specific probes and/or primers configured for detecting at least 40 sRNAs listed in Table 1 (SEQ ID NOS: 1-206). In embodiments, the kit comprises sRNA-specific probes and/or primers configured for detecting at least 50, at least 60, or at least 75 sRNAs listed in Table 1 (SEQ ID NOS: 1-206).

In some embodiments, the kit comprises sRNA-specific probes and/or primers configured for detecting one or a plurality of sRNAs listed in Table 1 (e.g., from SEQ ID NOS: 1-42 or from SEQ ID NO: 43-84). In embodiments, the kit comprises sRNA-specific probes and/or primers configured for detecting at least 10 sRNAs listed in Table 1 (from SEQ ID NOS: 1-42 or SEQ ID NOS: 43-84). In embodiments, the kit comprises sRNA-specific probes and/or primers configured for detecting at least 20 sRNAs listed in Table 1 (from SEQ ID NOS: 1-42 or SEQ ID NOS: 43-84). In embodiments, the kit comprises sRNA-specific probes and/or primers configured for detecting at least 30 sRNAs listed in Table 1 (from SEQ ID NOS: 1-42 or 43-84). In embodiments, the kit comprises sRNA-specific probes and/or primers configured for detecting at least 40 sRNAs listed in Table 1 (from SEQ ID NOS: 1-42 or 43-84). In embodiments, the kit comprises sRNA-specific probes and/or primers configured for detecting at least 50, at least 60, or at least 75 sRNAs listed in Table 1 (from SEQ ID NOS: 1-42 or 43-84). In some embodiments, the probes and/or primers are configured to detect the sRNAs of Formula 1 or Formula 2.

In some embodiments, the kit comprises probes and primers for detecting at least SEQ ID NO: 86 (“Iso126”), for example, in blood, serum, or plasma.

In some embodiments, the kit comprises sRNA-specific stem loop RT primers. Exemplary stem loop primers are listed in Table 2. The stem-loop primer comprises a constant region that forms a stem loop and a variable nucleotide extension (e.g., of about 5-8 nucleotides, such as 6 nucleotides). The constant region acts as a priming region for a reverse primer during amplification. The variable region of the stem-loop RT primer is configured to specifically reverse transcribe a sRNA of Table 1. In embodiments, the kit comprises forward and reverse primers to amplify the reverse transcripts (i.e., resulting from reverse transcription with the stem loop primer). In some embodiments, the reverse primer can be a universal primer. In some embodiments, forward primers are listed in Table 2. A universal reverse primer is also listed in Table 2. In embodiments, the kit comprises sRNA-specific probes that are fluorescent-labeled, for detecting amplicons in real time. In some embodiments, the probe further comprises a quencher moiety. The probe can be a TAQMAN probe.

In embodiments, the kit comprises an array of sRNA-specific hybridization probes, configured to detect sRNAs of Table 1 (e.g., at least 20, at least 30, at least 40, at least 50, or at least 75 sRNAs of Table 1. In some embodiments, the probes are configured to detect the sRNAs of Formula 1 or Formula 2.

In other aspects, the present disclosure provides therapeutic molecules that target the dysregulated sRNAs of Table 1. In some embodiments, present disclosure provides therapeutic molecules that target the dysregulated miRNAs of Table 1. Specifically, the present disclosure provides pharmaceutical compositions that comprise molecules designed to mimic the action of sRNAs (such as miRNAs) that are down regulated in subjects having CMD or subjects that have a CFR of less than 2.5. Such molecules in some embodiments induce RNA interference of target RNAs (e.g., target miRNAs) in a cell. In some embodiments, the present disclosure provides pharmaceutical compositions that comprise antisense oligonucleotides designed to reduce the expression of sRNAs (such as miRNAs) that are upregulated in subjects having CMD that have a CFR of less than 2.5. Such antisense oligonucleotides can induce degradation of or hinder the action of the target sRNA. In some embodiments, the therapeutic molecule targets or mimics a miRNA that is dysregulated in HFpEF patients having CMD.

RNA interference (RNAi) is a sequence-specific RNA degradation process to knockdown, or silence, theoretically any gene containing the homologous sequence. In naturally occurring RNAi, a double-stranded RNA (dsRNA) is cleaved by an RNase III/helicase protein (Dicer) into small interfering RNA (siRNA) molecules, which are dsRNAs of 19-27 nucleotides (nt) with 2-nt overhangs at the 3′ ends. Afterwards, the siRNAs are incorporated into a multicomponent-ribonuclease called RNA-induced-silencing-complex (RISC). One strand of siRNA remains associated with RISC to guide the complex towards a cognate RNA that has a sequence complementary to the guider SS-siRNA in RISC. This siRNA-directed endonuclease digests the RNA, resulting in truncation and inactivation of the targeted RNA.

In various embodiments, the present disclosure provides a composition comprising a small interfering RNA (siRNA) that comprises an antisense strand and a sense strand, where the antisense strand comprises a nucleotide sequence selected from Table 1 (or at least 12, 13, 14, 15, 16, 17, 18, 19, 20, or 21 consecutive nucleotides of a sequence selected from Table 1). In some embodiments, the siRNA targets a miRNA in Table 1 having a positive Spearman rho correlation (a positive Spearman correlation in Table 1 indicates that the abundance of the small RNA is low in CMD subjects). The siRNA, when introduced into cells either in vivo or ex vivo, induces the degradation of one or more target RNAs (e.g., target mRNAs) in cells. In some embodiments, the siRNA induces the degradation of a plurality of mRNAs (e.g., 2, 3, 4, 5 or more) in target cells. In some embodiments, target cells are cardiomyocytes.

In some embodiments, the siRNA mimics the action of: hsa-mir-7-5p, hsa-mir-146a-5p, hsa-mir-92a-3p, hsa-mir-423-5p, hsa-let-7b-5p, hsa-mir-30d-5p, hsa-mir-30a-5p, hsa-let-7a-5p, hsa-mir-4429, hsa-mir320c, hsa-mir-92b-3p, hsa-mir-486-5p, hsa-mir-25-3p, hsa-let-7d-3p, hsa-mir-16-5p, hsa-let-7c-5p, hsa-mir-6130, or hsa-let-7g-5p.

In embodiments, the siRNA comprises a chemical modification, including any of the well-known chemical modifications for siRNA. In embodiments, the chemical modifications increase stability, reduce endonuclease degradation, reduce immunogenicity, and/or reduce Toll-like receptor recognition. In embodiments, the chemical modification is a nucleobase modification, a backbone modification, and/or a sugar modification.

In embodiments, the nucleobase modification suppresses RNA recognition by a Toll-like receptor (TLR). In embodiments, the nucleobase modification is selected from pseudouridine (ψ), N1-methyl-pseudouridine (N1mΨ), 5-methylcytidine (m5C), 2′-thiouridine (s2U), N6′-methyladenosine (m6A), and 5′-fluorouridine.

In embodiments, the siRNA may have one or more backbone modification(s) selected from phosphorothioate, phosphorodithioate, methylphosphonate, and methoxypropylphosphonate. For example, such modifications may be placed at and/or near the 3′ end of the antisense strand and/or the sense strand. Other modified linkages are described elsewhere herein.

In embodiments, the siRNA comprises one or more sugar modifications, such as those selected from 2′-methoxy (2′-Ome), 2′-O-methoxyethyl(2′-O-MOE), 2′-fluoro (2′-F), 2′-arabino-fluoro (2′-Ara-F), constrained ethyl (cEt), bridged nucleic acid (BNA) and locked nucleic acid (LNA). BNA and LNA nucleotides are described elsewhere herein.

In embodiments, the siRNA comprises a sense and antisense strain, each having a length of from about 12 to about 40 nucleotides. In embodiments, the siRNA comprises two substantially complementary RNA strands with a duplex length of about 12 to about 40 base pairs (such as from 16 to 24 base pairs). In some embodiments, the antisense strand is 21, 22, 23 or 24 nucleotides in length. In embodiments, the siRNA comprises a sense strand overhang and an antisense strand overhang at the 3′ ends. The overhangs may be RNA overhangs or may be deoxythymidine (dT-dT) overhangs. In embodiments, the siRNA is in a 19+2 format (19 base pair duplex, with dTdT overhangs on each strand). In embodiments, the siRNA is an asymmetric siRNA (asiRNA) having a blunt end at the 5′ end of the antisense strand.

Other siRNA formats, including but not limited to short-hairpin RNAs (shRNAs), that may be used are described in US 2008/0188430, which is hereby incorporated by reference. Exemplary siRNA molecules designed based on the sRNAs of Table 1 (including the miRNAs in Table 1) are disclosed in Table 3.

In various aspects and embodiments, the present disclosure provides a composition comprising an antisense oligonucleotide that is at least 10 linked nucleotides in length, and which has a sequence that is complementary to a nucleotide sequence selected from Table 1 (SEQ ID NOS: 1 to 206). In embodiments, the present disclosure provides a composition comprising an antisense oligonucleotide that is at least 10 linked nucleotides in length, and which has a sequence that is complementary to a nucleotide sequence of a miRNA of Table 1. In embodiments, the oligonucleotide is at least 10, at least 20, at least 30, or at least 40 nucleotides in length. In embodiments, the oligonucleotide is about 12 to about 40 nucleotides in length or about 12 to about 25 nucleotides in length, or is 12 to 16 nucleotides in length. In embodiments, the oligonucleotide is 12, 13, 14, 15, 16, 17, 18, 19, or 20 nucleotides in length. In various embodiments, the antisense oligonucleotide can reduce the expression levels of the target sRNAs (e.g., miRNA) in a cell either ex vivo or in vivo. In embodiments, the oligonucleotide consists of a nucleotide sequence that is complementary to a sequence selected from SEQ ID Nos: 1 to 206. In embodiments, the oligonucleotide consists of a nucleotide sequence that is complementary to a sequence selected from SEQ ID NOS: 1, 13, 40, 42, 44, 50-63, 65, 67, 74-77, 81, 83-88, 91-98, 101-103, 106, 107, 133-155, 160-176, 178-187, and 189-200. In some embodiments, the antisense oligonucleotide targets a miRNA in Table 1 having a negative Spearman rho correlation (a negative Spearman correlation in Table 1 indicates that the abundance of the small RNA is high in CMD subjects).

For example, the antisense oligonucleotide targets (i.e., is complementary to a sequence of a miRNA selected from): hsa-mir-320c, hsa-mir-483-5p, hsa-let-7d-5p, hsa-mir-30e-5p, hsa-mir-21-5p, hsa-mir-143-3p, hsa-mir-423-5p, has-mir-423-5p, hsa-mir-122-5p, hsa-mir-181a-5p, hsa-mir-21-3p, hsa-mir-191-5p, hsa-mir-22-3p, hsa-mir-221-3p, hsa-mir-423-3p, hsa-mir-21-5p, hsa-mir-146a-5p, hsa-mir-629-5p, hsa-mir-30d-5p, and hsa-mir-30e-5p, or a precursor RNA or isoform of any of the foregoing. In some embodiments, the antisense oligonucleotide targets (e.g., is complementary to a sequence within) miR-22-3p, or a RNA precursor or isoform thereof. In some embodiments, the antisense oligonucleotide is specific for (is complementary to a sequence within) SEQ ID NO: 86.

In some embodiments, the oligonucleotide has a contiguous sequence of at least six, or at least eight, or at least 10 DNA nucleotides sufficient to recruit an endogenous nuclease such as RNaseH. RNaseH is a non-sequence-specific endonuclease enzyme that catalyzes the cleavage of RNA in a hybridized RNA/DNA substrate. In some embodiments, the oligonucleotide of the present disclosure is a “gapmer,” that is, the oligonucleotide comprises a central block of deoxynucleotides (also referred to herein as “DNA nucleotides”). In embodiments, the term “DNA nucleotide” refers to a nucleotide that is not an RNA nucleotide. DNA nucleotides typically have a 2′ H, but may alternatively have various 2′ chemical modifications, including 2′-halo and 2′-lower alkyl (e.g., C1-4). In some embodiments, the 2′ chemical modifications of DNA nucleotides are independently selected from 2′-Fluoro, 2′-Methyl, and 2′-Ethyl. A gapmer will further comprise a 5′ segment and/or a 3′ segment, each of the 5′ and/or 3′ segments being from 2 to 6 nucleotides or from 2 to 4 nucleotides, and where the 5′ and 3′ segments contain RNA nucleotides. In embodiments, one or more nucleotides of the 5′ segment and/or the 3′ segment comprise 2′-O substituents, optionally where all of the nucleotides of the 5′ segment and the 3′ segment comprise 2′-O substituents. In embodiments, the 2′-O substituents are selected from 2′-O alkyl (e.g., 2′-O methyl, 2′-O ethyl), 2′-O methoxyethyl (MOE), and a bridged nucleotide having a 2′ to 4′ bridge. In embodiments, the bridged nucleotide has a methylene bridge (LNA) or a constrained ethyl bridge (cEt).

In some embodiments, the oligonucleotide comprises one or more locked or bicyclic nucleotides, e.g., bridging the 2′ and 4′ positions (“a bridged nucleotide”). Locked nucleic acid (LNA) or “locked nucleotides” are described, for example, in U.S. Pat. Nos. 6,268,490; 6,316,198; 6,403,566; 6,770,748; 6,998,484; 6,670,461; and 7,034,133, all of which are hereby incorporated by reference in their entireties. LNAs are modified nucleotides that contain a bridge between the 2′ and 4′ carbons of the sugar moiety resulting in a “locked” conformation, and/or bicyclic structure. Other suitable locked nucleotides that can be incorporated in the oligonucleotides of this disclosure include those described in U.S. Pat. Nos. 6,403,566 and 6,833,361, both of which are hereby incorporated by reference in their entireties. In exemplary embodiments, the locked nucleotides are independently selected from a 2′ to 4′ methylene bridge and a constrained ethyl (cEt) bridge (see U.S. Pat. Nos. 7,399,845 and 7,569,686, which are hereby incorporated by reference in their entireties).

In embodiments, the oligonucleotide has a modified polynucleotide backbone or modified internucleotide linkages. The term “internucleotide linkage” refers to the linkage between two adjacent nucleosides in a polynucleotide molecule. Naturally, the internucleotide linkage is a phosphodiester bond that forms between two oxygen atoms of the phosphate group and an oxygen atom of the sugar (either at 3′ or 5′ position) to form two ester bonds bridging between the two adjacent nucleosides. Modification of the internucleotide linkage may provide different characteristics, including but not limited to enhanced stability. For example, phosphorothioate or phosphorodithioate linkages increase the resistance of the internucleotide linkage to nucleases. Another example is phosphoacetate linkage (PACE), which improves transfection characteristics and enhances nuclease resistance. Internucleotide linkages and oligonucleotide backbone modifications which may be employed in the oligonucleotides of the present description include, but are not limited to, phosphodiester, phosphorothioate, phosphorodithioate, methylphosphonate, alkylphosphonate, alkylphosphonothioate, phosphotriester, phosphoramidate, phosphoramidite, phosphorodiamidate, siloxane, carbonate, carboalkoxy, acetamidate, carbamate, morpholino, peptide nucleic acid, borano, thioether, bridged phosphoramidate, bridged methylene phosphonate, bridged phosphorothioate, and sulfone internucleoside linkages.

In some embodiments, the oligonucleotide comprises one or more phosphorothioate or phosphorodithioate internucleotide linkages. These bonds substitute a sulfur atom for a non-bridging oxygen in the phosphate backbone of the oligonucleotide, and can be effective for reducing nuclease digestion. In some embodiments, phosphorothioate or phosphorodithioate bonds can be introduced between the last three to five nucleotides at the 5′- and/or 3′-end of the oligonucleotide to inhibit exonuclease degradation. In some embodiments, the oligonucleotides have a combination of phosphodiester and phosphorothioate/phosphorodithioate linkages. In some embodiments, the oligonucleotides contain at least two, at least three, at least four, at least five, at least six, at least seven, at least eight, at least nine, or at least ten phosphorothioate or phosphorodithioate internucleotide linkages. In some embodiments, the oligonucleotides comprise substantially alternating phosphodiester and phosphorothioate internucleotide linkages. In some embodiments, the oligonucleotides are fully phosphorothioate/phosphorodithioate linked (i.e., all bonds are either phosphorothioate or phosphorodithioate).

In some embodiments, particularly where RNaseH recruitment is not desired, the oligonucleotides have a morpholino backbone. Morpholino oligonucleotides do not trigger the degradation of their target RNA molecules, and can be effective for steric blocking of a target RNA sequence. Morpholino oligonucleotides and their synthesis are disclosed generally in U.S. Pat. Nos. 11,028,386, 10,947,533, and 10,927,378, each of which is hereby incorporated by reference in its entirety. Other backbones that may be used include thiomorpholino and peptide nucleic acid (PNA).

In embodiments, the oligonucleotide targets (i.e., is complementary to) a mapped miRNA in Table 1 (i.e., the annotated form of the miRNA). In some embodiments, the oligonucleotide is complementary to a miRNA isoform shown in Table 1, in which the polymorphism is targeted by the oligonucleotide. In some embodiments, both the isoform and the mapped miRNA (i.e., the annotated miRNA) are reduced in expression by treatment of cells with the oligonucleotide, although the oligonucleotide may impact the abundance of the isoform to a greater degree.

In some embodiments, the oligonucleotide is a pan-targeting oligonucleotide that targets the mapped (i.e. annotated miRNA) as well as one or more isoforms, including one or more isoforms of the miRNA whose expression is increased in CMD. Some such isoforms are shown in Table 1. In embodiments, the oligonucleotide does not target the 3′ and/or 5′ end of the miRNA, to avoid these common polymorphic sites. In some embodiments, the oligonucleotide does not target the 3′ end of the miRNA. In embodiments, the oligonucleotide does not target from 1 to 5 or from 1 to 3 nucleotides at the 3′ end of the miRNA (e.g., the mapped miRNA or an isoform shown in Table 1). Alternatively or in addition, the oligonucleotide does not target the 5′ end of the miRNA. In embodiments, the oligonucleotide does not target from 1 to 5 or from 1 to 3 nucleotides at the 5′ end of the miRNA (e.g., the mapped miRNA or an isoform shown in Table 1). In some embodiments, the oligonucleotide is specific to the corresponding miRNA precursor.

In embodiments, the melting temperature of the oligonucleotide hybridized to its target sequence is at least about 35° C. The Tm of an oligonucleotide is the temperature at which 50% of the oligonucleotide is duplexed with its perfect complement and 50% is free in solution. The Tm can be determined experimentally by measuring the absorbance change of the oligonucleotide with its complement as a function of temperature. The Tm can also be estimated using known publicly available Tm calculators. In some embodiments, the Tm of the oligonucleotide hybridized to its target sequence is at least about 40° C., or at least about 45° C., or at least about 50° C. In some embodiments, the Tm of the oligonucleotide hybridized to its target sequence is from about 35° C. to about 60° C. In some embodiments, the Tm of the oligonucleotide hybridized to its target sequence is from about 40° C. to about 60° C., or from about 50° C. to about 60° C.

In some embodiments, the siRNA or oligonucleotide further comprises a targeting or cell penetrating moiety that increases distribution or accumulation of the agent in certain cells or tissues (e.g., cardiac muscle tissue). For example, such a targeting or cell penetrating moiety may be conjugated directly or indirectly to the 3′ end of the oligonucleotides, optionally though a linker which may be biologically cleavable. In some embodiments, the conjugate comprises a sterol conjugate (e.g., cholesterol conjugate) or fatty acid conjugate such as a palmitoyl or stearyl lipid conjugate. Other conjugates are well known.

In various embodiments, the targeting or cell penetrating moiety comprises an antibody or antigen-binding fragment thereof, an aptamer, a peptide, a biological ligand (e.g., including a glycoconjugate), lipid, sterol, cholesterol or derivative thereof, integrin, RGD peptide, or cell-penetrating peptide (CPP). More specifically, the targeting moiety may be selected from a single-domain antibody, a single chain antibody, a bi-specific antibody, a recombinant heavy-chain-only antibody (VHH), a single-chain antibody (scFv), a shark heavy-chain-only antibody (VNAR), a microprotein (cysteine knot protein, knottin), a DARPin, a Tetranectin, an Affibody; a Transbody, an Anticalin, an AdNectin, an Affilin, a Microbody, a phylomer, a stradobody, a maxibody, an evibody, a fynomer, an armadillo repeat protein, a Kunitz domain, an avimer, an atrimer, a probody, an immunobody, a triomab, a troybody, a pepbody, a vaccibody, a UniBody, a DuoBody, a Fv, a Fab, a Fab′, a F(ab′) 2, and a peptide mimetic molecule. Various ligand-binding platforms are described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties.

In still other embodiments, the siRNA or oligonucleotide are encapsulated in liposomes, polymeric nanoparticles, or lipid nanoparticles, as is known in the art. In various embodiments, the LNPs comprise a cationic or ionizable lipid, a neutral lipid, a cholesterol or cholesterol moiety, and a PEGylated lipid. Lipid particle formulations that find use with embodiments of the present disclosure include those described in U.S. Pat. Nos. 9,738,593; 10,221,127; 10,166,298, which are hereby incorporated by reference in their entirety. In some embodiments, the liposomes or nanoparticles further comprise a targeting moiety as described. In embodiments, a targeting moiety can be conjugated to a population of PEG termini, e.g., on the PEG-lipid.

In other aspects, the present disclosure provides a method (or use of composition) for treating a subject having CMD. The method comprises administering an effective amount of a composition (e.g., comprising an antisense oligonucleotide) sufficient for decreasing the expression of a small RNA (such as a miRNA) selected from Table 1. The composition may target an isoform and/or mapped small RNA or miRNA from Table 1 (e.g., as described herein). In embodiments, the target small RNA is a miRNA in Table 1 having a negative Spearman rho Correlation Coefficient in Table 1. In some embodiments, the composition comprises an antisense oligonucleotide targeting SEQ ID NO: 1, 40, 44, 50-52, 54, 55, 58, 60, 63, 75, 76, 77, 81, 84, 86, 87, 88, 136, 137, 138, 175, 178, 192, 193, 194, 195, 196, and 197. In some embodiments, the antisense oligonucleotide targets (i.e., is complementary to a sequence of a miRNA selected from): hsa-mir-320c, hsa-mir-483-5p, hsa-let-7d-5p, hsa-mir-30e-5p, hsa-mir-21-5p, hsa-mir-143-3p, hsa-mir-423-5p, has-mir-423-5p, hsa-mir-122-5p, hsa-mir-181a-5p, hsa-mir-21-3p, hsa-mir-191-5p, hsa-mir-22-3p, hsa-mir-221-3p, hsa-mir-423-3p, hsa-mir-21-5p, hsa-mir-146a-5p, hsa-mir-629-5p, hsa-mir-30d-5p, and hsa-mir-30e-5p, or a precursor RNA or isoform of any of the foregoing. In some embodiments, the antisense oligonucleotide targets (e.g., is complementary to a sequence within) miR-22-3p, or a RNA precursor or isoform thereof. In some embodiments, the antisense oligonucleotide is specific for SEQ ID NO: 86.

In various embodiments, the composition comprises an oligonucleotide (or composition thereof) as described herein. Exemplary oligonucleotide sequences are described in Table 3. These nucleotides can comprise chemical modification patterns as described herein.

In other aspects, the present disclosure provides a method (or use of composition) for treating a subject having CMD, comprising administering an effective amount of a composition (e.g. an siRNA) sufficient to mimic the action of a small RNA selected from Table 1. The composition may mimic an isoform and/or mapped small RNA or miRNA from Table 1. In embodiments, the small RNA is a miRNA in Table 1 having a positive Spearman rho Correlation Coefficient in Table 1. In some embodiments, the composition comprises an siRNA (e.g., as described herein) mimicking the action of: SEQ ID NO: 13, 42, 53, 56, 57, 59, 61, 62, 74, 83, 85, 91-98, 101-103, 106, 107, 133-135, 139-155, 160-174, 176, 179-187, and 189-191, and 198-200. In some embodiments, the siRNA mimics the action of: hsa-mir-7-5p, hsa-mir-146a-5p, hsa-mir-92a-3p, hsa-mir-423-5p, hsa-let-7b-5p, hsa-mir-30d-5p, hsa-mir-30a-5p, hsa-let-7a-5p, hsa-mir-4429, hsa-mir320c, hsa-mir-92b-3p, hsa-mir-486-5p, hsa-mir-25-3p, hsa-let-7d-3p, hsa-mir-16-5p, hsa-let-7c-5p, hsa-mir-6130, or hsa-let-7g-5p.

In various embodiments, the composition comprises an siRNA or shRNA, or another suitable format for inducing RNAi as described herein. In some embodiments, the pharmaceutical composition comprising the siRNA or shRNA is as described herein. Exemplary siRNAs are described in Table 3. These nucleotides can comprise chemical modification patterns as described herein.

In embodiments, the subject having CMD also has HFpEF. In some embodiments, the subject having CMD is not diagnosed as having HFpEF. CMD can be classified in the following four groups: (i) CMD occurring in the absence of obstructive epicardial CAD and myocardial diseases (type A); (ii) CMD occurring in the context of cardiomyopathies (type B); (iii) CMD occurring in the presence of obstructive epicardial CAD (type C); and (iv) iatrogenic CMD (type D). See, Camici P G, Crea F. Coronary microvascular dysfunction. N Engl J Med 2007; 356:830-840.

There are two endotypes of CMD, functional CMD and structural CMD. Patients with functional CMD were found to have a heightened resting coronary blood flow (CBF). Patients with structural CMD have a normal resting CBF. Both endotypes have an impaired augmentation of CBF in response to intravenous adenosine (CFR <2.5). However, patients with structural CMD have an elevated minimal microvascular resistance (which translates to reduced maximal CBF), whereas patients with functional CMD have a normal minimal microvascular resistance, but nevertheless have reduced vasodilatory reserve as the patients have reduced tone at rest. These endotypes have a similar core phenotype, with both groups demonstrating high prevalence of stress perfusion defects on cardiac magnetic resonance (CMR) imaging and reduced coronary perfusion efficiency, on wave intensity analysis, during physical exercise. However, the endotypes differ in their pathogenesis at the microvascular level. Subjects with functional CMD have a heightened resting CBF which suggests a submaximal vasodilatory state at rest, leading to an attenuated vasodilatory capacity in response to physiological stress. The elevated resting CBF in these subject appears to be a response to an increased myocardial oxygen demand or disordered autoregulation of the neuronal nitric oxide synthase (nNOS) pathway, which has been shown to regulate the resting CBF in both healthy and diseased states. Subjects with structural CMD have a normal resting CBF, similar to patients with preserved CFR, but they have an impaired ability to augment their CBF in response to physiological stress, leading to ischemia. Subjects with structural CMD appear to have more established cardiovascular risk factors, including poorly controlled hypertension, type 2 diabetes mellitus (T2DM) and a higher prevalence of exercise-induced hypertension. See, Sinha A, Rahman H, Perera D. Coronary microvascular disease: current concepts of pathophysiology, diagnosis and management. Cardiovasc Endocrinol Metab. 2020 Jul. 16; 10(1): 22-30.

In embodiments, the subject has functional CMD. In embodiments, the subject has structural CMD. In embodiments, the subject has Type A CMD. In embodiments, the subject has Type B CMD. In embodiments, the subject has Type C CMD. In embodiments, the subject has Type D CMD.

In some embodiments, the subject is identified as having a CFR of less than 2.5, according to the methods described herein (e.g., sRNA expression profiling). In embodiments, a subject with a CRF less than 2.5 is identified to have coronary microvascular dysfunction. In embodiments, a subject with a CRF less than 2.5 correlates to higher probability of CMD-related morbidity. In embodiments, the subject with a CRF less than 2.5 is treated by surgical or pharmaceutical intervention, such as by administering a composition described herein. In embodiments, the subject diagnosed with CMD is treated by surgical or pharmaceutical intervention (e.g., by administering a composition described herein).

Dosing and administration schedules can vary, depending on the condition of the patient, and the chemistry of the composition. In various embodiments, the compositions are administered about weekly, about bimonthly (i.e., about every other week), about monthly, or about quarterly. Dosing and administration schedules can further include varying dosing and administration frequency based on the route or delivery (e.g., parenterally, or direct administration to target tissues) and the patient's response. In some embodiments, the compositions are administered intravenous administration. In some embodiments, the compositions are delivered directly or locally to the heart.

Other aspects and embodiments of the present disclosure will be apparent from the following Examples.

As used herein, the term “about”, unless the context requires otherwise, means=10% of an associated value.

### EXAMPLES

### Example 1: Finding sRNA-Signature that Models CFR

Half of all patients with heart failure (HF) have a preserved ejection fraction (HFpEF). A common attribute in HFpEF is coronary microvascular dysfunction (CMD), where an estimation of coronary flow reserve (CFR) is indicative of disease progression and outcome. Indeed, a higher morbidity rate is observed in patients with a CFR <2.5 compared to a normal range of ≥2.5. Although CFR is informative, measurement requires specialized techniques that impede use in longitudinal or large-scale studies. Thus, the discovery of blood-based biomarkers that predict CFR would be a valuable research tool and may provide insights into HFpEF biology. It was discovered that small, non-coding (sRNA) biomarkers could predict CFR in patients with HFpEF.

Serum samples from 180 unique subjects (127 with CMD [CFR <2.5] and 53 without CMD [CFR ≥2.5]) were selected from the Prevalence of Microvascular Dysfunction in HFpEF (PROMIS) study. Samples were batch processed in 3 groups of 16 samples per day. Small RNAs were extracted using the miRNeasy Serum/Plasma Advanced Kit (Qiagen). Isolated nucleic acid was eluted in 12.0 μL of Tris pH 8.0. Small RNAs were cloned from 10.0 μL of nucleic acid eluate using the NextFlex Small RNA Library Prep Kit (BIOO) with a 16-hour incubation with 3′-adaptor and 22 cycles of polymerase chain reaction were used to incorporate i7/i5 molecular indexes. Unique libraries were normalized and pooled at concentration of 2.5 ug/uL. Pooled libraries between 160 and 195 base pairs were purified using a 4% agarose gel. Samples were sequenced at a target depth of 20 million paired-end reads using an S4 patterned flow cell on a NovaSeq 6000 (Illumina).

Sequencing files were converted from a .sra to .fastq format using the SRA Tool Kit v2.8.0 for Centos, and fastq formatted files were processed as described in U.S. Pat. No. 10,889,862 (which is hereby incorporated by reference in its entirety). Specifically, all .fastq data files were processed by trimming adaptor sequence using the (Regex) regular expression-based search and trim algorithm, where 5′ TGGAATTCCTCGGGTGCCAAGG 3′ (containing up to a 15 nucleotide 3′-end truncation) was input to identify the 3′ cloning adaptor sequence, and a Levenshtein Distance of 2 or Hamming Distance of 5. Parameters for Regex searching requires that the 1^{st} nucleotide of the user-specified search term be unaltered with respect to nucleotide insertions, deletions, and/or swaps; 5′ TCTTTCCCTACACGACGCTCTTCCGATCT 3′ (containing up to a 15 nucleotide 5′-end truncation) was input to identify the 5′ cloning adaptor sequence, and a Levenshtein Distance of 2 or Hamming Distance of 5. Parameters for Regex searching requires that the 29th nucleotide of the user-specified search term be unaltered with respect to nucleotide insertions, deletions, and/or swaps. Paired-end reads were removed if they were not an exact match. A 4-nucleotide NNNN prefix and NNNN postfix were used as a Unique Molecular Index (UMI) to quantify reads of unique small RNAs. The UMIs were removed after quantification. Reads were aligned to a 17-95 nucleotide tiled array of the human genome (hG38) with a Levenshtein distance of 2. The number of trimmed reads per million was calculated for each unique small RNA cloned in the dataset.

Three distinct approaches were used to identify small RNA features that correlate to CFR, resulting in three unique panels small RNAs.

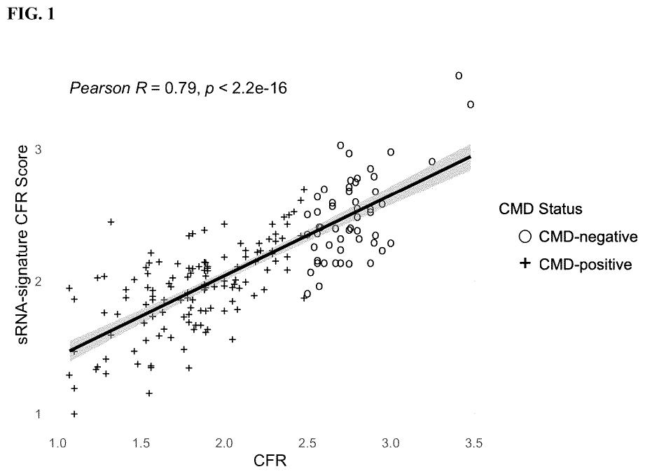

The first feature set was identified by selecting small RNA features with at least one of the following characteristics: (1) statistically significant correlation to either CFR v Max, or CFR v Mean, or CFR VTI, (2) Differentially expressed (p adjusted <0.1) for any threshold of CFR v Max, or CFR v Mean, or CFR VTI, or (3) referenced in a published research article on HFpEF. This resulted in a candidate feature set of 2,652 unique sequences. Next, the subjects were split into an 80% training set (144 subjects) and a 20% test set (36 subjects). A linear regression with elastic net penalty (alpha=0.5, lambda=0.002) selected 42 distinct features with the best performance in the training set using 10-fold cross-validation. Integration of age, sex, and NT-proBNP as features did not impact performance during parameter testing. Models comprised of multiple small RNA features gave higher Spearman rho values compared to individual small RNA features. The final linear regression with elastic net penalty model was fit to the full dataset using the 42 selected small RNAs. The equation for the final model is detailed in Equation 1. The model achieved a Spearman rho=0.790, p<0.0001 (FIG. 1). Accuracy, sensitivity, and specificity for the sRNA model were determined as a tool to predict presence or absence of CMD. Model testing results in a sensitivity of 82%, a specificity of 72%, and accuracy of 79% for distinguishing CMD in the setting of HFpEF. The 42 sRNA sequences used in the model are shown in Table 1 (SEQ ID NO: 1 to 42).

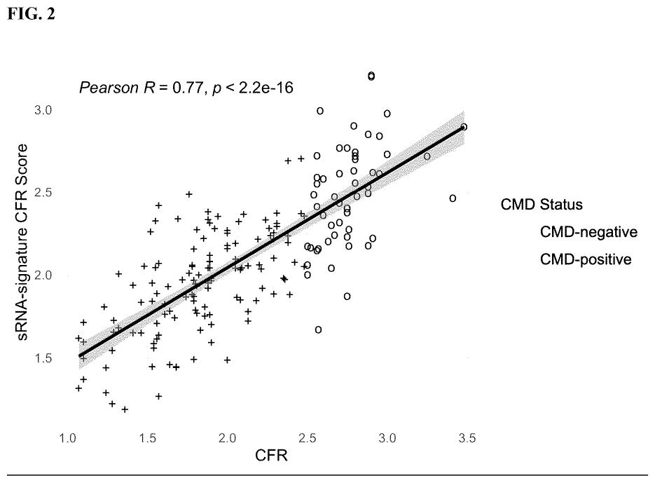

The second feature set was identified by selecting small RNA features expressed in at least 10% of the sample cohort. Next, a linear regression with elastic net penalty (alpha=0.5, lambda=0.003) selected 42 distinct features with best performance in the 80% trainset using 10-fold cross-validation. Integration of age, sex, and NT-proBNP as features did not impact performance during parameter testing. Models comprised of multiple sRNA features gave higher Spearman rho values compared to individual sRNA features. The final linear regression with elastic net penalty model was fit to the full dataset using these 42 features. The equation for the final model is detailed in Equation 2. The model achieved a Spearman rho=0.770, p<0.0001 (FIG. 2). Accuracy, sensitivity, and specificity for the sRNA model were determined as a tool to predict presence or absence of CMD. Model testing results in a sensitivity of 87%, a specificity of 68%, and accuracy of 82% for distinguishing CMD in the setting of HFpEF. The 42 sRNA sequences used in the model are shown in Table 1 (SEQ ID NO: 43 to 84).

The third feature set was identified by selecting small RNA that: (1) mapped to miRNA, piRNA, tRNA, snoRNA, or esiRNAs, (2) have a read count greater than 0 in greater than or equal to 70% of the study cohort and, (3) have a uncorrected Spearman rho correlation to either CFR v Mean, or CFR v Max, or CFR VTI with a P-value less than 0.1, (4) have a batch corrected Spearman rho correlation to either CFR v Mean, or CFR v Max, or CFR VTI with an absolute value great than or equal to 0.097 with a P-value less than 0.21. The Spearman correlation was computed using expression value normalized for read depth: log 2 (trimmed reads per million+1) and base only on the samples for which the small RNA has been sequenced: read counts >0. A positive Spearman correlation with any CFR type indicates that the abundance of small RNA is low in HFpEF patients.

The three features sets are shown in Table 1. Table 1 shows the sequence of the small RNA (in DNA sequence) as well as the mapped name and sRNA class. The mapped name is the existing annotated gene where the read aligns. Table 1 further lists the population frequency of each sRNA feature, that is, the percent of the cohort population where the read was detected.

For each selected small RNA, batch effect on expression was corrected essentially as described in Ritchie, M. E., et al. (2015). Limma powers differential expression analyses for RNA-sequencing and microarray studies. Nucleic Acids Research 43(7), e47.

The corrected expression was then used to compute sRNA abundance correlation with patient's parameters: CFR v Mean, or CFR v Max, or CFR VTI using Spearman correlation. The extensive list of selected small RNAs, correlation coefficients, and P-value is provided in Table 1.

RT-qPCR primers and probes were designed for specific detection of the 43 sRNAs. RT stem loop primers, PCR primers, and qPCR probes are shown in Table 2. Accordingly, this example provides a surrogate, RT-qPCR assay to predict CFR, which provides a scalable, blood-based alternative to the adenosine stress transthoracic Doppler echocardiograph (as was used in the PROMIS trial).

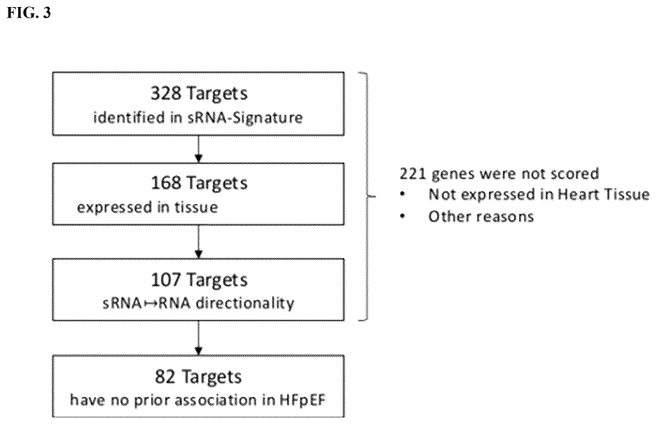

sRNAs are master regulators of gene expression that suppress translation of complementary target RNAs. Prediction algorithms identified 328 regulatory targets with a p<0.0001 and q<. 0.05 using a reverse-complement lookup into RefSeq seeded with nucleotides 2-10(±2) of each sRNA in the final model. A flow chart for analyzing the targets is illustrated in FIG. 3, which includes analyzing tissue expression, correlations with mRNA expression, and prior associations with HFpEF.

The sRNA features identified in this example were derived from serum samples, thereby reflecting peripheral effects and not necessarily the biology of diseased tissue in the heart and other organs involved in HFpEF. To address this, RNA-seq data from Hahn et al. was analyzed, generated from right ventricle tissue biopsies collected from HFpEF patients and age-matched controls., to determine the number of predicted targets (based on our sRNA findings) that were expressed in cardiac tissue. A total of 33% (107 of 328) predicted targets were expressed in cardiac tissue with p<0.05. In patients with CMD, 100 genes were down-regulated, and 7 genes were up-regulated. The inferred directionality of identified sRNA features aligned with cardiac expression.

### Example 2: Therapeutic Modalities Based on the sRNA Signature

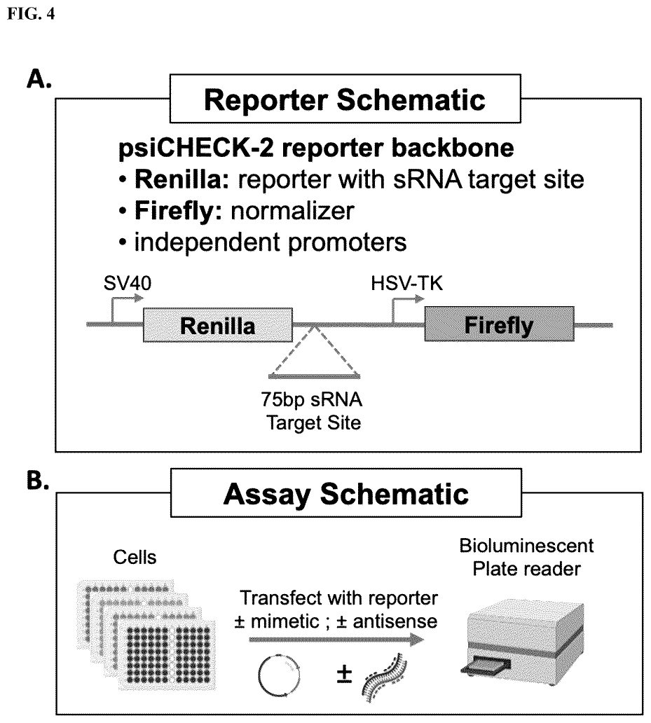

Antisense oligonucleotide compositions targeting each sRNA were designed (Table 3). siRNAs mimicking each sRNA of the model were also designed (Table 3). These designs are screened to evaluate the putative sRNA-RNA interactions. An exemplary reporter assay is shown in FIGS. 4A-B. Specifically, FIGS. 4A and 4B illustrate a reporter-based assay to develop therapeutic compositions based on the small RNA signature by validating sRNA-mRNA interactions. Renilla luciferase is transcribed in mammalian cells using the SV40 promoter, and Firefly luciferase is transcribed using the HSV-TK promoter. A 75 base pair target site corresponding to a small RNA feature associated with CFR is subcloned into the 3′ UTR of the Renilla luciferase gene. Cells are transfected with reporter plasmids in the presence or absence of either mimetic or antisense oligonucleotide, and after an incubation period, the cells are lysed and dual-luciferase activity is measured.

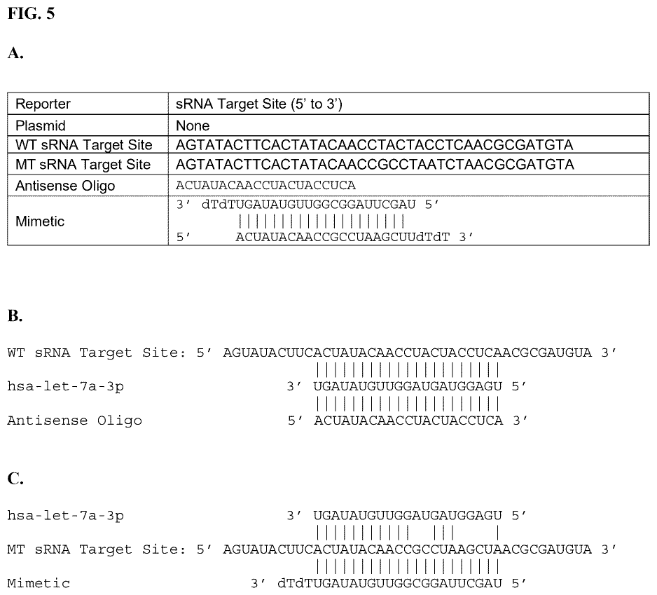

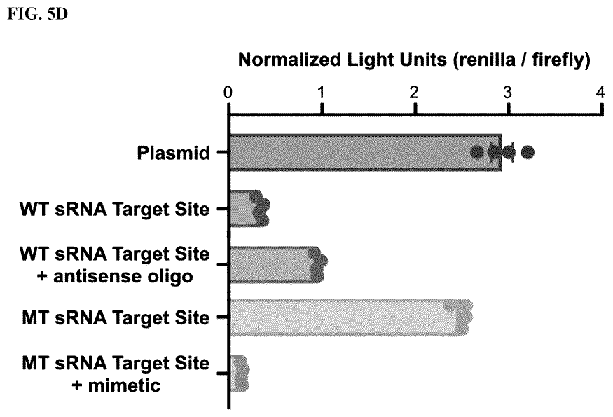

FIGS. 5A to 5D illustrate validation of the luciferase reporter assay using the small RNA mimetics and antisense oligonucleotides. Sequences of a wild type (WT) and mutant (MT) sRNA Target Site were cloned into dual-luciferase reporter construct. Antisense oligonucleotide and double-stranded mimetic were used in the validation experiment. FIG. 5B shows the binding interaction of the endogenous hsa-let-7a-3p sRNA to the WT sRNA Target Site, as well as the binding of the antisense oligonucleotide to the endogenous hsa-let-7a-3p sRNA. FIG. 5C shows the binding interaction of the endogenous hsa-let-7a-3p sRNA to the MT sRNA Target Site, as well as the binding of the mimetic to the MT sRNA Target Site.

FIG. 5D shows analysis of Normalized Light Units (Renilla/Firefly) of A549 cells co-transfected with a dual-luciferase reporter harboring a 75 base pair fully complementary (WT) or a mutated (MT) target site for hsa-let-7a-5p, with or without 1nM of sRNA mimetics or 10 nM of antisense oligonucleotide. Co-transfection of cells with the WT sRNA target site and antisense oligonucleotide showed inhibition of let-7a-mediated suppression. Co-transfection of cells with the MT sRNA target site and a mimetic showed knockdown.

The mimetics and antisense oligonucleotides described herein represent a novel class of therapeutics targeting sRNA dysregulation, with the potential to regulate numerous downstream RNA targets simultaneously.

### Example 3: Analysis of Iso126 Mimetic and Iso126 Antisense Oligonucleotide (ASO126)

The PROMIS study was designed to investigate the prevalence of coronary microvascular dysfunction (CMD) and its association with systemic endothelial dysfunction, heart failure severity, and myocardial dysfunction in a well-defined, multi-center HFpEF cohort. Metadata included sex, age at collection, CFR v mean, NT-proBNP and 6MWT (FIG. 6). Subjects with a CFR ≤2.5 were diagnosed with CMD, whereas subjects with a CFR >2.5 were diagnosed without CMD.

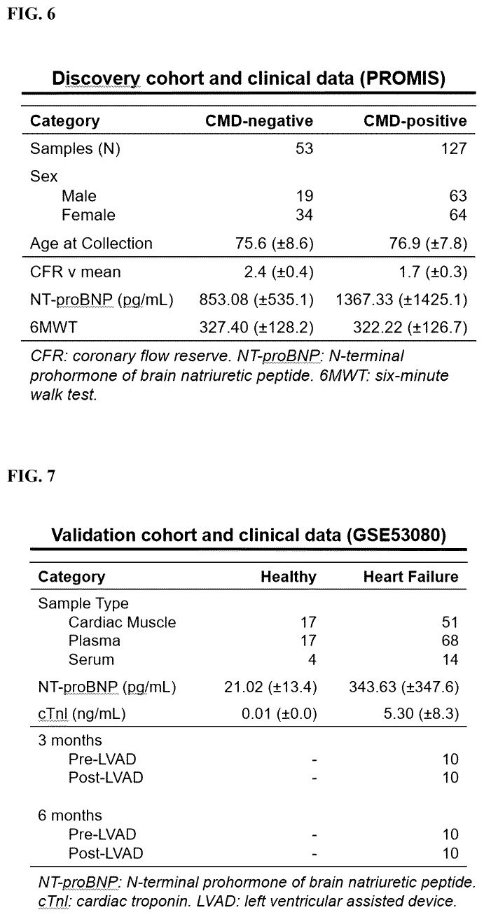

The GSE53080 study was designed to characterize small RNA expression profiles in left ventricle cardiac muscle biopsies, as well as plasma and serum samples collected from healthy donors and subjects diagnosed with advanced heart failure. Metadata included NT-proBNP and cTn1 levels (FIG. 7).

Small RNA sequencing data was generated from 0.5 mL of serum collected from subjects enrolled in PROMIS-HFpEF. Adaptors were removed from paired-end reads, unique small RNA sequences were aggregated and quantified, and sequences were mapped to either the human genome (hg38), miRbase (v22.1), snoDB (v2.0), GtRNAdb (v21), and piRBase (v3.0). Data was filtered to include small RNAs with a read count ≥1 trimmed reads per million (TRPM) and a frequency in the cohort of >75%. Differential expression was determined by comparing the TRPM read counts of small RNAs in CMD-positive subjects to the TRPM read counts of small RNAs in CMD-negative subjects. Results were filtered to small RNAs with a log 2 fold-change ≥0.75 and a P-value≤0.05.

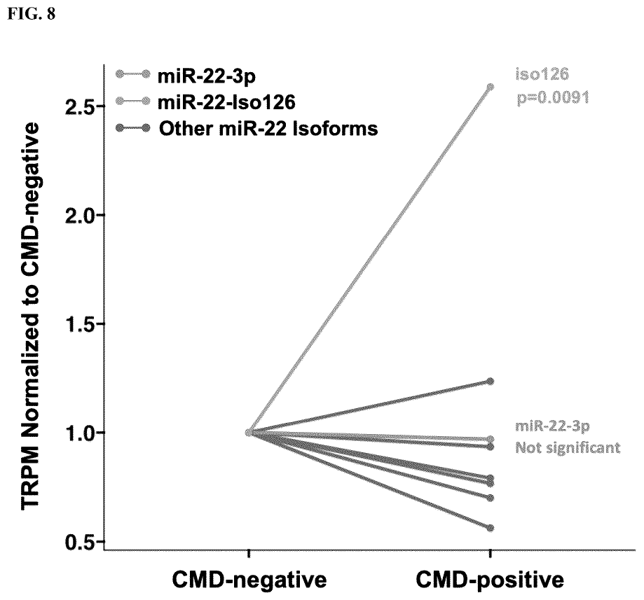

Results showed that 8 small RNAs mapped to the miR-22-3p loci in chr17, when applying these filters. Of these 8 small RNAs an isoform termed ‘Iso126’ was significantly over-expressed in patients with CMD compared to patients without CMD (Fold Change=2.63, P-value=0.0091). Expression of miR-22-3p, the canonical reference small RNA, and other miR-22 isoforms were not significantly different in subjects with or without CMD (FIG. 8). Without being bound to a particular theory, Iso126 variant is associated with CMD.

Peripheral blood serum was extracted from 180 patients in the PROMIS-HFpEF study (FIG. 9). sRNA expression was measured using Next-generation sequencing (NGS). Results showed a significant positive correlation between expression levels of ISO126 (measured in TRPM) and both CFR Score and NT-proBNP. Statistical significance was determined using Spearman rank order correlation. Additionally, results showed a significant positive log 2-fold change for ISO126 in microvascular dysfunction (MVD)-positive patients with respect to MVD-negative patients. Statistical significance was determined using the Wald test with BH-adjustment. When comparing these results to the same comparisons for the annotated miR-22-3p, no significant fold change or correlation to CFR Score was observed, but a significant positive correlation to NT-proBNP was observed. Statistical significance was determined using Spearman rank order correlation. (See FIG. 9)

Myocardium tissue, peripheral blood plasma, and/or peripheral blood serum was extracted from patients with either dilated or ischemic cardiomyopathy or healthy controls. sRNA expression was measured using NGS. Results showed a significant positive log 2-fold change for ISO126 in patients with heart failure vs. healthy controls in both cardiac tissue and biofluid (plasma and serum). Statistical significance was determined using the Wald test with BH-adjustment. When comparing these results to the same comparisons for the annotated miR-22-3p, a similar fold change was observed in cardiac tissue but no significant fold change was observed in biofluid (See FIG. 9).

Significant positive log 2-fold change was observed for ISO126 in plasma when comparing heart failure patients with either 3 or 6 months post-implantation of an LVAD with these same patients prior to LVAD implantation. Statistical significance was determined using the Wald test with BH-adjustment. Additionally, significant positive correlation between expression levels of ISO126 and both NT-proBNP and Cardiac Troponin (cTnI) was observed. When comparing these results to the same comparisons for the annotated miR-22-3p, no significant fold change or correlation to NT-proBNP was observed, but a significant positive correlation to cTnI was observed. (See FIG. 9)

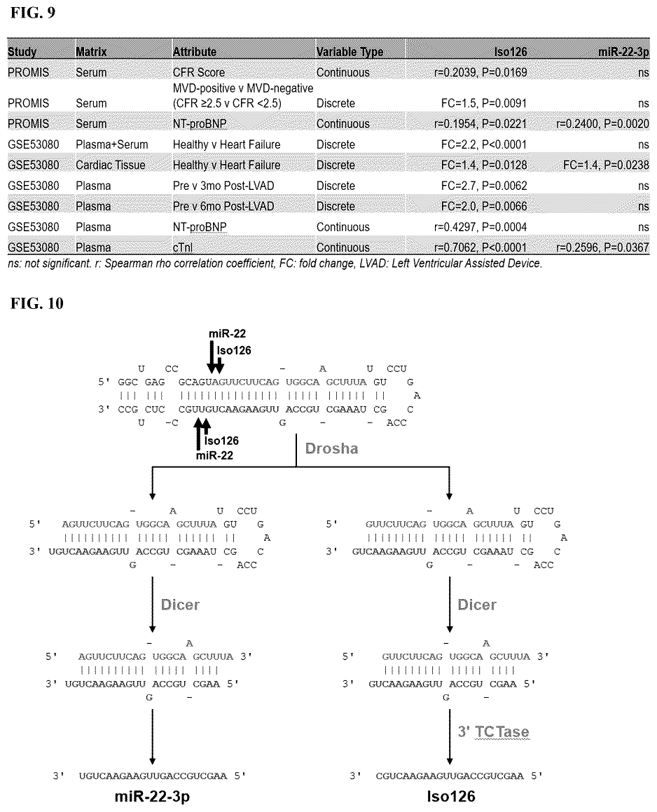

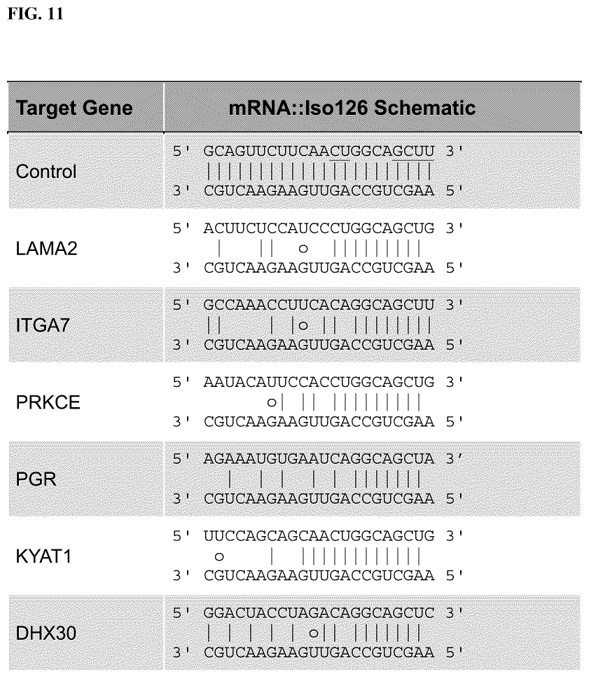

FIG. 10 shows a proposed mechanism for Iso126 biogenesis. In this proposed mechanism, Drosha cleaves the pri-miR-22 transcript between the indicated UpA and UpU bonds to form pre-miR-22, or between the indicated ApG and UpG bonds to form Iso126. Following Dicer cleavage, a template-independent 3′ terminal cytosine transferase (TENT) ligates a single cytosine nucleotide to the 3′ end to generate Iso126.

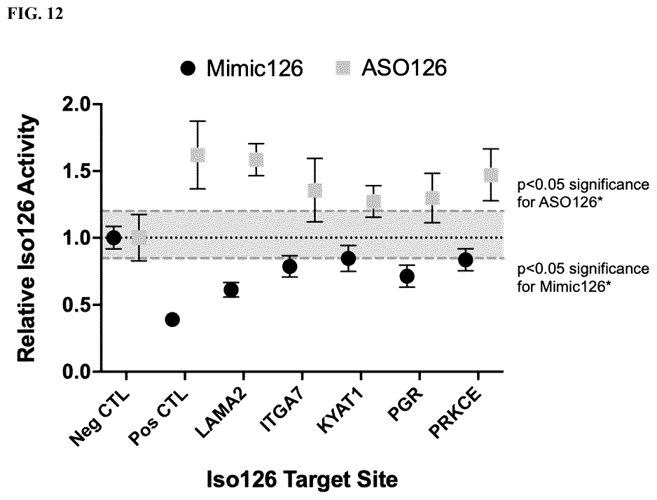

FIG. 11 shows the predicted Iso126 target sites in genes associated with microvascular dysfunction. Target sites were subcloned into the 3′ untranslated region of the renilla messenger RNA in the psiCHECK2 dual-luciferase reporter plasmid. A549 cells were co-transfected with dual-luciferase reporters (100 ng) and an Iso126 mimetic or Iso126 antisense oligonucleotide (ASO126). Over-expression of Iso126 using Mimic126 (100 nM) (see Table 3) showed statistically significant knock-down of reporters containing Iso126 target sites (ordinary, one-way ANOVA with correction for multiple comparisons, compared to positive control (Pos CTL)). Inhibition of Iso126 using ASO126 (100 nM) showed statistically significant up-regulation of reporters containing Iso126 target sites (ordinary, one-way ANOVA with correction for multiple comparisons, compared to negative control (Neg CTL). Results are the mean and standard deviation of two-independent experiments, run in quadruplicate. (See FIG. 12).

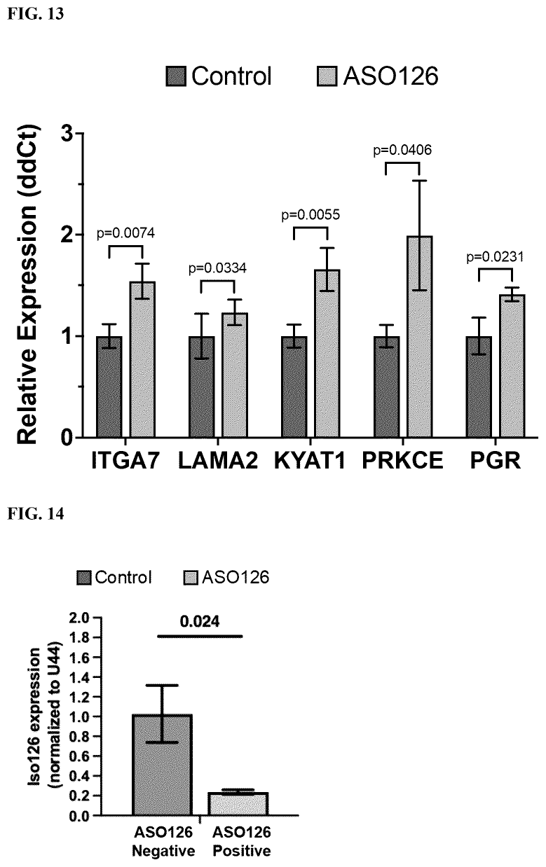

Human, primary cardiac fibroblasts were treated with 100 nM of ASO126 or a Control, non-targeting antisense oligo. Following a 48-hour incubation, RNA levels for ITGA7, LAMA2, KYAT1, PRKCE and PGR were analyzed by RT-qPCR. Relative mRNA expression in cells treated with ASO126 was calculated using the delta-delta Ct (ddCT) method; the global mean of HPRTI and GAPDH mRNAs was used as a normalizer. Results showed that all mRNAs were significantly up-regulated following ASO126 treatment thereby validating the endogenous regulation of Iso126 on these mRNAs. Statistical significance was determined using a one-way student's t-test with Welch's correction. (See FIG. 13).

Knockdown of Iso126 expression was confirmed using isoform-specific RT-qPCR. Results showed that Iso126 expression was significantly reduced 80% (p=0.024) in cells treated with ASO126 compared to a Control, non-targeting antisense oligo. Statistical significance was determined using a one-way student's t-test with Welch's correction. (See FIG. 14).

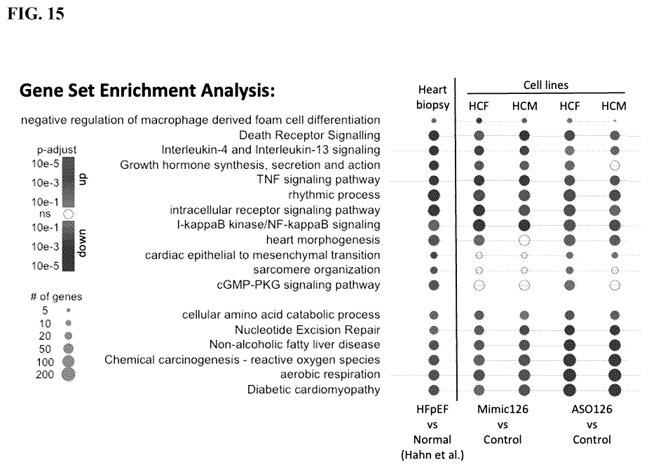

Human, primary cardiac fibroblasts (HCF) and cardiac myocytes (HCM) were treated with 100 nM of Mimic126 and ASO126 and a non-targeting Control. Following 48 h incubation, gene expression was measured by RNA sequencing and pathway enrichment analysis was calculated using GO biological process, Kegg pathway and Reactome pathway as Gene sets (R package: clusterProfiler). FIG. 15 summarizes the results for a subset of significantly (FDR <0.05) differentially expressed pathways relevant to heart failure and/or CMD. Increasing Iso126 expression (using Mimic compound) triggers pathway activation and repression similar to the disease, while Iso knockdown (with ASO126) triggers inverted pathway response compared to the disease. Hahn et al. (2021) heart biopsy expression data were used as HFpEF reference.

TABLE 1
Small RNA Features for Evaluating CFR

| SEQ | Feature | | | Small |
|---|---|---|---|---|
| ID NO | Set | Small RNA Sequence 5′ to 3′ | Mapped Name | RNA Class |
| 1 | 1 | GAAAAGCTGGGTTGAGGGGG | hsa-mir-320c | MIR |
| 2 | 1 | GAAACTCTGGTGGAGGTCCGTAGCGGTCCTGACGT | RNA28SN2 | R_RNA |
| 3 | 1 | GAATTGTGAGCGGATAA | LIFR-AS1 | LNC_RNA |
| 4 | 1 | GATAAGGGAAGTCGGCAAAATA | RNA28SN2 | R_RNA |
| 5 | 1 | GCATGAGTGGTTCAGTGGTAGAATTCTCGCC | tRNA-Gly-GCC-2-6 | T |
| 6 | 1 | GGCCAGTGGCGCAATGGA | tRNA-Arg-ACG-1-3 | T |
| 7 | 1 | GGCTGGGTCGGTCGGGCTGGGGCGCGAAGCGGGGCC | RNA45SN2 | R_RNA |
| 8 | 1 | GGTGATGTCGGCGATATAGG | piR-hsa-370812 | PI |
| 9 | 1 | TAACCCGTATCGTGAGCATCC | piR-hsa-6712275 | PI |
| 10 | 1 | TACAACCCCCCACTGCTAAATTTGACTGGCTTC | RNY4 | Y_RNA |
| 11 | 1 | TATCCCGCCGTTAGCCACCATC | piR-hsa-7139978 | PI |
| 12 | 1 | TCATGCAACTCGTAGGACAGGT | piR-hsa-2291645 | PI |
| 13 | 1 | TGGAAGACTAGTGATTCTGTTGTT | hsa-mir-7-5p | MIR |
| 14 | 1 | TGGTCCGAAGGTAGTGGGTTATCTCAAT | piR-hsa-1276990 | PI |
| 15 | 1 | TTGGCGGATGAGATAAGATTT | piR-hsa-2427414 | PI |
| 16 | 1 | TTTCCGTAGTGTAGTGGTTATCACGTTCGCCTG | piR-hsa-4382421 | PI |
| 17 | 1 | TTTCGCCACCTCTGACTTGGGC | piR-hsa-663873 | PI |
| 18 | 1 | ACTACGATACGGGAGGGCTTACC | LOC107986094 | LNC_RNA |
| 19 | 1 | AAGAACGTCGCGCACTGCTAG | piR-hsa-5362873 | PI |
| 20 | 1 | CATGCTCGTGAACTGGCGTATG | piR-hsa-5965833 | PI |
| 21 | 1 | CGTGTGGAATTCTCGGGTGCCAAA | LOC107985698 | LNC_RNA |
| 22 | 1 | TCCGGGTAGTCTCGAGAAACA | piR-hsa-4325054 | PI |
| 23 | 1 | TGAACACCCGGACCTGGCTAT | piR-hsa-7660744 | PI |
| 24 | 1 | AAACGACGAGCGTGGCACCACG | piR-hsa-3237688 | PI |
| 25 | 1 | CTTTCGCTTCGTTATCTACTC | piR-hsa-5246964 | PI |
| 26 | 1 | AAAGGAAGGAATCGAAC | piR-hsa-202978 | PI |
| 27 | 1 | AGCAGAGTGGCGCAGCGGAAGCGTGCTAGGC | tRNA-iMet-CAT-1- | T |
| | | | 2 | |
| 28 | 1 | AGCAGAGTTGCGCAGCGGAAGCGTGCTGGAC | tRNA-iMet-CAT-1- | T |
| | | | 2 | |
| 29 | 1 | AGGCTGGTCCGATGGTAGTGGGTTATTAGAAC | RNY4 | Y_RNA |
| 30 | 1 | ATAACCAGGTCCGGGTGTTCA | piR-hsa-6955843 | PI |
| 31 | 1 | AGCGTGGTGGTGCCGATGGT | piR-hsa-6722306 | PI |
| 32 | 1 | TGAACACCCGGACCCGGTTAT | piR-hsa-2986077 | PI |
| 33 | 1 | AACAGCAGTTGGACATG | piR-hsa-3679640 | PI |
| 34 | 1 | ATTGGTCGTGGCTGTAGTCCGTGCGAGAATACCA | piR-hsa-2359734 | PI |
| 35 | 1 | CATGGAACGGGTTGGCATGG | piR-hsa-1992427 | PI |
| 36 | 1 | CATTCGATGGTGTCTGGGATC | piR-hsa-2074585 | PI |
| 37 | 1 | CATTGGTGGTTCAGTAGTAGAATTCTCGCC | piR-hsa-7541972 | PI |
| 38 | 1 | CCCGAGACTCAGTGAAATTG | piR-hsa-1137951 | PI |
| 39 | 1 | AAGAAGGAAAGAAGAAGGGGAAGAAAAGAAGGGGA | piR-hsa-2350548 | PI |
| 40 | 1 | AAGACGGGAGGAAAGAAGGGAGAT | hsa-mir-483-5p | MIR |
| 41 | 1 | CGGCGGCGGCGGCGGTGGCGGCGGCGGCGGCGGCGGGAC | RNA28SN1 | R_RNA |
| 42 | 1 | GAGAACTGAATTCCATGGGTTT | hsa-mir-146a-5p | MIR |
| 43 | 2 | GAGGAGGAGGAGGAGGACGG | piR-hsa-6091880 | PI |
| 44 | 2 | GAGGTAGTAGGTTGCATAGT | hsa-let-7d-5p | MIR |
| 45 | 2 | GAGTAAGACGCTGCCGGAAT | piR-hsa-7050174 | PI |
| 46 | 2 | GATGGGGAAGATCGGGCTCGCCA | piR-hsa-8009464 | PI |
| 47 | 2 | GCATTGGTGGTTCAGTGGTAGAATTCTCGCCTG | tRNA-Gly-GCC-2-6 | T |
| 48 | 2 | GCATTTGTGGTTCAGTGGTAGAATTC | tRNA-Gly-GCC-2-6 | T |
| 49 | 2 | GGCTGGTCCGAAGGTAGTGAGTTATCTCA | RNY1 | Y_RNA |
| 50 | 2 | GTAAACATCCTTGACTGGAAGCTT | hsa-mir-30e-5p | MIR |
| 51 | 2 | TAGCTTATCAGACTGACGTTGAC | hsa-mir-21-5p | MIR |
| 52 | 2 | TAGCTTATCAGACTGATGTTGACT | hsa-mir-21-5p | MIR |
| 53 | 2 | TATTGCAGTTGTCCCGGCCTGT | hsa-mir-92a-3p | MIR |
| 54 | 2 | TGAGATGAAGCACTGTAGCAA | hsa-mir-143-3p | MIR |
| 55 | 2 | TGAGGGGCAGAGAGCGAGAT | hsa-mir-423-5p | MIR |
| 56 | 2 | TGAGGGGCAGAGAGCGAGGCT | hsa-mir-423-5p | MIR |
| 57 | 2 | TGAGGGGCAGAGAGCGAGACCTT | hsa-mir-423-5p | MIR |
| 58 | 2 | TGAGGGGCAGAGAGCGAGAAGA | hsa-mir-423-5p | MIR |
| 59 | 2 | TGAGGTAGTAGGTTGTGTGGTCT | hsa-let-7b-5p | MIR |
| 60 | 2 | TGGAGTGTGACAATGGTGA | hsa-mir-122-5p | MIR |
| 61 | 2 | TGTAAACATCCCCGACTGAAAG | hsa-mir-30d-5p | MIR |
| 62 | 2 | TGTAAATATCCCCGACTGGAAGC | hsa-mir-30a-5p | MIR |
| 63 | 2 | TGTAAACATCCTTGACTGGAAGT | hsa-mir-30e-5p | MIR |
| 64 | 2 | TTGGGAGCGGGGGGCGG | piR-hsa-342722 | PI |
| 65 | 2 | TTTAGTAATGGTAATGGTTCT | hsa-mir-451a | MIR |
| 66 | 2 | TTTCCGTAGTGTAGTGGTTATCACGTTCGC | tRNA-Val-CAC-1-6 | T |
| 67 | 2 | TTTGTTCGTTCGGCTCGCG | hsa-mir-375-3p | MIR |
| 68 | 2 | ACGGCGGCGGCGGCGGCGGCGGGGG | piR-hsa-2812342 | PI |
| 69 | 2 | AGAGGAGGAAGAGGAGGAGGA | piR-hsa-3851213 | PI |
| 70 | 2 | AGGCTGGTCCGATGGTAGTGGGTTATCAGAA | piR-hsa-159865 | PI |
| 71 | 2 | AGGGCGGCGGCGGCGGCGGCGGGGG | piR-hsa-2812342 | PI |
| 72 | 2 | AGGGCGGCGGCGGCGGCGGCGGGG | piR-hsa-2812342 | PI |
| 73 | 2 | ATTCCGGTTGTTCTTCTC | piR-hsa-8117738 | PI |
| 74 | 2 | ATGAGGTAGTAGGTTGTGTGG | hsa-let-7a-5p | MIR |
| 75 | 2 | AACATTCAACGCTGTCGGTGT | hsa-mir-181a-5p | MIR |
| 76 | 2 | CAACACCAGTCGATGGGCTGT | hsa-mir-21-3p | MIR |
| 77 | 2 | CAACGGAATCCCAAAAGC | hsa-mir-191-5p | MIR |
| 78 | 2 | AACCGGGCGGAAACACCAAA | tRNA-Val-CAC-1-5 | T |
| 79 | 2 | CATTCTTGCGACCCGGGT | tRNA-Gly-CCC-2-2 | T |
| 80 | 2 | CCCCTGTGGTCTAGTGGTTAGGATTCGGCGCTC | tRNA-Glu-CTC-1-6 | T |
| 81 | 2 | AAAAGCTGGGTTGAGAGGGA | hsa-mir-320c | MIR |
| 82 | 2 | CTGGTCCGAAGGTAGTGAGTTATCTCAA | RNY1 | Y_RNA |
| 83 | 2 | AAAAGCTGGGCTGAGAGGGCG | hsa-mir-4429 | MIR |
| 84 | 2 | AAAAGCTGGGTTGAGAGGGA | hsa-mir-320c | MIR |
| 85 | 3 | AAAAGCTGGGTTGAGAGGGCGAG | hsa-mir-320c | MIR |
| 86 | 3 | AAGCTGCCAGTTGAAGAACTGC | hsa-mir-22-3p | MIR |
| 87 | 3 | AGCTACATTGTCTGCTGGGTTT | hsa-mir-221-3p | MIR |
| 88 | 3 | AGCTCGGCCTGAGGCCCCTCAGT | hsa-mir-423-3p | MIR |
| 89 | 3 | AGGCTGGTCCGATGGTAGTGGGTTATCAGAACT | piR-hsa-85601 | PI |
| 90 | 3 | AGGGCGGCGGCGGCGGCGGCGGGGG | piR-hsa-2812342 | PI |
| 91 | 3 | ATATTGCACTTGTCCCGGCCTGT | hsa-mir-92b-3p | MIR |
| 92 | 3 | ATCCTGTACTGAGCTGCCCCGA | hsa-mir-486-5p | MIR |
| 93 | 3 | ATCCTGTACTGAGCTGCCCCGAG | hsa-mir-486-5p | MIR |
| 94 | 3 | ATTGCACTTGTCCCGGCCTG | hsa-mir-92a-3p | MIR |
| 95 | 3 | ATTGCACTTGTCCCGGCCTGT | hsa-mir-92a-3p | MIR |
| 96 | 3 | ATTGCACTTGCCCCGGCCTGT | hsa-mir-92a-3p | MIR |
| 97 | 3 | CATTGCACTTGTCCCGGCCTG | hsa-mir-92a-3p | MIR |
| 98 | 3 | CATTGCACTTGTCTCGGTCTGG | hsa-mir-25-3p | MIR |
| 99 | 3 | CATTGGTGGTTCAGTGGTAGAATTCTCGCC | tRNA-Gly-GCC-2-6 | T |
| 100 | 3 | CCCCCCGCTGCTAAATTTGACTGGCTT | piR-hsa-7922425 | PI |
| 101 | 3 | CCCTGTACTGAGCTGCCCCGAGA | hsa-mir-486-5p | MIR |
| 102 | 3 | CCTGTACTGAGCTGCCCCGAGA | hsa-mir-486-5p | MIR |
| 103 | 3 | CCTGTACTGAGCTGCCCCGAG | hsa-mir-486-5p | MIR |
| 104 | 3 | CGAGGAGGAAGAGGAGGACG | ANO8 | EXON |
| 105 | 3 | CGAGGTAGTAGGTTGTATAG | piR-hsa-1071760 | PI |
| 106 | 3 | CTATACGACCTGCTGCCTTT | hsa-let-7d-3p | MIR |
| 107 | 3 | CTGTACTGAGCTGCCCCGAG | hsa-mir-486-5p | MIR |
| 108 | 3 | GAATTGGTGGTTCAGTGGTAGAATTCTCGC | tRNA-Gly-GCC-2-6 | T |
| 109 | 3 | GCATCGGTGGTTCAGTGGTAGAATTCTCGCCT | tRNA-Gly-CCC-3-1 | T |
| 110 | 3 | GCATTAGTGGTTCAGTGGTAGAATTCTCGCCT | tRNA-Gly-CCC-1-2 | T |
| 111 | 3 | GCATTGATGGTTCAGTGGTAGAATTCTCGCC | tRNA-Gly-GCC-2-6 | T |
| 112 | 3 | GCATTGGTAGTTCAGTGGTAGAATTCTCGCC | tRNA-Gly-CCC-1-2 | T |
| 113 | 3 | GCATTGGTGGTTCAGTGGTAGAATTCTCGCCCG | tRNA-Gly-GCC-2-6 | T |
| 114 | 3 | GCATTGGTGGTTCAGTGGTAGAATTCTCGTC | tRNA-Gly-GCC-2-6 | T |
| 115 | 3 | GCATTGGTGGTTCAGTGGTAGAATTTTCGCCT | tRNA-Gly-CCC-1-2 | T |
| 116 | 3 | GCATTGGTGGTTCAGTGGTAGGATTCTCGCC | tRNA-Gly-GCC-2-6 | T |
| 117 | 3 | GCATTGGTGGTTCAGTGGTAGAGTTCTCGCC | tRNA-Gly-GCC-2-6 | T |
| 118 | 3 | GCATTGGTGGTTCGGTGGTAGAATTCTCGCC | tRNA-Gly-GCC-2-6 | T |
| 119 | 3 | GCGCCGCTGGTGTAGTGGCATCATGCAAGATT | tRNA-Gly-CCC-2-2 | T |
| 120 | 3 | GCGTTGGTGGTTCAGTGGTAGAATTCTCGCC | tRNA-Gly-GCC-2-6 | T |
| 121 | 3 | GGCTGGCCCGATGGTAGTGGGTTATCAGAACTTA | piR-hsa-162163 | PI |
| 122 | 3 | GGCTGGTCCGAAGGTAGTGAGTTACCTCAA | piR-hsa-499053 | PI |
| 123 | 3 | GGCTGGTCCGAAGGCAGTGAGTTATCTCAA | piR-hsa-1880526 | PI |
| 124 | 3 | GGCTGGTCCGATAGTAGTGGGTTATCAGAACTT | piR-hsa-2501884 | PI |
| 125 | 3 | GGCTGGTCCGATGGTAGTGGGTCATCAGAA | piR-hsa-149378 | PI |
| 126 | 3 | GGCTGGTCCGATGGTAGCGGGTTATCAGAA | piR-hsa-162163 | PI |
| 127 | 3 | GGCTGGTCCGATGGTAGTGGGTTGTCAGAACTT | piR-hsa-336206 | PI |
| 128 | 3 | GGCTGGTCCGATGGTAGTAGGTTATCAGAAC | piR-hsa-136616 | PI |
| 129 | 3 | GGGGAATTGCGAGCGGATAAC | piR-hsa-7820652 | PI |
| 130 | 3 | GGGGAATTGTGAGCGGATAAT | piR-hsa-1144682 | PI |
| 131 | 3 | GGGGAATTGTGAGCGGACAAC | piR-hsa-6632066 | PI |
| 132 | 3 | TAAGCGGCGGGGTCGGAACAGG | SLFN12L | EXON |
| 133 | 3 | TAAGGGGCAGAGAGCGAGACT | hsa-mir-423-5p | MIR |
| 134 | 3 | TAATGCACTTGTCCCGGCCTGT | hsa-mir-92a-3p | MIR |
| 135 | 3 | TAGCAGCACGTAAATATCGGCG | hsa-mir-16-5p | MIR |
| 136 | 3 | TAGCTTATCAGACTGACGTTGA | hsa-mir-21-5p | MIR |
| 137 | 3 | TAGCTTATCAGACTGATGTTAA | hsa-mir-21-5p | MIR |
| 138 | 3 | TAGCTTATCAGACTGATGTT | hsa-mir-21-5p | MIR |
| 139 | 3 | TATTGCACTTGTCACGGCCTGT | hsa-mir-92a-3p | MIR |
| 140 | 3 | TATTGCACTTGCCCCGGCCTGT | hsa-mir-92a-3p | MIR |
| 141 | 3 | TATTGCACTTGTCCCGGTCTG | hsa-mir-92a-3p | MIR |
| 142 | 3 | TATTGCACTTGTCCCAGCCTGT | hsa-mir-92a-3p | MIR |
| 143 | 3 | TATTGCACTCGTCCCGGCCTGT | hsa-mir-92b-3p | MIR |
| 144 | 3 | TATTGCACTTGTCCCGGCCAGT | hsa-mir-92a-3p | MIR |
| 145 | 3 | TATTGCACTTGTCCCGGCCCG | hsa-mir-92a-3p | MIR |
| 146 | 3 | TATTGCACTTGTCCCGGCCTG | hsa-mir-92a-3p | MIR |
| 147 | 3 | TATTGCGCTTGTCCCGGCCTG | hsa-mir-92a-3p | MIR |
| 148 | 3 | TATTGCACTTGTCCCTGCCTGT | hsa-mir-92a-3p | MIR |
| 149 | 3 | TATTGCACTTATCCCGGCCTGT | hsa-mir-92a-3p | MIR |
| 150 | 3 | TATTGCACTTGTCCAGGCCTGT | hsa-mir-92a-3p | MIR |
| 151 | 3 | TATTGCTCTTGTCCCGGCCTGT | hsa-mir-92a-3p | MIR |
| 152 | 3 | TATTGCACTTGTCCCGGCCTGTG | hsa-mir-92a-3p | MIR |
| 153 | 3 | TCCCGTACTGAGCTGCCCCGAGA | hsa-mir-486-5p | MIR |
| 154 | 3 | TCCCGTACTGAGCTGCCCCGAG | hsa-mir-486-5p | MIR |
| 155 | 3 | TCCCGTACTGAGCTGCCCCG | hsa-mir-486-5p | MIR |
| 156 | 3 | TCCCTGGTGGTCTAATGGTTAGGATTCGGCGC | piR-hsa-1907567 | PI |
| 157 | 3 | TCCCTGGTGGTCTAGTAGTTAGGATTCGGCGC | piR-hsa-1907567 | PI |
| 158 | 3 | TCCCTGGTGATCTAGTGGTTAGGATTCGGCGCT | tRNA-Glu-TTC-14-1 | T |
| 159 | 3 | TCCCTGGTGGTCTAGTGGTCAGGATTCGGCGCTCT | tRNA-Glu-TTC-14-1 | T |
| 160 | 3 | TCCTGCACTGAGCTGCCCCGAGA | hsa-mir-486-5p | MIR |
| 161 | 3 | TCCTGTACTAAGCTGCCCCGAGA | hsa-mir-486-5p | MIR |
| 162 | 3 | TCCTGTACCGAGCTGCCCCGAGT | hsa-mir-486-5p | MIR |
| 163 | 3 | TCCTGTACTGAGCTGCCCCAAG | hsa-mir-486-5p | MIR |
| 164 | 3 | TCCTGTACTGAGCTGCCCCGGGA | hsa-mir-486-5p | MIR |
| 165 | 3 | TCCTGTACTGAGCTGCCCCGTG | hsa-mir-486-5p | MIR |
| 166 | 3 | TCCTGTACCGAGCTGCCCCGAGA | hsa-mir-486-5p | MIR |
| 167 | 3 | TCCTGTACTGAGCTGCCACGA | hsa-mir-486-5p | MIR |
| 168 | 3 | TCCTGTACCGAGCTGCCCCGAG | hsa-mir-486-5p | MIR |
| 169 | 3 | TCCTGTACTGAGCTGCCCCGT | hsa-mir-486-5p | MIR |
| 170 | 3 | TCCTGTACTGAGCTGCCCCAAGA | hsa-mir-486-5p | MIR |
| 171 | 3 | TCCTGTACTGAGTTGCCCCGAG | hsa-mir-486-5p | MIR |
| 172 | 3 | TCCTGTACTGGGCTGCCCCGAGT | hsa-mir-486-5p | MIR |
| 173 | 3 | TCCTGTACTGGGCTGCCCCGAG | hsa-mir-486-5p | MIR |
| 174 | 3 | TCCTGTACTGAGCTGCCCGAG | hsa-mir-486-5p | MIR |
| 175 | 3 | TGAGAACTGAATTCCATGGGT | hsa-mir-146a-5p | MIR |
| 176 | 3 | TGAGGGGCAGAGAGCGAGACC | hsa-mir-423-5p | MIR |
| 177 | 3 | TGAGGGACAGAGAGCGAGACT | piR-hsa-1749141 | PI |
| 178 | 3 | TGAGGGGCAGAGAGCGGGACTTTT | hsa-mir-423-5p | MIR |
| 179 | 3 | TGAGGGGCAGAGGGCGAGACTTT | hsa-mir-423-5p | MIR |
| 180 | 3 | TGAGGTAGTAGGTCGTGTGGTTT | hsa-let-7c-5p | MIR |
| 181 | 3 | TGAGGTAGTAGGTTGTGTGGTCT | hsa-let-7b-5p | MIR |
| 182 | 3 | TGAGGTAGTAGGTCGTGTGG | hsa-let-7c-5p | MIR |
| 183 | 3 | TGAGGTAGTAGGTTGTATAG | hsa-let-7a-5p | MIR |
| 184 | 3 | TGAGGTAGTAGTTTGCGCTGT | hsa-let-7g-5p | MIR |
| 185 | 3 | TGAGGTAGTAGGTCGTATAG | hsa-let-7c-5p | MIR |
| 186 | 3 | TGAGGTAGTAGGTCGTATAGT | hsa-let-7c-5p | MIR |
| 187 | 3 | TGAGGTAGTAGGTTGTGTG | hsa-let-7b-5p | MIR |
| 188 | 3 | TGAGGTAGTAGGTTATGTGGTT | ARHGAP26 | PROTEIN_ |
| | | | | CODING_RNA |
| 189 | 3 | TGAGGTAGTGGATTGTATAGTT | hsa-mir-6130 | MIR |
| 190 | 3 | TGAGGTAGTAGTTTGCACAGTT | hsa-let-7g-5p | MIR |
| 191 | 3 | TGAGGTAGTAGGTTGTGTGG | hsa-let-7b-5p | MIR |
| 192 | 3 | TGGAGTGTGACAATGGTGTCTG | hsa-mir-122-5p | MIR |
| 193 | 3 | TGGGTTTACGTTGGGAGAACT | hsa-mir-629-5p | MIR |
| 194 | 3 | TGTAAACATCCTTGACTGGAAG | hsa-mir-30e-5p | MIR |
| 195 | 3 | TGTAAACATCCCCGACTGGAAA | hsa-mir-30d-5p | MIR |
| 196 | 3 | TGTAAACATCCTACACTCTCA | hsa-mir-30c-5p | MIR |
| 197 | 3 | TGTAGACATCCTTGACTGGAAGCT | hsa-mir-30e-5p | MIR |
| 198 | 3 | TGTTGCACTTGTCCCGGCCTGT | hsa-mir-92a-3p | MIR |
| 199 | 3 | TTCTGTACTGAGCTGCCCCGAGA | hsa-mir-486-5p | MIR |
| 200 | 3 | TTGCACTTGTCCCGGCCTGT | hsa-mir-92a-3p | MIR |
| 201 | 3 | TTGGGAGCGGGGGGCGG | piR-hsa-342722 | PI |
| 202 | 3 | ATTCCGGTTGTTCTTCTC | piR-hsa-8117738 | PI |
| 203 | 3 | TTGTAGCACTGGGGCCAGATGG | piR-hsa-2103082 | PI |
| 204 | 3 | AGATGAGAGGACGGGACGCC | RSRC1 | PROTEIN_ |
| | | | | CODING_RNA |
| 205 | 3 | TATGGAAGTGCCGCCGCTGAAA | piR-hsa-7278832 | PI |
| 206 | 3 | TATTAAATCAATCTCGGCTGGC | piR-hsa-5905601 | PI |

| SEQ | | | |
|---|---|---|---|
| ID NO | Population Frequency | Spearman rho Correlation | P-value |
| 1 | 16% | −0.379 | 0.043 |
| 2 | 6% | −0.373 | 0.261 |
| 3 | 3% | −0.900 | 0.083 |
| 4 | 8% | −0.393 | 0.165 |
| 5 | 26% | 0.348 | 0.016 |
| 6 | 8% | 0.257 | 0.374 |
| 7 | 7% | −0.503 | 0.079 |
| 8 | 67% | 0.125 | 0.172 |
| 9 | 15% | 0.146 | 0.469 |
| 10 | 9% | 0.325 | 0.219 |
| 11 | 7% | 0.301 | 0.342 |
| 12 | 6% | −0.261 | 0.466 |
| 13 | 3% | 0.657 | 0.175 |
| 14 | 6% | −0.212 | 0.560 |
| 15 | 3% | −0.543 | 0.297 |
| 16 | 6% | −0.492 | 0.148 |
| 17 | 13% | 0.317 | 0.140 |
| 18 | 35% | 0.100 | 0.434 |
| 19 | 23% | −0.359 | 0.019 |
| 20 | 18% | 0.156 | 0.387 |
| 21 | 7% | 0.210 | 0.514 |
| 22 | 17% | 0.130 | 0.487 |
| 23 | 8% | 0.236 | 0.397 |
| 24 | 22% | 0.381 | 0.017 |
| 25 | 4% | −0.714 | 0.088 |
| 26 | 13% | 0.226 | 0.289 |
| 27 | 26% | 0.170 | 0.253 |
| 28 | 13% | −0.324 | 0.132 |
| 29 | 13% | 0.056 | 0.796 |
| 30 | 4% | 0.313 | 0.450 |
| 31 | 36% | 0.156 | 0.219 |
| 32 | 5% | −0.483 | 0.194 |
| 33 | 22% | 0.202 | 0.217 |
| 34 | 27% | 0.179 | 0.225 |
| 35 | 36% | −0.217 | 0.085 |
| 36 | 3% | 0.943 | 0.017 |
| 37 | 12% | 0.300 | 0.175 |
| 38 | 3% | 0.700 | 0.233 |
| 39 | 11% | 0.029 | 0.906 |
| 40 | 12% | −0.290 | 0.203 |
| 41 | 8% | −0.290 | 0.314 |
| 42 | 34% | 0.020 | 0.878 |
| 43 | 45% | 0.262 | 0.018 |
| 44 | 34% | −0.258 | 0.045 |
| 45 | 36% | −0.268 | 0.032 |
| 46 | 40% | 0.324 | 0.006 |
| 47 | 96% | −0.155 | 0.043 |
| 48 | 37% | 0.365 | 0.002 |
| 49 | 66% | 0.337 | 0.000 |
| 50 | 33% | −0.106 | 0.424 |
| 51 | 60% | −0.124 | 0.201 |
| 52 | 64% | −0.148 | 0.114 |
| 53 | 36% | 0.245 | 0.051 |
| 54 | 40% | −0.205 | 0.085 |
| 55 | 55% | −0.231 | 0.021 |
| 56 | 93% | 0.052 | 0.503 |
| 57 | 92% | 0.114 | 0.144 |
| 58 | 36% | −0.058 | 0.649 |
| 59 | 78% | 0.230 | 0.006 |
| 60 | 49% | −0.161 | 0.133 |
| 61 | 53% | 0.023 | 0.824 |
| 62 | 53% | 0.091 | 0.381 |
| 63 | 41% | −0.373 | 0.001 |
| 64 | 77% | 0.259 | 0.002 |
| 65 | 37% | 0.194 | 0.115 |
| 66 | 39% | 0.145 | 0.229 |
| 67 | 47% | 0.094 | 0.390 |
| 68 | 35% | 0.289 | 0.021 |
| 69 | 37% | 0.323 | 0.008 |
| 70 | 37% | 0.149 | 0.229 |
| 71 | 73% | 0.240 | 0.006 |
| 72 | 52% | 0.273 | 0.008 |
| 73 | 79% | 0.233 | 0.005 |
| 74 | 44% | 0.403 | 0.000 |
| 75 | 37% | −0.291 | 0.017 |
| 76 | 44% | −0.308 | 0.006 |
| 77 | 47% | −0.304 | 0.005 |
| 78 | 41% | −0.324 | 0.005 |
| 79 | 42% | −0.274 | 0.017 |
| 80 | 42% | 0.284 | 0.013 |
| 81 | 70% | −0.214 | 0.016 |
| 82 | 47% | 0.224 | 0.040 |
| 83 | 76% | 0.210 | 0.014 |
| 84 | 70% | −0.214 | 0.016 |
| 85 | 78% | 0.134 | 0.113 |
| 86 | 79% | −0.184 | 0.027 |
| 87 | 76% | −0.202 | 0.018 |
| 88 | 78% | −0.138 | 0.103 |
| 89 | 72% | 0.146 | 0.098 |
| 90 | 73% | 0.240 | 0.006 |
| 91 | 71% | 0.250 | 0.005 |
| 92 | 82% | 0.141 | 0.087 |
| 93 | 80% | 0.161 | 0.054 |
| 94 | 77% | 0.107 | 0.212 |
| 95 | 90% | 0.098 | 0.215 |
| 96 | 77% | 0.159 | 0.063 |
| 97 | 75% | 0.128 | 0.139 |
| 98 | 79% | 0.184 | 0.028 |
| 99 | 73% | 0.135 | 0.123 |
| 100 | 80% | 0.158 | 0.059 |
| 101 | 91% | 0.156 | 0.046 |
| 102 | 82% | 0.220 | 0.007 |
| 103 | 90% | 0.265 | 0.001 |
| 104 | 70% | 0.170 | 0.057 |
| 105 | 76% | 0.326 | 0.000 |
| 106 | 80% | 0.150 | 0.073 |
| 107 | 80% | 0.162 | 0.052 |
| 108 | 71% | 0.167 | 0.061 |
| 109 | 97% | 0.139 | 0.068 |
| 110 | 93% | 0.177 | 0.022 |
| 111 | 71% | 0.171 | 0.055 |
| 112 | 75% | 0.138 | 0.110 |
| 113 | 81% | 0.183 | 0.027 |
| 114 | 74% | 0.160 | 0.064 |
| 115 | 89% | 0.126 | 0.112 |
| 116 | 77% | 0.133 | 0.120 |
| 117 | 77% | 0.123 | 0.150 |
| 118 | 76% | 0.309 | 0.000 |
| 119 | 83% | −0.190 | 0.020 |
| 120 | 71% | 0.157 | 0.077 |
| 121 | 70% | 0.176 | 0.049 |
| 122 | 84% | 0.200 | 0.014 |
| 123 | 79% | 0.209 | 0.012 |
| 124 | 72% | 0.136 | 0.124 |
| 125 | 79% | 0.161 | 0.056 |
| 126 | 81% | 0.170 | 0.041 |
| 127 | 71% | 0.173 | 0.052 |
| 128 | 84% | 0.127 | 0.121 |
| 129 | 86% | −0.103 | 0.204 |
| 130 | 78% | −0.136 | 0.109 |
| 131 | 85% | −0.213 | 0.008 |
| 132 | 70% | −0.154 | 0.085 |
| 133 | 76% | 0.239 | 0.005 |
| 134 | 72% | 0.289 | 0.001 |
| 135 | 76% | 0.232 | 0.006 |
| 136 | 78% | −0.272 | 0.001 |
| 137 | 71% | −0.248 | 0.005 |
| 138 | 72% | −0.205 | 0.019 |
| 139 | 78% | 0.229 | 0.006 |
| 140 | 98% | 0.250 | 0.001 |
| 141 | 73% | 0.243 | 0.005 |
| 142 | 93% | 0.204 | 0.008 |
| 143 | 92% | 0.137 | 0.079 |
| 144 | 70% | 0.203 | 0.022 |
| 145 | 86% | 0.256 | 0.001 |
| 146 | 97% | 0.144 | 0.057 |
| 147 | 71% | 0.160 | 0.071 |
| 148 | 74% | 0.141 | 0.105 |
| 149 | 88% | 0.244 | 0.002 |
| 150 | 73% | 0.191 | 0.029 |
| 151 | 72% | 0.191 | 0.029 |
| 152 | 70% | 0.158 | 0.077 |
| 153 | 96% | 0.147 | 0.054 |
| 154 | 99% | 0.158 | 0.035 |
| 155 | 90% | 0.184 | 0.019 |
| 156 | 87% | −0.255 | 0.001 |
| 157 | 87% | −0.226 | 0.004 |
| 158 | 83% | 0.143 | 0.081 |
| 159 | 79% | 0.259 | 0.002 |
| 160 | 92% | 0.193 | 0.013 |
| 161 | 78% | 0.136 | 0.109 |
| 162 | 72% | 0.235 | 0.007 |
| 163 | 93% | 0.129 | 0.095 |
| 164 | 87% | 0.138 | 0.084 |
| 165 | 70% | 0.174 | 0.052 |
| 166 | 91% | 0.140 | 0.074 |
| 167 | 73% | 0.219 | 0.012 |
| 168 | 95% | 0.120 | 0.117 |
| 169 | 79% | 0.142 | 0.091 |
| 170 | 77% | 0.168 | 0.048 |
| 171 | 94% | 0.151 | 0.049 |
| 172 | 88% | 0.178 | 0.025 |
| 173 | 98% | 0.183 | 0.015 |
| 174 | 72% | 0.208 | 0.017 |
| 175 | 77% | −0.180 | 0.035 |
| 176 | 89% | 0.239 | 0.002 |
| 177 | 88% | 0.160 | 0.044 |
| 178 | 83% | −0.159 | 0.052 |
| 179 | 94% | 0.146 | 0.058 |
| 180 | 74% | 0.202 | 0.019 |
| 181 | 78% | 0.230 | 0.006 |
| 182 | 74% | 0.336 | 0.000 |
| 183 | 95% | 0.156 | 0.041 |
| 184 | 83% | 0.211 | 0.010 |
| 185 | 75% | 0.173 | 0.045 |
| 186 | 78% | 0.201 | 0.017 |
| 187 | 80% | 0.191 | 0.022 |
| 188 | 80% | 0.187 | 0.025 |
| 189 | 83% | 0.152 | 0.065 |
| 190 | 83% | 0.141 | 0.086 |
| 191 | 97% | 0.156 | 0.039 |
| 192 | 80% | −0.158 | 0.058 |
| 193 | 74% | −0.153 | 0.079 |
| 194 | 74% | −0.179 | 0.039 |
| 195 | 71% | −0.211 | 0.017 |
| 196 | 74% | −0.211 | 0.014 |
| 197 | 73% | −0.265 | 0.002 |
| 198 | 89% | 0.196 | 0.013 |
| 199 | 78% | 0.114 | 0.180 |
| 200 | 75% | 0.188 | 0.029 |
| 201 | 77% | 0.259 | 0.002 |
| 202 | 79% | 0.233 | 0.005 |
| 203 | 73% | −0.139 | 0.114 |
| 204 | 79% | −0.247 | 0.003 |
| 205 | 78% | −0.189 | 0.024 |
| 206 | 81% | −0.159 | 0.055 |

TABLE 2
Primers and Probes For RT-qPCR Analysis of Small RNA Features Correlated to CFR

| SEQ | | Gene Specific Primer |
|---|---|---|
| ID NO. | RT-primer 5′ to 3′ | 5′ to 3′ |
| 1 | GTCGTATCCAGTGCAGGGTCCGAGGTATTCGCACTGGATACGACCCCCCT | GAAAAGCTGGGTTGAGGGGG |
| 2 | GTCGTATCCAGTGCAGGGTCCGAGGTATTCGCACTGGATACGACACGTCA | GAAACTCTGGTGGAGGTCCG |
| 3 | GTCGTATCCAGTGCAGGGTCCGAGGTATTCGCACTGGATACGACTTATCC | GAATTGTGAGCGGATAA |
| 4 | GTCGTATCCAGTGCAGGGTCCGAGGTATTCGCACTGGATACGACTATTTT | GATAAGGGAAGTCGGCAAAA |
| 5 | GTCGTATCCAGTGCAGGGTCCGAGGTATTCGCACTGGATACGACGGCGAG | GCATGAGTGGTTCAGTGGTA |
| 6 | GTCGTATCCAGTGCAGGGTCCGAGGTATTCGCACTGGATACGACTCCATT | GGCCAGTGGCGCAATGGA |
| 7 | GTCGTATCCAGTGCAGGGTCCGAGGTATTCGCACTGGATACGACGGCCCC | GGCTGGGTCGGTCGGGCTGG |
| 8 | GTCGTATCCAGTGCAGGGTCCGAGGTATTCGCACTGGATACGACCCTATA | GGTGATGTCGGCGATATAGG |
| 9 | GTCGTATCCAGTGCAGGGTCCGAGGTATTCGCACTGGATACGACGGATGC | TAACCCGTATCGTGAGCATC |
| 10 | GTCGTATCCAGTGCAGGGTCCGAGGTATTCGCACTGGATACGACGAAGCC | TACAACCCCCCACTGCTAAA |
| 11 | GTCGTATCCAGTGCAGGGTCCGAGGTATTCGCACTGGATACGACGATGGT | TATCCCGCCGTTAGCCACCA |
| 12 | GTCGTATCCAGTGCAGGGTCCGAGGTATTCGCACTGGATACGACACCTGT | TCATGCAACTCGTAGGACAG |
| 13 | GTCGTATCCAGTGCAGGGTCCGAGGTATTCGCACTGGATACGACAACAAC | TGGAAGACTAGTGATTCTGT |
| 14 | GTCGTATCCAGTGCAGGGTCCGAGGTATTCGCACTGGATACGACATTGAG | TGGTCCGAAGGTAGTGGGTT |
| 15 | GTCGTATCCAGTGCAGGGTCCGAGGTATTCGCACTGGATACGACAAATCT | TTGGCGGATGAGATAAGATT |
| 16 | GTCGTATCCAGTGCAGGGTCCGAGGTATTCGCACTGGATACGACCAGGCG | TTTCCGTAGTGTAGTGGTTA |
| 17 | GTCGTATCCAGTGCAGGGTCCGAGGTATTCGCACTGGATACGACGCCCAA | TTTCGCCACCTCTGACTTGG |
| 18 | GTCGTATCCAGTGCAGGGTCCGAGGTATTCGCACTGGATACGACGGTAAG | ACTACGATACGGGAGGGCTT |
| 19 | GTCGTATCCAGTGCAGGGTCCGAGGTATTCGCACTGGATACGACCTAGCA | AAGAACGTCGCGCACTGCTA |
| 20 | GTCGTATCCAGTGCAGGGTCCGAGGTATTCGCACTGGATACGACCATACG | CATGCTCGTGAACTGGCGTA |
| 21 | GTCGTATCCAGTGCAGGGTCCGAGGTATTCGCACTGGATACGACTTTGGC | CGTGTGGAATTCTCGGGTGC |
| 22 | GTCGTATCCAGTGCAGGGTCCGAGGTATTCGCACTGGATACGACTGTTTC | TCCGGGTAGTCTCGAGAAAC |
| 23 | GTCGTATCCAGTGCAGGGTCCGAGGTATTCGCACTGGATACGACATAGCC | TGAACACCCGGACCTGGCTA |
| 24 | GTCGTATCCAGTGCAGGGTCCGAGGTATTCGCACTGGATACGACCGTGGT | AAACGACGAGCGTGGCACCA |
| 25 | GTCGTATCCAGTGCAGGGTCCGAGGTATTCGCACTGGATACGACGAGTAG | CTTTCGCTTCGTTATCTACT |
| 26 | GTCGTATCCAGTGCAGGGTCCGAGGTATTCGCACTGGATACGACGTTCGA | AAAGGAAGGAATCGAAC |
| 27 | GTCGTATCCAGTGCAGGGTCCGAGGTATTCGCACTGGATACGACGCCTAG | AGCAGAGTGGCGCAGCGGAA |
| 28 | GTCGTATCCAGTGCAGGGTCCGAGGTATTCGCACTGGATACGACGTCCAG | AGCAGAGTTGCGCAGCGGAA |
| 29 | GTCGTATCCAGTGCAGGGTCCGAGGTATTCGCACTGGATACGACGTTCTA | AGGCTGGTCCGATGGTAGTG |
| 30 | GTCGTATCCAGTGCAGGGTCCGAGGTATTCGCACTGGATACGACTGAACA | ATAACCAGGTCCGGGTGTTC |
| 31 | GTCGTATCCAGTGCAGGGTCCGAGGTATTCGCACTGGATACGACACCATC | AGCGTGGTGGTGCCGATGGT |
| 32 | GTCGTATCCAGTGCAGGGTCCGAGGTATTCGCACTGGATACGACATAACC | TGAACACCCGGACCCGGTTA |
| 33 | GTCGTATCCAGTGCAGGGTCCGAGGTATTCGCACTGGATACGACCATGTC | AACAGCAGTTGGACATG |
| 34 | GTCGTATCCAGTGCAGGGTCCGAGGTATTCGCACTGGATACGACTGGTAT | ATTGGTCGTGGCTGTAGTCC |
| 35 | GTCGTATCCAGTGCAGGGTCCGAGGTATTCGCACTGGATACGACCCATGC | CATGGAACGGGTTGGCATGG |
| 36 | GTCGTATCCAGTGCAGGGTCCGAGGTATTCGCACTGGATACGACGATCCC | CATTCGATGGTGTCTGGGAT |
| 37 | GTCGTATCCAGTGCAGGGTCCGAGGTATTCGCACTGGATACGACGGCGAG | CATTGGTGGTTCAGTAGTAG |
| 38 | GTCGTATCCAGTGCAGGGTCCGAGGTATTCGCACTGGATACGACCAATTT | CCCGAGACTCAGTGAAATTG |
| 39 | GTCGTATCCAGTGCAGGGTCCGAGGTATTCGCACTGGATACGACTCCCCT | AAGAAGGAAAGAAGAAGGGG |
| 40 | GTCGTATCCAGTGCAGGGTCCGAGGTATTCGCACTGGATACGACATCTCC | AAGACGGGAGGAAAGAAGGG |
| 41 | GTCGTATCCAGTGCAGGGTCCGAGGTATTCGCACTGGATACGACGTCCCG | CGGCGGCGGCGGCGGTGGCG |
| 42 | GTCGTATCCAGTGCAGGGTCCGAGGTATTCGCACTGGATACGACAAACCC | GAGAACTGAATTCCATGGGT |
| 43 | GTCGTATCCAGTGCAGGGTCCGAGGTATTCGCACTGGATACGACCCGTCC | GAGGAGGAGGAGGAGGACGG |
| 44 | GTCGTATCCAGTGCAGGGTCCGAGGTATTCGCACTGGATACGACACTATG | GAGGTAGTAGGTTGCATAGT |
| 45 | GTCGTATCCAGTGCAGGGTCCGAGGTATTCGCACTGGATACGACATTCCG | GAGTAAGACGCTGCCGGAAT |
| 46 | GTCGTATCCAGTGCAGGGTCCGAGGTATTCGCACTGGATACGACTGGCGA | GATGGGGAAGATCGGGCTCG |
| 47 | GTCGTATCCAGTGCAGGGTCCGAGGTATTCGCACTGGATACGACCAGGCG | GCATTGGTGGTTCAGTGGTA |
| 48 | GTCGTATCCAGTGCAGGGTCCGAGGTATTCGCACTGGATACGACGAATTC | GCATTTGTGGTTCAGTGGTA |
| 49 | GTCGTATCCAGTGCAGGGTCCGAGGTATTCGCACTGGATACGACTGAGAT | GGCTGGTCCGAAGGTAGTGA |
| 50 | GTCGTATCCAGTGCAGGGTCCGAGGTATTCGCACTGGATACGACAAGCTT | GTAAACATCCTTGACTGGAA |
| 51 | GTCGTATCCAGTGCAGGGTCCGAGGTATTCGCACTGGATACGACGTCAAC | TAGCTTATCAGACTGACGTT |
| 52 | GTCGTATCCAGTGCAGGGTCCGAGGTATTCGCACTGGATACGACAGTCAA | TAGCTTATCAGACTGATGTT |
| 53 | GTCGTATCCAGTGCAGGGTCCGAGGTATTCGCACTGGATACGACACAGGC | TATTGCAGTTGTCCCGGCCT |
| 54 | GTCGTATCCAGTGCAGGGTCCGAGGTATTCGCACTGGATACGACTTGCTA | TGAGATGAAGCACTGTAGCA |
| 55 | GTCGTATCCAGTGCAGGGTCCGAGGTATTCGCACTGGATACGACATCTCG | TGAGGGGCAGAGAGCGAGAT |
| 56 | GTCGTATCCAGTGCAGGGTCCGAGGTATTCGCACTGGATACGACAGCCTC | TGAGGGGCAGAGAGCGAGGC |
| 57 | GTCGTATCCAGTGCAGGGTCCGAGGTATTCGCACTGGATACGACAAGGTC | TGAGGGGCAGAGAGCGAGAC |
| 58 | GTCGTATCCAGTGCAGGGTCCGAGGTATTCGCACTGGATACGACTCTTCT | TGAGGGGCAGAGAGCGAGAA |
| 59 | GTCGTATCCAGTGCAGGGTCCGAGGTATTCGCACTGGATACGACAGACCA | TGAGGTAGTAGGTTGTGTGG |
| 60 | GTCGTATCCAGTGCAGGGTCCGAGGTATTCGCACTGGATACGACTCACCA | TGGAGTGTGACAATGGTGA |
| 61 | GTCGTATCCAGTGCAGGGTCCGAGGTATTCGCACTGGATACGACCTTTCA | TGTAAACATCCCCGACTGAA |
| 62 | GTCGTATCCAGTGCAGGGTCCGAGGTATTCGCACTGGATACGACGCTTCC | TGTAAATATCCCCGACTGGA |
| 63 | GTCGTATCCAGTGCAGGGTCCGAGGTATTCGCACTGGATACGACACTTCC | TGTAAACATCCTTGACTGGA |
| 64 | GTCGTATCCAGTGCAGGGTCCGAGGTATTCGCACTGGATACGACCCGCCC | TTGGGAGCGGGCGGGCGG |
| 65 | GTCGTATCCAGTGCAGGGTCCGAGGTATTCGCACTGGATACGACAGAACC | TTTAGTAATGGTAATGGTTC |
| 66 | GTCGTATCCAGTGCAGGGTCCGAGGTATTCGCACTGGATACGACGCGAAC | TTTCCGTAGTGTAGTGGTTA |
| 67 | GTCGTATCCAGTGCAGGGTCCGAGGTATTCGCACTGGATACGACCGCGAG | TTTGTTCGTTCGGCTCGCG |
| 68 | GTCGTATCCAGTGCAGGGTCCGAGGTATTCGCACTGGATACGACCCCCCG | ACGGCGGCGGCGGCGGCGGC |
| 69 | GTCGTATCCAGTGCAGGGTCCGAGGTATTCGCACTGGATACGACTCCTCC | AGAGGAGGAAGAGGAGGAGG |
| 70 | GTCGTATCCAGTGCAGGGTCCGAGGTATTCGCACTGGATACGACTTCTGA | AGGCTGGTCCGATGGTAGTG |
| 71 | GTCGTATCCAGTGCAGGGTCCGAGGTATTCGCACTGGATACGACCCCCCG | AGGGCGGCGGCGGCGGCGGC |
| 72 | GTCGTATCCAGTGCAGGGTCCGAGGTATTCGCACTGGATACGACCCCCGC | AGGGCGGCGGCGGCGGCGGC |
| 73 | GTCGTATCCAGTGCAGGGTCCGAGGTATTCGCACTGGATACGACGAGAAG | ATTCCGGTTGTTCTTCTC |
| 74 | GTCGTATCCAGTGCAGGGTCCGAGGTATTCGCACTGGATACGACCCACAC | ATGAGGTAGTAGGTTGTGTG |
| 75 | GTCGTATCCAGTGCAGGGTCCGAGGTATTCGCACTGGATACGACACACCG | AACATTCAACGCTGTCGGTG |
| 76 | GTCGTATCCAGTGCAGGGTCCGAGGTATTCGCACTGGATACGACACAGCC | CAACACCAGTCGATGGGCTG |
| 77 | GTCGTATCCAGTGCAGGGTCCGAGGTATTCGCACTGGATACGACGCTTTT | CAACGGAATCCCAAAAGC |
| 78 | GTCGTATCCAGTGCAGGGTCCGAGGTATTCGCACTGGATACGACTTTGGT | AACCGGGCGGAAACACCAAA |
| 79 | GTCGTATCCAGTGCAGGGTCCGAGGTATTCGCACTGGATACGACACCCGG | CATTCTTGCGACCCGGGT |
| 80 | GTCGTATCCAGTGCAGGGTCCGAGGTATTCGCACTGGATACGACGAGCGC | CCCCTGTGGTCTAGTGGTTA |
| 81 | GTCGTATCCAGTGCAGGGTCCGAGGTATTCGCACTGGATACGACTCCCTC | AAAAGCTGGGTTGAGAGGGA |
| 82 | GTCGTATCCAGTGCAGGGTCCGAGGTATTCGCACTGGATACGACTTGAGA | CTGGTCCGAAGGTAGTGAGT |
| 83 | GTCGTATCCAGTGCAGGGTCCGAGGTATTCGCACTGGATACGACCGCCCT | AAAAGCTGGGCTGAGAGGGC |
| 84 | GTCGTATCCAGTGCAGGGTCCGAGGTATTCGCACTGGATACGACTCCCTC | AAAAGCTGGGTTGAGAGGGA |
| 85 | GTCGTATCCAGTGCAGGGTCCGAGGTATTCGCACTGGATACGACCTOGCC | AAAAGCTGGGTTGAGAGGGC |
| 86 | GTCGTATCCAGTGCAGGGTCCGAGGTATTCGCACTGGATACGACGCAGTT | AAGCTGCCAGTTGAAGAACT |
| 87 | GTCGTATCCAGTGCAGGGTCCGAGGTATTCGCACTGGATACGACAAACCC | AGCTACATTGTCTGCTGGGT |
| 88 | GTCGTATCCAGTGCAGGGTCCGAGGTATTCGCACTGGATACGACACTGAG | AGCTCGGCCTGAGGCCCCTC |
| 89 | GTCGTATCCAGTGCAGGGTCCGAGGTATTCGCACTGGATACGACAGTTCT | AGGCTGGTCCGATGGTAGTG |
| 90 | GTCGTATCCAGTGCAGGGTCCGAGGTATTCGCACTGGATACGACCCCCCG | AGGGCGGCGGCGGCGGCGGC |
| 91 | GTCGTATCCAGTGCAGGGTCCGAGGTATTCGCACTGGATACGACACAGGC | ATATTGCACTTGTCCCGGCC |
| 92 | GTCGTATCCAGTGCAGGGTCCGAGGTATTCGCACTGGATACGACTCGGGG | ATCCTGTACTGAGCTGCCCC |
| 93 | GTCGTATCCAGTGCAGGGTCCGAGGTATTCGCACTGGATACGACCTCGGG | ATCCTGTACTGAGCTGCCCC |
| 94 | GTCGTATCCAGTGCAGGGTCCGAGGTATTCGCACTGGATACGACCAGGCC | ATTGCACTTGTCCCGGCCTG |
| 95 | GTCGTATCCAGTGCAGGGTCCGAGGTATTCGCACTGGATACGACACAGGC | ATTGCACTTGTCCCGGCCTG |
| 96 | GTCGTATCCAGTGCAGGGTCCGAGGTATTCGCACTGGATACGACACAGGC | ATTGCACTTGCCCCGGCCTG |
| 97 | GTCGTATCCAGTGCAGGGTCCGAGGTATTCGCACTGGATACGACCAGGCC | CATTGCACTTGTCCCGGCCT |
| 98 | GTCGTATCCAGTGCAGGGTCCGAGGTATTCGCACTGGATACGACCCAGAC | CATTGCACTTGTCTCGGTCT |
| 99 | GTCGTATCCAGTGCAGGGTCCGAGGTATTCGCACTGGATACGACGGCGAG | CATTGGTGGTTCAGTGGTAG |
| 100 | GTCGTATCCAGTGCAGGGTCCGAGGTATTCGCACTGGATACGACAAGCCA | CCCCCCGCTGCTAAATTTGA |
| 101 | GTCGTATCCAGTGCAGGGTCCGAGGTATTCGCACTGGATACGACTCTCGG | CCCTGTACTGAGCTGCCCCG |
| 102 | GTCGTATCCAGTGCAGGGTCCGAGGTATTCGCACTGGATACGACTCTCGG | CCTGTACTGAGCTGCCCCGA |
| 103 | GTCGTATCCAGTGCAGGGTCCGAGGTATTCGCACTGGATACGACCTCGGG | CCTGTACTGAGCTGCCCCGA |
| 104 | GTCGTATCCAGTGCAGGGTCCGAGGTATTCGCACTGGATACGACCGTCCT | CGAGGAGGAAGAGGAGGACG |
| 105 | GTCGTATCCAGTGCAGGGTCCGAGGTATTCGCACTGGATACGACCTATAC | CGAGGTAGTAGGTTGTATAG |
| 106 | GTCGTATCCAGTGCAGGGTCCGAGGTATTCGCACTGGATACGACAAAGGC | CTATACGACCTGCTGCCTTT |
| 107 | GTCGTATCCAGTGCAGGGTCCGAGGTATTCGCACTGGATACGACCTCGGG | CTGTACTGAGCTGCCCCGAG |
| 108 | GTCGTATCCAGTGCAGGGTCCGAGGTATTCGCACTGGATACGACGCGAGA | GAATTGGTGGTTCAGTGGTA |
| 109 | GTCGTATCCAGTGCAGGGTCCGAGGTATTCGCACTGGATACGACAGGCGA | GCATCGGTGGTTCAGTGGTA |
| 110 | GTCGTATCCAGTGCAGGGTCCGAGGTATTCGCACTGGATACGACAGGCGÅ | GCATTAGTGGTTCAGTGGTA |
| 111 | GTCGTATCCAGTGCAGGGTCCGAGGTATTCGCACTGGATACGACGGCGAG | GCATTGATGGTTCAGTGGTA |
| 112 | GTCGTATCCAGTGCAGGGTCCGAGGTATTCGCACTGGATACGACGGCGAG | GCATTGGTAGTTCAGTGGTA |
| 113 | GTCGTATCCAGTGCAGGGTCCGAGGTATTCGCACTGGATACGACCGGGCG | GCATTGGTGGTTCAGTGGTA |
| 114 | GTCGTATCCAGTGCAGGGTCCGAGGTATTCGCACTGGATACGACGACGAG | GCATTGGTGGTTCAGTGGTA |
| 115 | GTCGTATCCAGTGCAGGGTCCGAGGTATTCGCACTGGATACGACAGGCGÅ | GCATTGGTGGTTCAGTGGTA |
| 116 | GTCGTATCCAGTGCAGGGTCCGAGGTATTCGCACTGGATACGACGGCGAG | GCATTGGTGGTTCAGTGGTA |
| 117 | GTCGTATCCAGTGCAGGGTCCGAGGTATTCGCACTGGATACGACGGCGAG | GCATTGGTGGTTCAGTGGTA |
| 118 | GTCGTATCCAGTGCAGGGTCCGAGGTATTCGCACTGGATACGACGGCGAG | GCATTGGTGGTTCGGTGGTA |
| 119 | GTCGTATCCAGTGCAGGGTCCGAGGTATTCGCACTGGATACGACAATCTT | GCGCCGCTGGTGTAGTGGCA |
| 120 | GTCGTATCCAGTGCAGGGTCCGAGGTATTCGCACTGGATACGACGGCGAG | GCGTTGGTGGTTCAGTGGTA |
| 121 | GTCGTATCCAGTGCAGGGTCCGAGGTATTCGCACTGGATACGACTAAGTT | GGCTGGCCCGATGGTAGTGG |
| 122 | GTCGTATCCAGTGCAGGGTCCGAGGTATTCGCACTGGATACGACTTGAGG | GGCTGGTCCGAAGGTAGTGA |
| 123 | GTCGTATCCAGTGCAGGGTCCGAGGTATTCGCACTGGATACGACTTGAGA | GGCTGGTCCGAAGGCAGTGA |
| 124 | GTCGTATCCAGTGCAGGGTCCGAGGTATTCGCACTGGATACGACAAGTTC | GGCTGGTCCGATAGTAGTGG |
| 125 | GTCGTATCCAGTGCAGGGTCCGAGGTATTCGCACTGGATACGACTTCTGA | GGCTGGTCCGATGGTAGTGG |
| 126 | GTCGTATCCAGTGCAGGGTCCGAGGTATTCGCACTGGATACGACTTCTGA | GGCTGGTCCGATGGTAGCGG |
| 127 | GTCGTATCCAGTGCAGGGTCCGAGGTATTCGCACTGGATACGACAAGTTC | GGCTGGTCCGATGGTAGTGG |
| 128 | GTCGTATCCAGTGCAGGGTCCGAGGTATTCGCACTGGATACGACGTTCTG | GGCTGGTCCGATGGTAGTAG |
| 129 | GTCGTATCCAGTGCAGGGTCCGAGGTATTCGCACTGGATACGACGTTATC | GGGGAATTGCGAGCGGATAA |
| 130 | GTCGTATCCAGTGCAGGGTCCGAGGTATTCGCACTGGATACGACATTATC | GGGGAATTGTGAGCGGATAA |
| 131 | GTCGTATCCAGTGCAGGGTCCGAGGTATTCGCACTGGATACGACGTTGTC | GGGGAATTGTGAGCGGACAA |
| 132 | GTCGTATCCAGTGCAGGGTCCGAGGTATTCGCACTGGATACGACCCTGTT | TAAGCGGCGGGGTCGGAACA |
| 133 | GTCGTATCCAGTGCAGGGTCCGAGGTATTCGCACTGGATACGACAGTCTC | TAAGGGGCAGAGAGCGAGAC |
| 134 | GTCGTATCCAGTGCAGGGTCCGAGGTATTCGCACTGGATACGACACAGGC | TAATGCACTTGTCCCGGCCT |
| 135 | GTCGTATCCAGTGCAGGGTCCGAGGTATTCGCACTGGATACGACCGCCGA | TAGCAGCACGTAAATATCGG |
| 136 | GTCGTATCCAGTGCAGGGTCCGAGGTATTCGCACTGGATACGACTCAACG | TAGCTTATCAGACTGACGTT |
| 137 | GTCGTATCCAGTGCAGGGTCCGAGGTATTCGCACTGGATACGACTTAACA | TAGCTTATCAGACTGATGTT |
| 138 | GTCGTATCCAGTGCAGGGTCCGAGGTATTCGCACTGGATACGACAACATC | TAGCTTATCAGACTGATGTT |
| 139 | GTCGTATCCAGTGCAGGGTCCGAGGTATTCGCACTGGATACGACACAGGC | TATTGCACTTGTCACGGCCT |
| 140 | GTCGTATCCAGTGCAGGGTCCGAGGTATTCGCACTGGATACGACACAGGC | TATTGCACTTGCCCCGGCCT |
| 141 | GTCGTATCCAGTGCAGGGTCCGAGGTATTCGCACTGGATACGACCAGACC | TATTGCACTTGTCCCGGTCT |
| 142 | GTCGTATCCAGTGCAGGGTCCGAGGTATTCGCACTGGATACGACACAGGC | TATTGCACTTGTCCCAGCCT |
| 143 | GTCGTATCCAGTGCAGGGTCCGAGGTATTCGCACTGGATACGACACAGGC | TATTGCACTCGTCCCGGCCT |
| 144 | GTCGTATCCAGTGCAGGGTCCGAGGTATTCGCACTGGATACGACACTGGC | TATTGCACTTGTCCCGGCCA |
| 145 | GTCGTATCCAGTGCAGGGTCCGAGGTATTCGCACTGGATACGACCGGGCC | TATTGCACTTGTCCCGGCCC |
| 146 | GTCGTATCCAGTGCAGGGTCCGAGGTATTCGCACTGGATACGACCAGGCC | TATTGCACTTGTCCCGGCCT |
| 147 | GTCGTATCCAGTGCAGGGTCCGAGGTATTCGCACTGGATACGACCAGGCC | TATTGCGCTTGTCCCGGCCT |
| 148 | GTCGTATCCAGTGCAGGGTCCGAGGTATTCGCACTGGATACGACACAGGC | TATTGCACTTGTCCCTGCCT |
| 149 | GTCGTATCCAGTGCAGGGTCCGAGGTATTCGCACTGGATACGACACAGGC | TATTGCACTTATCCCGGCCT |
| 150 | GTCGTATCCAGTGCAGGGTCCGAGGTATTCGCACTGGATACGACACAGGC | TATTGCACTTGTCCAGGCCT |
| 151 | GTCGTATCCAGTGCAGGGTCCGAGGTATTCGCACTGGATACGACACAGGC | TATTGCTCTTGTCCCGGCCT |
| 152 | GTCGTATCCAGTGCAGGGTCCGAGGTATTCGCACTGGATACGACCACAGG | TATTGCACTTGTCCCGGCCT |
| 153 | GTCGTATCCAGTGCAGGGTCCGAGGTATTCGCACTGGATACGACTCTCGG | TCCCGTACTGAGCTGCCCCG |
| 154 | GTCGTATCCAGTGCAGGGTCCGAGGTATTCGCACTGGATACGACCTCGGG | TCCCGTACTGAGCTGCCCCG |
| 155 | GTCGTATCCAGTGCAGGGTCCGAGGTATTCGCACTGGATACGACCGGGGC | TCCCGTACTGAGCTGCCCCG |
| 156 | GTCGTATCCAGTGCAGGGTCCGAGGTATTCGCACTGGATACGACGCGCCG | TCCCTGGTGGTCTAATGGTT |
| 157 | GTCGTATCCAGTGCAGGGTCCGAGGTATTCGCACTGGATACGACGCGCCG | TCCCTGGTGGTCTAGTAGTT |
| 158 | GTCGTATCCAGTGCAGGGTCCGAGGTATTCGCACTGGATACGACAGCGCC | TCCCTGGTGATCTAGTGGTT |
| 159 | GTCGTATCCAGTGCAGGGTCCGAGGTATTCGCACTGGATACGACAGAGCG | TCCCTGGTGGTCTAGTGGTC |
| 160 | GTCGTATCCAGTGCAGGGTCCGAGGTATTCGCACTGGATACGACTCTCGG | TCCTGCACTGAGCTGCCCCG |
| 161 | GTCGTATCCAGTGCAGGGTCCGAGGTATTCGCACTGGATACGACTCTCGG | TCCTGTACTAAGCTGCCCCG |
| 162 | GTCGTATCCAGTGCAGGGTCCGAGGTATTCGCACTGGATACGACACTCGG | TCCTGTACCGAGCTGCCCCG |
| 163 | GTCGTATCCAGTGCAGGGTCCGAGGTATTCGCACTGGATACGACCTTGGG | TCCTGTACTGAGCTGCCCCA |
| 164 | GTCGTATCCAGTGCAGGGTCCGAGGTATTCGCACTGGATACGACTCCCGG | TCCTGTACTGAGCTGCCCCG |
| 165 | GTCGTATCCAGTGCAGGGTCCGAGGTATTCGCACTGGATACGACCACGGG | TCCTGTACTGAGCTGCCCCG |
| 166 | GTCGTATCCAGTGCAGGGTCCGAGGTATTCGCACTGGATACGACTCTCGG | TCCTGTACCGAGCTGCCCCG |
| 167 | GTCGTATCCAGTGCAGGGTCCGAGGTATTCGCACTGGATACGACTCGTGG | TCCTGTACTGAGCTGCCACG |
| 168 | GTCGTATCCAGTGCAGGGTCCGAGGTATTCGCACTGGATACGACCTCGGG | TCCTGTACCGAGCTGCCCCG |
| 169 | GTCGTATCCAGTGCAGGGTCCGAGGTATTCGCACTGGATACGACACGGGG | TCCTGTACTGAGCTGCCCCG |
| 170 | GTCGTATCCAGTGCAGGGTCCGAGGTATTCGCACTGGATACGACTCTTGG | TCCTGTACTGAGCTGCCCCA |
| 171 | GTCGTATCCAGTGCAGGGTCCGAGGTATTCGCACTGGATACGACCTCGGG | TCCTGTACTGAGTTGCCCCG |
| 172 | GTCGTATCCAGTGCAGGGTCCGAGGTATTCGCACTGGATACGACACTCGG | TCCTGTACTGGGCTGCCCCG |
| 173 | GTCGTATCCAGTGCAGGGTCCGAGGTATTCGCACTGGATACGACCTCGGG | TCCTGTACTGGGCTGCCCCG |
| 174 | GTCGTATCCAGTGCAGGGTCCGAGGTATTCGCACTGGATACGACCTCGGG | TCCTGTACTGAGCTGCCCGA |
| 175 | GTCGTATCCAGTGCAGGGTCCGAGGTATTCGCACTGGATACGACACCCAT | TGAGAACTGAATTCCATGGG |
| 176 | GTCGTATCCAGTGCAGGGTCCGAGGTATTCGCACTGGATACGACGGTCTC | TGAGGGGCAGAGAGCGAGAC |
| 177 | GTCGTATCCAGTGCAGGGTCCGAGGTATTCGCACTGGATACGACAGTCTC | TGAGGGACAGAGAGCGAGAC |
| 178 | GTCGTATCCAGTGCAGGGTCCGAGGTATTCGCACTGGATACGACAAAAGT | TGAGGGGCAGAGAGCGGGAC |
| 179 | GTCGTATCCAGTGCAGGGTCCGAGGTATTCGCACTGGATACGACAAAGTC | TGAGGGGCAGAGGGCGAGAC |
| 180 | GTCGTATCCAGTGCAGGGTCCGAGGTATTCGCACTGGATACGACAAACCA | TGAGGTAGTAGGTCGTGTGG |
| 181 | GTCGTATCCAGTGCAGGGTCCGAGGTATTCGCACTGGATACGACAGACCA | TGAGGTAGTAGGTTGTGTGG |
| 182 | GTCGTATCCAGTGCAGGGTCCGAGGTATTCGCACTGGATACGACCCACAC | TGAGGTAGTAGGTCGTGTGG |
| 183 | GTCGTATCCAGTGCAGGGTCCGAGGTATTCGCACTGGATACGACCTATAC | TGAGGTAGTAGGTTGTATAG |
| 184 | GTCGTATCCAGTGCAGGGTCCGAGGTATTCGCACTGGATACGACACAGCG | TGAGGTAGTAGTTTGCGCTG |
| 185 | GTCGTATCCAGTGCAGGGTCCGAGGTATTCGCACTGGATACGACCTATAC | TGAGGTAGTAGGTCGTATAG |
| 186 | GTCGTATCCAGTGCAGGGTCCGAGGTATTCGCACTGGATACGACACTATA | TGAGGTAGTAGGTCGTATAG |
| 187 | GTCGTATCCAGTGCAGGGTCCGAGGTATTCGCACTGGATACGACCACACA | TGAGGTAGTAGGTTGTGTG |
| 188 | GTCGTATCCAGTGCAGGGTCCGAGGTATTCGCACTGGATACGACAACCAC | TGAGGTAGTAGGTTATGTGG |
| 189 | GTCGTATCCAGTGCAGGGTCCGAGGTATTCGCACTGGATACGACAACTAT | TGAGGTAGTGGATTGTATAG |
| 190 | GTCGTATCCAGTGCAGGGTCCGAGGTATTCGCACTGGATACGACAACTGT | TGAGGTAGTAGTTTGCACAG |
| 191 | GTCGTATCCAGTGCAGGGTCCGAGGTATTCGCACTGGATACGACCCACAC | TGAGGTAGTAGGTTGTGTGG |
| 192 | GTCGTATCCAGTGCAGGGTCCGAGGTATTCGCACTGGATACGACCAGACA | TGGAGTGTGACAATGGTGTC |
| 193 | GTCGTATCCAGTGCAGGGTCCGAGGTATTCGCACTGGATACGACAGTTCT | TGGGTTTACGTTGGGAGAAC |
| 194 | GTCGTATCCAGTGCAGGGTCCGAGGTATTCGCACTGGATACGACCTTCCA | TGTAAACATCCTTGACTGGA |
| 195 | GTCGTATCCAGTGCAGGGTCCGAGGTATTCGCACTGGATACGACTTTCCA | TGTAAACATCCCCGACTGGA |
| 196 | GTCGTATCCAGTGCAGGGTCCGAGGTATTCGCACTGGATACGACTGAGAG | TGTAAACATCCTACACTCTC |
| 197 | GTCGTATCCAGTGCAGGGTCCGAGGTATTCGCACTGGATACGACAGCTTC | TGTAGACATCCTTGACTGGA |
| 198 | GTCGTATCCAGTGCAGGGTCCGAGGTATTCGCACTGGATACGACACAGGC | TGTTGCACTTGTCCCGGCCT |
| 199 | GTCGTATCCAGTGCAGGGTCCGAGGTATTCGCACTGGATACGACTCTCGG | TTCTGTACTGAGCTGCCCCG |
| 200 | GTCGTATCCAGTGCAGGGTCCGAGGTATTCGCACTGGATACGACACAGGC | TTGCACTTGTCCCGGCCTGT |
| 201 | GTCGTATCCAGTGCAGGGTCCGAGGTATTCGCACTGGATACGACCCGCCC | TTGGGAGCGGGGGGCGG |
| 202 | GTCGTATCCAGTGCAGGGTCCGAGGTATTCGCACTGGATACGACGAGAAG | ATTCCGGTTGTTCTTCTC |
| 203 | GTCGTATCCAGTGCAGGGTCCGAGGTATTCGCACTGGATACGACCCATCT | TTGTAGCACTGGGGCCAGAT |
| 204 | GTCGTATCCAGTGCAGGGTCCGAGGTATTCGCACTGGATACGACGGCGTC | AGATGAGAGGACGGGACGCC |
| 205 | GTCGTATCCAGTGCAGGGTCCGAGGTATTCGCACTGGATACGACTTTCAG | TATGGAAGTGCCGCCGCTGA |
| 206 | GTCGTATCCAGTGCAGGGTCCGAGGTATTCGCACTGGATACGACGCCAGC | TATTAAATCAATCTCGGCTG |

| SEQ | | |
|---|---|---|
| ID NO. | Reverse Primer 5′ to 3′ | TaqMan Probe 5′ to 3′ |
| 1 | GTGCAGGGTCCGAGGT | CCCCCTCTTCAGCAT |
| 2 | GTGCAGGGTCCGAGGT | TCGTCTGGTCAGCAT |
| 3 | GTGCAGGGTCCGAGGT | TTTTCCGCTCAGCAT |
| 4 | GTGCAGGGTCCGAGGT | TTTTTTGCCCAGCAT |
| 5 | GTGCAGGGTCCGAGGT | GGCGTGTTTCAGCAT |
| 6 | GTGCAGGGTCCGAGGT | TCCTTTGCGCAGCAT |
| 7 | GTGCAGGGTCCGAGGT | GGCCCCGCTCAGCAT |
| 8 | GTGCAGGGTCCGAGGT | CCTTTTTCGCAGCAT |
| 9 | GTGCAGGGTCCGAGGT | GGTTGCTCTCAGCAT |
| 10 | GTGCAGGGTCCGAGGT | GTTGCCTGTCAGCAT |
| 11 | GTGCAGGGTCCGAGGT | GTTGGTGGCCAGCAT |
| 12 | GTGCAGGGTCCGAGGT | TCCTGTCCTCAGCAT |
| 13 | GTGCAGGGTCCGAGGT | TTCTTCTGTCAGCAT |
| 14 | GTGCAGGGTCCGAGGT | TTTGTGTTTCAGCAT |
| 15 | GTGCAGGGTCCGAGGT | TTTTCTTTTCAGCAT |
| 16 | GTGCAGGGTCCGAGGT | CTGGCGTTCCAGCAT |
| 17 | GTGCAGGGTCCGAGGT | GCCCTTGTCCAGCAT |
| 18 | GTGCAGGGTCCGAGGT | GGTTTGCCCCAGCAT |
| 19 | GTGCAGGGTCCGAGGT | CTTGCTGTGCAGCAT |
| 20 | GTGCAGGGTCCGAGGT | CTTTCGCCTCAGCAT |
| 21 | GTGCAGGGTCCGAGGT | TTTGGCTCCCAGCAT |
| 22 | GTGCAGGGTCCGAGGT | TGTTTCTCGCAGCAT |
| 23 | GTGCAGGGTCCGAGGT | TTTGCCTGGCAGCAT |
| 24 | GTGCAGGGTCCGAGGT | CGTGGTGCCCAGCAT |
| 25 | GTGCAGGGTCCGAGGT | GTGTTGTTTCAGCAT |
| 26 | GTGCAGGGTCCGAGGT | GTTCGTTTCCAGCAT |
| 27 | GTGCAGGGTCCGAGGT | GCCTTGCTCCAGCAT |
| 28 | GTGCAGGGTCCGAGGT | GTCCTGCTCCAGCAT |
| 29 | GTGCAGGGTCCGAGGT | GTTCTTTTTCAGCAT |
| 30 | GTGCAGGGTCCGAGGT | TGTTCTCCCCAGCAT |
| 31 | GTGCAGGGTCCGAGGT | TCCTTCGGCCAGCAT |
| 32 | GTGCAGGGTCCGAGGT | TTTTCCGGGCAGCAT |
| 33 | GTGCAGGGTCCGAGGT | CTTGTCCTTCAGCAT |
| 34 | GTGCAGGGTCCGAGGT | TGGTTTTCTCAGCAT |
| 35 | GTGCAGGGTCCGAGGT | CCTTGCCTTCAGCAT |
| 36 | GTGCAGGGTCCGAGGT | GTTCCCTGTCAGCAT |
| 37 | GTGCAGGGTCCGAGGT | GGCGTGTTTCAGCAT |
| 38 | GTGCAGGGTCCGAGGT | CTTTTTCTCCAGCAT |
| 39 | GTGCAGGGTCCGAGGT | TCCCCTTCTCAGCAT |
| 40 | GTGCAGGGTCCGAGGT | TTCTCCCTTCAGCAT |
| 41 | GTGCAGGGTCCGAGGT | GTCCCGCCGCAGCAT |
| 42 | GTGCAGGGTCCGAGGT | TTTCCCTTGCAGCAT |
| 43 | GTGCAGGGTCCGAGGT | CCGTCCTCCCAGCAT |
| 44 | GTGCAGGGTCCGAGGT | TCTTTGCTTCAGCAT |
| 45 | GTGCAGGGTCCGAGGT | TTTCCGGCTCAGCAT |
| 46 | GTGCAGGGTCCGAGGT | TGGCGTGCCCAGCAT |
| 47 | GTGCAGGGTCCGAGGT | CTGGCGTGTCAGCAT |
| 48 | GTGCAGGGTCCGAGGT | GTTTTCTTCCAGCAT |
| 49 | GTGCAGGGTCCGAGGT | TGTGTTTTCCAGCAT |
| 50 | GTGCAGGGTCCGAGGT | TTGCTTCCTCAGCAT |
| 51 | GTGCAGGGTCCGAGGT | GTCTTCGTCCAGCAT |
| 52 | GTGCAGGGTCCGAGGT | TGTCTTCTTCAGCAT |
| 53 | GTGCAGGGTCCGAGGT | TCTGGCCGGCAGCAT |
| 54 | GTGCAGGGTCCGAGGT | TTGCTTCTGCAGCAT |
| 55 | GTGCAGGGTCCGAGGT | TTCTCGCTCCAGCAT |
| 56 | GTGCAGGGTCCGAGGT | TGCCTCGCTCAGCAT |
| 57 | GTGCAGGGTCCGAGGT | TTGGTCTCGCAGCAT |
| 58 | GTGCAGGGTCCGAGGT | TCTTCTCGCCAGCAT |
| 59 | GTGCAGGGTCCGAGGT | TGTCCTCTCCAGCAT |
| 60 | GTGCAGGGTCCGAGGT | TCTCCTTTGCAGCAT |
| 61 | GTGCAGGGTCCGAGGT | CTTTCTGTCCAGCAT |
| 62 | GTGCAGGGTCCGAGGT | GCTTCCTGTCAGCAT |
| 63 | GTGCAGGGTCCGAGGT | TCTTCCTGTCAGCAT |
| 64 | GTGCAGGGTCCGAGGT | CCGCCCGCCCAGCAT |
| 65 | GTGCAGGGTCCGAGGT | TGTTCCTTTCAGCAT |
| 66 | GTGCAGGGTCCGAGGT | GCGTTCGTGCAGCAT |
| 67 | GTGCAGGGTCCGAGGT | CGCGTGCCGCAGCAT |
| 68 | GTGCAGGGTCCGAGGT | CCCCCGCCGCAGCAT |
| 69 | GTGCAGGGTCCGAGGT | TCCTCCTCCCAGCAT |
| 70 | GTGCAGGGTCCGAGGT | TTCTGTTTTCAGCAT |
| 71 | GTGCAGGGTCCGAGGT | CCCCCGCCGCAGCAT |
| 72 | GTGCAGGGTCCGAGGT | CCCCGCCGCCAGCAT |
| 73 | GTGCAGGGTCCGAGGT | GTGTTGTTCCAGCAT |
| 74 | GTGCAGGGTCCGAGGT | CCTCTCTTCCAGCAT |
| 75 | GTGCAGGGTCCGAGGT | TCTCCGTCTCAGCAT |
| 76 | GTGCAGGGTCCGAGGT | TCTGCCCTTCAGCAT |
| 77 | GTGCAGGGTCCGAGGT | GCTTTTGGGCAGCAT |
| 78 | GTGCAGGGTCCGAGGT | TTTGGTGTTCAGCAT |
| 79 | GTGCAGGGTCCGAGGT | TCCCGGGTCCAGCAT |
| 80 | GTGCAGGGTCCGAGGT | GTGCGCCGTCAGCAT |
| 81 | GTGCAGGGTCCGAGGT | TCCCTCTCTCAGCAT |
| 82 | GTGCAGGGTCCGAGGT | TTGTGTTTTCAGCAT |
| 83 | GTGCAGGGTCCGAGGT | CGCCCTCTCCAGCAT |
| 84 | GTGCAGGGTCCGAGGT | TCCCTCTCTCAGCAT |
| 85 | GTGCAGGGTCCGAGGT | CTCGCCCTCCAGCAT |
| 86 | GTGCAGGGTCCGAGGT | GCTGTTCTTCAGCAT |
| 87 | GTGCAGGGTCCGAGGT | TTTCCCTGCCAGCAT |
| 88 | GTGCAGGGTCCGAGGT | TCTGTGGGGCAGCAT |
| 89 | GTGCAGGGTCCGAGGT | TGTTCTGTTCAGCAT |
| 90 | GTGCAGGGTCCGAGGT | CCCCCGCCGCAGCAT |
| 91 | GTGCAGGGTCCGAGGT | TCTGGCCGGCAGCAT |
| 92 | GTGCAGGGTCCGAGGT | TCGGGGCTGCAGCAT |
| 93 | GTGCAGGGTCCGAGGT | CTCGGGGCTCAGCAT |
| 94 | GTGCAGGGTCCGAGGT | CTGGCCGGGCAGCAT |
| 95 | GTGCAGGGTCCGAGGT | TCTGGCCGGCAGCAT |
| 96 | GTGCAGGGTCCGAGGT | TCTGGCCGGCAGCAT |
| 97 | GTGCAGGGTCCGAGGT | CTGGCCGGGCAGCAT |
| 98 | GTGCAGGGTCCGAGGT | CCTGTCCGTCAGCAT |
| 99 | GTGCAGGGTCCGAGGT | GGCGTGTTTCAGCAT |
| 100 | GTGCAGGGTCCGAGGT | TTGCCTGTCCAGCAT |
| 101 | GTGCAGGGTCCGAGGT | TCTCGGGGCCAGCAT |
| 102 | GTGCAGGGTCCGAGGT | TCTCGGGGCCAGCAT |
| 103 | GTGCAGGGTCCGAGGT | CTCGGGGCTCAGCAT |
| 104 | GTGCAGGGTCCGAGGT | CGTCCTCCTCAGCAT |
| 105 | GTGCAGGGTCCGAGGT | CTTTTCTTCCAGCAT |
| 106 | GTGCAGGGTCCGAGGT | TTTGGCTGCCAGCAT |
| 107 | GTGCAGGGTCCGAGGT | CTCGGGGCTCAGCAT |
| 108 | GTGCAGGGTCCGAGGT | GCGTGTTTTCAGCAT |
| 109 | GTGCAGGGTCCGAGGT | TGGCGTGTTCAGCAT |
| 110 | GTGCAGGGTCCGAGGT | TGGCGTGTTCAGCAT |
| 111 | GTGCAGGGTCCGAGGT | GGCGTGTTTCAGCAT |
| 112 | GTGCAGGGTCCGAGGT | GGCGTGTTTCAGCAT |
| 113 | GTGCAGGGTCCGAGGT | CGGGCGTGTCAGCAT |
| 114 | GTGCAGGGTCCGAGGT | GTCGTGTTTCAGCAT |
| 115 | GTGCAGGGTCCGAGGT | TGGCGTTTTCAGCAT |
| 116 | GTGCAGGGTCCGAGGT | GGCGTGTTTCAGCAT |
| 117 | GTGCAGGGTCCGAGGT | GGCGTGTTCCAGCAT |
| 118 | GTGCAGGGTCCGAGGT | GGCGTGTTTCAGCAT |
| 119 | GTGCAGGGTCCGAGGT | TTTCTTGCTCAGCAT |
| 120 | GTGCAGGGTCCGAGGT | GGCGTGTTTCAGCAT |
| 121 | GTGCAGGGTCCGAGGT | TTTGTTCTGCAGCAT |
| 122 | GTGCAGGGTCCGAGGT | TTGTGGTTTCAGCAT |
| 123 | GTGCAGGGTCCGAGGT | TTGTGTTTTCAGCAT |
| 124 | GTGCAGGGTCCGAGGT | TTGTTCTGTCAGCAT |
| 125 | GTGCAGGGTCCGAGGT | TTCTGTTGTCAGCAT |
| 126 | GTGCAGGGTCCGAGGT | TTCTGTTTTCAGCAT |
| 127 | GTGCAGGGTCCGAGGT | TTGTTCTGTCAGCAT |
| 128 | GTGCAGGGTCCGAGGT | GTTCTGTTTCAGCAT |
| 129 | GTGCAGGGTCCGAGGT | GTTTTCCGCCAGCAT |
| 130 | GTGCAGGGTCCGAGGT | TTTTTCCGCCAGCAT |
| 131 | GTGCAGGGTCCGAGGT | GTTGTCCGCCAGCAT |
| 132 | GTGCAGGGTCCGAGGT | CCTGTTCCGCAGCAT |
| 133 | GTGCAGGGTCCGAGGT | TGTCTCGCTCAGCAT |
| 134 | GTGCAGGGTCCGAGGT | TCTGGCCGGCAGCAT |
| 135 | GTGCAGGGTCCGAGGT | CGCCGTTTTCAGCAT |
| 136 | GTGCAGGGTCCGAGGT | TCTTCGTCTCAGCAT |
| 137 | GTGCAGGGTCCGAGGT | TTTTCTTCTCAGCAT |
| 138 | GTGCAGGGTCCGAGGT | TTCTTCTGTCAGCAT |
| 139 | GTGCAGGGTCCGAGGT | TCTGGCCGTCAGCAT |
| 140 | GTGCAGGGTCCGAGGT | TCTGGCCGGCAGCAT |
| 141 | GTGCAGGGTCCGAGGT | CTGTCCGGGCAGCAT |
| 142 | GTGCAGGGTCCGAGGT | TCTGGCTGGCAGCAT |
| 143 | GTGCAGGGTCCGAGGT | TCTGGCCGGCAGCAT |
| 144 | GTGCAGGGTCCGAGGT | TCTGGCCGGCAGCAT |
| 145 | GTGCAGGGTCCGAGGT | CGGGCCGGGCAGCAT |
| 146 | GTGCAGGGTCCGAGGT | CTGGCCGGGCAGCAT |
| 147 | GTGCAGGGTCCGAGGT | CTGGCCGGGCAGCAT |
| 148 | GTGCAGGGTCCGAGGT | TCTGGCTGGCAGCAT |
| 149 | GTGCAGGGTCCGAGGT | TCTGGCCGGCAGCAT |
| 150 | GTGCAGGGTCCGAGGT | TCTGGCCTGCAGCAT |
| 151 | GTGCAGGGTCCGAGGT | TCTGGCCGGCAGCAT |
| 152 | GTGCAGGGTCCGAGGT | CTCTGGCCGCAGCAT |
| 153 | GTGCAGGGTCCGAGGT | TCTCGGGGCCAGCAT |
| 154 | GTGCAGGGTCCGAGGT | CTCGGGGCTCAGCAT |
| 155 | GTGCAGGGTCCGAGGT | CGGGGCTGCCAGCAT |
| 156 | GTGCAGGGTCCGAGGT | GCGCCGTTTCAGCAT |
| 157 | GTGCAGGGTCCGAGGT | GCGCCGTTTCAGCAT |
| 158 | GTGCAGGGTCCGAGGT | TGCGCCGTTCAGCAT |
| 159 | GTGCAGGGTCCGAGGT | TGTGCGCCGCAGCAT |
| 160 | GTGCAGGGTCCGAGGT | TCTCGGGGCCAGCAT |
| 161 | GTGCAGGGTCCGAGGT | TCTCGGGGCCAGCAT |
| 162 | GTGCAGGGTCCGAGGT | TCTCGGGGCCAGCAT |
| 163 | GTGCAGGGTCCGAGGT | CTTGGGGCTCAGCAT |
| 164 | GTGCAGGGTCCGAGGT | TCCCGGGGCCAGCAT |
| 165 | GTGCAGGGTCCGAGGT | CTCGGGGCTCAGCAT |
| 166 | GTGCAGGGTCCGAGGT | TCTCGGGGCCAGCAT |
| 167 | GTGCAGGGTCCGAGGT | TCGTGGCTGCAGCAT |
| 168 | GTGCAGGGTCCGAGGT | CTCGGGGCTCAGCAT |
| 169 | GTGCAGGGTCCGAGGT | TCGGGGCTGCAGCAT |
| 170 | GTGCAGGGTCCGAGGT | TCTTGGGGCCAGCAT |
| 171 | GTGCAGGGTCCGAGGT | CTCGGGGCTCAGCAT |
| 172 | GTGCAGGGTCCGAGGT | TCTCGGGGCCAGCAT |
| 173 | GTGCAGGGTCCGAGGT | CTCGGGGCTCAGCAT |
| 174 | GTGCAGGGTCCGAGGT | CTCGGGCTGCAGCAT |
| 175 | GTGCAGGGTCCGAGGT | TCCCTTGGTCAGCAT |
| 176 | GTGCAGGGTCCGAGGT | GGTCTCGCTCAGCAT |
| 177 | GTGCAGGGTCCGAGGT | TGTCTCGCTCAGCAT |
| 178 | GTGCAGGGTCCGAGGT | TTTTGTCCCCAGCAT |
| 179 | GTGCAGGGTCCGAGGT | TTTGTCTCGCAGCAT |
| 180 | GTGCAGGGTCCGAGGT | TTTCCTCTCCAGCAT |
| 181 | GTGCAGGGTCCGAGGT | TGTCCTCTCCAGCAT |
| 182 | GTGCAGGGTCCGAGGT | CCTCTCGTCCAGCAT |
| 183 | GTGCAGGGTCCGAGGT | CTTTTCTTCCAGCAT |
| 184 | GTGCAGGGTCCGAGGT | TCTGCGCTTCAGCAT |
| 185 | GTGCAGGGTCCGAGGT | CTTTTCGTCCAGCAT |
| 186 | GTGCAGGGTCCGAGGT | TCTTTTCGTCAGCAT |
| 187 | GTGCAGGGTCCGAGGT | CTCTCTTCCCAGCAT |
| 188 | GTGCAGGGTCCGAGGT | TTCCTCTTTCAGCAT |
| 189 | GTGCAGGGTCCGAGGT | TTCTTTTCTCAGCAT |
| 190 | GTGCAGGGTCCGAGGT | TTCTGTGCTCAGCAT |
| 191 | GTGCAGGGTCCGAGGT | CCTCTCTTCCAGCAT |
| 192 | GTGCAGGGTCCGAGGT | CTGTCTCCTCAGCAT |
| 193 | GTGCAGGGTCCGAGGT | TGTTCTCCCCAGCAT |
| 194 | GTGCAGGGTCCGAGGT | CTTCCTGTCCAGCAT |
| 195 | GTGCAGGGTCCGAGGT | TTTCCTGTCCAGCAT |
| 196 | GTGCAGGGTCCGAGGT | TGTGTGTGTCAGCAT |
| 197 | GTGCAGGGTCCGAGGT | TGCTTCCTGCAGCAT |
| 198 | GTGCAGGGTCCGAGGT | TCTGGCCGGCAGCAT |
| 199 | GTGCAGGGTCCGAGGT | TCTCGGGGCCAGCAT |
| 200 | GTGCAGGGTCCGAGGT | TCTGGCCGGCAGCAT |
| 201 | GTGCAGGGTCCGAGGT | CCGCCCGCCCAGCAT |
| 202 | GTGCAGGGTCCGAGGT | GTGTTGTTCCAGCAT |
| 203 | GTGCAGGGTCCGAGGT | CCTTCTGGCCAGCAT |
| 204 | GTGCAGGGTCCGAGGT | GGCGTCCCGCAGCAT |
| 205 | GTGCAGGGTCCGAGGT | TTTCTGCGGCAGCAT |
| 206 | GTGCAGGGTCCGAGGT | GCCTGCCGTCAGCAT |

TABLE 3
Therapeutic Molecules Targeting Small RNA Features Correlated to CFR

| SEQ ID NO. | Mimetic |
|---|---|
| 1 | GAAAAGCUGGGUUGAGGGGGdTdT |
| | \|\|\|\|\|\|\|\|\|\|\|\|\|\|\|\|\|\|\| |
| | dTdTGUUUUCGACCCAACUCCCCC |
| 2 | GAAACUCUGGUGGAGGUCCGUAGCGGUCCUGACGUdTdT |
| | \|\|\|\|\|\|\|\|\|\|\|\|\|\|\|\|\|\|\|\|\|\|\|\|\|\|\|\|\|\|\|\|\|\| |
| | dTdTGUUUGAGACCACCUCCAGGCAUCGCCAGGACUGCA |
| 3 | GAAUUGUGAGCGGAUAAdTdT |
| | \|\|\|\|\|\|\|\|\|\|\|\|\|\|\|\| |
| | dTdTGUUAACACUCGCCUAUU |
| 4 | GAUAAGGGAAGUCGGCAAAAUAdTdT |
| | \|\|\|\|\|\|\|\|\|\|\|\|\|\|\|\|\|\|\|\|\| |
| | dTdTGUAUUCCCUUCAGCCGUUUUAU |
| 5 | GCAUGAGUGGUUCAGUGGUAGAAUUCUCGCCdTdT |
| | \|\|\|\|\|\|\|\|\|\|\|\|\|\|\|\|\|\|\|\|\|\|\|\|\|\|\|\|\|\| |
| | dTdTGGUACUCACCAAGUCACCAUCUUAAGAGCGG |
| 6 | GGCCAGUGGCGCAAUGGAdTdT |
| | \|\|\|\|\|\|\|\|\|\|\|\|\|\|\|\|\| |
| | dTdTGCGGUCACCGCGUUACCU |
| 7 | GGCUGGGUCGGUCGGGCUGGGGCGCGAAGCGGGGCCdTdT |
| | \|\|\|\|\|\|\|\|\|\|\|\|\|\|\|\|\|\|\|\|\|\|\|\|\|\|\|\|\|\|\|\|\|\|\| |
| | dTdTGCGACCCAGCCAGCCCGACCCCGCGCUUCGCCCCGG |
| 8 | GGUGAUGUCGGCGAUAUAGGdTdT |
| | \|\|\|\|\|\|\|\|\|\|\|\|\|\|\|\|\|\|\| |
| | dTdTGCACUACAGCCGCUAUAUCC |
| 9 | UAACCCGUAUCGUGAGCAUCCdTdT |
| | \|\|\|\|\|\|\|\|\|\|\|\|\|\|\|\|\|\|\|\| |
| | dTdTUUUGGGCAUAGCACUCGUAGG |
| 10 | UACAACCCCCCACUGCUAAAUUUGACUGGCUUCdTdT |
| | \|\|\|\|\|\|\|\|\|\|\|\|\|\|\|\|\|\|\|\|\|\|\|\|\|\|\|\|\|\|\|\| |
| | dTdTUUGUUGGGGGGUGACGAUUUAAACUGACCGAAG |
| 11 | UAUCCCGCCGUUAGCCACCAUCdTdT |
| | \|\|\|\|\|\|\|\|\|\|\|\|\|\|\|\|\|\|\|\|\| |
| | dTdTUUAGGGCGGCAAUCGGUGGUAG |
| 12 | UCAUGCAACUCGUAGGACAGGUdTdT |
| | \|\|\|\|\|\|\|\|\|\|\|\|\|\|\|\|\|\|\|\|\| |
| | dTdTUGUACGUUGAGCAUCCUGUCCA |
| 13 | UGGAAGACUAGUGAUUCUGUUGUUdTdT |
| | \|\|\|\|\|\|\|\|\|\|\|\|\|\|\|\|\|\|\|\|\|\|\| |
| | dTdTUCCUUCUGAUCACUAAGACAACAA |
| 14 | UGGUCCGAAGGUAGUGGGUUAUCUCAAUdTdT |
| | \|\|\|\|\|\|\|\|\|\|\|\|\|\|\|\|\|\|\|\|\|\|\|\|\|\|\| |
| | dTdTUCCAGGCUUCCAUCACCCAAUAGAGUUA |
| 15 | UUGGCGGAUGAGAUAAGAUUUdTdT |
| | \|\|\|\|\|\|\|\|\|\|\|\|\|\|\|\|\|\|\|\| |
| | dTdTUACCGCCUACUCUAUUCUAAA |
| 16 | UUUCCGUAGUGUAGUGGUUAUCACGUUCGCCUGdTdT |
| | \|\|\|\|\|\|\|\|\|\|\|\|\|\|\|\|\|\|\|\|\|\|\|\|\|\|\|\|\|\|\|\| |
| | dTdTUAAGGCAUCACAUCACCAAUAGUGCAAGCGGAC |
| 17 | UUUCGCCACCUCUGACUUGGGCdTdT |
| | \|\|\|\|\|\|\|\|\|\|\|\|\|\|\|\|\|\|\|\|\| |
| | dTdTUAAGCGGUGGAGACUGAACCCG |
| 18 | ACUACGAUACGGGAGGGCUUACCdTdT |
| | \|\|\|\|\|\|\|\|\|\|\|\|\|\|\|\|\|\|\|\|\|\| |
| | dTdTAGAUGCUAUGCCCUCCCGAAUGG |
| 19 | AAGAACGUCGCGCACUGCUAGdTdT |
| | \|\|\|\|\|\|\|\|\|\|\|\|\|\|\|\|\|\|\|\| |
| | dTdTAUCUUGCAGCGCGUGACGAUC |
| 20 | CAUGCUCGUGAACUGGCGUAUGdTdT |
| | \|\|\|\|\|\|\|\|\|\|\|\|\|\|\|\|\|\|\|\|\| |
| | dTdTCUACGAGCACUUGACCGCAUAC |
| 21 | CGUGUGGAAUUCUCGGGUGCCAAAdTdT |
| | \|\|\|\|\|\|\|\|\|\|\|\|\|\|\|\|\|\|\|\|\|\|\| |
| | dTdTCCACACCUUAAGAGCCCACGGUUU |
| 22 | UCCGGGUAGUCUCGAGAAACAdTdT |
| | \|\|\|\|\|\|\|\|\|\|\|\|\|\|\|\|\|\|\|\| |
| | dTdTUGGCCCAUCAGAGCUCUUUGU |
| 23 | UGAACACCCGGACCUGGCUAUdTdT |
| | \|\|\|\|\|\|\|\|\|\|\|\|\|\|\|\|\|\|\|\| |
| | dTdTUCUUGUGGGCCUGGACCGAUA |
| 24 | AAACGACGAGCGUGGCACCACGdTdT |
| | \|\|\|\|\|\|\|\|\|\|\|\|\|\|\|\|\|\|\|\|\| |
| | dTdTAUUGCUGCUCGCACCGUGGUGC |
| 25 | CUUUCGCUUCGUUAUCUACUCdTdT |
| | \|\|\|\|\|\|\|\|\|\|\|\|\|\|\|\|\|\|\|\| |
| | dTdTCAAAGCGAAGCAAUAGAUGAG |
| 26 | AAAGGAAGGAAUCGAACdTdT |
| | \|\|\|\|\|\|\|\|\|\|\|\|\|\|\|\| |
| | dTdTAUUCCUUCCUUAGCUUG |
| 27 | AGCAGAGUGGCGCAGCGGAAGCGUGCUAGGCdTdT |
| | \|\|\|\|\|\|\|\|\|\|\|\|\|\|\|\|\|\|\|\|\|\|\|\|\|\|\|\|\|\| |
| | dTdTACGUCUCACCGCGUCGCCUUCGCACGAUCCG |
| 28 | AGCAGAGUUGCGCAGCGGAAGCGUGCUGGACdTdT |
| | \|\|\|\|\|\|\|\|\|\|\|\|\|\|\|\|\|\|\|\|\|\|\|\|\|\|\|\|\|\| |
| | dTdTACGUCUCAACGCGUCGCCUUCGCACGACCUG |
| 29 | AGGCUGGUCCGAUGGUAGUGGGUUAUUAGAACdTdT |
| | \|\|\|\|\|\|\|\|\|\|\|\|\|\|\|\|\|\|\|\|\|\|\|\|\|\|\|\|\|\|\| |
| | dTdTACCGACCAGGCUACCAUCACCCAAUAAUCUUG |
| 30 | AUAACCAGGUCCGGGUGUUCAdTdT |
| | \|\|\|\|\|\|\|\|\|\|\|\|\|\|\|\|\|\|\|\| |
| | dTdTAAUUGGUCCAGGCCCACAAGU |
| 31 | AGCGUGGUGGUGCCGAUGGUdTdT |
| | \|\|\|\|\|\|\|\|\|\|\|\|\|\|\|\|\|\|\| |
| | dTdTACGCACCACCACGGCUACCA |
| 32 | UGAACACCCGGACCCGGUUAUdTdT |
| | \|\|\|\|\|\|\|\|\|\|\|\|\|\|\|\|\|\|\|\| |
| | dTdTUCUUGUGGGCCUGGGCCAAUA |
| 33 | AACAGCAGUUGGACAUGdTdT |
| | \|\|\|\|\|\|\|\|\|\|\|\|\|\|\|\| |
| | dTdTAUGUCGUCAACCUGUAC |
| 34 | AUUGGUCGUGGCUGUAGUCCGUGCGAGAAUACCAdTdT |
| | \|\|\|\|\|\|\|\|\|\|\|\|\|\|\|\|\|\|\|\|\|\|\|\|\|\|\|\|\|\|\|\|\| |
| | dTdTAAACCAGCACCGACAUCAGGCACGCUCUUAUGGU |
| 35 | CAUGGAACGGGUUGGCAUGGdTdT |
| | \|\|\|\|\|\|\|\|\|\|\|\|\|\|\|\|\|\|\| |
| | dTdTCUACCUUGCCCAACCGUACC |
| 36 | CAUUCGAUGGUGUCUGGGAUCdTdT |
| | \|\|\|\|\|\|\|\|\|\|\|\|\|\|\|\|\|\|\|\| |
| | dTdTCUAAGCUACCACAGACCCUAG |
| 37 | CAUUGGUGGUUCAGUAGUAGAAUUCUCGCCdTdT |
| | \|\|\|\|\|\|\|\|\|\|\|\|\|\|\|\|\|\|\|\|\|\|\|\|\|\|\|\|\| |
| | dTdTCUAACCACCAAGUCAUCAUCUUAAGAGCGG |
| 38 | CCCGAGACUCAGUGAAAUUGdTdT |
| | \|\|\|\|\|\|\|\|\|\|\|\|\|\|\|\|\|\|\| |
| | dTdTCGGCUCUGAGUCACUUUAAC |
| 39 | AAGAAGGAAAGAAGAAGGGGAAGAAAAGAAGGGGAdTdT |
| | \|\|\|\|\|\|\|\|\|\|\|\|\|\|\|\|\|\|\|\|\|\|\|\|\|\|\|\|\|\|\|\|\|\| |
| | dTdTAUCUUCCUUUCUUCUUCCCCUUCUUUUCUUCCCCU |
| 40 | AAGACGGGAGGAAAGAAGGGAGAUdTdT |
| | \|\|\|\|\|\|\|\|\|\|\|\|\|\|\|\|\|\|\|\|\|\|\| |
| | dTdTAUCUGCCCUCCUUUCUUCCCUCUA |
| 41 | CGGCGGCGGCGGCGGUGGCGGCGGCGGCGGCGGCGGGACdTdT |
| | \|\|\|\|\|\|\|\|\|\|\|\|\|\|\|\|\|\|\|\|\|\|\|\|\|\|\|\|\|\|\|\|\|\|\|\|\|\| |
| | dTdTCCCGCCGCCGCCGCCACCGCCGCCGCCGCCGCCGCCCUG |
| 42 | GAGAACUGAAUUCCAUGGGUUUdTdT |
| | \|\|\|\|\|\|\|\|\|\|\|\|\|\|\|\|\|\|\|\|\| |
| | dTdTGUCUUGACUUAAGGUACCCAAA |
| 43 | GAGGAGGAGGAGGAGGACGGdTdT |
| | \|\|\|\|\|\|\|\|\|\|\|\|\|\|\|\|\|\|\| |
| | dTdTGUCCUCCUCCUCCUCCUGCC |
| 44 | GAGGUAGUAGGUUGCAUAGUdTdT |
| | \|\|\|\|\|\|\|\|\|\|\|\|\|\|\|\|\|\|\| |
| | dTdTGUCCAUCAUCCAACGUAUCA |
| 45 | GAGUAAGACGCUGCCGGAAUdTdT |
| | \|\|\|\|\|\|\|\|\|\|\|\|\|\|\|\|\|\|\| |
| | dTdTGUCAUUCUGCGACGGCCUUA |
| 46 | GAUGGGGAAGAUCGGGCUCGCCAdTdT |
| | \|\|\|\|\|\|\|\|\|\|\|\|\|\|\|\|\|\|\|\|\|\| |
| | dTdTGUACCCCUUCUAGCCCGAGCGGU |
| 47 | GCAUUGGUGGUUCAGUGGUAGAAUUCUCGCCUGdTdT |
| | \|\|\|\|\|\|\|\|\|\|\|\|\|\|\|\|\|\|\|\|\|\|\|\|\|\|\|\|\|\|\|\| |
| | dTdTGGUAACCACCAAGUCACCAUCUUAAGAGCGGAC |
| 48 | GCAUUUGUGGUUCAGUGGUAGAAUUCdTdT |
| | \|\|\|\|\|\|\|\|\|\|\|\|\|\|\|\|\|\|\|\|\|\|\|\|\| |
| | dTdTGGUAAACACCAAGUCACCAUCUUAAG |
| 49 | GGCUGGUCCGAAGGUAGUGAGUUAUCUCAdTdT |
| | \|\|\|\|\|\|\|\|\|\|\|\|\|\|\|\|\|\|\|\|\|\|\|\|\|\|\|\| |
| | dTdTGCGACCAGGCUUCCAUCACUCAAUAGAGU |
| 50 | GUAAACAUCCUUGACUGGAAGCUUdTdT |
| | \|\|\|\|\|\|\|\|\|\|\|\|\|\|\|\|\|\|\|\|\|\|\| |
| | dTdTGAUUUGUAGGAACUGACCUUCGAA |
| 51 | UAGCUUAUCAGACUGACGUUGACdTdT |
| | \|\|\|\|\|\|\|\|\|\|\|\|\|\|\|\|\|\|\|\|\|\| |
| | dTdTUUCGAAUAGUCUGACUGCAACUG |
| 52 | UAGCUUAUCAGACUGAUGUUGACUdTdT |
| | \|\|\|\|\|\|\|\|\|\|\|\|\|\|\|\|\|\|\|\|\|\|\| |
| | dTdTUUCGAAUAGUCUGACUACAACUGA |
| 53 | UAUUGCAGUUGUCCCGGCCUGUdTdT |
| | \|\|\|\|\|\|\|\|\|\|\|\|\|\|\|\|\|\|\|\|\| |
| | dTdTUUAACGUCAACAGGGCCGGACA |
| 54 | UGAGAUGAAGCACUGUAGCAAdTdT |
| | \|\|\|\|\|\|\|\|\|\|\|\|\|\|\|\|\|\|\|\| |
| | dTdTUCUCUACUUCGUGACAUCGUU |
| 55 | UGAGGGGCAGAGAGCGAGAUdTdT |
| | \|\|\|\|\|\|\|\|\|\|\|\|\|\|\|\|\|\|\| |
| | dTdTUCUCCCCGUCUCUCGCUCUA |
| 56 | UGAGGGGCAGAGAGCGAGGCUdTdT |
| | \|\|\|\|\|\|\|\|\|\|\|\|\|\|\|\|\|\|\|\| |
| | dTdTUCUCCCCGUCUCUCGCUCCGA |
| 57 | UGAGGGGCAGAGAGCGAGACCUUdTdT |
| | \|\|\|\|\|\|\|\|\|\|\|\|\|\|\|\|\|\|\|\|\|\| |
| | dTdTUCUCCCCGUCUCUCGCUCUGGAA |
| 58 | UGAGGGGCAGAGAGCGAGAAGAdTdT |
| | \|\|\|\|\|\|\|\|\|\|\|\|\|\|\|\|\|\|\|\|\| |
| | dTdTUCUCCCCGUCUCUCGCUCUUCU |
| 59 | UGAGGUAGUAGGUUGUGUGGUCUdTdT |
| | \|\|\|\|\|\|\|\|\|\|\|\|\|\|\|\|\|\|\|\|\|\| |
| | dTdTUCUCCAUCAUCCAACACACCAGA |
| 60 | UGGAGUGUGACAAUGGUGAdTdT |
| | \|\|\|\|\|\|\|\|\|\|\|\|\|\|\|\|\|\| |
| | dTdTUCCUCACACUGUUACCACU |
| 61 | UGUAAACAUCCCCGACUGAAAGdTdT |
| | \|\|\|\|\|\|\|\|\|\|\|\|\|\|\|\|\|\|\|\|\| |
| | dTdTUCAUUUGUAGGGGCUGACUUUC |
| 62 | UGUAAAUAUCCCCGACUGGAAGCdTdT |
| | \|\|\|\|\|\|\|\|\|\|\|\|\|\|\|\|\|\|\|\|\|\| |
| | dTdTUCAUUUAUAGGGGCUGACCUUCG |
| 63 | UGUAAACAUCCUUGACUGGAAGUdTdT |
| | \|\|\|\|\|\|\|\|\|\|\|\|\|\|\|\|\|\|\|\|\|\| |
| | dTdTUCAUUUGUAGGAACUGACCUUCA |
| 64 | UUGGGAGCGGGCGGGGGGdTdT |
| | \|\|\|\|\|\|\|\|\|\|\|\|\|\|\|\|\| |
| | dTdTUACCCUCGCCCGCCCGCC |
| 65 | UUUAGUAAUGGUAAUGGUUCUdTdT |
| | \|\|\|\|\|\|\|\|\|\|\|\|\|\|\|\|\|\|\|\| |
| | dTdTUAAUCAUUACCAUUACCAAGA |
| 66 | UUUCCGUAGUGUAGUGGUUAUCACGUUCGCdTdT |
| | \|\|\|\|\|\|\|\|\|\|\|\|\|\|\|\|\|\|\|\|\|\|\|\|\|\|\|\|\| |
| | dTdTUAAGGCAUCACAUCACCAAUAGUGCAAGCG |
| 67 | UUUGUUCGUUCGGCUCGCGdTdT |
| | \|\|\|\|\|\|\|\|\|\|\|\|\|\|\|\|\|\| |
| | dTdTUAACAAGCAAGCCGAGCGC |
| 68 | ACGGCGGCGGCGGCGGCGGCGGGGGdTdT |
| | \|\|\|\|\|\|\|\|\|\|\|\|\|\|\|\|\|\|\|\|\|\|\|\| |
| | dTdTAGCCGCCGCCGCCGCCGCCGCCCCC |
| 69 | AGAGGAGGAAGAGGAGGAGGAdTdT |
| | \|\|\|\|\|\|\|\|\|\|\|\|\|\|\|\|\|\|\|\| |
| | dTdTACUCCUCCUUCUCCUCCUCCU |
| 70 | AGGCUGGUCCGAUGGUAGUGGGUUAUCAGAAdTdT |
| | \|\|\|\|\|\|\|\|\|\|\|\|\|\|\|\|\|\|\|\|\|\|\|\|\|\|\|\|\|\| |
| | dTdTACCGACCAGGCUACCAUCACCCAAUAGUCUU |
| 71 | AGGGCGGCGGCGGCGGCGGCGGGGGdTdT |
| | \|\|\|\|\|\|\|\|\|\|\|\|\|\|\|\|\|\|\|\|\|\|\|\| |
| | dTdTACCCGCCGCCGCCGCCGCCGCCCCC |
| 72 | AGGGCGGCGGCGGCGGCGGCGGGGdTdT |
| | \|\|\|\|\|\|\|\|\|\|\|\|\|\|\|\|\|\|\|\|\|\|\| |
| | dTdTACCCGCCGCCGCCGCCGCCGCCCC |
| 73 | AUUCCGGUUGUUCUUCUCdTdT |
| | \|\|\|\|\|\|\|\|\|\|\|\|\|\|\|\|\| |
| | dTdTAAAGGCCAACAAGAAGAG |
| 74 | AUGAGGUAGUAGGUUGUGUGGdTdT |
| | \|\|\|\|\|\|\|\|\|\|\|\|\|\|\|\|\|\|\|\| |
| | dTdTAACUCCAUCAUCCAACACACC |
| 75 | AACAUUCAACGCUGUCGGUGUdTdT |
| | \|\|\|\|\|\|\|\|\|\|\|\|\|\|\|\|\|\|\|\| |
| | dTdTAUGUAAGUUGCGACAGCCACA |
| 76 | CAACACCAGUCGAUGGGCUGUdTdT |
| | \|\|\|\|\|\|\|\|\|\|\|\|\|\|\|\|\|\|\|\| |
| | dTdTCUUGUGGUCAGCUACCCGACA |
| 77 | CAACGGAAUCCCAAAAGCdTdT |
| | \|\|\|\|\|\|\|\|\|\|\|\|\|\|\|\|\| |
| | dTdTCUUGCCUUAGGGUUUUCG |
| 78 | AACCGGGCGGAAACACCAAAdTdT |
| | \|\|\|\|\|\|\|\|\|\|\|\|\|\|\|\|\|\|\| |
| | dTdTAUGGCCCGCCUUUGUGGUUU |
| 79 | CAUUCUUGCGACCCGGGUdTdT |
| | \|\|\|\|\|\|\|\|\|\|\|\|\|\|\|\|\| |
| | dTdTCUAAGAACGCUGGGCCCA |
| 80 | CCCCUGUGGUCUAGUGGUUAGGAUUCGGCGCUCdTdT |
| | \|\|\|\|\|\|\|\|\|\|\|\|\|\|\|\|\|\|\|\|\|\|\|\|\|\|\|\|\|\|\|\| |
| | dTdTCGGGACACCAGAUCACCAAUCCUAAGCCGCGAG |
| 81 | AAAAGCUGGGUUGAGAGGGAdTdT |
| | \|\|\|\|\|\|\|\|\|\|\|\|\|\|\|\|\|\|\| |
| | dTdTAUUUCGACCCAACUCUCCCU |
| 82 | CUGGUCCGAAGGUAGUGAGUUAUCUCAAdTdT |
| | \|\|\|\|\|\|\|\|\|\|\|\|\|\|\|\|\|\|\|\|\|\|\|\|\|\|\| |
| | dTdTCACCAGGCUUCCAUCACUCAAUAGAGUU |
| 83 | AAAAGCUGGGCUGAGAGGGCGdTdT |
| | \|\|\|\|\|\|\|\|\|\|\|\|\|\|\|\|\|\|\|\| |
| | dTdTAUUUCGACCCGACUCUCCCGC |
| 84 | AAAAGCUGGGUUGAGAGGGAdTdT |
| | \|\|\|\|\|\|\|\|\|\|\|\|\|\|\|\|\|\|\| |
| | dTdTAUUUCGACCCAACUCUCCCU |
| 85 | AAAAGCUGGGUUGAGAGGGCGAGdTdT |
| | \|\|\|\|\|\|\|\|\|\|\|\|\|\|\|\|\|\|\|\|\|\| |
| | dTdTAUUUCGACCCAACUCUCCCGCUC |
| 86 | AAGCUGCCAGUUGAAGAACUGCdTdT |
| | \|\|\|\|\|\|\|\|\|\|\|\|\|\|\|\|\|\|\|\|\| |
| | dTdTAUCGACGGUCAACUUCUUGACG |
| 87 | AGCUACAUUGUCUGCUGGGUUUdTdT |
| | \|\|\|\|\|\|\|\|\|\|\|\|\|\|\|\|\|\|\|\|\| |
| | dTdTACGAUGUAACAGACGACCCAAA |
| 88 | AGCUCGGCCUGAGGCCCCUCAGUdTdT |
| | \|\|\|\|\|\|\|\|\|\|\|\|\|\|\|\|\|\|\|\|\|\| |
| | dTdTACGAGCCGGACUCCGGGGAGUCA |
| 89 | AGGCUGGUCCGAUGGUAGUGGGUUAUCAGAACUdTdT |
| | \|\|\|\|\|\|\|\|\|\|\|\|\|\|\|\|\|\|\|\|\|\|\|\|\|\|\|\|\|\|\|\| |
| | dTdTACCGACCAGGCUACCAUCACCCAAUAGUCUUGA |
| 90 | AGGGCGGCGGCGGCGGCGGCGGGGGdTdT |
| | \|\|\|\|\|\|\|\|\|\|\|\|\|\|\|\|\|\|\|\|\|\|\|\| |
| | dTdTACCCGCCGCCGCCGCCGCCGCCCCC |
| 91 | AUAUUGCACUUGUCCCGGCCUGUdTdT |
| | \|\|\|\|\|\|\|\|\|\|\|\|\|\|\|\|\|\|\|\|\|\| |
| | dTdTAAUAACGUGAACAGGGCCGGACA |
| 92 | AUCCUGUACUGAGCUGCCCCGAdTdT |
| | \|\|\|\|\|\|\|\|\|\|\|\|\|\|\|\|\|\|\|\|\| |
| | dTdTAAGGACAUGACUCGACGGGGCU |
| 93 | AUCCUGUACUGAGCUGCCCCGAGdTdT |
| | \|\|\|\|\|\|\|\|\|\|\|\|\|\|\|\|\|\|\|\|\|\| |
| | dTdTAAGGACAUGACUCGACGGGGCUC |
| 94 | AUUGCACUUGUCCCGGCCUGdTdT |
| | \|\|\|\|\|\|\|\|\|\|\|\|\|\|\|\|\|\|\| |
| | dTdTAAACGUGAACAGGGCCGGAC |
| 95 | AUUGCACUUGUCCCGGCCUGUdTdT |
| | \|\|\|\|\|\|\|\|\|\|\|\|\|\|\|\|\|\|\|\| |
| | dTdTAAACGUGAACAGGGCCGGACA |
| 96 | AUUGCACUUGCCCCGGCCUGUdTdT |
| | \|\|\|\|\|\|\|\|\|\|\|\|\|\|\|\|\|\|\|\| |
| | dTdTAAACGUGAACGGGGCCGGACA |
| 97 | CAUUGCACUUGUCCCGGCCUGdTdT |
| | \|\|\|\|\|\|\|\|\|\|\|\|\|\|\|\|\|\|\|\| |
| | dTdTCUAACGUGAACAGGGCCGGAC |
| 98 | CAUUGCACUUGUCUCGGUCUGGdTdT |
| | \|\|\|\|\|\|\|\|\|\|\|\|\|\|\|\|\|\|\|\|\| |
| | dTdTCUAACGUGAACAGAGCCAGACC |
| 99 | CAUUGGUGGUUCAGUGGUAGAAUUCUCGCCdTdT |
| | \|\|\|\|\|\|\|\|\|\|\|\|\|\|\|\|\|\|\|\|\|\|\|\|\|\|\|\|\| |
| | dTdTCUAACCACCAAGUCACCAUCUUAAGAGCGG |
| 100 | CCCCCCGCUGCUAAAUUUGACUGGCUUdTdT |
| | \|\|\|\|\|\|\|\|\|\|\|\|\|\|\|\|\|\|\|\|\|\|\|\|\|\| |
| | dTdTCGGGGGCGACGAUUUAAACUGACCGAA |
| 101 | CCCUGUACUGAGCUGCCCCGAGAdTdT |
| | \|\|\|\|\|\|\|\|\|\|\|\|\|\|\|\|\|\|\|\|\|\| |
| | dTdTCGGACAUGACUCGACGGGGCUCU |
| 102 | CCUGUACUGAGCUGCCCCGAGAdTdT |
| | \|\|\|\|\|\|\|\|\|\|\|\|\|\|\|\|\|\|\|\|\| |
| | dTdTCGACAUGACUCGACGGGGCUCU |
| 103 | CCUGUACUGAGCUGCCCCGAGdTdT |
| | \|\|\|\|\|\|\|\|\|\|\|\|\|\|\|\|\|\|\|\| |
| | dTdTCGACAUGACUCGACGGGGCUC |
| 104 | CGAGGAGGAAGAGGAGGACGdTdT |
| | \|\|\|\|\|\|\|\|\|\|\|\|\|\|\|\|\|\|\| |
| | dTdTCCUCCUCCUUCUCCUCCUGC |
| 105 | CGAGGUAGUAGGUUGUAUAGdTdT |
| | \|\|\|\|\|\|\|\|\|\|\|\|\|\|\|\|\|\|\| |
| | dTdTCCUCCAUCAUCCAACAUAUC |
| 106 | CUAUACGACCUGCUGCCUUUdTdT |
| | \|\|\|\|\|\|\|\|\|\|\|\|\|\|\|\|\|\|\| |
| | dTdTCAUAUGCUGGACGACGGAAA |
| 107 | CUGUACUGAGCUGCCCCGAGdTdT |
| | \|\|\|\|\|\|\|\|\|\|\|\|\|\|\|\|\|\|\| |
| | dTdTCACAUGACUCGACGGGGCUC |
| 108 | GAAUUGGUGGUUCAGUGGUAGAAUUCUCGCdTdT |
| | \|\|\|\|\|\|\|\|\|\|\|\|\|\|\|\|\|\|\|\|\|\|\|\|\|\|\|\|\| |
| | dTdTGUUAACCACCAAGUCACCAUCUUAAGAGCG |
| 109 | GCAUCGGUGGUUCAGUGGUAGAAUUCUCGCCUdTdT |
| | \|\|\|\|\|\|\|\|\|\|\|\|\|\|\|\|\|\|\|\|\|\|\|\|\|\|\|\|\|\|\| |
| | dTdTGGUAGCCACCAAGUCACCAUCUUAAGAGCGGA |
| 110 | GCAUUAGUGGUUCAGUGGUAGAAUUCUCGCCUdTdT |
| | \|\|\|\|\|\|\|\|\|\|\|\|\|\|\|\|\|\|\|\|\|\|\|\|\|\|\|\|\|\|\| |
| | dTdTGGUAAUCACCAAGUCACCAUCUUAAGAGCGGA |
| 111 | GCAUUGAUGGUUCAGUGGUAGAAUUCUCGCCdTdT |
| | \|\|\|\|\|\|\|\|\|\|\|\|\|\|\|\|\|\|\|\|\|\|\|\|\|\|\|\|\|\| |
| | dTdTGGUAACUACCAAGUCACCAUCUUAAGAGCGG |
| 112 | GCAUUGGUAGUUCAGUGGUAGAAUUCUCGCCdTdT |
| | \|\|\|\|\|\|\|\|\|\|\|\|\|\|\|\|\|\|\|\|\|\|\|\|\|\|\|\|\|\| |
| | dTdTGGUAACCAUCAAGUCACCAUCUUAAGAGCGG |
| 113 | GCAUUGGUGGUUCAGUGGUAGAAUUCUCGCCCGdTdT |
| | \|\|\|\|\|\|\|\|\|\|\|\|\|\|\|\|\|\|\|\|\|\|\|\|\|\|\|\|\|\|\|\| |
| | dTdTGGUAACCACCAAGUCACCAUCUUAAGAGCGGGC |
| 114 | GCAUUGGUGGUUCAGUGGUAGAAUUCUCGUCdTdT |
| | \|\|\|\|\|\|\|\|\|\|\|\|\|\|\|\|\|\|\|\|\|\|\|\|\|\|\|\|\|\| |
| | dTdTGGUAACCACCAAGUCACCAUCUUAAGAGCAG |
| 115 | GCAUUGGUGGUUCAGUGGUAGAAUUUUCGCCUdTdT |
| | \|\|\|\|\|\|\|\|\|\|\|\|\|\|\|\|\|\|\|\|\|\|\|\|\|\|\|\|\|\|\| |
| | dTdTGGUAACCACCAAGUCACCAUCUUAAAAGCGGA |
| 116 | GCAUUGGUGGUUCAGUGGUAGGAUUCUCGCCdTdT |
| | \|\|\|\|\|\|\|\|\|\|\|\|\|\|\|\|\|\|\|\|\|\|\|\|\|\|\|\|\|\| |
| | dTdTGGUAACCACCAAGUCACCAUCCUAAGAGCGG |
| 117 | GCAUUGGUGGUUCAGUGGUAGAGUUCUCGCCdTdT |
| | \|\|\|\|\|\|\|\|\|\|\|\|\|\|\|\|\|\|\|\|\|\|\|\|\|\|\|\|\|\| |
| | dTdTGGUAACCACCAAGUCACCAUCUCAAGAGCGG |
| 118 | GCAUUGGUGGUUCGGUGGUAGAAUUCUCGCCdTdT |
| | \|\|\|\|\|\|\|\|\|\|\|\|\|\|\|\|\|\|\|\|\|\|\|\|\|\|\|\|\|\| |
| | dTdTGGUAACCACCAAGCCACCAUCUUAAGAGCGG |
| 119 | GCGCCGCUGGUGUAGUGGCAUCAUGCAAGAUUdTdT |
| | \|\|\|\|\|\|\|\|\|\|\|\|\|\|\|\|\|\|\|\|\|\|\|\|\|\|\|\|\|\|\| |
| | dTdTGGCGGCGACCACAUCACCGUAGUACGUUCUAA |
| 120 | GCGUUGGUGGUUCAGUGGUAGAAUUCUCGCCdTdT |
| | \|\|\|\|\|\|\|\|\|\|\|\|\|\|\|\|\|\|\|\|\|\|\|\|\|\|\|\|\|\| |
| | dTdTGGCAACCACCAAGUCACCAUCUUAAGAGCGG |
| 121 | GGCUGGCCCGAUGGUAGUGGGUUAUCAGAACUUAdTdT |
| | \|\|\|\|\|\|\|\|\|\|\|\|\|\|\|\|\|\|\|\|\|\|\|\|\|\|\|\|\|\|\|\|\| |
| | dTdTGCGACCGGGCUACCAUCACCCAAUAGUCUUGAAU |
| 122 | GGCUGGUCCGAAGGUAGUGAGUUACCUCAAdTdT |
| | \|\|\|\|\|\|\|\|\|\|\|\|\|\|\|\|\|\|\|\|\|\|\|\|\|\|\|\|\| |
| | dTdTGCGACCAGGCUUCCAUCACUCAAUGGAGUU |
| 123 | GGCUGGUCCGAAGGCAGUGAGUUAUCUCAAdTdT |
| | \|\|\|\|\|\|\|\|\|\|\|\|\|\|\|\|\|\|\|\|\|\|\|\|\|\|\|\|\| |
| | dTdTGCGACCAGGCUUCCGUCACUCAAUAGAGUU |
| 124 | GGCUGGUCCGAUAGUAGUGGGUUAUCAGAACUUdTdT |
| | \|\|\|\|\|\|\|\|\|\|\|\|\|\|\|\|\|\|\|\|\|\|\|\|\|\|\|\|\|\|\|\| |
| | dTdTGCGACCAGGCUAUCAUCACCCAAUAGUCUUGAA |
| 125 | GGCUGGUCCGAUGGUAGUGGGUCAUCAGAAdTdT |
| | \|\|\|\|\|\|\|\|\|\|\|\|\|\|\|\|\|\|\|\|\|\|\|\|\|\|\|\|\| |
| | dTdTGCGACCAGGCUACCAUCACCCAGUAGUCUU |
| 126 | GGCUGGUCCGAUGGUAGCGGGUUAUCAGAAdTdT |
| | \|\|\|\|\|\|\|\|\|\|\|\|\|\|\|\|\|\|\|\|\|\|\|\|\|\|\|\|\| |
| | dTdTGCGACCAGGCUACCAUCGCCCAAUAGUCUU |
| 127 | GGCUGGUCCGAUGGUAGUGGGUUGUCAGAACUUdTdT |
| | \|\|\|\|\|\|\|\|\|\|\|\|\|\|\|\|\|\|\|\|\|\|\|\|\|\|\|\|\|\|\|\| |
| | dTdTGCGACCAGGCUACCAUCACCCAACAGUCUUGAA |
| 128 | GGCUGGUCCGAUGGUAGUAGGUUAUCAGAACdTdT |
| | \|\|\|\|\|\|\|\|\|\|\|\|\|\|\|\|\|\|\|\|\|\|\|\|\|\|\|\|\|\| |
| | dTdTGCGACCAGGCUACCAUCAUCCAAUAGUCUUG |
| 129 | GGGGAAUUGCGAGCGGAUAACdTdT |
| | \|\|\|\|\|\|\|\|\|\|\|\|\|\|\|\|\|\|\|\| |
| | dTdTGCCCUUAACGCUCGCCUAUUG |
| 130 | GGGGAAUUGUGAGCGGAUAAUdTdT |
| | \|\|\|\|\|\|\|\|\|\|\|\|\|\|\|\|\|\|\|\| |
| | dTdTGCCCUUAACACUCGCCUAUUA |
| 131 | GGGGAAUUGUGAGCGGACAACdTdT |
| | \|\|\|\|\|\|\|\|\|\|\|\|\|\|\|\|\|\|\|\| |
| | dTdTGCCCUUAACACUCGCCUGUUG |
| 132 | UAAGCGGCGGGGUCGGAACAGGdTdT |
| | \|\|\|\|\|\|\|\|\|\|\|\|\|\|\|\|\|\|\|\|\| |
| | dTdTUUUCGCCGCCCCAGCCUUGUCC |
| 133 | UAAGGGGCAGAGAGCGAGACUdTdT |
| | \|\|\|\|\|\|\|\|\|\|\|\|\|\|\|\|\|\|\|\| |
| | dTdTUUUCCCCGUCUCUCGCUCUGA |
| 134 | UAAUGCACUUGUCCCGGCCUGUdTdT |
| | \|\|\|\|\|\|\|\|\|\|\|\|\|\|\|\|\|\|\|\|\| |
| | dTdTUUUACGUGAACAGGGCCGGACA |
| 135 | UAGCAGCACGUAAAUAUCGGCGdTdT |
| | \|\|\|\|\|\|\|\|\|\|\|\|\|\|\|\|\|\|\|\|\| |
| | dTdTUUCGUCGUGCAUUUAUAGCCGC |
| 136 | UAGCUUAUCAGACUGACGUUGAdTdT |
| | \|\|\|\|\|\|\|\|\|\|\|\|\|\|\|\|\|\|\|\|\| |
| | dTdTUUCGAAUAGUCUGACUGCAACU |
| 137 | UAGCUUAUCAGACUGAUGUUAAdTdT |
| | \|\|\|\|\|\|\|\|\|\|\|\|\|\|\|\|\|\|\|\|\| |
| | dTdTUUCGAAUAGUCUGACUACAAUU |
| 138 | UAGCUUAUCAGACUGAUGUUdTdT |
| | \|\|\|\|\|\|\|\|\|\|\|\|\|\|\|\|\|\|\| |
| | dTdTUUCGAAUAGUCUGACUACAA |
| 139 | UAUUGCACUUGUCACGGCCUGUdTdT |
| | \|\|\|\|\|\|\|\|\|\|\|\|\|\|\|\|\|\|\|\|\| |
| | dTdTUUAACGUGAACAGUGCCGGACA |
| 140 | UAUUGCACUUGCCCCGGCCUGUdTdT |
| | \|\|\|\|\|\|\|\|\|\|\|\|\|\|\|\|\|\|\|\|\| |
| | dTdTUUAACGUGAACGGGGCCGGACA |
| 141 | UAUUGCACUUGUCCCGGUCUGdTdT |
| | \|\|\|\|\|\|\|\|\|\|\|\|\|\|\|\|\|\|\|\| |
| | dTdTUUAACGUGAACAGGGCCAGAC |
| 142 | UAUUGCACUUGUCCCAGCCUGUdTdT |
| | \|\|\|\|\|\|\|\|\|\|\|\|\|\|\|\|\|\|\|\|\| |
| | dTdTUUAACGUGAACAGGGUCGGACA |
| 143 | UAUUGCACUCGUCCCGGCCUGUdTdT |
| | \|\|\|\|\|\|\|\|\|\|\|\|\|\|\|\|\|\|\|\|\| |
| | dTdTUUAACGUGAGCAGGGCCGGACA |
| 144 | UAUUGCACUUGUCCCGGCCAGUdTdT |
| | \|\|\|\|\|\|\|\|\|\|\|\|\|\|\|\|\|\|\|\|\| |
| | dTdTUUAACGUGAACAGGGCCGGUCA |
| 145 | UAUUGCACUUGUCCCGGCCCGdTdT |
| | \|\|\|\|\|\|\|\|\|\|\|\|\|\|\|\|\|\|\|\| |
| | dTdTUUAACGUGAACAGGGCCGGGC |
| 146 | UAUUGCACUUGUCCCGGCCUGdTdT |
| | \|\|\|\|\|\|\|\|\|\|\|\|\|\|\|\|\|\|\|\| |
| | dTdTUUAACGUGAACAGGGCCGGAC |
| 147 | UAUUGCGCUUGUCCCGGCCUGdTdT |
| | \|\|\|\|\|\|\|\|\|\|\|\|\|\|\|\|\|\|\|\| |
| | dTdTUUAACGCGAACAGGGCCGGAC |
| 148 | UAUUGCACUUGUCCCUGCCUGUdTdT |
| | \|\|\|\|\|\|\|\|\|\|\|\|\|\|\|\|\|\|\|\|\| |
| | dTdTUUAACGUGAACAGGGACGGACA |
| 149 | UAUUGCACUUAUCCCGGCCUGUdTdT |
| | \|\|\|\|\|\|\|\|\|\|\|\|\|\|\|\|\|\|\|\|\| |
| | dTdTUUAACGUGAAUAGGGCCGGACA |
| 150 | UAUUGCACUUGUCCAGGCCUGUdTdT |
| | \|\|\|\|\|\|\|\|\|\|\|\|\|\|\|\|\|\|\|\|\| |
| | dTdTUUAACGUGAACAGGUCCGGACA |
| 151 | UAUUGCUCUUGUCCCGGCCUGUdTdT |
| | \|\|\|\|\|\|\|\|\|\|\|\|\|\|\|\|\|\|\|\|\| |
| | dTdTUUAACGAGAACAGGGCCGGACA |
| 152 | UAUUGCACUUGUCCCGGCCUGUGdTdT |
| | \|\|\|\|\|\|\|\|\|\|\|\|\|\|\|\|\|\|\|\|\|\| |
| | dTdTUUAACGUGAACAGGGCCGGACAC |
| 153 | UCCCGUACUGAGCUGCCCCGAGAdTdT |
| | \|\|\|\|\|\|\|\|\|\|\|\|\|\|\|\|\|\|\|\|\|\| |
| | dTdTUGGGCAUGACUCGACGGGGCUCU |
| 154 | UCCCGUACUGAGCUGCCCCGAGdTdT |
| | \|\|\|\|\|\|\|\|\|\|\|\|\|\|\|\|\|\|\|\|\| |
| | dTdTUGGGCAUGACUCGACGGGGCUC |
| 155 | UCCCGUACUGAGCUGCCCCGdTdT |
| | \|\|\|\|\|\|\|\|\|\|\|\|\|\|\|\|\|\|\| |
| | dTdTUGGGCAUGACUCGACGGGGC |
| 156 | UCCCUGGUGGUCUAAUGGUUAGGAUUCGGCGCdTdT |
| | \|\|\|\|\|\|\|\|\|\|\|\|\|\|\|\|\|\|\|\|\|\|\|\|\|\|\|\|\|\|\| |
| | dTdTUGGGACCACCAGAUUACCAAUCCUAAGCCGCG |
| 157 | UCCCUGGUGGUCUAGUAGUUAGGAUUCGGCGCdTdT |
| | \|\|\|\|\|\|\|\|\|\|\|\|\|\|\|\|\|\|\|\|\|\|\|\|\|\|\|\|\|\|\|\| |
| | dTdTUGGGACCACCAGAUCAUCAAUCCUAAGCCGCG |
| 158 | UCCCUGGUGAUCUAGUGGUUAGGAUUCGGCGCUdTdT |
| | \|\|\|\|\|\|\|\|\|\|\|\|\|\|\|\|\|\|\|\|\|\|\|\|\|\|\|\|\|\|\|\| |
| | dTdTUGGGACCACUAGAUCACCAAUCCUAAGCCGCGA |
| 159 | UCCCUGGUGGUCUAGUGGUCAGGAUUCGGCGCUCUdTdT |
| | \|\|\|\|\|\|\|\|\|\|\|\|\|\|\|\|\|\|\|\|\|\|\|\|\|\|\|\|\|\|\|\|\|\| |
| | dTdTUGGGACCACCAGAUCACCAGUCCUAAGCCGCGAGA |
| 160 | UCCUGCACUGAGCUGCCCCGAGAdTdT |
| | \|\|\|\|\|\|\|\|\|\|\|\|\|\|\|\|\|\|\|\|\|\| |
| | dTdTUGGACGUGACUCGACGGGGCUCU |
| 161 | UCCUGUACUAAGCUGCCCCGAGAdTdT |
| | \|\|\|\|\|\|\|\|\|\|\|\|\|\|\|\|\|\|\|\|\|\| |
| | dTdTUGGACAUGAUUCGACGGGGCUCU |
| 162 | UCCUGUACCGAGCUGCCCCGAGUdTdT |
| | \|\|\|\|\|\|\|\|\|\|\|\|\|\|\|\|\|\|\|\|\|\| |
| | dTdTUGGACAUGGCUCGACGGGGCUCA |
| 163 | UCCUGUACUGAGCUGCCCCAAGdTdT |
| | \|\|\|\|\|\|\|\|\|\|\|\|\|\|\|\|\|\|\|\|\| |
| | dTdTUGGACAUGACUCGACGGGGUUC |
| 164 | UCCUGUACUGAGCUGCCCCGGGAdTdT |
| | \|\|\|\|\|\|\|\|\|\|\|\|\|\|\|\|\|\|\|\|\|\| |
| | dTdTUGGACAUGACUCGACGGGGCCCU |
| 165 | UCCUGUACUGAGCUGCCCCGUGdTdT |
| | \|\|\|\|\|\|\|\|\|\|\|\|\|\|\|\|\|\|\|\|\| |
| | dTdTUGGACAUGACUCGACGGGGCAC |
| 166 | UCCUGUACCGAGCUGCCCCGAGAdTdT |
| | \|\|\|\|\|\|\|\|\|\|\|\|\|\|\|\|\|\|\|\|\|\| |
| | dTdTUGGACAUGGCUCGACGGGGCUCU |
| 167 | UCCUGUACUGAGCUGCCACGAdTdT |
| | \|\|\|\|\|\|\|\|\|\|\|\|\|\|\|\|\|\|\|\| |
| | dTdTUGGACAUGACUCGACGGUGCU |
| 168 | UCCUGUACCGAGCUGCCCCGAGdTdT |
| | \|\|\|\|\|\|\|\|\|\|\|\|\|\|\|\|\|\|\|\|\| |
| | dTdTUGGACAUGGCUCGACGGGGCUC |
| 169 | UCCUGUACUGAGCUGCCCCGUdTdT |
| | \|\|\|\|\|\|\|\|\|\|\|\|\|\|\|\|\|\|\|\| |
| | dTdTUGGACAUGACUCGACGGGGCA |
| 170 | UCCUGUACUGAGCUGCCCCAAGAdTdT |
| | \|\|\|\|\|\|\|\|\|\|\|\|\|\|\|\|\|\|\|\|\|\| |
| | dTdTUGGACAUGACUCGACGGGGUUCU |
| 171 | UCCUGUACUGAGUUGCCCCGAGdTdT |
| | \|\|\|\|\|\|\|\|\|\|\|\|\|\|\|\|\|\|\|\|\| |
| | dTdTUGGACAUGACUCAACGGGGCUC |
| 172 | UCCUGUACUGGGCUGCCCCGAGUdTdT |
| | \|\|\|\|\|\|\|\|\|\|\|\|\|\|\|\|\|\|\|\|\|\| |
| | dTdTUGGACAUGACCCGACGGGGCUCA |
| 173 | UCCUGUACUGGGCUGCCCCGAGdTdT |
| | \|\|\|\|\|\|\|\|\|\|\|\|\|\|\|\|\|\|\|\|\| |
| | dTdTUGGACAUGACCCGACGGGGCUC |
| 174 | UCCUGUACUGAGCUGCCCGAGdTdT |
| | \|\|\|\|\|\|\|\|\|\|\|\|\|\|\|\|\|\|\|\| |
| | dTdTUGGACAUGACUCGACGGGCUC |
| 175 | UGAGAACUGAAUUCCAUGGGUdTdT |
| | \|\|\|\|\|\|\|\|\|\|\|\|\|\|\|\|\|\|\|\| |
| | dTdTUCUCUUGACUUAAGGUACCCA |
| 176 | UGAGGGGCAGAGAGCGAGACCdTdT |
| | \|\|\|\|\|\|\|\|\|\|\|\|\|\|\|\|\|\|\|\| |
| | dTdTUCUCCCCGUCUCUCGCUCUGG |
| 177 | UGAGGGACAGAGAGCGAGACUdTdT |
| | \|\|\|\|\|\|\|\|\|\|\|\|\|\|\|\|\|\|\|\| |
| | dTdTUCUCCCUGUCUCUCGCUCUGA |
| 178 | UGAGGGGCAGAGAGCGGGACUUUUdTdT |
| | \|\|\|\|\|\|\|\|\|\|\|\|\|\|\|\|\|\|\|\|\|\|\| |
| | dTdTUCUCCCCGUCUCUCGCCCUGAAAA |
| 179 | UGAGGGGCAGAGGGCGAGACUUUdTdT |
| | \|\|\|\|\|\|\|\|\|\|\|\|\|\|\|\|\|\|\|\|\|\| |
| | dTdTUCUCCCCGUCUCCCGCUCUGAAA |
| 180 | UGAGGUAGUAGGUCGUGUGGUUUdTdT |
| | \|\|\|\|\|\|\|\|\|\|\|\|\|\|\|\|\|\|\|\|\|\| |
| | dTdTUCUCCAUCAUCCAGCACACCAAA |
| 181 | UGAGGUAGUAGGUUGUGUGGUCUdTdT |
| | \|\|\|\|\|\|\|\|\|\|\|\|\|\|\|\|\|\|\|\|\|\| |
| | dTdTUCUCCAUCAUCCAACACACCAGA |
| 182 | UGAGGUAGUAGGUCGUGUGGdTdT |
| | \|\|\|\|\|\|\|\|\|\|\|\|\|\|\|\|\|\|\| |
| | dTdTUCUCCAUCAUCCAGCACACC |
| 183 | UGAGGUAGUAGGUUGUAUAGdTdT |
| | \|\|\|\|\|\|\|\|\|\|\|\|\|\|\|\|\|\|\| |
| | dTdTUCUCCAUCAUCCAACAUAUC |
| 184 | UGAGGUAGUAGUUUGCGCUGUdTdT |
| | \|\|\|\|\|\|\|\|\|\|\|\|\|\|\|\|\|\|\|\| |
| | dTdTUCUCCAUCAUCAAACGCGACA |
| 185 | UGAGGUAGUAGGUCGUAUAGdTdT |
| | \|\|\|\|\|\|\|\|\|\|\|\|\|\|\|\|\|\|\| |
| | dTdTUCUCCAUCAUCCAGCAUAUC |
| 186 | UGAGGUAGUAGGUCGUAUAGUdTdT |
| | \|\|\|\|\|\|\|\|\|\|\|\|\|\|\|\|\|\|\|\| |
| | dTdTUCUCCAUCAUCCAGCAUAUCA |
| 187 | UGAGGUAGUAGGUUGUGUGdTdT |
| | \|\|\|\|\|\|\|\|\|\|\|\|\|\|\|\|\|\| |
| | dTdTUCUCCAUCAUCCAACACAC |
| 188 | UGAGGUAGUAGGUUAUGUGGUUdTdT |
| | \|\|\|\|\|\|\|\|\|\|\|\|\|\|\|\|\|\|\|\|\| |
| | dTdTUCUCCAUCAUCCAAUACACCAA |
| 189 | UGAGGUAGUGGAUUGUAUAGUUdTdT |
| | \|\|\|\|\|\|\|\|\|\|\|\|\|\|\|\|\|\|\|\|\| |
| | dTdTUCUCCAUCACCUAACAUAUCAA |
| 190 | UGAGGUAGUAGUUUGCACAGUUdTdT |
| | \|\|\|\|\|\|\|\|\|\|\|\|\|\|\|\|\|\|\|\|\| |
| | dTdTUCUCCAUCAUCAAACGUGUCAA |
| 191 | UGAGGUAGUAGGUUGUGUGGdTdT |
| | \|\|\|\|\|\|\|\|\|\|\|\|\|\|\|\|\|\|\| |
| | dTdTUCUCCAUCAUCCAACACACC |
| 192 | UGGAGUGUGACAAUGGUGUCUGdTdT |
| | \|\|\|\|\|\|\|\|\|\|\|\|\|\|\|\|\|\|\|\|\| |
| | dTdTUCCUCACACUGUUACCACAGAC |
| 193 | UGGGUUUACGUUGGGAGAACUdTdT |
| | \|\|\|\|\|\|\|\|\|\|\|\|\|\|\|\|\|\|\|\| |
| | dTdTUCCCAAAUGCAACCCUCUUGA |
| 194 | UGUAAACAUCCUUGACUGGAAGdTdT |
| | \|\|\|\|\|\|\|\|\|\|\|\|\|\|\|\|\|\|\|\|\| |
| | dTdTUCAUUUGUAGGAACUGACCUUC |
| 195 | UGUAAACAUCCCCGACUGGAAAdTdT |
| | \|\|\|\|\|\|\|\|\|\|\|\|\|\|\|\|\|\|\|\|\| |
| | dTdTUCAUUUGUAGGGGCUGACCUUU |
| 196 | UGUAAACAUCCUACACUCUCAdTdT |
| | \|\|\|\|\|\|\|\|\|\|\|\|\|\|\|\|\|\|\|\| |
| | dTdTUCAUUUGUAGGAUGUGAGAGU |
| 197 | UGUAGACAUCCUUGACUGGAAGCUdTdT |
| | \|\|\|\|\|\|\|\|\|\|\|\|\|\|\|\|\|\|\|\|\|\|\| |
| | dTdTUCAUCUGUAGGAACUGACCUUCGA |
| 198 | UGUUGCACUUGUCCCGGCCUGUdTdT |
| | \|\|\|\|\|\|\|\|\|\|\|\|\|\|\|\|\|\|\|\|\| |
| | dTdTUCAACGUGAACAGGGCCGGACA |
| 199 | UUCUGUACUGAGCUGCCCCGAGAdTdT |
| | \|\|\|\|\|\|\|\|\|\|\|\|\|\|\|\|\|\|\|\|\|\| |
| | dTdTUAGACAUGACUCGACGGGGCUCU |
| 200 | UUGCACUUGUCCCGGCCUGUdTdT |
| | \|\|\|\|\|\|\|\|\|\|\|\|\|\|\|\|\|\|\| |
| | dTdTUACGUGAACAGGGCCGGACA |
| 201 | UUGGGAGCGGGCGGGCGGdTdT |
| | \|\|\|\|\|\|\|\|\|\|\|\|\|\|\|\|\| |
| | dTdTUACCCUCGCCCGCCCGCC |
| 202 | AUUCCGGUUGUUCUUCUCdTdT |
| | \|\|\|\|\|\|\|\|\|\|\|\|\|\|\|\|\| |
| | dTdTAAAGGCCAACAAGAAGAG |
| 203 | UUGUAGCACUGGGGCCAGAUGGdTdT |
| | \|\|\|\|\|\|\|\|\|\|\|\|\|\|\|\|\|\|\|\|\| |
| | dTdTUACAUCGUGACCCCGGUCUACC |
| 204 | AGAUGAGAGGACGGGACGCCdTdT |
| | \|\|\|\|\|\|\|\|\|\|\|\|\|\|\|\|\|\|\| |
| | dTdTACUACUCUCCUGCCCUGCGG |
| 205 | UAUGGAAGUGCCGCCGCUGAAAdTdT |
| | \|\|\|\|\|\|\|\|\|\|\|\|\|\|\|\|\|\|\|\|\| |
| | dTdTUUACCUUCACGGCGGCGACUUU |
| 206 | UAUUAAAUCAAUCUCGGCUGGCdTdT |
| | \|\|\|\|\|\|\|\|\|\|\|\|\|\|\|\|\|\|\|\|\| |
| | dTdTUUAAUUUAGUUAGAGCCGACCG |
| SEQ ID NO. | Antisense 5′ to 3′ |
| 1 | CCCCCTCAACCCAGCTTTTC |
| 2 | ACGTCAGGACCGCTACGGACCTCCACCAGAGTTTC |
| 3 | TTATCCGCTCACAATTC |
| 4 | TATTTTGCCGACTTCCCTTATC |
| 5 | GGCGAGAATTCTACCACTGAACCACTCATGC |
| 6 | TCCATTGCGCCACTGGCC |
| 7 | GGCCCCGCTTCGCGCCCCAGCCCGACCGACCCAGCC |
| 8 | CCTATATCGCCGACATCACC |
| 9 | GGATGCTCACGATACGGGTTA |
| 10 | GAAGCCAGTCAAATTTAGCAGTGGGGGGTTGTA |
| 11 | GATGGTGGCTAACGGCGGGATA |
| 12 | ACCTGTCCTACGAGTTGCATGA |
| 13 | AACAACAGAATCACTAGTCTTCCA |
| 14 | ATTGAGATAACCCACTACCTTCGGACCA |
| 15 | AAATCTTATCTCATCCGCCAA |
| 16 | CAGGCGAACGTGATAACCACTACACTACGGAAA |
| 17 | GCCCAAGTCAGAGGTGGCGAAA |
| 18 | GGTAAGCCCTCCCGTATCGTAGT |
| 19 | CTAGCAGTGCGCGACGTTCTT |
| 20 | CATACGCCAGTTCACGAGCATG |
| 21 | TTTGGCACCCGAGAATTCCACACG |
| 22 | TGTTTCTCGAGACTACCCGGA |
| 23 | ATAGCCAGGTCCGGGTGTTCA |
| 24 | CGTGGTGCCACGCTCGTCGTTT |
| 25 | GAGTAGATAACGAAGCGAAAG |
| 26 | GTTCGATTCCTTCCTTT |
| 27 | GCCTAGCACGCTTCCGCTGCGCCACTCTGCT |
| 28 | GTCCAGCACGCTTCCGCTGCGCAACTCTGCT |
| 29 | GTTCTAATAACCCACTACCATCGGACCAGCCT |
| 30 | TGAACACCCGGACCTGGTTAT |
| 31 | ACCATCGGCACCACCACGCT |
| 32 | ATAACCGGGTCCGGGTGTTCA |
| 33 | CATGTCCAACTGCTGTT |
| 34 | TGGTATTCTCGCACGGACTACAGCCACGACCAAT |
| 35 | CCATGCCAACCCGTTCCATG |
| 36 | GATCCCAGACACCATCGAATG |
| 37 | GGCGAGAATTCTACTACTGAACCACCAATG |
| 38 | CAATTTCACTGAGTCTCGGG |
| 39 | TCCCCTTCTTTTCTTCCCCTTCTTCTTTCCTTCTT |
| 40 | ATCTCCCTTCTTTCCTCCCGTCTT |
| 41 | GTCCCGCCGCCGCCGCCGCCGCCACCGCCGCCGCCGCCG |
| 42 | AAACCCATGGAATTCAGTTCTC |
| 43 | CCGTCCTCCTCCTCCTCCTC |
| 44 | ACTATGCAACCTACTACCTC |
| 45 | ATTCCGGCAGCGTCTTACTC |
| 46 | TGGCGAGCCCGATCTTCCCCATC |
| 47 | CAGGCGAGAATTCTACCACTGAACCACCAATGC |
| 48 | GAATTCTACCACTGAACCACAAATGC |
| 49 | TGAGATAACTCACTACCTTCGGACCAGCC |
| 50 | AAGCTTCCAGTCAAGGATGTTTAC |
| 51 | GTCAACGTCAGTCTGATAAGCTA |
| 52 | AGTCAACATCAGTCTGATAAGCTA |
| 53 | ACAGGCCGGGACAACTGCAATA |
| 54 | TTGCTACAGTGCTTCATCTCA |
| 55 | ATCTCGCTCTCTGCCCCTCA |
| 56 | AGCCTCGCTCTCTGCCCCTCA |
| 57 | AAGGTCTCGCTCTCTGCCCCTCA |
| 58 | TCTTCTCGCTCTCTGCCCCTCA |
| 59 | AGACCACACAACCTACTACCTCA |
| 60 | TCACCATTGTCACACTCCA |
| 61 | CTTTCAGTCGGGGATGTTTACA |
| 62 | GCTTCCAGTCGGGGATATTTACA |
| 63 | ACTTCCAGTCAAGGATGTTTACA |
| 64 | CCGCCCGCCCGCTCCCAA |
| 65 | AGAACCATTACCATTACTAAA |
| 66 | GCGAACGTGATAACCACTACACTACGGAAA |
| 67 | CGCGAGCCGAACGAACAAA |
| 68 | CCCCCGCCGCCGCCGCCGCCGCCGT |
| 69 | TCCTCCTCCTCTTCCTCCTCT |
| 70 | TTCTGATAACCCACTACCATCGGACCAGCCT |
| 71 | CCCCCGCCGCCGCCGCCGCCGCCCT |
| 72 | CCCCGCCGCCGCCGCCGCCGCCCT |
| 73 | GAGAAGAACAACCGGAAT |
| 74 | CCACACAACCTACTACCTCAT |
| 75 | ACACCGACAGCGTTGAATGTT |
| 76 | ACAGCCCATCGACTGGTGTTG |
| 77 | GCTTTTGGGATTCCGTTG |
| 78 | TTTGGTGTTTCCGCCCGGTT |
| 79 | ACCCGGGTCGCAAGAATG |
| 80 | GAGCGCCGAATCCTAACCACTAGACCACAGGGG |
| 81 | TCCCTCTCAACCCAGCTTTT |
| 82 | TTGAGATAACTCACTACCTTCGGACCAG |
| 83 | CGCCCTCTCAGCCCAGCTTTT |
| 84 | TCCCTCTCAACCCAGCTTTT |
| 85 | CTCGCCCTCTCAACCCAGCTTTT |
| 86 | GCAGTTCTTCAACTGGCAGCTT |
| 87 | AAACCCAGCAGACAATGTAGCT |
| 88 | ACTGAGGGGCCTCAGGCCGAGCT |
| 89 | AGTTCTGATAACCCACTACCATCGGACCAGCCT |
| 90 | CCCCCGCCGCCGCCGCCGCCGCCCT |
| 91 | ACAGGCCGGGACAAGTGCAATAT |
| 92 | TCGGGGCAGCTCAGTACAGGAT |
| 93 | CTCGGGGCAGCTCAGTACAGGAT |
| 94 | CAGGCCGGGACAAGTGCAAT |
| 95 | ACAGGCCGGGACAAGTGCAAT |
| 96 | ACAGGCCGGGGCAAGTGCAAT |
| 97 | CAGGCCGGGACAAGTGCAATG |
| 98 | CCAGACCGAGACAAGTGCAATG |
| 99 | GGCGAGAATTCTACCACTGAACCACCAATG |
| 100 | AAGCCAGTCAAATTTAGCAGGGGGGGG |
| 101 | TCTCGGGGCAGCTCAGTACAGGG |
| 102 | TCTCGGGGCAGCTCAGTACAGG |
| 103 | CTCGGGGCAGCTCAGTACAGG |
| 104 | CGTCCTCCTCTTCCTCCTCG |
| 105 | CTATACAACCTACTACCTCG |
| 106 | AAAGGCAGCAGGTCGTATAG |
| 107 | CTCGGGGCAGCTCAGTACAG |
| 108 | GCGAGAATTCTACCACTGAACCACCAATTC |
| 109 | AGGCGAGAATTCTACCACTGAACCACCGATGC |
| 110 | AGGCGAGAATTCTACCACTGAACCACTAATGC |
| 111 | GGCGAGAATTCTACCACTGAACCATCAATGC |
| 112 | GGCGAGAATTCTACCACTGAACTACCAATGC |
| 113 | CGGGCGAGAATTCTACCACTGAACCACCAATGC |
| 114 | GACGAGAATTCTACCACTGAACCACCAATGC |
| 115 | AGGCGAAAATTCTACCACTGAACCACCAATGC |
| 116 | GGCGAGAATCCTACCACTGAACCACCAATGC |
| 117 | GGCGAGAACTCTACCACTGAACCACCAATGC |
| 118 | GGCGAGAATTCTACCACCGAACCACCAATGC |
| 119 | AATCTTGCATGATGCCACTACACCAGCGGCGC |
| 120 | GGCGAGAATTCTACCACTGAACCACCAACGC |
| 121 | TAAGTTCTGATAACCCACTACCATCGGGCCAGCC |
| 122 | TTGAGGTAACTCACTACCTTCGGACCAGCC |
| 123 | TTGAGATAACTCACTGCCTTCGGACCAGCC |
| 124 | AAGTTCTGATAACCCACTACTATCGGACCAGCC |
| 125 | TTCTGATGACCCACTACCATCGGACCAGCC |
| 126 | TTCTGATAACCCGCTACCATCGGACCAGCC |
| 127 | AAGTTCTGACAACCCACTACCATCGGACCAGCC |
| 128 | GTTCTGATAACCTACTACCATCGGACCAGCC |
| 129 | GTTATCCGCTCGCAATTCCCC |
| 130 | ATTATCCGCTCACAATTCCCC |
| 131 | GTTGTCCGCTCACAATTCCCC |
| 132 | CCTGTTCCGACCCCGCCGCTTA |
| 133 | AGTCTCGCTCTCTGCCCCTTA |
| 134 | ACAGGCCGGGACAAGTGCATTA |
| 135 | CGCCGATATTTACGTGCTGCTA |
| 136 | TCAACGTCAGTCTGATAAGCTA |
| 137 | TTAACATCAGTCTGATAAGCTA |
| 138 | AACATCAGTCTGATAAGCTA |
| 139 | ACAGGCCGTGACAAGTGCAATA |
| 140 | ACAGGCCGGGGCAAGTGCAATA |
| 141 | CAGACCGGGACAAGTGCAATA |
| 142 | ACAGGCTGGGACAAGTGCAATA |
| 143 | ACAGGCCGGGACGAGTGCAATA |
| 144 | ACTGGCCGGGACAAGTGCAATA |
| 145 | CGGGCCGGGACAAGTGCAATA |
| 146 | CAGGCCGGGACAAGTGCAATA |
| 147 | CAGGCCGGGACAAGCGCAATA |
| 148 | ACAGGCAGGGACAAGTGCAATA |
| 149 | ACAGGCCGGGATAAGTGCAATA |
| 150 | ACAGGCCTGGACAAGTGCAATA |
| 151 | ACAGGCCGGGACAAGAGCAATA |
| 152 | CACAGGCCGGGACAAGTGCAATA |
| 153 | TCTCGGGGCAGCTCAGTACGGGA |
| 154 | CTCGGGGCAGCTCAGTACGGGA |
| 155 | CGGGGCAGCTCAGTACGGGA |
| 156 | GCGCCGAATCCTAACCATTAGACCACCAGGGA |
| 157 | GCGCCGAATCCTAACTACTAGACCACCAGGGA |
| 158 | AGCGCCGAATCCTAACCACTAGATCACCAGGGA |
| 159 | AGAGCGCCGAATCCTGACCACTAGACCACCAGGGA |
| 160 | TCTCGGGGCAGCTCAGTGCAGGA |
| 161 | TCTCGGGGCAGCTTAGTACAGGA |
| 162 | ACTCGGGGCAGCTCGGTACAGGA |
| 163 | CTTGGGGCAGCTCAGTACAGGA |
| 164 | TCCCGGGGCAGCTCAGTACAGGA |
| 165 | CACGGGGCAGCTCAGTACAGGA |
| 166 | TCTCGGGGCAGCTCGGTACAGGA |
| 167 | TCGTGGCAGCTCAGTACAGGA |
| 168 | CTCGGGGCAGCTCGGTACAGGA |
| 169 | ACGGGGCAGCTCAGTACAGGA |
| 170 | TCTTGGGGCAGCTCAGTACAGGA |
| 171 | CTCGGGGCAACTCAGTACAGGA |
| 172 | ACTCGGGGCAGCCCAGTACAGGA |
| 173 | CTCGGGGCAGCCCAGTACAGGA |
| 174 | CTCGGGCAGCTCAGTACAGGA |
| 175 | ACCCATGGAATTCAGTTCTCA |
| 176 | GGTCTCGCTCTCTGCCCCTCA |
| 177 | AGTCTCGCTCTCTGTCCCTCA |
| 178 | AAAAGTCCCGCTCTCTGCCCCTCA |
| 179 | AAAGTCTCGCCCTCTGCCCCTCA |
| 180 | AAACCACACGACCTACTACCTCA |
| 181 | AGACCACACAACCTACTACCTCA |
| 182 | CCACACGACCTACTACCTCA |
| 183 | CTATACAACCTACTACCTCA |
| 184 | ACAGCGCAAACTACTACCTCA |
| 185 | CTATACGACCTACTACCTCA |
| 186 | ACTATACGACCTACTACCTCA |
| 187 | CACACAACCTACTACCTCA |
| 188 | AACCACATAACCTACTACCTCA |
| 189 | AACTATACAATCCACTACCTCA |
| 190 | AACTGTGCAAACTACTACCTCA |
| 191 | CCACACAACCTACTACCTCA |
| 192 | CAGACACCATTGTCACACTCCA |
| 193 | AGTTCTCCCAACGTAAACCCA |
| 194 | CTTCCAGTCAAGGATGTTTACA |
| 195 | TTTCCAGTCGGGGATGTTTACA |
| 196 | TGAGAGTGTAGGATGTTTACA |
| 197 | AGCTTCCAGTCAAGGATGTCTACA |
| 198 | ACAGGCCGGGACAAGTGCAACA |
| 199 | TCTCGGGGCAGCTCAGTACAGAA |
| 200 | ACAGGCCGGGACAAGTGCAA |
| 201 | CCGCCCGCCCGCTCCCAA |
| 202 | GAGAAGAACAACCGGAAT |
| 203 | CCATCTGGCCCCAGTGCTACAA |
| 204 | GGCGTCCCGTCCTCTCATCT |
| 205 | TTTCAGCGGCGGCACTTCCATA |
| 206 | GCCAGCCGAGATTGATTTAATA |

### REFERENCES

1. Hage, C. et al., Association of Coronary Microvascular Dysfunction With Heart Failure Hospitalizations and Mortality in Heart Failure With Preserved Ejection Fraction: A Follow-up in the PROMIS-HF-pEF Study. Journal of Cardiac Failure 26, 1016-1021 (2020).
2. Shah S J et al., Research Priorities for Heart Failure With Preserved Ejection Fraction: National Heart, Lung, and Blood Institute Working Group Summary. Circulation 141, 1001-1026 (2020).
3. Sanders-van Wijk, S. et al., Proteomic Evaluation of the Comorbidity-Inflammation Paradigm in Heart Failure With Preserved Ejection Fraction: Results from the PROMIS-HFpEF Study. Circulation 142, 2029-2044 (2020).
4. Hahn, V S et al., Myocardial Gene Expression Signatures in Human Heart Failure With Preserved Ejection Fraction. Circulation 143, 120-134 (2021).
5. Sava, R., Pepine, C, and March K., Immune Dysregulation in HFpEF: A Target for Mesenchymal Stem/Stromal Cell Therapy. JCM 9, 241 (2020).
6. Wagdy K. and Nagy S., EMPEROR-Preserved: SGLT2 inhibitors breakthrough in the management of heart failure with preserved ejection fraction. Global cardiology science & practice vol. 2021, 3 e202117 (2021).
7. Pitt B, et al., Spironolactone for heart failure with preserved ejection fraction. N Engl J Med. 2014 Apr. 10; 370(15): 1383-92.
8. Sinha A, Rahman H, Perera D., Coronary microvascular disease: current concepts of pathophysiology, diagnosis and management. Cardiovasc Endocrinol Metab. 2020 Jul. 16; 10(1):22-30.
9. Camici P G, Crea F. Coronary microvascular dysfunction. N Engl J Med 2007; 356:830-840.

Equations

CFR=2.1013+1.668*(log₁₀(U₁+1)−0.025)+0.1*(log₁₀(U₂+1)−0.062)+0.525*(log₁₀(U₃+1)−0.089)−0.982*(log₁₀(U₄+1)−0.087)−0.45*(log₁₀(U₅+1)−0.128)−0.462*(log₁₀(U₆+1)−0.107)−1.22*(log₁₀(U₇+1)−0.028)−0.878*(log₁₀(U₈+1)−0.059)+0.498*(log₁₀(U₉+1)−0.021)+0.666*(log₁₀(U₁₀+1)−0.012)+0.11*(log₁₀(U₁₁+1)−0.032)−0.034*(log₁₀(U₁₂+1)−0.113)−0.675*(log₁₀(U₁₃+1)−0.054)+0.453*(log₁₀(U₁₄+1)−0.015)−0.19*(log₁₀(U₁₅+1)−0.2)+0.091*(log₁₀(U₁₆+1)−0.013)−0.118*(log₁₀(U₁₇+1)−0.044)+0.226*(log₁₀(U₁₈+1)−0.029)−0.807*(log₁₀(U₁₉+1)−0.051)+0.148*(log₁₀(U₂₀+1)−0.01)+0.62*(log₁₀(U₂₁+1)−0.034)−0.083*(log₁₀(U₂₂+1)−0.074)−0.097*(log₁₀(U₂₃+1)−0.047)+0.663*(log₁₀(U₂₄+1)−0.027)+0.512*(log₁₀(U₂₅+1)−0.094)+1.541*(log₁₀(U₂₆+1)−0.048)+0.068*(log₁₀(U₂₇+1)−0.542)+0.681*(log₁₀(U₂₈+1)−0.012)+0.57*(log₁₀(U₂₉+1)−0.027)−0.247*(log₁₀(U₃₀+1)−0.073)+1.546*(log₁₀(U₃₁+1)−0.019)+0.734*(log₁₀(U₃₂+1)−0.017)−0.291*(log₁₀(U₃₃+1)−0.058)+0.527*(log₁₀(U₃₄+1)−0.01)+1.808*(log₁₀(U₃₅+1)−0.01)+0.498*(log₁₀(U₃₆+1)−0.045)−0.368*(log₁₀(U₃₇+1)−0.033)+0.45*(log₁₀(U₃₈+1)−0.042)−1.029*(log₁₀(U₃₉+1)−0.112)−0.195*(log₁₀(U₄₀+1)−0.02)+0.113*(log₁₀(U₄₁+1)−0.196)+1.695*(log₁₀(U₄₂+1)−0.023) Formula 1

CFR=2.1013+0.833*(log₁₀(U₄₃₊₁)−0.404)+0.434*(log₁₀(U₄₄₊₁)−1.369)−0.758*(log₁₀(U₄₅₊₁)−0.305)+0.697*(log₁₀(U₄₆₊₁)−0.404)+0.817*(log₁₀(U₄₇₊₁)−0.324)−0.316*(log₁₀(U₄₈₊₁)−1.091)−0.73*(log₁₀(U₄₉₊₁)−0.225)+0.559*(log₁₀(U₅₀₊₁)−0.313)−0.539*(log₁₀(U₅₁₊₁)−0.312)−0.743*(log₁₀(U₅₂₊₁)−0.196)+0.429*(log₁₀(U₅₃₊₁)−0.404)−0.511*(log₁₀(U₅₄₊₁)−0.244)−0.297*(log₁₀(U₅₅₊₁)−1.33)+0.328*(log₁₀(U₅₆₊₁)−0.492)−0.261*(log₁₀(U₅₇₊₁)−1.638)+0.492*(log₁₀(U₅₈₊₁)−0.236)+0.361*(log₁₀(U₅₉₊₁)−0.31)−0.401*(log₁₀(UI₆₀₊₁)−0.373)−0.372*(log₁₀(U₆₁₊₁)−0.316)−0.208*(log₁₀(U₆₂₊₁)−0.7)−0.29*(log₁₀(U₆₃₊₁)−0.317)−0.196*(log₁₀(U₆₄₊₁)−0.736)+0.339*(log₁₀(U₆₅₊₁)−0.277)−0.308*(log₁₀(U₆₆₊₁)−0.292)+0.318*(log₁₀(U₆₇₊₁)−0.266)+0.429*(log₁₀(U₆₈₊₁)−0.165)+0.245*(log₁₀(U₆₉₊₁)−0.307)+0.355*(log₁₀(U₇₀₊₁)−0.235)−0.26*(log₁₀(U₇₁₊₁)−0.261)+0.248*(log₁₀(U₇₂₊₁)−0.254)−0.226*(log₁₀(U₇₃₊₁)−0.157)+0.117*(log₁₀(U₇₄₊₁)−0.318)−0.196*(log₁₀(U₇₅₊₁)−0.208)+0.124*(log₁₀(U₇₆₊₁)−0.277)−0.147*(log₁₀(U₇₇₊₁)−0.181)−0.115*(log₁₀(U₇₈₊₁)−0.284)+0.125*(log₁₀(U₇₉₊₁)−0.552)−0.106*(log₁₀(U₈₀₊₁)−0.278)−0.113*(log₁₀(U₈₁₊₁)−0.241)−0.079*(log₁₀(U₈₂₊₁)−0.282)−0.068*(log₁₀(U₈₃₊₁)−0.308)−0.09*(log₁₀(U₈₄₊₁)−0.167) Formula 2

1. A method for evaluating coronary flow reserve (CFR) in a subject, comprising:
providing a blood, serum, or plasma sample from the subject,
determining an expression profile of one or more small RNAs listed in Table 1 in the blood or serum sample, and
based on the expression profile identifying the subject as having CFR of less than 2.5 or a CFR of 2.5 or greater.

2. The method of claim 1, wherein the subject has Heart Failure with Preserved Ejection Fraction (HFpEF).

3. The method of claim 2, wherein a subject with a CRF less than 2.5 is identified to have coronary microvascular dysfunction.

4. The method of claim 2, wherein a subject with a CRF less than 2.5 correlates to higher probability of HFpEF-related morbidity.

5. The method of claim 3 or 4, wherein the subject with a CRF less than 2.5 is treated by surgical or pharmaceutical intervention.

6. The method of claim any one of claims 1 to 5, wherein the expression profile comprises the expression level of at least 10 sRNAs from Table 1.

7. The method of claim 6, wherein the expression profile comprises the expression level of at least 20 sRNAs from Table 1.

8. The method of claim 6, wherein the expression profile comprises the expression level of at least 30 sRNAs from Table 1.

9. The method of claim 6, wherein the expression profile comprises the expression level of at least 40 sRNAs from Table 1.

10. The method of claim 6, wherein the expression profile comprises, consists essentially of, or consists of the expression levels of sRNAs from Table 1.

11. The method of any one of claims 1 to 10, wherein the expression profile is determined by a quantitative PCR assay.

12. The method of claim 11, wherein the sRNAs are reverse transcribed using stem-loop RT primer.

13. The method of claim 12, wherein reverse transcripts are amplified with forward and reverse primers.

14. The method of any one of claims 11 to 13, wherein the quantitative PCR assay employs a fluorescent dye or fluorescent-labeled probe.

15. The method of claim 14, wherein the quantitative PCR assay employs a fluorescent-labeled probe, the probe further comprising a quencher moiety.

16. The method of any one of claims 1 to 10, wherein the expression profile is determined using a hybridization assay.

17. The method of claim 16, wherein the hybridization assay employs a hybridization array comprising sRNA-specific probes.

18. The method of any one of claims 1 to 10, wherein the expression profile is determined by nucleic acid sequencing, and sRNAs are identified in the sample by a process that comprises trimming 5′ and 3′ sequencing adaptors from sRNA sequences.

19. The method of claim 18, wherein RNA from multiple samples are pooled for determining expression profiles, with sequences from different samples containing an identifying sample tag sequence.

20. The method of any one of claims 1 to 19, wherein the expression profile further comprises the expression level of one or more expression normalization controls.

21. The method of any one of claims 1 to 20, wherein the expression profile comprises sRNAs of SEQ ID NOS: 1-42 or 43-84.

22. The method of claim 21, wherein the expression profile is scored according to Formula 1 or Formula 2.

23. A method for risk stratifying a subject diagnosed with Heart Failure Preserved Ejection Fraction (HFpEF), comprising:
providing a blood or serum sample from the subject;
evaluating CFR according to any one of claims 1 to 23, and
based on the expression profile stratifying the subject as at risk of HF progression.

24. The method of claim 23, wherein the subject is determined to have a CRF less than 2.5, and is treated by surgical or pharmaceutical intervention.

25. The method of claim 24, wherein the pharmaceutical intervention is an antisense oligonucleotide or siRNA targeting or mimicking the action of a sRNA of Table 1.

26. A kit for evaluating samples for HFpEF and/or CMD, comprising:
sRNA-specific probes and/or primers configured for detecting one or more sRNAs listed in Table 1 (SEQ ID NOS: 1 to 206).

27. The kit of claim 26, comprising: sRNA-specific probes and/or primers configured for detecting at least 10 sRNAs listed in Table 1 (SEQ ID NOS: 1 to 206), and wherein the probes and/or primers are listed in Table 2.

28. The kit of claim 27, comprising: sRNA-specific probes and/or primers configured for detecting at least 20 sRNAs listed in Table 1 (SEQ ID NOS: 1 to 206), and wherein the probes and/or primers are listed in Table 2.

29. The kit of claim 27, comprising: sRNA-specific probes and/or primers configured for detecting at least 30 sRNAs listed in Table 1 (SEQ ID NOS: 1 to 206), and wherein the probes and/or primers are listed in Table 2.

30. The kit of claim 27, comprising: sRNA-specific probes and/or primers configured for detecting at least 40 sRNAs listed in Table 1 (SEQ ID NOS: 1 to 206), and wherein the probes and/or primers are listed in Table 2.

31. The kit of claim 27, comprising sRNA-specific probes and/or primers configured for detecting at least 50 sRNAs listed in Table 1 (SEQ ID NOS: 1 to 206), and wherein the probes and/or primers are listed in Table 2.

32. The kit of any one of claims 26 to 31, comprising sRNA-specific stem loop RT primers, which are optionally from Table 2.

33. The kit of any one of claims 26 to 32, comprising forward and reverse primer pairs for amplifying sRNA reverse transcripts, which are optionally as shown in Table 2.

34. The kit of claim any one of claims 27 to 31, comprising sRNA-specific probes for detecting amplicons, and which are optionally listed in Table 2.

35. The kit of claim 34, wherein the sRNA-specific probes are fluorescent-labeled probes.

36. The kit of claim 35, wherein the probe further comprising a quencher moiety.

37. The kit of any one of claims 26 to 31, comprising an array of sRNA-specific hybridization probes.

38. A composition comprising a small interfering RNA (siRNA) comprising an antisense strand and a sense strand, the antisense strand comprising at least 15 consecutive nucleotides of a sequence of Table 1.

39. The composition of claim 38, wherein the siRNA mimics the action of: hsa-mir-7-5p, hsa-mir-146a-5p, hsa-mir-92a-3p, hsa-mir-423-5p, hsa-let-7b-5p, hsa-mir-30d-5p, hsa-mir-30a-5p, hsa-let-7a-5p, hsa-mir-4429, hsa-mir320c, hsa-mir-92b-3p, hsa-mir-486-5p, hsa-mir-25-3p, hsa-let-7d-3p, hsa-mir-16-5p, hsa-let-7c-5p, hsa-mir-6130, or hsa-let-7g-5p.

40. The composition of claim 38 or 39, wherein the siRNA comprises a chemical modification.

41. The composition of any of claims 38 to 40, wherein the chemical modification increases stability, reduces endonuclease degradation, reduces immunogenicity, and/or reduces Toll-like receptor recognition.

42. The composition of claim 41, wherein the chemical modification is a nucleobase modification, a backbone modification, and/or a sugar modification.

43. The compositions of claim 42, wherein the nucleobase modification suppresses RNA recognition by a Toll-like receptor (TLR).

44. The composition of claim 42, wherein the backbone modification is selected from phosphorothioate, phosphorodithioate, methylphosphonate, and methoxypropylphosphonate.

45. The composition of claim 42, wherein the sugar modification is selected from 2′-methoxy (2′-OMe), 2′-O-methoxyethyl(2′-O-MOE), 2′-fluoro (2′-F), constrained ethyl (cEt), bridged nucleic acid (BNA) and locked nucleic acid (LNA).

46. The composition of any one of claims 38 to 45, wherein the siRNA comprises a sense and antisense strain, each having a length of about 12 to about 40 nucleotides.

47. The composition of claim 46, wherein the siRNA comprises two substantially complementary RNA strands with a duplex length of about 12 to about 40 base pairs.

48. The composition of any one of claims 38 to 47, wherein the siRNA comprises one or two 3′ end overhangs.

49. The composition of claim 48, wherein the siRNA comprises a sense strand overhang and an antisense strand overhang.

50. The composition of claim 48 or 49, wherein the overhangs are deoxythymidine (dT-dT) overhangs.

51. The composition of claim 50, wherein the siRNA is in a 19+2 format.

52. The composition of any one of claims 38 to 48, wherein the siRNA is an asymmetric siRNA having a blunt end corresponding to the 5′ end of the antisense strand.

53. The composition of claim 52, wherein the siRNA has an antisense strand with a nucleotide at its 5′ end that is not base paired with the sense strand.

54. A composition comprising an antisense oligonucleotide that is at least 10 linked nucleotides in length and having a sequence that is complementary to a nucleotide sequence selected from Table 1 (SEQ ID NOS: 1 to 206).

55. The composition of claim 54, wherein the oligonucleotide is fully complementary to a nucleotide sequence selected from Table 1 (SEQ ID NOS: 1 to 206).

56. The composition of claim 54 or 55, wherein the antisense oligonucleotide is complementary to a sequence within SEQ ID NO: 1, 40, 44, 50-52, 54, 55, 58, 60, 63, 75, 76, 77, 81, 84, 86, 87, 88, 136, 137, 138, 175, 178, 192, 193, 194, 195, 196, and 197.

57. The composition of any one of claims 54 to 56, wherein the antisense oligonucleotide is complementary to a sequence of: hsa-mir-320c, hsa-mir-483-5p, hsa-let-7d-5p, hsa-mir-30e-5p, hsa-mir-21-5p, hsa-mir-143-3p, hsa-mir-423-5p, has-mir-423-5p, hsa-mir-122-5p, hsa-mir-181a-5p, hsa-mir-21-3p, hsa-mir-191-5p, hsa-mir-22-3p, hsa-mir-221-3p, hsa-mir-423-3p, hsa-mir-21-5p, hsa-mir-146a-5p, hsa-mir-629-5p, hsa-mir-30d-5p, and hsa-mir-30e-5p, or a precursor RNA or isoform of any of the foregoing.

58. The composition of claim 57, wherein the antisense oligonucleotide is complementary to a sequence of miR-22-3p, or a RNA precursor or isoform thereof.

59. The composition of claim 58, wherein the antisense oligonucleotide is complementary to SEQ ID NO: 86 or a sequence within SEQ ID NO: 86.

60. The composition of any one of claims 54 to 59, wherein the oligonucleotide is about 12 to about 40 nucleotides in length.

61. The composition of claim 60, wherein the oligonucleotide is 12, 13, 14, 15, 16, 17, 18, 19, or 20 nucleotides in length, and optionally from 12 to 16 nucleotides in length.

62. The composition of any one of claims 54 to 61, wherein the oligonucleotide has a contiguous sequence of at least 6 DNA nucleotides sufficient to recruit RNaseH.

63. The composition of claim 62, wherein the oligonucleotide has a 5′ segment, a 3′ segment, and a middle segment that recruiting RNaseH; wherein each of the 5′ and 3′ segments is from 2 to 6 nucleotides or from 2 to 4 nucleotides in length, and where the 5′ and 3′ segments contain RNA nucleotides.

64. The composition of claim 63, wherein one or more nucleotides of the 5′ segment and/or the 3′ segment comprise 2′-O substituents, optionally wherein all of the nucleotides of the 5′ segment and/or the 3′ segment comprise 2′-O substituents.

65. The composition of any one of claims 54 to 64, wherein 2′-O substituents are selected from 2′-O methyl, 2′-O ethyl, 2′-O methoxyethyl (MOE), and a bridged nucleotide having a 2′ to 4′ bridge.

66. The composition of claim 65, wherein the bridged nucleotide has a methylene bridge (LNA) or a constrained ethyl bridge (cEt).

67. The composition of any one of claims 54 to 66, wherein the oligonucleotide has a modified backbone.

68. The composition of claim 67, wherein the oligonucleotide comprises one or more phosphorothioate or phosphorodithioate nucleotides.

69. The composition of claim 68, wherein the oligonucleotide is fully phosphorothioate or phosphorodithioate linked.

70. The composition of any one of claims 54 to 69, wherein cytidine nucleobases are 5-methyl cytidine.

71. The composition of any one of claims 54 to 61, wherein the oligonucleotide has a morpholino, thiomorpholino, or PNA backbone.

72. The composition of any one of claims 54 to 71, wherein the Tm of the oligonucleotide hybridized to its target sequence is at least about 35° C.

73. The composition of claim 72, wherein the Tm of the oligonucleotide hybridized to its target sequence is at least about 40° C., or at least about 45° C., or at least about 50° C.

74. The composition of claims 72 to 73, wherein the Tm of the oligonucleotide hybridized to its target sequence is from about 35° C. to about 60° C., or from about 40° C. to about 60° C., or from about 50° C. to about 60° C.

75. The composition of any one of claims 54 to 74, wherein the composition further comprises a targeting or cell penetrating moiety.

76. A pharmaceutical composition comprising an effective amount of a composition of any one of claims 38 to 75 and one or more pharmaceutically acceptable excipients or carriers.

77. The pharmaceutical composition of claim 76, wherein the composition comprises liposomes, polymeric nanoparticles, or lipid nanoparticles.

78. A method for treating a subject having coronary microvascular dysfunction (CMD), comprising administering an effective amount of a composition sufficient for decreasing the expression of a small RNA selected from Table 1 having a negative Spearman correlation.

79. The method of claim 78, comprising administering a composition of any one of claims 54 to 76.

80. The composition of claim 79, wherein the antisense oligonucleotide is complementary to a sequence of miR-22-3p, or a RNA precursor or isoform thereof.

81. The composition of claim 80, wherein the antisense oligonucleotide is complementary to SEQ ID NO: 86 or a sequence within SEQ ID NO: 86.

82. The method of any one of claims 78 to 81, wherein the subject further has HFpEF.

83. The method of any one of claims 78 to 81, wherein the subject does not have HFpEF.

84. The method of any one of claims 78 to 83, wherein the subject has a CFR less than 2.5, which is optionally predicted by the method of any one of claims 1 to 22.


SEQUENCE LISTING (1239 sequences)

SEQ ID NO: 1 - RNA, 20 nt, synthetic construct
  source 1..20: mol_type = other RNA
gaaaagctgggttgaggggg

SEQ ID NO: 2 - RNA, 35 nt, synthetic construct
  source 1..35: mol_type = other RNA
gaaactctggtggaggtccgtagcggtcctgacgt

SEQ ID NO: 3 - RNA, 17 nt, synthetic construct
  source 1..17: mol_type = other RNA
gaattgtgagcggataa

SEQ ID NO: 4 - RNA, 22 nt, synthetic construct
  source 1..22: mol_type = other RNA
gataagggaagtcggcaaaata

SEQ ID NO: 5 - RNA, 31 nt, synthetic construct
  source 1..31: mol_type = other RNA
gcatgagtggttcagtggtagaattctcgcc

SEQ ID NO: 6 - RNA, 18 nt, synthetic construct
  source 1..18: mol_type = other RNA
ggccagtggcgcaatgga

SEQ ID NO: 7 - RNA, 36 nt, synthetic construct
  source 1..36: mol_type = other RNA
ggctgggtcggtcgggctggggcgcgaagcggggcc

SEQ ID NO: 8 - RNA, 20 nt, synthetic construct
  source 1..20: mol_type = other RNA
ggtgatgtcggcgatatagg

SEQ ID NO: 9 - RNA, 21 nt, synthetic construct
  source 1..21: mol_type = other RNA
taacccgtatcgtgagcatcc

SEQ ID NO: 10 - RNA, 33 nt, synthetic construct
  source 1..33: mol_type = other RNA
tacaaccccccactgctaaatttgactggcttc

SEQ ID NO: 11 - RNA, 22 nt, synthetic construct
  source 1..22: mol_type = other RNA
tatcccgccgttagccaccatc

SEQ ID NO: 12 - RNA, 22 nt, synthetic construct
  source 1..22: mol_type = other RNA
tcatgcaactcgtaggacaggt

SEQ ID NO: 13 - RNA, 24 nt, synthetic construct
  source 1..24: mol_type = other RNA
tggaagactagtgattctgttgtt

SEQ ID NO: 14 - RNA, 28 nt, synthetic construct
  source 1..28: mol_type = other RNA
tggtccgaaggtagtgggttatctcaat

SEQ ID NO: 15 - RNA, 21 nt, synthetic construct
  source 1..21: mol_type = other RNA
ttggcggatgagataagattt

SEQ ID NO: 16 - RNA, 33 nt, synthetic construct
  source 1..33: mol_type = other RNA
tttccgtagtgtagtggttatcacgttcgcctg

SEQ ID NO: 17 - RNA, 22 nt, synthetic construct
  source 1..22: mol_type = other RNA
tttcgccacctctgacttgggc

SEQ ID NO: 18 - RNA, 23 nt, synthetic construct
  source 1..23: mol_type = other RNA
actacgatacgggagggcttacc

SEQ ID NO: 19 - RNA, 21 nt, synthetic construct
  source 1..21: mol_type = other RNA
aagaacgtcgcgcactgctag

SEQ ID NO: 20 - RNA, 22 nt, synthetic construct
  source 1..22: mol_type = other RNA
catgctcgtgaactggcgtatg

SEQ ID NO: 21 - RNA, 24 nt, synthetic construct
  source 1..24: mol_type = other RNA
cgtgtggaattctcgggtgccaaa

SEQ ID NO: 22 - RNA, 21 nt, synthetic construct
  source 1..21: mol_type = other RNA
tccgggtagtctcgagaaaca

SEQ ID NO: 23 - RNA, 21 nt, synthetic construct
  source 1..21: mol_type = other RNA
tgaacacccggacctggctat

SEQ ID NO: 24 - RNA, 22 nt, synthetic construct
  source 1..22: mol_type = other RNA
aaacgacgagcgtggcaccacg

SEQ ID NO: 25 - RNA, 21 nt, synthetic construct
  source 1..21: mol_type = other RNA
ctttcgcttcgttatctactc

SEQ ID NO: 26 - RNA, 17 nt, synthetic construct
  source 1..17: mol_type = other RNA
aaaggaaggaatcgaac

SEQ ID NO: 27 - RNA, 31 nt, synthetic construct
  source 1..31: mol_type = other RNA
agcagagtggcgcagcggaagcgtgctaggc

SEQ ID NO: 28 - RNA, 31 nt, synthetic construct
  source 1..31: mol_type = other RNA
agcagagttgcgcagcggaagcgtgctggac

SEQ ID NO: 29 - RNA, 32 nt, synthetic construct
  source 1..32: mol_type = other RNA
aggctggtccgatggtagtgggttattagaac

SEQ ID NO: 30 - RNA, 21 nt, synthetic construct
  source 1..21: mol_type = other RNA
ataaccaggtccgggtgttca

SEQ ID NO: 31 - RNA, 20 nt, synthetic construct
  source 1..20: mol_type = other RNA
agcgtggtggtgccgatggt

SEQ ID NO: 32 - RNA, 21 nt, synthetic construct
  source 1..21: mol_type = other RNA
tgaacacccggacccggttat

SEQ ID NO: 33 - RNA, 17 nt, synthetic construct
  source 1..17: mol_type = other RNA
aacagcagttggacatg

SEQ ID NO: 34 - RNA, 34 nt, synthetic construct
  source 1..34: mol_type = other RNA
attggtcgtggctgtagtccgtgcgagaatacca

SEQ ID NO: 35 - RNA, 20 nt, synthetic construct
  source 1..20: mol_type = other RNA
catggaacgggttggcatgg

SEQ ID NO: 36 - RNA, 21 nt, synthetic construct
  source 1..21: mol_type = other RNA
cattcgatggtgtctgggatc

SEQ ID NO: 37 - RNA, 30 nt, synthetic construct
  source 1..30: mol_type = other RNA
cattggtggttcagtagtagaattctcgcc

SEQ ID NO: 38 - RNA, 20 nt, synthetic construct
  source 1..20: mol_type = other RNA
cccgagactcagtgaaattg

SEQ ID NO: 39 - RNA, 35 nt, synthetic construct
  source 1..35: mol_type = other RNA
aagaaggaaagaagaaggggaagaaaagaagggga

SEQ ID NO: 40 - RNA, 24 nt, synthetic construct
  source 1..24: mol_type = other RNA
aagacgggaggaaagaagggagat

SEQ ID NO: 41 - RNA, 39 nt, synthetic construct
  source 1..39: mol_type = other RNA
cggcggcggcggcggtggcggcggcggcggcggcgggac

SEQ ID NO: 42 - RNA, 22 nt, synthetic construct
  source 1..22: mol_type = other RNA
gagaactgaattccatgggttt

SEQ ID NO: 43 - RNA, 20 nt, synthetic construct
  source 1..20: mol_type = other RNA
gaggaggaggaggaggacgg

SEQ ID NO: 44 - RNA, 20 nt, synthetic construct
  source 1..20: mol_type = other RNA
gaggtagtaggttgcatagt

SEQ ID NO: 45 - RNA, 20 nt, synthetic construct
  source 1..20: mol_type = other RNA
gagtaagacgctgccggaat

SEQ ID NO: 46 - RNA, 23 nt, synthetic construct
  source 1..23: mol_type = other RNA
gatggggaagatcgggctcgcca

SEQ ID NO: 47 - RNA, 33 nt, synthetic construct
  source 1..33: mol_type = other RNA
gcattggtggttcagtggtagaattctcgcctg

SEQ ID NO: 48 - RNA, 26 nt, synthetic construct
  source 1..26: mol_type = other RNA
gcatttgtggttcagtggtagaattc

SEQ ID NO: 49 - RNA, 29 nt, synthetic construct
  source 1..29: mol_type = other RNA
ggctggtccgaaggtagtgagttatctca

SEQ ID NO: 50 - RNA, 24 nt, synthetic construct
  source 1..24: mol_type = other RNA
gtaaacatccttgactggaagctt

SEQ ID NO: 51 - RNA, 23 nt, synthetic construct
  source 1..23: mol_type = other RNA
tagcttatcagactgacgttgac

SEQ ID NO: 52 - RNA, 24 nt, synthetic construct
  source 1..24: mol_type = other RNA
tagcttatcagactgatgttgact

SEQ ID NO: 53 - RNA, 22 nt, synthetic construct
  source 1..22: mol_type = other RNA
tattgcagttgtcccggcctgt

SEQ ID NO: 54 - RNA, 21 nt, synthetic construct
  source 1..21: mol_type = other RNA
tgagatgaagcactgtagcaa

SEQ ID NO: 55 - RNA, 20 nt, synthetic construct
  source 1..20: mol_type = other RNA
tgaggggcagagagcgagat

SEQ ID NO: 56 - RNA, 21 nt, synthetic construct
  source 1..21: mol_type = other RNA
tgaggggcagagagcgaggct

SEQ ID NO: 57 - RNA, 23 nt, synthetic construct
  source 1..23: mol_type = other RNA
tgaggggcagagagcgagacctt

SEQ ID NO: 58 - RNA, 22 nt, synthetic construct
  source 1..22: mol_type = other RNA
tgaggggcagagagcgagaaga

SEQ ID NO: 59 - RNA, 23 nt, synthetic construct
  source 1..23: mol_type = other RNA
tgaggtagtaggttgtgtggtct

SEQ ID NO: 60 - RNA, 19 nt, synthetic construct
  source 1..19: mol_type = other RNA
tggagtgtgacaatggtga

SEQ ID NO: 61 - RNA, 22 nt, synthetic construct
  source 1..22: mol_type = other RNA
tgtaaacatccccgactgaaag

SEQ ID NO: 62 - RNA, 23 nt, synthetic construct
  source 1..23: mol_type = other RNA
tgtaaatatccccgactggaagc

SEQ ID NO: 63 - RNA, 23 nt, synthetic construct
  source 1..23: mol_type = other RNA
tgtaaacatccttgactggaagt

SEQ ID NO: 64 - RNA, 18 nt, synthetic construct
  source 1..18: mol_type = other RNA
ttgggagcgggcgggcgg

SEQ ID NO: 65 - RNA, 21 nt, synthetic construct
  source 1..21: mol_type = other RNA
tttagtaatggtaatggttct

SEQ ID NO: 66 - RNA, 30 nt, synthetic construct
  source 1..30: mol_type = other RNA
tttccgtagtgtagtggttatcacgttcgc

SEQ ID NO: 67 - RNA, 19 nt, synthetic construct
  source 1..19: mol_type = other RNA
tttgttcgttcggctcgcg

SEQ ID NO: 68 - RNA, 25 nt, synthetic construct
  source 1..25: mol_type = other RNA
acggcggcggcggcggcggcggggg

SEQ ID NO: 69 - RNA, 21 nt, synthetic construct
  source 1..21: mol_type = other RNA
agaggaggaagaggaggagga

SEQ ID NO: 70 - RNA, 31 nt, synthetic construct
  source 1..31: mol_type = other RNA
aggctggtccgatggtagtgggttatcagaa

SEQ ID NO: 71 - RNA, 25 nt, synthetic construct
  source 1..25: mol_type = other RNA
agggcggcggcggcggcggcggggg

SEQ ID NO: 72 - RNA, 24 nt, synthetic construct
  source 1..24: mol_type = other RNA
agggcggcggcggcggcggcgggg

SEQ ID NO: 73 - RNA, 18 nt, synthetic construct
  source 1..18: mol_type = other RNA
attccggttgttcttctc

SEQ ID NO: 74 - RNA, 21 nt, synthetic construct
  source 1..21: mol_type = other RNA
atgaggtagtaggttgtgtgg

SEQ ID NO: 75 - RNA, 21 nt, synthetic construct
  source 1..21: mol_type = other RNA
aacattcaacgctgtcggtgt

SEQ ID NO: 76 - RNA, 21 nt, synthetic construct
  source 1..21: mol_type = other RNA
caacaccagtcgatgggctgt

SEQ ID NO: 77 - RNA, 18 nt, synthetic construct
  source 1..18: mol_type = other RNA
caacggaatcccaaaagc

SEQ ID NO: 78 - RNA, 20 nt, synthetic construct
  source 1..20: mol_type = other RNA
aaccgggcggaaacaccaaa

SEQ ID NO: 79 - RNA, 18 nt, synthetic construct
  source 1..18: mol_type = other RNA
cattcttgcgacccgggt

SEQ ID NO: 80 - RNA, 33 nt, synthetic construct
  source 1..33: mol_type = other RNA
cccctgtggtctagtggttaggattcggcgctc

SEQ ID NO: 81 - RNA, 20 nt, synthetic construct
  source 1..20: mol_type = other RNA
aaaagctgggttgagaggga

SEQ ID NO: 82 - RNA, 28 nt, synthetic construct
  source 1..28: mol_type = other RNA
ctggtccgaaggtagtgagttatctcaa

SEQ ID NO: 83 - RNA, 21 nt, synthetic construct
  source 1..21: mol_type = other RNA
aaaagctgggctgagagggcg

SEQ ID NO: 84 - RNA, 20 nt, synthetic construct
  source 1..20: mol_type = other RNA
aaaagctgggttgagaggga

SEQ ID NO: 85 - RNA, 23 nt, synthetic construct
  source 1..23: mol_type = other RNA
aaaagctgggttgagagggcgag

SEQ ID NO: 86 - RNA, 22 nt, synthetic construct
  source 1..22: mol_type = other RNA
aagctgccagttgaagaactgc

SEQ ID NO: 87 - RNA, 22 nt, synthetic construct
  source 1..22: mol_type = other RNA
agctacattgtctgctgggttt

SEQ ID NO: 88 - RNA, 23 nt, synthetic construct
  source 1..23: mol_type = other RNA
agctcggcctgaggcccctcagt

SEQ ID NO: 89 - RNA, 33 nt, synthetic construct
  source 1..33: mol_type = other RNA
aggctggtccgatggtagtgggttatcagaact

SEQ ID NO: 90 - RNA, 25 nt, synthetic construct
  source 1..25: mol_type = other RNA
agggcggcggcggcggcggcggggg

SEQ ID NO: 91 - RNA, 23 nt, synthetic construct
  source 1..23: mol_type = other RNA
atattgcacttgtcccggcctgt

SEQ ID NO: 92 - RNA, 22 nt, synthetic construct
  source 1..22: mol_type = other RNA
atcctgtactgagctgccccga

SEQ ID NO: 93 - RNA, 23 nt, synthetic construct
  source 1..23: mol_type = other RNA
atcctgtactgagctgccccgag

SEQ ID NO: 94 - RNA, 20 nt, synthetic construct
  source 1..20: mol_type = other RNA
attgcacttgtcccggcctg

SEQ ID NO: 95 - RNA, 21 nt, synthetic construct
  source 1..21: mol_type = other RNA
attgcacttgtcccggcctgt

SEQ ID NO: 96 - RNA, 21 nt, synthetic construct
  source 1..21: mol_type = other RNA
attgcacttgccccggcctgt

SEQ ID NO: 97 - RNA, 21 nt, synthetic construct
  source 1..21: mol_type = other RNA
cattgcacttgtcccggcctg

SEQ ID NO: 98 - RNA, 22 nt, synthetic construct
  source 1..22: mol_type = other RNA
cattgcacttgtctcggtctgg

SEQ ID NO: 99 - RNA, 30 nt, synthetic construct
  source 1..30: mol_type = other RNA
cattggtggttcagtggtagaattctcgcc

SEQ ID NO: 100 - RNA, 27 nt, synthetic construct
  source 1..27: mol_type = other RNA
ccccccgctgctaaatttgactggctt

SEQ ID NO: 101 - RNA, 23 nt, synthetic construct
  source 1..23: mol_type = other RNA
ccctgtactgagctgccccgaga

SEQ ID NO: 102 - RNA, 22 nt, synthetic construct
  source 1..22: mol_type = other RNA
cctgtactgagctgccccgaga

SEQ ID NO: 103 - RNA, 21 nt, synthetic construct
  source 1..21: mol_type = other RNA
cctgtactgagctgccccgag

SEQ ID NO: 104 - RNA, 20 nt, synthetic construct
  source 1..20: mol_type = other RNA
cgaggaggaagaggaggacg

SEQ ID NO: 105 - RNA, 20 nt, synthetic construct
  source 1..20: mol_type = other RNA
cgaggtagtaggttgtatag

SEQ ID NO: 106 - RNA, 20 nt, synthetic construct
  source 1..20: mol_type = other RNA
ctatacgacctgctgccttt

SEQ ID NO: 107 - RNA, 20 nt, synthetic construct
  source 1..20: mol_type = other RNA
ctgtactgagctgccccgag

SEQ ID NO: 108 - RNA, 30 nt, synthetic construct
  source 1..30: mol_type = other RNA
gaattggtggttcagtggtagaattctcgc

SEQ ID NO: 109 - RNA, 32 nt, synthetic construct
  source 1..32: mol_type = other RNA
gcatcggtggttcagtggtagaattctcgcct

SEQ ID NO: 110 - RNA, 32 nt, synthetic construct
  source 1..32: mol_type = other RNA
gcattagtggttcagtggtagaattctcgcct

SEQ ID NO: 111 - RNA, 31 nt, synthetic construct
  source 1..31: mol_type = other RNA
gcattgatggttcagtggtagaattctcgcc

SEQ ID NO: 112 - RNA, 31 nt, synthetic construct
  source 1..31: mol_type = other RNA
gcattggtagttcagtggtagaattctcgcc

SEQ ID NO: 113 - RNA, 33 nt, synthetic construct
  source 1..33: mol_type = other RNA
gcattggtggttcagtggtagaattctcgcccg

SEQ ID NO: 114 - RNA, 31 nt, synthetic construct
  source 1..31: mol_type = other RNA
gcattggtggttcagtggtagaattctcgtc

SEQ ID NO: 115 - RNA, 32 nt, synthetic construct
  source 1..32: mol_type = other RNA
gcattggtggttcagtggtagaattttcgcct

SEQ ID NO: 116 - RNA, 31 nt, synthetic construct
  source 1..31: mol_type = other RNA
gcattggtggttcagtggtaggattctcgcc

SEQ ID NO: 117 - RNA, 31 nt, synthetic construct
  source 1..31: mol_type = other RNA
gcattggtggttcagtggtagagttctcgcc

SEQ ID NO: 118 - RNA, 31 nt, synthetic construct
  source 1..31: mol_type = other RNA
gcattggtggttcggtggtagaattctcgcc

SEQ ID NO: 119 - RNA, 32 nt, synthetic construct
  source 1..32: mol_type = other RNA
gcgccgctggtgtagtggcatcatgcaagatt

SEQ ID NO: 120 - RNA, 31 nt, synthetic construct
  source 1..31: mol_type = other RNA
gcgttggtggttcagtggtagaattctcgcc

SEQ ID NO: 121 - RNA, 34 nt, synthetic construct
  source 1..34: mol_type = other RNA
ggctggcccgatggtagtgggttatcagaactta

SEQ ID NO: 122 - RNA, 30 nt, synthetic construct
  source 1..30: mol_type = other RNA
ggctggtccgaaggtagtgagttacctcaa

SEQ ID NO: 123 - RNA, 30 nt, synthetic construct
  source 1..30: mol_type = other RNA
ggctggtccgaaggcagtgagttatctcaa

SEQ ID NO: 124 - RNA, 33 nt, synthetic construct
  source 1..33: mol_type = other RNA
ggctggtccgatagtagtgggttatcagaactt

SEQ ID NO: 125 - RNA, 30 nt, synthetic construct
  source 1..30: mol_type = other RNA
ggctggtccgatggtagtgggtcatcagaa

SEQ ID NO: 126 - RNA, 30 nt, synthetic construct
  source 1..30: mol_type = other RNA
ggctggtccgatggtagcgggttatcagaa

SEQ ID NO: 127 - RNA, 33 nt, synthetic construct
  source 1..33: mol_type = other RNA
ggctggtccgatggtagtgggttgtcagaactt

SEQ ID NO: 128 - RNA, 31 nt, synthetic construct
  source 1..31: mol_type = other RNA
ggctggtccgatggtagtaggttatcagaac

SEQ ID NO: 129 - RNA, 21 nt, synthetic construct
  source 1..21: mol_type = other RNA
ggggaattgcgagcggataac

SEQ ID NO: 130 - RNA, 21 nt, synthetic construct
  source 1..21: mol_type = other RNA
ggggaattgtgagcggataat

SEQ ID NO: 131 - RNA, 21 nt, synthetic construct
  source 1..21: mol_type = other RNA
ggggaattgtgagcggacaac

SEQ ID NO: 132 - RNA, 22 nt, synthetic construct
  source 1..22: mol_type = other RNA
taagcggcggggtcggaacagg

SEQ ID NO: 133 - RNA, 21 nt, synthetic construct
  source 1..21: mol_type = other RNA
taaggggcagagagcgagact

SEQ ID NO: 134 - RNA, 22 nt, synthetic construct
  source 1..22: mol_type = other RNA
taatgcacttgtcccggcctgt

SEQ ID NO: 135 - RNA, 22 nt, synthetic construct
  source 1..22: mol_type = other RNA
tagcagcacgtaaatatcggcg

SEQ ID NO: 136 - RNA, 22 nt, synthetic construct
  source 1..22: mol_type = other RNA
tagcttatcagactgacgttga

SEQ ID NO: 137 - RNA, 22 nt, synthetic construct
  source 1..22: mol_type = other RNA
tagcttatcagactgatgttaa

SEQ ID NO: 138 - RNA, 20 nt, synthetic construct
  source 1..20: mol_type = other RNA
tagcttatcagactgatgtt

SEQ ID NO: 139 - RNA, 22 nt, synthetic construct
  source 1..22: mol_type = other RNA
tattgcacttgtcacggcctgt

SEQ ID NO: 140 - RNA, 22 nt, synthetic construct
  source 1..22: mol_type = other RNA
tattgcacttgccccggcctgt

SEQ ID NO: 141 - RNA, 21 nt, synthetic construct
  source 1..21: mol_type = other RNA
tattgcacttgtcccggtctg

SEQ ID NO: 142 - RNA, 22 nt, synthetic construct
  source 1..22: mol_type = other RNA
tattgcacttgtcccagcctgt

SEQ ID NO: 143 - RNA, 22 nt, synthetic construct
  source 1..22: mol_type = other RNA
tattgcactcgtcccggcctgt

SEQ ID NO: 144 - RNA, 22 nt, synthetic construct
  source 1..22: mol_type = other RNA
tattgcacttgtcccggccagt

SEQ ID NO: 145 - RNA, 21 nt, synthetic construct
  source 1..21: mol_type = other RNA
tattgcacttgtcccggcccg

SEQ ID NO: 146 - RNA, 21 nt, synthetic construct
  source 1..21: mol_type = other RNA
tattgcacttgtcccggcctg

SEQ ID NO: 147 - RNA, 21 nt, synthetic construct
  source 1..21: mol_type = other RNA
tattgcgcttgtcccggcctg

SEQ ID NO: 148 - RNA, 22 nt, synthetic construct
  source 1..22: mol_type = other RNA
tattgcacttgtccctgcctgt

SEQ ID NO: 149 - RNA, 22 nt, synthetic construct
  source 1..22: mol_type = other RNA
tattgcacttatcccggcctgt

SEQ ID NO: 150 - RNA, 22 nt, synthetic construct
  source 1..22: mol_type = other RNA
tattgcacttgtccaggcctgt

SEQ ID NO: 151 - RNA, 22 nt, synthetic construct
  source 1..22: mol_type = other RNA
tattgctcttgtcccggcctgt

SEQ ID NO: 152 - RNA, 23 nt, synthetic construct
  source 1..23: mol_type = other RNA
tattgcacttgtcccggcctgtg

SEQ ID NO: 153 - RNA, 23 nt, synthetic construct
  source 1..23: mol_type = other RNA
tcccgtactgagctgccccgaga

SEQ ID NO: 154 - RNA, 22 nt, synthetic construct
  source 1..22: mol_type = other RNA
tcccgtactgagctgccccgag

SEQ ID NO: 155 - RNA, 20 nt, synthetic construct
  source 1..20: mol_type = other RNA
tcccgtactgagctgccccg

SEQ ID NO: 156 - RNA, 32 nt, synthetic construct
  source 1..32: mol_type = other RNA
tccctggtggtctaatggttaggattcggcgc

SEQ ID NO: 157 - RNA, 32 nt, synthetic construct
  source 1..32: mol_type = other RNA
tccctggtggtctagtagttaggattcggcgc

SEQ ID NO: 158 - RNA, 33 nt, synthetic construct
  source 1..33: mol_type = other RNA
tccctggtgatctagtggttaggattcggcgct

SEQ ID NO: 159 - RNA, 35 nt, synthetic construct
  source 1..35: mol_type = other RNA
tccctggtggtctagtggtcaggattcggcgctct

SEQ ID NO: 160 - RNA, 23 nt, synthetic construct
  source 1..23: mol_type = other RNA
tcctgcactgagctgccccgaga

SEQ ID NO: 161 - RNA, 23 nt, synthetic construct
  source 1..23: mol_type = other RNA
tcctgtactaagctgccccgaga

SEQ ID NO: 162 - RNA, 23 nt, synthetic construct
  source 1..23: mol_type = other RNA
tcctgtaccgagctgccccgagt

SEQ ID NO: 163 - RNA, 22 nt, synthetic construct
  source 1..22: mol_type = other RNA
tcctgtactgagctgccccaag

SEQ ID NO: 164 - RNA, 23 nt, synthetic construct
  source 1..23: mol_type = other RNA
tcctgtactgagctgccccggga

SEQ ID NO: 165 - RNA, 22 nt, synthetic construct
  source 1..22: mol_type = other RNA
tcctgtactgagctgccccgtg

SEQ ID NO: 166 - RNA, 23 nt, synthetic construct
  source 1..23: mol_type = other RNA
tcctgtaccgagctgccccgaga

SEQ ID NO: 167 - RNA, 21 nt, synthetic construct
  source 1..21: mol_type = other RNA
tcctgtactgagctgccacga

SEQ ID NO: 168 - RNA, 22 nt, synthetic construct
  source 1..22: mol_type = other RNA
tcctgtaccgagctgccccgag

SEQ ID NO: 169 - RNA, 21 nt, synthetic construct
  source 1..21: mol_type = other RNA
tcctgtactgagctgccccgt

SEQ ID NO: 170 - RNA, 23 nt, synthetic construct
  source 1..23: mol_type = other RNA
tcctgtactgagctgccccaaga

SEQ ID NO: 171 - RNA, 22 nt, synthetic construct
  source 1..22: mol_type = other RNA
tcctgtactgagttgccccgag

SEQ ID NO: 172 - RNA, 23 nt, synthetic construct
  source 1..23: mol_type = other RNA
tcctgtactgggctgccccgagt

SEQ ID NO: 173 - RNA, 22 nt, synthetic construct
  source 1..22: mol_type = other RNA
tcctgtactgggctgccccgag

SEQ ID NO: 174 - RNA, 21 nt, synthetic construct
  source 1..21: mol_type = other RNA
tcctgtactgagctgcccgag

SEQ ID NO: 175 - RNA, 21 nt, synthetic construct
  source 1..21: mol_type = other RNA
tgagaactgaattccatgggt

SEQ ID NO: 176 - RNA, 21 nt, synthetic construct
  source 1..21: mol_type = other RNA
tgaggggcagagagcgagacc

SEQ ID NO: 177 - RNA, 21 nt, synthetic construct
  source 1..21: mol_type = other RNA
tgagggacagagagcgagact

SEQ ID NO: 178 - RNA, 24 nt, synthetic construct
  source 1..24: mol_type = other RNA
tgaggggcagagagcgggactttt

SEQ ID NO: 179 - RNA, 23 nt, synthetic construct
  source 1..23: mol_type = other RNA
tgaggggcagagggcgagacttt

SEQ ID NO: 180 - RNA, 23 nt, synthetic construct
  source 1..23: mol_type = other RNA
tgaggtagtaggtcgtgtggttt

SEQ ID NO: 181 - RNA, 23 nt, synthetic construct
  source 1..23: mol_type = other RNA
tgaggtagtaggttgtgtggtct

SEQ ID NO: 182 - RNA, 20 nt, synthetic construct
  source 1..20: mol_type = other RNA
tgaggtagtaggtcgtgtgg

SEQ ID NO: 183 - RNA, 20 nt, synthetic construct
  source 1..20: mol_type = other RNA
tgaggtagtaggttgtatag

SEQ ID NO: 184 - RNA, 21 nt, synthetic construct
  source 1..21: mol_type = other RNA
tgaggtagtagtttgcgctgt

SEQ ID NO: 185 - RNA, 20 nt, synthetic construct
  source 1..20: mol_type = other RNA
tgaggtagtaggtcgtatag

SEQ ID NO: 186 - RNA, 21 nt, synthetic construct
  source 1..21: mol_type = other RNA
tgaggtagtaggtcgtatagt

SEQ ID NO: 187 - RNA, 19 nt, synthetic construct
  source 1..19: mol_type = other RNA
tgaggtagtaggttgtgtg

SEQ ID NO: 188 - RNA, 22 nt, synthetic construct
  source 1..22: mol_type = other RNA
tgaggtagtaggttatgtggtt

SEQ ID NO: 189 - RNA, 22 nt, synthetic construct
  source 1..22: mol_type = other RNA
tgaggtagtggattgtatagtt

SEQ ID NO: 190 - RNA, 22 nt, synthetic construct
  source 1..22: mol_type = other RNA
tgaggtagtagtttgcacagtt

SEQ ID NO: 191 - RNA, 20 nt, synthetic construct
  source 1..20: mol_type = other RNA
tgaggtagtaggttgtgtgg

SEQ ID NO: 192 - RNA, 22 nt, synthetic construct
  source 1..22: mol_type = other RNA
tggagtgtgacaatggtgtctg

SEQ ID NO: 193 - RNA, 21 nt, synthetic construct
  source 1..21: mol_type = other RNA
tgggtttacgttgggagaact

SEQ ID NO: 194 - RNA, 22 nt, synthetic construct
  source 1..22: mol_type = other RNA
tgtaaacatccttgactggaag

SEQ ID NO: 195 - RNA, 22 nt, synthetic construct
  source 1..22: mol_type = other RNA
tgtaaacatccccgactggaaa

SEQ ID NO: 196 - RNA, 21 nt, synthetic construct
  source 1..21: mol_type = other RNA
tgtaaacatcctacactctca

SEQ ID NO: 197 - RNA, 24 nt, synthetic construct
  source 1..24: mol_type = other RNA
tgtagacatccttgactggaagct

SEQ ID NO: 198 - RNA, 22 nt, synthetic construct
  source 1..22: mol_type = other RNA
tgttgcacttgtcccggcctgt

SEQ ID NO: 199 - RNA, 23 nt, synthetic construct
  source 1..23: mol_type = other RNA
ttctgtactgagctgccccgaga

SEQ ID NO: 200 - RNA, 20 nt, synthetic construct
  source 1..20: mol_type = other RNA
ttgcacttgtcccggcctgt

SEQ ID NO: 201 - RNA, 18 nt, synthetic construct
  source 1..18: mol_type = other RNA
ttgggagcgggcgggcgg

SEQ ID NO: 202 - RNA, 18 nt, synthetic construct
  source 1..18: mol_type = other RNA
attccggttgttcttctc

SEQ ID NO: 203 - RNA, 22 nt, synthetic construct
  source 1..22: mol_type = other RNA
ttgtagcactggggccagatgg

SEQ ID NO: 204 - RNA, 20 nt, synthetic construct
  source 1..20: mol_type = other RNA
agatgagaggacgggacgcc

SEQ ID NO: 205 - RNA, 22 nt, synthetic construct
  source 1..22: mol_type = other RNA
tatggaagtgccgccgctgaaa

SEQ ID NO: 206 - RNA, 22 nt, synthetic construct
  source 1..22: mol_type = other RNA
tattaaatcaatctcggctggc

SEQ ID NO: 207 - RNA, 50 nt, synthetic construct
  source 1..50: mol_type = other RNA
gtcgtatccagtgcagggtccgaggtattcgcactggatacgacccccct

SEQ ID NO: 208 - RNA, 20 nt, synthetic construct
  source 1..20: mol_type = other RNA
gaaaagctgggttgaggggg

SEQ ID NO: 209 - RNA, 50 nt, synthetic construct
  source 1..50: mol_type = other RNA
gtcgtatccagtgcagggtccgaggtattcgcactggatacgacacgtca

SEQ ID NO: 210 - RNA, 20 nt, synthetic construct
  source 1..20: mol_type = other RNA
gaaactctggtggaggtccg

SEQ ID NO: 211 - RNA, 50 nt, synthetic construct
  source 1..50: mol_type = other RNA
gtcgtatccagtgcagggtccgaggtattcgcactggatacgacttatcc

SEQ ID NO: 212 - RNA, 17 nt, synthetic construct
  source 1..17: mol_type = other RNA
gaattgtgagcggataa

SEQ ID NO: 213 - RNA, 50 nt, synthetic construct
  source 1..50: mol_type = other RNA
gtcgtatccagtgcagggtccgaggtattcgcactggatacgactatttt

SEQ ID NO: 214 - RNA, 20 nt, synthetic construct
  source 1..20: mol_type = other RNA
gataagggaagtcggcaaaa

SEQ ID NO: 215 - RNA, 50 nt, synthetic construct
  source 1..50: mol_type = other RNA
gtcgtatccagtgcagggtccgaggtattcgcactggatacgacggcgag

SEQ ID NO: 216 - RNA, 20 nt, synthetic construct
  source 1..20: mol_type = other RNA
gcatgagtggttcagtggta

SEQ ID NO: 217 - RNA, 50 nt, synthetic construct
  source 1..50: mol_type = other RNA
gtcgtatccagtgcagggtccgaggtattcgcactggatacgactccatt

SEQ ID NO: 218 - RNA, 18 nt, synthetic construct
  source 1..18: mol_type = other RNA
ggccagtggcgcaatgga

SEQ ID NO: 219 - RNA, 50 nt, synthetic construct
  source 1..50: mol_type = other RNA
gtcgtatccagtgcagggtccgaggtattcgcactggatacgacggcccc

SEQ ID NO: 220 - RNA, 20 nt, synthetic construct
  source 1..20: mol_type = other RNA
ggctgggtcggtcgggctgg

SEQ ID NO: 221 - RNA, 50 nt, synthetic construct
  source 1..50: mol_type = other RNA
gtcgtatccagtgcagggtccgaggtattcgcactggatacgaccctata

SEQ ID NO: 222 - RNA, 20 nt, synthetic construct
  source 1..20: mol_type = other RNA
ggtgatgtcggcgatatagg

SEQ ID NO: 223 - RNA, 50 nt, synthetic construct
  source 1..50: mol_type = other RNA
gtcgtatccagtgcagggtccgaggtattcgcactggatacgacggatgc

SEQ ID NO: 224 - RNA, 20 nt, synthetic construct
  source 1..20: mol_type = other RNA
taacccgtatcgtgagcatc

SEQ ID NO: 225 - RNA, 50 nt, synthetic construct
  source 1..50: mol_type = other RNA
gtcgtatccagtgcagggtccgaggtattcgcactggatacgacgaagcc

SEQ ID NO: 226 - RNA, 20 nt, synthetic construct
  source 1..20: mol_type = other RNA
tacaaccccccactgctaaa

SEQ ID NO: 227 - RNA, 50 nt, synthetic construct
  source 1..50: mol_type = other RNA
gtcgtatccagtgcagggtccgaggtattcgcactggatacgacgatggt

SEQ ID NO: 228 - RNA, 20 nt, synthetic construct
  source 1..20: mol_type = other RNA
tatcccgccgttagccacca

SEQ ID NO: 229 - RNA, 50 nt, synthetic construct
  source 1..50: mol_type = other RNA
gtcgtatccagtgcagggtccgaggtattcgcactggatacgacacctgt

SEQ ID NO: 230 - RNA, 20 nt, synthetic construct
  source 1..20: mol_type = other RNA
tcatgcaactcgtaggacag

SEQ ID NO: 231 - RNA, 50 nt, synthetic construct
  source 1..50: mol_type = other RNA
gtcgtatccagtgcagggtccgaggtattcgcactggatacgacaacaac

SEQ ID NO: 232 - RNA, 20 nt, synthetic construct
  source 1..20: mol_type = other RNA
tggaagactagtgattctgt

SEQ ID NO: 233 - RNA, 50 nt, synthetic construct
  source 1..50: mol_type = other RNA
gtcgtatccagtgcagggtccgaggtattcgcactggatacgacattgag

SEQ ID NO: 234 - RNA, 20 nt, synthetic construct
  source 1..20: mol_type = other RNA
tggtccgaaggtagtgggtt

SEQ ID NO: 235 - RNA, 50 nt, synthetic construct
  source 1..50: mol_type = other RNA
gtcgtatccagtgcagggtccgaggtattcgcactggatacgacaaatct

SEQ ID NO: 236 - RNA, 20 nt, synthetic construct
  source 1..20: mol_type = other RNA
ttggcggatgagataagatt

SEQ ID NO: 237 - RNA, 50 nt, synthetic construct
  source 1..50: mol_type = other RNA
gtcgtatccagtgcagggtccgaggtattcgcactggatacgaccaggcg

SEQ ID NO: 238 - RNA, 20 nt, synthetic construct
  source 1..20: mol_type = other RNA
tttccgtagtgtagtggtta

SEQ ID NO: 239 - RNA, 50 nt, synthetic construct
  source 1..50: mol_type = other RNA
gtcgtatccagtgcagggtccgaggtattcgcactggatacgacgcccaa

SEQ ID NO: 240 - RNA, 20 nt, synthetic construct
  source 1..20: mol_type = other RNA
tttcgccacctctgacttgg

SEQ ID NO: 241 - RNA, 50 nt, synthetic construct
  source 1..50: mol_type = other RNA
gtcgtatccagtgcagggtccgaggtattcgcactggatacgacggtaag

SEQ ID NO: 242 - RNA, 20 nt, synthetic construct
  source 1..20: mol_type = other RNA
actacgatacgggagggctt

SEQ ID NO: 243 - RNA, 50 nt, synthetic construct
  source 1..50: mol_type = other RNA
gtcgtatccagtgcagggtccgaggtattcgcactggatacgacctagca

SEQ ID NO: 244 - RNA, 20 nt, synthetic construct
  source 1..20: mol_type = other RNA
aagaacgtcgcgcactgcta

SEQ ID NO: 245 - RNA, 50 nt, synthetic construct
  source 1..50: mol_type = other RNA
gtcgtatccagtgcagggtccgaggtattcgcactggatacgaccatacg

SEQ ID NO: 246 - RNA, 20 nt, synthetic construct
  source 1..20: mol_type = other RNA
catgctcgtgaactggcgta

SEQ ID NO: 247 - RNA, 50 nt, synthetic construct
  source 1..50: mol_type = other RNA
gtcgtatccagtgcagggtccgaggtattcgcactggatacgactttggc

SEQ ID NO: 248 - RNA, 20 nt, synthetic construct
  source 1..20: mol_type = other RNA
cgtgtggaattctcgggtgc

SEQ ID NO: 249 - RNA, 50 nt, synthetic construct
  source 1..50: mol_type = other RNA
gtcgtatccagtgcagggtccgaggtattcgcactggatacgactgtttc

SEQ ID NO: 250 - RNA, 20 nt, synthetic construct
  source 1..20: mol_type = other RNA
tccgggtagtctcgagaaac

SEQ ID NO: 251 - RNA, 50 nt, synthetic construct
  source 1..50: mol_type = other RNA
gtcgtatccagtgcagggtccgaggtattcgcactggatacgacatagcc

SEQ ID NO: 252 - RNA, 20 nt, synthetic construct
  source 1..20: mol_type = other RNA
tgaacacccggacctggcta

SEQ ID NO: 253 - RNA, 50 nt, synthetic construct
  source 1..50: mol_type = other RNA
gtcgtatccagtgcagggtccgaggtattcgcactggatacgaccgtggt

SEQ ID NO: 254 - RNA, 20 nt, synthetic construct
  source 1..20: mol_type = other RNA
aaacgacgagcgtggcacca

SEQ ID NO: 255 - RNA, 50 nt, synthetic construct
  source 1..50: mol_type = other RNA
gtcgtatccagtgcagggtccgaggtattcgcactggatacgacgagtag

SEQ ID NO: 256 - RNA, 20 nt, synthetic construct
  source 1..20: mol_type = other RNA
ctttcgcttcgttatctact

SEQ ID NO: 257 - RNA, 50 nt, synthetic construct
  source 1..50: mol_type = other RNA
gtcgtatccagtgcagggtccgaggtattcgcactggatacgacgttcga

SEQ ID NO: 258 - RNA, 17 nt, synthetic construct
  source 1..17: mol_type = other RNA
aaaggaaggaatcgaac

SEQ ID NO: 259 - RNA, 50 nt, synthetic construct
  source 1..50: mol_type = other RNA
gtcgtatccagtgcagggtccgaggtattcgcactggatacgacgcctag

SEQ ID NO: 260 - RNA, 20 nt, synthetic construct
  source 1..20: mol_type = other RNA
agcagagtggcgcagcggaa

SEQ ID NO: 261 - RNA, 50 nt, synthetic construct
  source 1..50: mol_type = other RNA
gtcgtatccagtgcagggtccgaggtattcgcactggatacgacgtccag

SEQ ID NO: 262 - RNA, 20 nt, synthetic construct
  source 1..20: mol_type = other RNA
agcagagttgcgcagcggaa

SEQ ID NO: 263 - RNA, 50 nt, synthetic construct
  source 1..50: mol_type = other RNA
gtcgtatccagtgcagggtccgaggtattcgcactggatacgacgttcta

SEQ ID NO: 264 - RNA, 20 nt, synthetic construct
  source 1..20: mol_type = other RNA
aggctggtccgatggtagtg

SEQ ID NO: 265 - RNA, 50 nt, synthetic construct
  source 1..50: mol_type = other RNA
gtcgtatccagtgcagggtccgaggtattcgcactggatacgactgaaca

SEQ ID NO: 266 - RNA, 20 nt, synthetic construct
  source 1..20: mol_type = other RNA
ataaccaggtccgggtgttc

SEQ ID NO: 267 - RNA, 50 nt, synthetic construct
  source 1..50: mol_type = other RNA
gtcgtatccagtgcagggtccgaggtattcgcactggatacgacaccatc

SEQ ID NO: 268 - RNA, 20 nt, synthetic construct
  source 1..20: mol_type = other RNA
agcgtggtggtgccgatggt

SEQ ID NO: 269 - RNA, 50 nt, synthetic construct
  source 1..50: mol_type = other RNA
gtcgtatccagtgcagggtccgaggtattcgcactggatacgacataacc

SEQ ID NO: 270 - RNA, 20 nt, synthetic construct
  source 1..20: mol_type = other RNA
tgaacacccggacccggtta

SEQ ID NO: 271 - RNA, 50 nt, synthetic construct
  source 1..50: mol_type = other RNA
gtcgtatccagtgcagggtccgaggtattcgcactggatacgaccatgtc

SEQ ID NO: 272 - RNA, 17 nt, synthetic construct
  source 1..17: mol_type = other RNA
aacagcagttggacatg

SEQ ID NO: 273 - RNA, 50 nt, synthetic construct
  source 1..50: mol_type = other RNA
gtcgtatccagtgcagggtccgaggtattcgcactggatacgactggtat

SEQ ID NO: 274 - RNA, 20 nt, synthetic construct
  source 1..20: mol_type = other RNA
attggtcgtggctgtagtcc

SEQ ID NO: 275 - RNA, 50 nt, synthetic construct
  source 1..50: mol_type = other RNA
gtcgtatccagtgcagggtccgaggtattcgcactggatacgacccatgc

SEQ ID NO: 276 - RNA, 20 nt, synthetic construct
  source 1..20: mol_type = other RNA
catggaacgggttggcatgg

SEQ ID NO: 277 - RNA, 50 nt, synthetic construct
  source 1..50: mol_type = other RNA
gtcgtatccagtgcagggtccgaggtattcgcactggatacgacgatccc

SEQ ID NO: 278 - RNA, 20 nt, synthetic construct
  source 1..20: mol_type = other RNA
cattcgatggtgtctgggat

SEQ ID NO: 279 - RNA, 50 nt, synthetic construct; identical to SEQ ID NO: 215
  source 1..50: mol_type = other RNA

SEQ ID NO: 280 - RNA, 20 nt, synthetic construct
  source 1..20: mol_type = other RNA
cattggtggttcagtagtag

SEQ ID NO: 281 - RNA, 50 nt, synthetic construct
  source 1..50: mol_type = other RNA
gtcgtatccagtgcagggtccgaggtattcgcactggatacgaccaattt

SEQ ID NO: 282 - RNA, 20 nt, synthetic construct
  source 1..20: mol_type = other RNA
cccgagactcagtgaaattg

SEQ ID NO: 283 - RNA, 50 nt, synthetic construct
  source 1..50: mol_type = other RNA
gtcgtatccagtgcagggtccgaggtattcgcactggatacgactcccct

SEQ ID NO: 284 - RNA, 20 nt, synthetic construct
  source 1..20: mol_type = other RNA
aagaaggaaagaagaagggg

SEQ ID NO: 285 - RNA, 50 nt, synthetic construct
  source 1..50: mol_type = other RNA
gtcgtatccagtgcagggtccgaggtattcgcactggatacgacatctcc

SEQ ID NO: 286 - RNA, 20 nt, synthetic construct
  source 1..20: mol_type = other RNA
aagacgggaggaaagaaggg

SEQ ID NO: 287 - RNA, 50 nt, synthetic construct
  source 1..50: mol_type = other RNA
gtcgtatccagtgcagggtccgaggtattcgcactggatacgacgtcccg

SEQ ID NO: 288 - RNA, 20 nt, synthetic construct
  source 1..20: mol_type = other RNA
cggcggcggcggcggtggcg

SEQ ID NO: 289 - RNA, 50 nt, synthetic construct
  source 1..50: mol_type = other RNA
gtcgtatccagtgcagggtccgaggtattcgcactggatacgacaaaccc

SEQ ID NO: 290 - RNA, 20 nt, synthetic construct
  source 1..20: mol_type = other RNA
gagaactgaattccatgggt

SEQ ID NO: 291 - RNA, 50 nt, synthetic construct
  source 1..50: mol_type = other RNA
gtcgtatccagtgcagggtccgaggtattcgcactggatacgacccgtcc

SEQ ID NO: 292 - RNA, 20 nt, synthetic construct
  source 1..20: mol_type = other RNA
gaggaggaggaggaggacgg

SEQ ID NO: 293 - RNA, 50 nt, synthetic construct
  source 1..50: mol_type = other RNA
gtcgtatccagtgcagggtccgaggtattcgcactggatacgacactatg

SEQ ID NO: 294 - RNA, 20 nt, synthetic construct
  source 1..20: mol_type = other RNA
gaggtagtaggttgcatagt

SEQ ID NO: 295 - RNA, 50 nt, synthetic construct
  source 1..50: mol_type = other RNA
gtcgtatccagtgcagggtccgaggtattcgcactggatacgacattccg

SEQ ID NO: 296 - RNA, 20 nt, synthetic construct
  source 1..20: mol_type = other RNA
gagtaagacgctgccggaat

SEQ ID NO: 297 - RNA, 50 nt, synthetic construct
  source 1..50: mol_type = other RNA
gtcgtatccagtgcagggtccgaggtattcgcactggatacgactggcga

SEQ ID NO: 298 - RNA, 20 nt, synthetic construct
  source 1..20: mol_type = other RNA
gatggggaagatcgggctcg

SEQ ID NO: 299 - RNA, 50 nt, synthetic construct; identical to SEQ ID NO: 237
  source 1..50: mol_type = other RNA

SEQ ID NO: 300 - RNA, 20 nt, synthetic construct
  source 1..20: mol_type = other RNA
gcattggtggttcagtggta

SEQ ID NO: 301 - RNA, 50 nt, synthetic construct
  source 1..50: mol_type = other RNA
gtcgtatccagtgcagggtccgaggtattcgcactggatacgacgaattc

SEQ ID NO: 302 - RNA, 20 nt, synthetic construct
  source 1..20: mol_type = other RNA
gcatttgtggttcagtggta

SEQ ID NO: 303 - RNA, 50 nt, synthetic construct
  source 1..50: mol_type = other RNA
gtcgtatccagtgcagggtccgaggtattcgcactggatacgactgagat

SEQ ID NO: 304 - RNA, 20 nt, synthetic construct
  source 1..20: mol_type = other RNA
ggctggtccgaaggtagtga

SEQ ID NO: 305 - RNA, 50 nt, synthetic construct
  source 1..50: mol_type = other RNA
gtcgtatccagtgcagggtccgaggtattcgcactggatacgacaagctt

SEQ ID NO: 306 - RNA, 20 nt, synthetic construct
  source 1..20: mol_type = other RNA
gtaaacatccttgactggaa

SEQ ID NO: 307 - RNA, 50 nt, synthetic construct
  source 1..50: mol_type = other RNA
gtcgtatccagtgcagggtccgaggtattcgcactggatacgacgtcaac

SEQ ID NO: 308 - RNA, 20 nt, synthetic construct
  source 1..20: mol_type = other RNA
tagcttatcagactgacgtt

SEQ ID NO: 309 - RNA, 50 nt, synthetic construct
  source 1..50: mol_type = other RNA
gtcgtatccagtgcagggtccgaggtattcgcactggatacgacagtcaa

SEQ ID NO: 310 - RNA, 20 nt, synthetic construct
  source 1..20: mol_type = other RNA
tagcttatcagactgatgtt

SEQ ID NO: 311 - RNA, 50 nt, synthetic construct
  source 1..50: mol_type = other RNA
gtcgtatccagtgcagggtccgaggtattcgcactggatacgacacaggc

SEQ ID NO: 312 - RNA, 20 nt, synthetic construct
  source 1..20: mol_type = other RNA
tattgcagttgtcccggcct

SEQ ID NO: 313 - RNA, 50 nt, synthetic construct
  source 1..50: mol_type = other RNA
gtcgtatccagtgcagggtccgaggtattcgcactggatacgacttgcta

SEQ ID NO: 314 - RNA, 20 nt, synthetic construct
  source 1..20: mol_type = other RNA
tgagatgaagcactgtagca

SEQ ID NO: 315 - RNA, 50 nt, synthetic construct
  source 1..50: mol_type = other RNA
gtcgtatccagtgcagggtccgaggtattcgcactggatacgacatctcg

SEQ ID NO: 316 - RNA, 20 nt, synthetic construct
  source 1..20: mol_type = other RNA
tgaggggcagagagcgagat

SEQ ID NO: 317 - RNA, 50 nt, synthetic construct
  source 1..50: mol_type = other RNA
gtcgtatccagtgcagggtccgaggtattcgcactggatacgacagcctc

SEQ ID NO: 318 - RNA, 20 nt, synthetic construct
  source 1..20: mol_type = other RNA
tgaggggcagagagcgaggc

SEQ ID NO: 319 - RNA, 50 nt, synthetic construct
  source 1..50: mol_type = other RNA
gtcgtatccagtgcagggtccgaggtattcgcactggatacgacaaggtc

SEQ ID NO: 320 - RNA, 20 nt, synthetic construct
  source 1..20: mol_type = other RNA
tgaggggcagagagcgagac

SEQ ID NO: 321 - RNA, 50 nt, synthetic construct
  source 1..50: mol_type = other RNA
gtcgtatccagtgcagggtccgaggtattcgcactggatacgactcttct

SEQ ID NO: 322 - RNA, 20 nt, synthetic construct
  source 1..20: mol_type = other RNA
tgaggggcagagagcgagaa

SEQ ID NO: 323 - RNA, 50 nt, synthetic construct
  source 1..50: mol_type = other RNA
gtcgtatccagtgcagggtccgaggtattcgcactggatacgacagacca

SEQ ID NO: 324 - RNA, 20 nt, synthetic construct
  source 1..20: mol_type = other RNA
tgaggtagtaggttgtgtgg

SEQ ID NO: 325 - RNA, 50 nt, synthetic construct
  source 1..50: mol_type = other RNA
gtcgtatccagtgcagggtccgaggtattcgcactggatacgactcacca

SEQ ID NO: 326 - RNA, 19 nt, synthetic construct
  source 1..19: mol_type = other RNA
tggagtgtgacaatggtga

SEQ ID NO: 327 - RNA, 50 nt, synthetic construct
  source 1..50: mol_type = other RNA
gtcgtatccagtgcagggtccgaggtattcgcactggatacgacctttca

SEQ ID NO: 328 - RNA, 20 nt, synthetic construct
  source 1..20: mol_type = other RNA
tgtaaacatccccgactgaa

SEQ ID NO: 329 - RNA, 50 nt, synthetic construct
  source 1..50: mol_type = other RNA
gtcgtatccagtgcagggtccgaggtattcgcactggatacgacgcttcc

SEQ ID NO: 330 - RNA, 20 nt, synthetic construct
  source 1..20: mol_type = other RNA
tgtaaatatccccgactgga

SEQ ID NO: 331 - RNA, 50 nt, synthetic construct
  source 1..50: mol_type = other RNA
gtcgtatccagtgcagggtccgaggtattcgcactggatacgacacttcc

SEQ ID NO: 332 - RNA, 20 nt, synthetic construct
  source 1..20: mol_type = other RNA
tgtaaacatccttgactgga

SEQ ID NO: 333 - RNA, 50 nt, synthetic construct
  source 1..50: mol_type = other RNA
gtcgtatccagtgcagggtccgaggtattcgcactggatacgacccgccc

SEQ ID NO: 334 - RNA, 18 nt, synthetic construct
  source 1..18: mol_type = other RNA
ttgggagcgggcgggcgg

SEQ ID NO: 335 - RNA, 50 nt, synthetic construct
  source 1..50: mol_type = other RNA
gtcgtatccagtgcagggtccgaggtattcgcactggatacgacagaacc

SEQ ID NO: 336 - RNA, 20 nt, synthetic construct
  source 1..20: mol_type = other RNA
tttagtaatggtaatggttc

SEQ ID NO: 337 - RNA, 50 nt, synthetic construct
  source 1..50: mol_type = other RNA
gtcgtatccagtgcagggtccgaggtattcgcactggatacgacgcgaac

SEQ ID NO: 338 - RNA, 20 nt, synthetic construct
  source 1..20: mol_type = other RNA
tttccgtagtgtagtggtta

SEQ ID NO: 339 - RNA, 50 nt, synthetic construct
  source 1..50: mol_type = other RNA
gtcgtatccagtgcagggtccgaggtattcgcactggatacgaccgcgag

SEQ ID NO: 340 - RNA, 19 nt, synthetic construct
  source 1..19: mol_type = other RNA
tttgttcgttcggctcgcg

SEQ ID NO: 341 - RNA, 50 nt, synthetic construct
  source 1..50: mol_type = other RNA
gtcgtatccagtgcagggtccgaggtattcgcactggatacgaccccccg

SEQ ID NO: 342 - RNA, 20 nt, synthetic construct
  source 1..20: mol_type = other RNA
acggcggcggcggcggcggc

SEQ ID NO: 343 - RNA, 50 nt, synthetic construct
  source 1..50: mol_type = other RNA
gtcgtatccagtgcagggtccgaggtattcgcactggatacgactcctcc

SEQ ID NO: 344 - RNA, 20 nt, synthetic construct
  source 1..20: mol_type = other RNA
agaggaggaagaggaggagg

SEQ ID NO: 345 - RNA, 50 nt, synthetic construct
  source 1..50: mol_type = other RNA
gtcgtatccagtgcagggtccgaggtattcgcactggatacgacttctga

SEQ ID NO: 346 - RNA, 20 nt, synthetic construct
  source 1..20: mol_type = other RNA
aggctggtccgatggtagtg

SEQ ID NO: 347 - RNA, 50 nt, synthetic construct; identical to SEQ ID NO: 341
  source 1..50: mol_type = other RNA

SEQ ID NO: 348 - RNA, 20 nt, synthetic construct
  source 1..20: mol_type = other RNA
agggcggcggcggcggcggc

SEQ ID NO: 349 - RNA, 50 nt, synthetic construct
  source 1..50: mol_type = other RNA
gtcgtatccagtgcagggtccgaggtattcgcactggatacgacccccgc

SEQ ID NO: 350 - RNA, 20 nt, synthetic construct
  source 1..20: mol_type = other RNA
agggcggcggcggcggcggc

SEQ ID NO: 351 - RNA, 50 nt, synthetic construct
  source 1..50: mol_type = other RNA
gtcgtatccagtgcagggtccgaggtattcgcactggatacgacgagaag

SEQ ID NO: 352 - RNA, 18 nt, synthetic construct
  source 1..18: mol_type = other RNA
attccggttgttcttctc

SEQ ID NO: 353 - RNA, 50 nt, synthetic construct
  source 1..50: mol_type = other RNA
gtcgtatccagtgcagggtccgaggtattcgcactggatacgacccacac

SEQ ID NO: 354 - RNA, 20 nt, synthetic construct
  source 1..20: mol_type = other RNA
atgaggtagtaggttgtgtg

SEQ ID NO: 355 - RNA, 50 nt, synthetic construct
  source 1..50: mol_type = other RNA
gtcgtatccagtgcagggtccgaggtattcgcactggatacgacacaccg

SEQ ID NO: 356 - RNA, 20 nt, synthetic construct
  source 1..20: mol_type = other RNA
aacattcaacgctgtcggtg

SEQ ID NO: 357 - RNA, 50 nt, synthetic construct
  source 1..50: mol_type = other RNA
gtcgtatccagtgcagggtccgaggtattcgcactggatacgacacagcc

SEQ ID NO: 358 - RNA, 20 nt, synthetic construct
  source 1..20: mol_type = other RNA
caacaccagtcgatgggctg

SEQ ID NO: 359 - RNA, 50 nt, synthetic construct
  source 1..50: mol_type = other RNA
gtcgtatccagtgcagggtccgaggtattcgcactggatacgacgctttt

SEQ ID NO: 360 - RNA, 18 nt, synthetic construct
  source 1..18: mol_type = other RNA
caacggaatcccaaaagc

SEQ ID NO: 361 - RNA, 50 nt, synthetic construct
  source 1..50: mol_type = other RNA
gtcgtatccagtgcagggtccgaggtattcgcactggatacgactttggt

SEQ ID NO: 362 - RNA, 20 nt, synthetic construct
  source 1..20: mol_type = other RNA
aaccgggcggaaacaccaaa

SEQ ID NO: 363 - RNA, 50 nt, synthetic construct
  source 1..50: mol_type = other RNA
gtcgtatccagtgcagggtccgaggtattcgcactggatacgacacccgg

SEQ ID NO: 364 - RNA, 18 nt, synthetic construct
  source 1..18: mol_type = other RNA
cattcttgcgacccgggt

SEQ ID NO: 365 - RNA, 50 nt, synthetic construct
  source 1..50: mol_type = other RNA
gtcgtatccagtgcagggtccgaggtattcgcactggatacgacgagcgc

SEQ ID NO: 366 - RNA, 20 nt, synthetic construct
  source 1..20: mol_type = other RNA
cccctgtggtctagtggtta

SEQ ID NO: 367 - RNA, 50 nt, synthetic construct
  source 1..50: mol_type = other RNA
gtcgtatccagtgcagggtccgaggtattcgcactggatacgactccctc

SEQ ID NO: 368 - RNA, 20 nt, synthetic construct
  source 1..20: mol_type = other RNA
aaaagctgggttgagaggga

SEQ ID NO: 369 - RNA, 50 nt, synthetic construct
  source 1..50: mol_type = other RNA
gtcgtatccagtgcagggtccgaggtattcgcactggatacgacttgaga

SEQ ID NO: 370 - RNA, 20 nt, synthetic construct
  source 1..20: mol_type = other RNA
ctggtccgaaggtagtgagt

SEQ ID NO: 371 - RNA, 50 nt, synthetic construct
  source 1..50: mol_type = other RNA
gtcgtatccagtgcagggtccgaggtattcgcactggatacgaccgccct

SEQ ID NO: 372 - RNA, 20 nt, synthetic construct
  source 1..20: mol_type = other RNA
aaaagctgggctgagagggc

SEQ ID NO: 373 - RNA, 50 nt, synthetic construct; identical to SEQ ID NO: 367
  source 1..50: mol_type = other RNA

SEQ ID NO: 374 - RNA, 20 nt, synthetic construct
  source 1..20: mol_type = other RNA
aaaagctgggttgagaggga

SEQ ID NO: 375 - RNA, 50 nt, synthetic construct
  source 1..50: mol_type = other RNA
gtcgtatccagtgcagggtccgaggtattcgcactggatacgacctcgcc

SEQ ID NO: 376 - RNA, 20 nt, synthetic construct
  source 1..20: mol_type = other RNA
aaaagctgggttgagagggc

SEQ ID NO: 377 - RNA, 50 nt, synthetic construct
  source 1..50: mol_type = other RNA
gtcgtatccagtgcagggtccgaggtattcgcactggatacgacgcagtt

SEQ ID NO: 378 - RNA, 20 nt, synthetic construct
  source 1..20: mol_type = other RNA
aagctgccagttgaagaact

SEQ ID NO: 379 - RNA, 50 nt, synthetic construct; identical to SEQ ID NO: 289
  source 1..50: mol_type = other RNA

SEQ ID NO: 380 - RNA, 20 nt, synthetic construct
  source 1..20: mol_type = other RNA
agctacattgtctgctgggt

SEQ ID NO: 381 - RNA, 50 nt, synthetic construct
  source 1..50: mol_type = other RNA
gtcgtatccagtgcagggtccgaggtattcgcactggatacgacactgag

SEQ ID NO: 382 - RNA, 20 nt, synthetic construct
  source 1..20: mol_type = other RNA
agctcggcctgaggcccctc

SEQ ID NO: 383 - RNA, 50 nt, synthetic construct
  source 1..50: mol_type = other RNA
gtcgtatccagtgcagggtccgaggtattcgcactggatacgacagttct

SEQ ID NO: 384 - RNA, 20 nt, synthetic construct
  source 1..20: mol_type = other RNA
aggctggtccgatggtagtg

SEQ ID NO: 385 - RNA, 50 nt, synthetic construct; identical to SEQ ID NO: 341
  source 1..50: mol_type = other RNA

SEQ ID NO: 386 - RNA, 20 nt, synthetic construct
  source 1..20: mol_type = other RNA
agggcggcggcggcggcggc

SEQ ID NO: 387 - RNA, 50 nt, synthetic construct; identical to SEQ ID NO: 311
  source 1..50: mol_type = other RNA

SEQ ID NO: 388 - RNA, 20 nt, synthetic construct
  source 1..20: mol_type = other RNA
atattgcacttgtcccggcc

SEQ ID NO: 389 - RNA, 50 nt, synthetic construct
  source 1..50: mol_type = other RNA
gtcgtatccagtgcagggtccgaggtattcgcactggatacgactcgggg

SEQ ID NO: 390 - RNA, 20 nt, synthetic construct
  source 1..20: mol_type = other RNA
atcctgtactgagctgcccc

SEQ ID NO: 391 - RNA, 50 nt, synthetic construct
  source 1..50: mol_type = other RNA
gtcgtatccagtgcagggtccgaggtattcgcactggatacgacctcggg

SEQ ID NO: 392 - RNA, 20 nt, synthetic construct
  source 1..20: mol_type = other RNA
atcctgtactgagctgcccc

SEQ ID NO: 393 - RNA, 50 nt, synthetic construct
  source 1..50: mol_type = other RNA
gtcgtatccagtgcagggtccgaggtattcgcactggatacgaccaggcc

SEQ ID NO: 394 - RNA, 20 nt, synthetic construct
  source 1..20: mol_type = other RNA
attgcacttgtcccggcctg

SEQ ID NO: 395 - RNA, 50 nt, synthetic construct; identical to SEQ ID NO: 311
  source 1..50: mol_type = other RNA

SEQ ID NO: 396 - RNA, 20 nt, synthetic construct
  source 1..20: mol_type = other RNA
attgcacttgtcccggcctg

SEQ ID NO: 397 - RNA, 50 nt, synthetic construct; identical to SEQ ID NO: 311
  source 1..50: mol_type = other RNA

SEQ ID NO: 398 - RNA, 20 nt, synthetic construct
  source 1..20: mol_type = other RNA
attgcacttgccccggcctg

SEQ ID NO: 399 - RNA, 50 nt, synthetic construct; identical to SEQ ID NO: 393
  source 1..50: mol_type = other RNA

SEQ ID NO: 400 - RNA, 20 nt, synthetic construct
  source 1..20: mol_type = other RNA
cattgcacttgtcccggcct

SEQ ID NO: 401 - RNA, 50 nt, synthetic construct
  source 1..50: mol_type = other RNA
gtcgtatccagtgcagggtccgaggtattcgcactggatacgacccagac

SEQ ID NO: 402 - RNA, 20 nt, synthetic construct
  source 1..20: mol_type = other RNA
cattgcacttgtctcggtct

SEQ ID NO: 403 - RNA, 50 nt, synthetic construct; identical to SEQ ID NO: 215
  source 1..50: mol_type = other RNA

SEQ ID NO: 404 - RNA, 20 nt, synthetic construct
  source 1..20: mol_type = other RNA
cattggtggttcagtggtag

SEQ ID NO: 405 - RNA, 50 nt, synthetic construct
  source 1..50: mol_type = other RNA
gtcgtatccagtgcagggtccgaggtattcgcactggatacgacaagcca

SEQ ID NO: 406 - RNA, 20 nt, synthetic construct
  source 1..20: mol_type = other RNA
ccccccgctgctaaatttga

SEQ ID NO: 407 - RNA, 50 nt, synthetic construct
  source 1..50: mol_type = other RNA
gtcgtatccagtgcagggtccgaggtattcgcactggatacgactctcgg

SEQ ID NO: 408 - RNA, 20 nt, synthetic construct
  source 1..20: mol_type = other RNA
ccctgtactgagctgccccg

SEQ ID NO: 409 - RNA, 50 nt, synthetic construct; identical to SEQ ID NO: 407
  source 1..50: mol_type = other RNA

SEQ ID NO: 410 - RNA, 20 nt, synthetic construct
  source 1..20: mol_type = other RNA
cctgtactgagctgccccga

SEQ ID NO: 411 - RNA, 50 nt, synthetic construct; identical to SEQ ID NO: 391
  source 1..50: mol_type = other RNA

SEQ ID NO: 412 - RNA, 20 nt, synthetic construct
  source 1..20: mol_type = other RNA
cctgtactgagctgccccga

SEQ ID NO: 413 - RNA, 50 nt, synthetic construct
  source 1..50: mol_type = other RNA
gtcgtatccagtgcagggtccgaggtattcgcactggatacgaccgtcct

SEQ ID NO: 414 - RNA, 20 nt, synthetic construct
  source 1..20: mol_type = other RNA
cgaggaggaagaggaggacg

SEQ ID NO: 415 - RNA, 50 nt, synthetic construct
  source 1..50: mol_type = other RNA
gtcgtatccagtgcagggtccgaggtattcgcactggatacgacctatac

SEQ ID NO: 416 - RNA, 20 nt, synthetic construct
  source 1..20: mol_type = other RNA
cgaggtagtaggttgtatag

SEQ ID NO: 417 - RNA, 50 nt, synthetic construct
  source 1..50: mol_type = other RNA
gtcgtatccagtgcagggtccgaggtattcgcactggatacgacaaaggc

SEQ ID NO: 418 - RNA, 20 nt, synthetic construct
  source 1..20: mol_type = other RNA
ctatacgacctgctgccttt

SEQ ID NO: 419 - RNA, 50 nt, synthetic construct; identical to SEQ ID NO: 391
  source 1..50: mol_type = other RNA

SEQ ID NO: 420 - RNA, 20 nt, synthetic construct
  source 1..20: mol_type = other RNA
ctgtactgagctgccccgag

SEQ ID NO: 421 - RNA, 50 nt, synthetic construct
  source 1..50: mol_type = other RNA
gtcgtatccagtgcagggtccgaggtattcgcactggatacgacgcgaga

SEQ ID NO: 422 - RNA, 20 nt, synthetic construct
  source 1..20: mol_type = other RNA
gaattggtggttcagtggta

SEQ ID NO: 423 - RNA, 50 nt, synthetic construct
  source 1..50: mol_type = other RNA
gtcgtatccagtgcagggtccgaggtattcgcactggatacgacaggcga

SEQ ID NO: 424 - RNA, 20 nt, synthetic construct
  source 1..20: mol_type = other RNA
gcatcggtggttcagtggta

SEQ ID NO: 425 - RNA, 50 nt, synthetic construct; identical to SEQ ID NO: 423
  source 1..50: mol_type = other RNA

SEQ ID NO: 426 - RNA, 20 nt, synthetic construct
  source 1..20: mol_type = other RNA
gcattagtggttcagtggta

SEQ ID NO: 427 - RNA, 50 nt, synthetic construct; identical to SEQ ID NO: 215
  source 1..50: mol_type = other RNA

SEQ ID NO: 428 - RNA, 20 nt, synthetic construct
  source 1..20: mol_type = other RNA
gcattgatggttcagtggta

SEQ ID NO: 429 - RNA, 50 nt, synthetic construct; identical to SEQ ID NO: 215
  source 1..50: mol_type = other RNA

SEQ ID NO: 430 - RNA, 20 nt, synthetic construct
  source 1..20: mol_type = other RNA
gcattggtagttcagtggta

SEQ ID NO: 431 - RNA, 50 nt, synthetic construct
  source 1..50: mol_type = other RNA
gtcgtatccagtgcagggtccgaggtattcgcactggatacgaccgggcg

SEQ ID NO: 432 - RNA, 20 nt, synthetic construct
  source 1..20: mol_type = other RNA
gcattggtggttcagtggta

SEQ ID NO: 433 - RNA, 50 nt, synthetic construct
  source 1..50: mol_type = other RNA
gtcgtatccagtgcagggtccgaggtattcgcactggatacgacgacgag

SEQ ID NO: 434 - RNA, 20 nt, synthetic construct
  source 1..20: mol_type = other RNA
gcattggtggttcagtggta

SEQ ID NO: 435 - RNA, 50 nt, synthetic construct; identical to SEQ ID NO: 423
  source 1..50: mol_type = other RNA

SEQ ID NO: 436 - RNA, 20 nt, synthetic construct
  source 1..20: mol_type = other RNA
gcattggtggttcagtggta

SEQ ID NO: 437 - RNA, 50 nt, synthetic construct; identical to SEQ ID NO: 215
  source 1..50: mol_type = other RNA

SEQ ID NO: 438 - RNA, 20 nt, synthetic construct
  source 1..20: mol_type = other RNA
gcattggtggttcagtggta

SEQ ID NO: 439 - RNA, 50 nt, synthetic construct; identical to SEQ ID NO: 215
  source 1..50: mol_type = other RNA

SEQ ID NO: 440 - RNA, 20 nt, synthetic construct
  source 1..20: mol_type = other RNA
gcattggtggttcagtggta

SEQ ID NO: 441 - RNA, 50 nt, synthetic construct; identical to SEQ ID NO: 215
  source 1..50: mol_type = other RNA

SEQ ID NO: 442 - RNA, 20 nt, synthetic construct
  source 1..20: mol_type = other RNA
gcattggtggttcggtggta

SEQ ID NO: 443 - RNA, 50 nt, synthetic construct
  source 1..50: mol_type = other RNA
gtcgtatccagtgcagggtccgaggtattcgcactggatacgacaatctt

SEQ ID NO: 444 - RNA, 20 nt, synthetic construct
  source 1..20: mol_type = other RNA
gcgccgctggtgtagtggca

SEQ ID NO: 445 - RNA, 50 nt, synthetic construct; identical to SEQ ID NO: 215
  source 1..50: mol_type = other RNA

SEQ ID NO: 446 - RNA, 20 nt, synthetic construct
  source 1..20: mol_type = other RNA
gcgttggtggttcagtggta

SEQ ID NO: 447 - RNA, 50 nt, synthetic construct
  source 1..50: mol_type = other RNA
gtcgtatccagtgcagggtccgaggtattcgcactggatacgactaagtt

SEQ ID NO: 448 - RNA, 20 nt, synthetic construct
  source 1..20: mol_type = other RNA
ggctggcccgatggtagtgg

SEQ ID NO: 449 - RNA, 50 nt, synthetic construct
  source 1..50: mol_type = other RNA
gtcgtatccagtgcagggtccgaggtattcgcactggatacgacttgagg

SEQ ID NO: 450 - RNA, 20 nt, synthetic construct
  source 1..20: mol_type = other RNA
ggctggtccgaaggtagtga

SEQ ID NO: 451 - RNA, 50 nt, synthetic construct; identical to SEQ ID NO: 369
  source 1..50: mol_type = other RNA

SEQ ID NO: 452 - RNA, 20 nt, synthetic construct
  source 1..20: mol_type = other RNA
ggctggtccgaaggcagtga

SEQ ID NO: 453 - RNA, 50 nt, synthetic construct
  source 1..50: mol_type = other RNA
gtcgtatccagtgcagggtccgaggtattcgcactggatacgacaagttc

SEQ ID NO: 454 - RNA, 20 nt, synthetic construct
  source 1..20: mol_type = other RNA
ggctggtccgatagtagtgg

SEQ ID NO: 455 - RNA, 50 nt, synthetic construct; identical to SEQ ID NO: 345
  source 1..50: mol_type = other RNA

SEQ ID NO: 456 - RNA, 20 nt, synthetic construct
  source 1..20: mol_type = other RNA
ggctggtccgatggtagtgg

SEQ ID NO: 457 - RNA, 50 nt, synthetic construct; identical to SEQ ID NO: 345
  source 1..50: mol_type = other RNA

SEQ ID NO: 458 - RNA, 20 nt, synthetic construct
  source 1..20: mol_type = other RNA
ggctggtccgatggtagcgg

SEQ ID NO: 459 - RNA, 50 nt, synthetic construct; identical to SEQ ID NO: 453
  source 1..50: mol_type = other RNA

SEQ ID NO: 460 - RNA, 20 nt, synthetic construct
  source 1..20: mol_type = other RNA
ggctggtccgatggtagtgg

SEQ ID NO: 461 - RNA, 50 nt, synthetic construct
  source 1..50: mol_type = other RNA
gtcgtatccagtgcagggtccgaggtattcgcactggatacgacgttctg

SEQ ID NO: 462 - RNA, 20 nt, synthetic construct
  source 1..20: mol_type = other RNA
ggctggtccgatggtagtag

SEQ ID NO: 463 - RNA, 50 nt, synthetic construct
  source 1..50: mol_type = other RNA
gtcgtatccagtgcagggtccgaggtattcgcactggatacgacgttatc

SEQ ID NO: 464 - RNA, 20 nt, synthetic construct
  source 1..20: mol_type = other RNA
ggggaattgcgagcggataa

SEQ ID NO: 465 - RNA, 50 nt, synthetic construct
  source 1..50: mol_type = other RNA
gtcgtatccagtgcagggtccgaggtattcgcactggatacgacattatc

SEQ ID NO: 466 - RNA, 20 nt, synthetic construct
  source 1..20: mol_type = other RNA
ggggaattgtgagcggataa

SEQ ID NO: 467 - RNA, 50 nt, synthetic construct
  source 1..50: mol_type = other RNA
gtcgtatccagtgcagggtccgaggtattcgcactggatacgacgttgtc

SEQ ID NO: 468 - RNA, 20 nt, synthetic construct
  source 1..20: mol_type = other RNA
ggggaattgtgagcggacaa

SEQ ID NO: 469 - RNA, 50 nt, synthetic construct
  source 1..50: mol_type = other RNA
gtcgtatccagtgcagggtccgaggtattcgcactggatacgaccctgtt

SEQ ID NO: 470 - RNA, 20 nt, synthetic construct
  source 1..20: mol_type = other RNA
taagcggcggggtcggaaca

SEQ ID NO: 471 - RNA, 50 nt, synthetic construct
  source 1..50: mol_type = other RNA
gtcgtatccagtgcagggtccgaggtattcgcactggatacgacagtctc

SEQ ID NO: 472 - RNA, 20 nt, synthetic construct
  source 1..20: mol_type = other RNA
taaggggcagagagcgagac

SEQ ID NO: 473 - RNA, 50 nt, synthetic construct; identical to SEQ ID NO: 311
  source 1..50: mol_type = other RNA

SEQ ID NO: 474 - RNA, 20 nt, synthetic construct
  source 1..20: mol_type = other RNA
taatgcacttgtcccggcct

SEQ ID NO: 475 - RNA, 50 nt, synthetic construct
  source 1..50: mol_type = other RNA
gtcgtatccagtgcagggtccgaggtattcgcactggatacgaccgccga

SEQ ID NO: 476 - RNA, 20 nt, synthetic construct
  source 1..20: mol_type = other RNA
tagcagcacgtaaatatcgg

SEQ ID NO: 477 - RNA, 50 nt, synthetic construct
  source 1..50: mol_type = other RNA
gtcgtatccagtgcagggtccgaggtattcgcactggatacgactcaacg

SEQ ID NO: 478 - RNA, 20 nt, synthetic construct
  source 1..20: mol_type = other RNA
tagcttatcagactgacgtt

SEQ ID NO: 479 - RNA, 50 nt, synthetic construct
  source 1..50: mol_type = other RNA
gtcgtatccagtgcagggtccgaggtattcgcactggatacgacttaaca

SEQ ID NO: 480 - RNA, 20 nt, synthetic construct
  source 1..20: mol_type = other RNA
tagcttatcagactgatgtt

SEQ ID NO: 481 - RNA, 50 nt, synthetic construct
  source 1..50: mol_type = other RNA
gtcgtatccagtgcagggtccgaggtattcgcactggatacgacaacatc

SEQ ID NO: 482 - RNA, 20 nt, synthetic construct
  source 1..20: mol_type = other RNA
tagcttatcagactgatgtt

SEQ ID NO: 483 - RNA, 50 nt, synthetic construct; identical to SEQ ID NO: 311
  source 1..50: mol_type = other RNA

SEQ ID NO: 484 - RNA, 20 nt, synthetic construct
  source 1..20: mol_type = other RNA
tattgcacttgtcacggcct

SEQ ID NO: 485 - RNA, 50 nt, synthetic construct; identical to SEQ ID NO: 311
  source 1..50: mol_type = other RNA

SEQ ID NO: 486 - RNA, 20 nt, synthetic construct
  source 1..20: mol_type = other RNA
tattgcacttgccccggcct

SEQ ID NO: 487 - RNA, 50 nt, synthetic construct
  source 1..50: mol_type = other RNA
gtcgtatccagtgcagggtccgaggtattcgcactggatacgaccagacc

SEQ ID NO: 488 - RNA, 20 nt, synthetic construct
  source 1..20: mol_type = other RNA
tattgcacttgtcccggtct

SEQ ID NO: 489 - RNA, 50 nt, synthetic construct; identical to SEQ ID NO: 311
  source 1..50: mol_type = other RNA

SEQ ID NO: 490 - RNA, 20 nt, synthetic construct
  source 1..20: mol_type = other RNA
tattgcacttgtcccagcct

SEQ ID NO: 491 - RNA, 50 nt, synthetic construct; identical to SEQ ID NO: 311
  source 1..50: mol_type = other RNA

SEQ ID NO: 492 - RNA, 20 nt, synthetic construct
  source 1..20: mol_type = other RNA
tattgcactcgtcccggcct

SEQ ID NO: 493 - RNA, 50 nt, synthetic construct
  source 1..50: mol_type = other RNA
gtcgtatccagtgcagggtccgaggtattcgcactggatacgacactggc

SEQ ID NO: 494 - RNA, 20 nt, synthetic construct
  source 1..20: mol_type = other RNA
tattgcacttgtcccggcca

SEQ ID NO: 495 - RNA, 50 nt, synthetic construct
  source 1..50: mol_type = other RNA
gtcgtatccagtgcagggtccgaggtattcgcactggatacgaccgggcc

SEQ ID NO: 496 - RNA, 20 nt, synthetic construct
  source 1..20: mol_type = other RNA
tattgcacttgtcccggccc

SEQ ID NO: 497 - RNA, 50 nt, synthetic construct; identical to SEQ ID NO: 393
  source 1..50: mol_type = other RNA

SEQ ID NO: 498 - RNA, 20 nt, synthetic construct
  source 1..20: mol_type = other RNA
tattgcacttgtcccggcct

SEQ ID NO: 499 - RNA, 50 nt, synthetic construct; identical to SEQ ID NO: 393
  source 1..50: mol_type = other RNA

SEQ ID NO: 500 - RNA, 20 nt, synthetic construct
  source 1..20: mol_type = other RNA
tattgcgcttgtcccggcct

SEQ ID NO: 501 - RNA, 50 nt, synthetic construct; identical to SEQ ID NO: 311
  source 1..50: mol_type = other RNA

SEQ ID NO: 502 - RNA, 20 nt, synthetic construct
  source 1..20: mol_type = other RNA
tattgcacttgtccctgcct

SEQ ID NO: 503 - RNA, 50 nt, synthetic construct; identical to SEQ ID NO: 311
  source 1..50: mol_type = other RNA

SEQ ID NO: 504 - RNA, 20 nt, synthetic construct
  source 1..20: mol_type = other RNA
tattgcacttatcccggcct

SEQ ID NO: 505 - RNA, 50 nt, synthetic construct; identical to SEQ ID NO: 311
  source 1..50: mol_type = other RNA

SEQ ID NO: 506 - RNA, 20 nt, synthetic construct
  source 1..20: mol_type = other RNA
tattgcacttgtccaggcct

SEQ ID NO: 507 - RNA, 50 nt, synthetic construct; identical to SEQ ID NO: 311
  source 1..50: mol_type = other RNA

SEQ ID NO: 508 - RNA, 20 nt, synthetic construct
  source 1..20: mol_type = other RNA
tattgctcttgtcccggcct

SEQ ID NO: 509 - RNA, 50 nt, synthetic construct
  source 1..50: mol_type = other RNA
gtcgtatccagtgcagggtccgaggtattcgcactggatacgaccacagg

SEQ ID NO: 510 - RNA, 20 nt, synthetic construct
  source 1..20: mol_type = other RNA
tattgcacttgtcccggcct

SEQ ID NO: 511 - RNA, 50 nt, synthetic construct; identical to SEQ ID NO: 407
  source 1..50: mol_type = other RNA

SEQ ID NO: 512 - RNA, 20 nt, synthetic construct
  source 1..20: mol_type = other RNA
tcccgtactgagctgccccg

SEQ ID NO: 513 - RNA, 50 nt, synthetic construct; identical to SEQ ID NO: 391
  source 1..50: mol_type = other RNA

SEQ ID NO: 514 - RNA, 20 nt, synthetic construct
  source 1..20: mol_type = other RNA
tcccgtactgagctgccccg

SEQ ID NO: 515 - RNA, 50 nt, synthetic construct
  source 1..50: mol_type = other RNA
gtcgtatccagtgcagggtccgaggtattcgcactggatacgaccggggc

SEQ ID NO: 516 - RNA, 20 nt, synthetic construct
  source 1..20: mol_type = other RNA
tcccgtactgagctgccccg

SEQ ID NO: 517 - RNA, 50 nt, synthetic construct
  source 1..50: mol_type = other RNA
gtcgtatccagtgcagggtccgaggtattcgcactggatacgacgcgccg

SEQ ID NO: 518 - RNA, 20 nt, synthetic construct
  source 1..20: mol_type = other RNA
tccctggtggtctaatggtt

SEQ ID NO: 519 - RNA, 50 nt, synthetic construct; identical to SEQ ID NO: 517
  source 1..50: mol_type = other RNA

SEQ ID NO: 520 - RNA, 20 nt, synthetic construct
  source 1..20: mol_type = other RNA
tccctggtggtctagtagtt

SEQ ID NO: 521 - RNA, 50 nt, synthetic construct
  source 1..50: mol_type = other RNA
gtcgtatccagtgcagggtccgaggtattcgcactggatacgacagcgcc

SEQ ID NO: 522 - RNA, 20 nt, synthetic construct
  source 1..20: mol_type = other RNA
tccctggtgatctagtggtt

SEQ ID NO: 523 - RNA, 50 nt, synthetic construct
  source 1..50: mol_type = other RNA
gtcgtatccagtgcagggtccgaggtattcgcactggatacgacagagcg

SEQ ID NO: 524 - RNA, 20 nt, synthetic construct
  source 1..20: mol_type = other RNA
tccctggtggtctagtggtc

SEQ ID NO: 525 - RNA, 50 nt, synthetic construct; identical to SEQ ID NO: 407
  source 1..50: mol_type = other RNA

SEQ ID NO: 526 - RNA, 20 nt, synthetic construct
  source 1..20: mol_type = other RNA
tcctgcactgagctgccccg

SEQ ID NO: 527 - RNA, 50 nt, synthetic construct; identical to SEQ ID NO: 407
  source 1..50: mol_type = other RNA

SEQ ID NO: 528 - RNA, 20 nt, synthetic construct
  source 1..20: mol_type = other RNA
tcctgtactaagctgccccg

SEQ ID NO: 529 - RNA, 50 nt, synthetic construct
  source 1..50: mol_type = other RNA
gtcgtatccagtgcagggtccgaggtattcgcactggatacgacactcgg

SEQ ID NO: 530 - RNA, 20 nt, synthetic construct
  source 1..20: mol_type = other RNA
tcctgtaccgagctgccccg

SEQ ID NO: 531 - RNA, 50 nt, synthetic construct
  source 1..50: mol_type = other RNA
gtcgtatccagtgcagggtccgaggtattcgcactggatacgaccttggg

SEQ ID NO: 532 - RNA, 20 nt, synthetic construct
  source 1..20: mol_type = other RNA
tcctgtactgagctgcccca

SEQ ID NO: 533 - RNA, 50 nt, synthetic construct
  source 1..50: mol_type = other RNA
gtcgtatccagtgcagggtccgaggtattcgcactggatacgactcccgg

SEQ ID NO: 534 - RNA, 20 nt, synthetic construct
  source 1..20: mol_type = other RNA
tcctgtactgagctgccccg

SEQ ID NO: 535 - RNA, 50 nt, synthetic construct
  source 1..50: mol_type = other RNA
gtcgtatccagtgcagggtccgaggtattcgcactggatacgaccacggg

SEQ ID NO: 536 - RNA, 20 nt, synthetic construct
  source 1..20: mol_type = other RNA
tcctgtactgagctgccccg

SEQ ID NO: 537 - RNA, 50 nt, synthetic construct; identical to SEQ ID NO: 407
  source 1..50: mol_type = other RNA

SEQ ID NO: 538 - RNA, 20 nt, synthetic construct
  source 1..20: mol_type = other RNA
tcctgtaccgagctgccccg

SEQ ID NO: 539 - RNA, 50 nt, synthetic construct
  source 1..50: mol_type = other RNA
gtcgtatccagtgcagggtccgaggtattcgcactggatacgactcgtgg

SEQ ID NO: 540 - RNA, 20 nt, synthetic construct
  source 1..20: mol_type = other RNA
tcctgtactgagctgccacg

SEQ ID NO: 541 - RNA, 50 nt, synthetic construct; identical to SEQ ID NO: 391
  source 1..50: mol_type = other RNA

SEQ ID NO: 542 - RNA, 20 nt, synthetic construct
  source 1..20: mol_type = other RNA
tcctgtaccgagctgccccg

SEQ ID NO: 543 - RNA, 50 nt, synthetic construct
  source 1..50: mol_type = other RNA
gtcgtatccagtgcagggtccgaggtattcgcactggatacgacacgggg

SEQ ID NO: 544 - RNA, 20 nt, synthetic construct
  source 1..20: mol_type = other RNA
tcctgtactgagctgccccg

SEQ ID NO: 545 - RNA, 50 nt, synthetic construct
  source 1..50: mol_type = other RNA
gtcgtatccagtgcagggtccgaggtattcgcactggatacgactcttgg

SEQ ID NO: 546 - RNA, 20 nt, synthetic construct
  source 1..20: mol_type = other RNA
tcctgtactgagctgcccca

SEQ ID NO: 547 - RNA, 50 nt, synthetic construct; identical to SEQ ID NO: 391
  source 1..50: mol_type = other RNA

SEQ ID NO: 548 - RNA, 20 nt, synthetic construct
  source 1..20: mol_type = other RNA
tcctgtactgagttgccccg

SEQ ID NO: 549 - RNA, 50 nt, synthetic construct; identical to SEQ ID NO: 529
  source 1..50: mol_type = other RNA

SEQ ID NO: 550 - RNA, 20 nt, synthetic construct
  source 1..20: mol_type = other RNA
tcctgtactgggctgccccg

SEQ ID NO: 551 - RNA, 50 nt, synthetic construct; identical to SEQ ID NO: 391
  source 1..50: mol_type = other RNA

SEQ ID NO: 552 - RNA, 20 nt, synthetic construct
  source 1..20: mol_type = other RNA
tcctgtactgggctgccccg

SEQ ID NO: 553 - RNA, 50 nt, synthetic construct; identical to SEQ ID NO: 391
  source 1..50: mol_type = other RNA

SEQ ID NO: 554 - RNA, 20 nt, synthetic construct
  source 1..20: mol_type = other RNA
tcctgtactgagctgcccga

SEQ ID NO: 555 - RNA, 50 nt, synthetic construct
  source 1..50: mol_type = other RNA
gtcgtatccagtgcagggtccgaggtattcgcactggatacgacacccat

SEQ ID NO: 556 - RNA, 20 nt, synthetic construct
  source 1..20: mol_type = other RNA
tgagaactgaattccatggg

SEQ ID NO: 557 - RNA, 50 nt, synthetic construct
  source 1..50: mol_type = other RNA
gtcgtatccagtgcagggtccgaggtattcgcactggatacgacggtctc

SEQ ID NO: 558 - RNA, 20 nt, synthetic construct
  source 1..20: mol_type = other RNA
tgaggggcagagagcgagac

SEQ ID NO: 559 - RNA, 50 nt, synthetic construct; identical to SEQ ID NO: 471
  source 1..50: mol_type = other RNA

SEQ ID NO: 560 - RNA, 20 nt, synthetic construct
  source 1..20: mol_type = other RNA
tgagggacagagagcgagac

SEQ ID NO: 561 - RNA, 50 nt, synthetic construct
  source 1..50: mol_type = other RNA
gtcgtatccagtgcagggtccgaggtattcgcactggatacgacaaaagt

SEQ ID NO: 562 - RNA, 20 nt, synthetic construct
  source 1..20: mol_type = other RNA
tgaggggcagagagcgggac

SEQ ID NO: 563 - RNA, 50 nt, synthetic construct
  source 1..50: mol_type = other RNA
gtcgtatccagtgcagggtccgaggtattcgcactggatacgacaaagtc

SEQ ID NO: 564 - RNA, 20 nt, synthetic construct
  source 1..20: mol_type = other RNA
tgaggggcagagggcgagac

SEQ ID NO: 565 - RNA, 50 nt, synthetic construct
  source 1..50: mol_type = other RNA
gtcgtatccagtgcagggtccgaggtattcgcactggatacgacaaacca

SEQ ID NO: 566 - RNA, 20 nt, synthetic construct
  source 1..20: mol_type = other RNA
tgaggtagtaggtcgtgtgg

SEQ ID NO: 567 - RNA, 50 nt, synthetic construct; identical to SEQ ID NO: 323
  source 1..50: mol_type = other RNA

SEQ ID NO: 568 - RNA, 20 nt, synthetic construct
  source 1..20: mol_type = other RNA
tgaggtagtaggttgtgtgg

SEQ ID NO: 569 - RNA, 50 nt, synthetic construct; identical to SEQ ID NO: 353
  source 1..50: mol_type = other RNA

SEQ ID NO: 570 - RNA, 20 nt, synthetic construct
  source 1..20: mol_type = other RNA
tgaggtagtaggtcgtgtgg

SEQ ID NO: 571 - RNA, 50 nt, synthetic construct; identical to SEQ ID NO: 415
  source 1..50: mol_type = other RNA

SEQ ID NO: 572 - RNA, 20 nt, synthetic construct
  source 1..20: mol_type = other RNA
tgaggtagtaggttgtatag

SEQ ID NO: 573 - RNA, 50 nt, synthetic construct
  source 1..50: mol_type = other RNA
gtcgtatccagtgcagggtccgaggtattcgcactggatacgacacagcg

SEQ ID NO: 574 - RNA, 20 nt, synthetic construct
  source 1..20: mol_type = other RNA
tgaggtagtagtttgcgctg

SEQ ID NO: 575 - RNA, 50 nt, synthetic construct; identical to SEQ ID NO: 415
  source 1..50: mol_type = other RNA

SEQ ID NO: 576 - RNA, 20 nt, synthetic construct
  source 1..20: mol_type = other RNA
tgaggtagtaggtcgtatag

SEQ ID NO: 577 - RNA, 50 nt, synthetic construct
  source 1..50: mol_type = other RNA
gtcgtatccagtgcagggtccgaggtattcgcactggatacgacactata

SEQ ID NO: 578 - RNA, 20 nt, synthetic construct
  source 1..20: mol_type = other RNA
tgaggtagtaggtcgtatag

SEQ ID NO: 579 - RNA, 50 nt, synthetic construct
  source 1..50: mol_type = other RNA
gtcgtatccagtgcagggtccgaggtattcgcactggatacgaccacaca

SEQ ID NO: 580 - RNA, 19 nt, synthetic construct
  source 1..19: mol_type = other RNA
tgaggtagtaggttgtgtg

SEQ ID NO: 581 - RNA, 50 nt, synthetic construct
  source 1..50: mol_type = other RNA
gtcgtatccagtgcagggtccgaggtattcgcactggatacgacaaccac

SEQ ID NO: 582 - RNA, 20 nt, synthetic construct
  source 1..20: mol_type = other RNA
tgaggtagtaggttatgtgg

SEQ ID NO: 583 - RNA, 50 nt, synthetic construct
  source 1..50: mol_type = other RNA
gtcgtatccagtgcagggtccgaggtattcgcactggatacgacaactat

SEQ ID NO: 584 - RNA, 20 nt, synthetic construct
  source 1..20: mol_type = other RNA
tgaggtagtggattgtatag

SEQ ID NO: 585 - RNA, 50 nt, synthetic construct
  source 1..50: mol_type = other RNA
gtcgtatccagtgcagggtccgaggtattcgcactggatacgacaactgt

SEQ ID NO: 586 - RNA, 20 nt, synthetic construct
  source 1..20: mol_type = other RNA
tgaggtagtagtttgcacag

SEQ ID NO: 587 - RNA, 50 nt, synthetic construct; identical to SEQ ID NO: 353
  source 1..50: mol_type = other RNA

SEQ ID NO: 588 - RNA, 20 nt, synthetic construct
  source 1..20: mol_type = other RNA
tgaggtagtaggttgtgtgg

SEQ ID NO: 589 - RNA, 50 nt, synthetic construct
  source 1..50: mol_type = other RNA
gtcgtatccagtgcagggtccgaggtattcgcactggatacgaccagaca

SEQ ID NO: 590 - RNA, 20 nt, synthetic construct
  source 1..20: mol_type = other RNA
tggagtgtgacaatggtgtc

SEQ ID NO: 591 - RNA, 50 nt, synthetic construct; identical to SEQ ID NO: 383
  source 1..50: mol_type = other RNA

SEQ ID NO: 592 - RNA, 20 nt, synthetic construct
  source 1..20: mol_type = other RNA
tgggtttacgttgggagaac

SEQ ID NO: 593 - RNA, 50 nt, synthetic construct
  source 1..50: mol_type = other RNA
gtcgtatccagtgcagggtccgaggtattcgcactggatacgaccttcca

SEQ ID NO: 594 - RNA, 20 nt, synthetic construct
  source 1..20: mol_type = other RNA
tgtaaacatccttgactgga

SEQ ID NO: 595 - RNA, 50 nt, synthetic construct
  source 1..50: mol_type = other RNA
gtcgtatccagtgcagggtccgaggtattcgcactggatacgactttcca

SEQ ID NO: 596 - RNA, 20 nt, synthetic construct
  source 1..20: mol_type = other RNA
tgtaaacatccccgactgga

SEQ ID NO: 597 - RNA, 50 nt, synthetic construct
  source 1..50: mol_type = other RNA
gtcgtatccagtgcagggtccgaggtattcgcactggatacgactgagag

SEQ ID NO: 598 - RNA, 20 nt, synthetic construct
  source 1..20: mol_type = other RNA
tgtaaacatcctacactctc

SEQ ID NO: 599 - RNA, 50 nt, synthetic construct
  source 1..50: mol_type = other RNA
gtcgtatccagtgcagggtccgaggtattcgcactggatacgacagcttc

SEQ ID NO: 600 - RNA, 20 nt, synthetic construct
  source 1..20: mol_type = other RNA
tgtagacatccttgactgga

SEQ ID NO: 601 - RNA, 50 nt, synthetic construct; identical to SEQ ID NO: 311
  source 1..50: mol_type = other RNA

SEQ ID NO: 602 - RNA, 20 nt, synthetic construct
  source 1..20: mol_type = other RNA
tgttgcacttgtcccggcct

SEQ ID NO: 603 - RNA, 50 nt, synthetic construct; identical to SEQ ID NO: 407
  source 1..50: mol_type = other RNA

SEQ ID NO: 604 - RNA, 20 nt, synthetic construct
  source 1..20: mol_type = other RNA
ttctgtactgagctgccccg

SEQ ID NO: 605 - RNA, 50 nt, synthetic construct; identical to SEQ ID NO: 311
  source 1..50: mol_type = other RNA

SEQ ID NO: 606 - RNA, 20 nt, synthetic construct
  source 1..20: mol_type = other RNA
ttgcacttgtcccggcctgt

SEQ ID NO: 607 - RNA, 50 nt, synthetic construct; identical to SEQ ID NO: 333
  source 1..50: mol_type = other RNA

SEQ ID NO: 608 - RNA, 18 nt, synthetic construct
  source 1..18: mol_type = other RNA
ttgggagcgggcgggcgg

SEQ ID NO: 609 - RNA, 50 nt, synthetic construct; identical to SEQ ID NO: 351
  source 1..50: mol_type = other RNA

SEQ ID NO: 610 - RNA, 18 nt, synthetic construct
  source 1..18: mol_type = other RNA
attccggttgttcttctc

SEQ ID NO: 611 - RNA, 50 nt, synthetic construct
  source 1..50: mol_type = other RNA
gtcgtatccagtgcagggtccgaggtattcgcactggatacgacccatct

SEQ ID NO: 612 - RNA, 20 nt, synthetic construct
  source 1..20: mol_type = other RNA
ttgtagcactggggccagat

SEQ ID NO: 613 - RNA, 50 nt, synthetic construct
  source 1..50: mol_type = other RNA
gtcgtatccagtgcagggtccgaggtattcgcactggatacgacggcgtc

SEQ ID NO: 614 - RNA, 20 nt, synthetic construct
  source 1..20: mol_type = other RNA
agatgagaggacgggacgcc

SEQ ID NO: 615 - RNA, 50 nt, synthetic construct
  source 1..50: mol_type = other RNA
gtcgtatccagtgcagggtccgaggtattcgcactggatacgactttcag

SEQ ID NO: 616 - RNA, 20 nt, synthetic construct
  source 1..20: mol_type = other RNA
tatggaagtgccgccgctga

SEQ ID NO: 617 - RNA, 50 nt, synthetic construct
  source 1..50: mol_type = other RNA
gtcgtatccagtgcagggtccgaggtattcgcactggatacgacgccagc

SEQ ID NO: 618 - RNA, 20 nt, synthetic construct
  source 1..20: mol_type = other RNA
tattaaatcaatctcggctg

SEQ ID NO: 619 - RNA, 16 nt, synthetic construct
  source 1..16: mol_type = other RNA
gtgcagggtccgaggt

SEQ ID NO: 620 - RNA, 15 nt, synthetic construct
  source 1..15: mol_type = other RNA
ccccctcttcagcat

SEQ ID NO: 621 - RNA, 15 nt, synthetic construct
  source 1..15: mol_type = other RNA
tcgtctggtcagcat

SEQ ID NO: 622 - RNA, 15 nt, synthetic construct
  source 1..15: mol_type = other RNA
ttttccgctcagcat

SEQ ID NO: 623 - RNA, 15 nt, synthetic construct
  source 1..15: mol_type = other RNA
ttttttgcccagcat

SEQ ID NO: 624 - RNA, 15 nt, synthetic construct
  source 1..15: mol_type = other RNA
ggcgtgtttcagcat

SEQ ID NO: 625 - RNA, 15 nt, synthetic construct
  source 1..15: mol_type = other RNA
tcctttgcgcagcat

SEQ ID NO: 626 - RNA, 15 nt, synthetic construct
  source 1..15: mol_type = other RNA
ggccccgctcagcat

SEQ ID NO: 627 - RNA, 15 nt, synthetic construct
  source 1..15: mol_type = other RNA
cctttttcgcagcat

SEQ ID NO: 628 - RNA, 15 nt, synthetic construct
  source 1..15: mol_type = other RNA
ggttgctctcagcat

SEQ ID NO: 629 - RNA, 15 nt, synthetic construct
  source 1..15: mol_type = other RNA
gttgcctgtcagcat

SEQ ID NO: 630 - RNA, 15 nt, synthetic construct
  source 1..15: mol_type = other RNA
gttggtggccagcat

SEQ ID NO: 631 - RNA, 15 nt, synthetic construct
  source 1..15: mol_type = other RNA
tcctgtcctcagcat

SEQ ID NO: 632 - RNA, 15 nt, synthetic construct
  source 1..15: mol_type = other RNA
ttcttctgtcagcat

SEQ ID NO: 633 - RNA, 15 nt, synthetic construct
  source 1..15: mol_type = other RNA
tttgtgtttcagcat

SEQ ID NO: 634 - RNA, 15 nt, synthetic construct
  source 1..15: mol_type = other RNA
ttttcttttcagcat

SEQ ID NO: 635 - RNA, 15 nt, synthetic construct
  source 1..15: mol_type = other RNA
ctggcgttccagcat

SEQ ID NO: 636 - RNA, 15 nt, synthetic construct
  source 1..15: mol_type = other RNA
gcccttgtccagcat

SEQ ID NO: 637 - RNA, 15 nt, synthetic construct
  source 1..15: mol_type = other RNA
ggtttgccccagcat

SEQ ID NO: 638 - RNA, 15 nt, synthetic construct
  source 1..15: mol_type = other RNA
cttgctgtgcagcat

SEQ ID NO: 639 - RNA, 15 nt, synthetic construct
  source 1..15: mol_type = other RNA
ctttcgcctcagcat

SEQ ID NO: 640 - RNA, 15 nt, synthetic construct
  source 1..15: mol_type = other RNA
tttggctcccagcat

SEQ ID NO: 641 - RNA, 15 nt, synthetic construct
  source 1..15: mol_type = other RNA
tgtttctcgcagcat

SEQ ID NO: 642 - RNA, 15 nt, synthetic construct
  source 1..15: mol_type = other RNA
tttgcctggcagcat

SEQ ID NO: 643 - RNA, 15 nt, synthetic construct
  source 1..15: mol_type = other RNA
cgtggtgcccagcat

SEQ ID NO: 644 - RNA, 15 nt, synthetic construct
  source 1..15: mol_type = other RNA
gtgttgtttcagcat

SEQ ID NO: 645 - RNA, 15 nt, synthetic construct
  source 1..15: mol_type = other RNA
gttcgtttccagcat

SEQ ID NO: 646 - RNA, 15 nt, synthetic construct
  source 1..15: mol_type = other RNA
gccttgctccagcat

SEQ ID NO: 647 - RNA, 15 nt, synthetic construct
  source 1..15: mol_type = other RNA
gtcctgctccagcat

SEQ ID NO: 648 - RNA, 15 nt, synthetic construct
  source 1..15: mol_type = other RNA
gttctttttcagcat

SEQ ID NO: 649 - RNA, 15 nt, synthetic construct
  source 1..15: mol_type = other RNA
tgttctccccagcat

SEQ ID NO: 650 - RNA, 15 nt, synthetic construct
  source 1..15: mol_type = other RNA
tccttcggccagcat

SEQ ID NO: 651 - RNA, 15 nt, synthetic construct
  source 1..15: mol_type = other RNA
ttttccgggcagcat

SEQ ID NO: 652 - RNA, 15 nt, synthetic construct
  source 1..15: mol_type = other RNA
cttgtccttcagcat

SEQ ID NO: 653 - RNA, 15 nt, synthetic construct
  source 1..15: mol_type = other RNA
tggttttctcagcat

SEQ ID NO: 654 - RNA, 15 nt, synthetic construct
  source 1..15: mol_type = other RNA
ccttgccttcagcat

SEQ ID NO: 655 - RNA, 15 nt, synthetic construct
  source 1..15: mol_type = other RNA
gttccctgtcagcat

SEQ ID NO: 656 - RNA, 15 nt, synthetic construct
  source 1..15: mol_type = other RNA
ggcgtgtttcagcat

SEQ ID NO: 657 - RNA, 15 nt, synthetic construct
  source 1..15: mol_type = other RNA
ctttttctccagcat

SEQ ID NO: 658 - RNA, 15 nt, synthetic construct
  source 1..15: mol_type = other RNA
tccccttctcagcat

SEQ ID NO: 659 - RNA, 15 nt, synthetic construct
  source 1..15: mol_type = other RNA
ttctcccttcagcat

SEQ ID NO: 660 - RNA, 15 nt, synthetic construct
  source 1..15: mol_type = other RNA
gtcccgccgcagcat

SEQ ID NO: 661 - RNA, 15 nt, synthetic construct
  source 1..15: mol_type = other RNA
tttcccttgcagcat

SEQ ID NO: 662 - RNA, 15 nt, synthetic construct
  source 1..15: mol_type = other RNA
ccgtcctcccagcat

SEQ ID NO: 663 - RNA, 15 nt, synthetic construct
  source 1..15: mol_type = other RNA
tctttgcttcagcat

SEQ ID NO: 664 - RNA, 15 nt, synthetic construct
  source 1..15: mol_type = other RNA
tttccggctcagcat

SEQ ID NO: 665 - RNA, 15 nt, synthetic construct
  source 1..15: mol_type = other RNA
tggcgtgcccagcat

SEQ ID NO: 666 - RNA, 15 nt, synthetic construct
  source 1..15: mol_type = other RNA
ctggcgtgtcagcat

SEQ ID NO: 667 - RNA, 15 nt, synthetic construct
  source 1..15: mol_type = other RNA
gttttcttccagcat

SEQ ID NO: 668 - RNA, 15 nt, synthetic construct
  source 1..15: mol_type = other RNA
tgtgttttccagcat

SEQ ID NO: 669 - RNA, 15 nt, synthetic construct
  source 1..15: mol_type = other RNA
ttgcttcctcagcat

SEQ ID NO: 670 - RNA, 15 nt, synthetic construct
  source 1..15: mol_type = other RNA
gtcttcgtccagcat

SEQ ID NO: 671 - RNA, 15 nt, synthetic construct
  source 1..15: mol_type = other RNA
tgtcttcttcagcat

SEQ ID NO: 672 - RNA, 15 nt, synthetic construct
  source 1..15: mol_type = other RNA
tctggccggcagcat

SEQ ID NO: 673 - RNA, 15 nt, synthetic construct
  source 1..15: mol_type = other RNA
ttgcttctgcagcat

SEQ ID NO: 674 - RNA, 15 nt, synthetic construct
  source 1..15: mol_type = other RNA
ttctcgctccagcat

SEQ ID NO: 675 - RNA, 15 nt, synthetic construct
  source 1..15: mol_type = other RNA
tgcctcgctcagcat

SEQ ID NO: 676 - RNA, 15 nt, synthetic construct
  source 1..15: mol_type = other RNA
ttggtctcgcagcat

SEQ ID NO: 677 - RNA, 15 nt, synthetic construct
  source 1..15: mol_type = other RNA
tcttctcgccagcat

SEQ ID NO: 678 - RNA, 15 nt, synthetic construct
  source 1..15: mol_type = other RNA
tgtcctctccagcat

SEQ ID NO: 679 - RNA, 15 nt, synthetic construct
  source 1..15: mol_type = other RNA
tctcctttgcagcat

SEQ ID NO: 680 - RNA, 15 nt, synthetic construct
  source 1..15: mol_type = other RNA
ctttctgtccagcat

SEQ ID NO: 681 - RNA, 15 nt, synthetic construct
  source 1..15: mol_type = other RNA
gcttcctgtcagcat

SEQ ID NO: 682 - RNA, 15 nt, synthetic construct
  source 1..15: mol_type = other RNA
tcttcctgtcagcat

SEQ ID NO: 683 - RNA, 15 nt, synthetic construct
  source 1..15: mol_type = other RNA
ccgcccgcccagcat

SEQ ID NO: 684 - RNA, 15 nt, synthetic construct
  source 1..15: mol_type = other RNA
tgttcctttcagcat

SEQ ID NO: 685 - RNA, 15 nt, synthetic construct
  source 1..15: mol_type = other RNA
gcgttcgtgcagcat

SEQ ID NO: 686 - RNA, 15 nt, synthetic construct
  source 1..15: mol_type = other RNA
cgcgtgccgcagcat

SEQ ID NO: 687 - RNA, 15 nt, synthetic construct
  source 1..15: mol_type = other RNA
cccccgccgcagcat

SEQ ID NO: 688 - RNA, 15 nt, synthetic construct
  source 1..15: mol_type = other RNA
tcctcctcccagcat

SEQ ID NO: 689 - RNA, 15 nt, synthetic construct
  source 1..15: mol_type = other RNA
ttctgttttcagcat

SEQ ID NO: 690 - RNA, 15 nt, synthetic construct
  source 1..15: mol_type = other RNA
cccccgccgcagcat

SEQ ID NO: 691 - RNA, 15 nt, synthetic construct
  source 1..15: mol_type = other RNA
ccccgccgccagcat

SEQ ID NO: 692 - RNA, 15 nt, synthetic construct
  source 1..15: mol_type = other RNA
gtgttgttccagcat

SEQ ID NO: 693 - RNA, 15 nt, synthetic construct
  source 1..15: mol_type = other RNA
cctctcttccagcat

SEQ ID NO: 694 - RNA, 15 nt, synthetic construct
  source 1..15: mol_type = other RNA
tctccgtctcagcat

SEQ ID NO: 695 - RNA, 15 nt, synthetic construct
  source 1..15: mol_type = other RNA
tctgcccttcagcat

SEQ ID NO: 696 - RNA, 15 nt, synthetic construct
  source 1..15: mol_type = other RNA
gcttttgggcagcat

SEQ ID NO: 697 - RNA, 15 nt, synthetic construct
  source 1..15: mol_type = other RNA
tttggtgttcagcat

SEQ ID NO: 698 - RNA, 15 nt, synthetic construct
  source 1..15: mol_type = other RNA
tcccgggtccagcat

SEQ ID NO: 699 - RNA, 15 nt, synthetic construct
  source 1..15: mol_type = other RNA
gtgcgccgtcagcat

SEQ ID NO: 700 - RNA, 15 nt, synthetic construct
  source 1..15: mol_type = other RNA
tccctctctcagcat

SEQ ID NO: 701 - RNA, 15 nt, synthetic construct
  source 1..15: mol_type = other RNA
ttgtgttttcagcat

SEQ ID NO: 702 - RNA, 15 nt, synthetic construct
  source 1..15: mol_type = other RNA
cgccctctccagcat

SEQ ID NO: 703 - RNA, 15 nt, synthetic construct
  source 1..15: mol_type = other RNA
tccctctctcagcat

SEQ ID NO: 704 - RNA, 15 nt, synthetic construct
  source 1..15: mol_type = other RNA
ctcgccctccagcat

SEQ ID NO: 705 - RNA, 15 nt, synthetic construct
  source 1..15: mol_type = other RNA
gctgttcttcagcat

SEQ ID NO: 706 - RNA, 15 nt, synthetic construct
  source 1..15: mol_type = other RNA
tttccctgccagcat

SEQ ID NO: 707 - RNA, 15 nt, synthetic construct
  source 1..15: mol_type = other RNA
tctgtggggcagcat

SEQ ID NO: 708 - RNA, 15 nt, synthetic construct
  source 1..15: mol_type = other RNA
tgttctgttcagcat

SEQ ID NO: 709 - RNA, 15 nt, synthetic construct
  source 1..15: mol_type = other RNA
cccccgccgcagcat

SEQ ID NO: 710 - RNA, 15 nt, synthetic construct
  source 1..15: mol_type = other RNA
tctggccggcagcat

SEQ ID NO: 711 - RNA, 15 nt, synthetic construct
  source 1..15: mol_type = other RNA
tcggggctgcagcat

SEQ ID NO: 712 - RNA, 15 nt, synthetic construct
  source 1..15: mol_type = other RNA
ctcggggctcagcat

SEQ ID NO: 713 - RNA, 15 nt, synthetic construct
  source 1..15: mol_type = other RNA
ctggccgggcagcat

SEQ ID NO: 714 - RNA, 15 nt, synthetic construct
  source 1..15: mol_type = other RNA
tctggccggcagcat

SEQ ID NO: 715 - RNA, 15 nt, synthetic construct
  source 1..15: mol_type = other RNA
tctggccggcagcat

SEQ ID NO: 716 - RNA, 15 nt, synthetic construct
  source 1..15: mol_type = other RNA
ctggccgggcagcat

SEQ ID NO: 717 - RNA, 15 nt, synthetic construct
  source 1..15: mol_type = other RNA
cctgtccgtcagcat

SEQ ID NO: 718 - RNA, 15 nt, synthetic construct
  source 1..15: mol_type = other RNA
ggcgtgtttcagcat

SEQ ID NO: 719 - RNA, 15 nt, synthetic construct
  source 1..15: mol_type = other RNA
ttgcctgtccagcat

SEQ ID NO: 720 - RNA, 15 nt, synthetic construct
  source 1..15: mol_type = other RNA
tctcggggccagcat

SEQ ID NO: 721 - RNA, 15 nt, synthetic construct
  source 1..15: mol_type = other RNA
tctcggggccagcat

SEQ ID NO: 722 - RNA, 15 nt, synthetic construct
  source 1..15: mol_type = other RNA
ctcggggctcagcat

SEQ ID NO: 723 - RNA, 15 nt, synthetic construct
  source 1..15: mol_type = other RNA
cgtcctcctcagcat

SEQ ID NO: 724 - RNA, 15 nt, synthetic construct
  source 1..15: mol_type = other RNA
cttttcttccagcat

SEQ ID NO: 725 - RNA, 15 nt, synthetic construct
  source 1..15: mol_type = other RNA
tttggctgccagcat

SEQ ID NO: 726 - RNA, 15 nt, synthetic construct
  source 1..15: mol_type = other RNA
ctcggggctcagcat

SEQ ID NO: 727 - RNA, 15 nt, synthetic construct
  source 1..15: mol_type = other RNA
gcgtgttttcagcat

SEQ ID NO: 728 - RNA, 15 nt, synthetic construct
  source 1..15: mol_type = other RNA
tggcgtgttcagcat

SEQ ID NO: 729 - RNA, 15 nt, synthetic construct
  source 1..15: mol_type = other RNA
tggcgtgttcagcat

SEQ ID NO: 730 - RNA, 15 nt, synthetic construct
  source 1..15: mol_type = other RNA
ggcgtgtttcagcat

SEQ ID NO: 731 - RNA, 15 nt, synthetic construct
  source 1..15: mol_type = other RNA
ggcgtgtttcagcat

SEQ ID NO: 732 - RNA, 15 nt, synthetic construct
  source 1..15: mol_type = other RNA
cgggcgtgtcagcat

SEQ ID NO: 733 - RNA, 15 nt, synthetic construct
  source 1..15: mol_type = other RNA
gtcgtgtttcagcat

SEQ ID NO: 734 - RNA, 15 nt, synthetic construct
  source 1..15: mol_type = other RNA
tggcgttttcagcat

SEQ ID NO: 735 - RNA, 15 nt, synthetic construct
  source 1..15: mol_type = other RNA
ggcgtgtttcagcat

SEQ ID NO: 736 - RNA, 15 nt, synthetic construct
  source 1..15: mol_type = other RNA
ggcgtgttccagcat

SEQ ID NO: 737 - RNA, 15 nt, synthetic construct
  source 1..15: mol_type = other RNA
ggcgtgtttcagcat

SEQ ID NO: 738 - RNA, 15 nt, synthetic construct
  source 1..15: mol_type = other RNA
tttcttgctcagcat

SEQ ID NO: 739 - RNA, 15 nt, synthetic construct
  source 1..15: mol_type = other RNA
ggcgtgtttcagcat

SEQ ID NO: 740 - RNA, 15 nt, synthetic construct
  source 1..15: mol_type = other RNA
tttgttctgcagcat

SEQ ID NO: 741 - RNA, 15 nt, synthetic construct
  source 1..15: mol_type = other RNA
ttgtggtttcagcat

SEQ ID NO: 742 - RNA, 15 nt, synthetic construct
  source 1..15: mol_type = other RNA
ttgtgttttcagcat

SEQ ID NO: 743 - RNA, 15 nt, synthetic construct
  source 1..15: mol_type = other RNA
ttgttctgtcagcat

SEQ ID NO: 744 - RNA, 15 nt, synthetic construct
  source 1..15: mol_type = other RNA
ttctgttgtcagcat

SEQ ID NO: 745 - RNA, 15 nt, synthetic construct
  source 1..15: mol_type = other RNA
ttctgttttcagcat

SEQ ID NO: 746 - RNA, 15 nt, synthetic construct
  source 1..15: mol_type = other RNA
ttgttctgtcagcat

SEQ ID NO: 747 - RNA, 15 nt, synthetic construct
  source 1..15: mol_type = other RNA
gttctgtttcagcat

SEQ ID NO: 748 - RNA, 15 nt, synthetic construct
  source 1..15: mol_type = other RNA
gttttccgccagcat

SEQ ID NO: 749 - RNA, 15 nt, synthetic construct
  source 1..15: mol_type = other RNA
tttttccgccagcat

SEQ ID NO: 750 - RNA, 15 nt, synthetic construct
  source 1..15: mol_type = other RNA
gttgtccgccagcat

SEQ ID NO: 751 - RNA, 15 nt, synthetic construct
  source 1..15: mol_type = other RNA
cctgttccgcagcat

SEQ ID NO: 752 - RNA, 15 nt, synthetic construct
  source 1..15: mol_type = other RNA
tgtctcgctcagcat

SEQ ID NO: 753 - RNA, 15 nt, synthetic construct
  source 1..15: mol_type = other RNA
tctggccggcagcat

SEQ ID NO: 754 - RNA, 15 nt, synthetic construct
  source 1..15: mol_type = other RNA
cgccgttttcagcat

SEQ ID NO: 755 - RNA, 15 nt, synthetic construct
  source 1..15: mol_type = other RNA
tcttcgtctcagcat

SEQ ID NO: 756 - RNA, 15 nt, synthetic construct
  source 1..15: mol_type = other RNA
ttttcttctcagcat

SEQ ID NO: 757 - RNA, 15 nt, synthetic construct
  source 1..15: mol_type = other RNA
ttcttctgtcagcat

SEQ ID NO: 758 - RNA, 15 nt, synthetic construct
  source 1..15: mol_type = other RNA
tctggccgtcagcat

SEQ ID NO: 759 - RNA, 15 nt, synthetic construct
  source 1..15: mol_type = other RNA
tctggccggcagcat

SEQ ID NO: 760 - RNA, 15 nt, synthetic construct
  source 1..15: mol_type = other RNA
ctgtccgggcagcat

SEQ ID NO: 761 - RNA, 15 nt, synthetic construct
  source 1..15: mol_type = other RNA
tctggctggcagcat

SEQ ID NO: 762 - RNA, 15 nt, synthetic construct
  source 1..15: mol_type = other RNA
tctggccggcagcat

SEQ ID NO: 763 - RNA, 15 nt, synthetic construct
  source 1..15: mol_type = other RNA
tctggccggcagcat

SEQ ID NO: 764 - RNA, 15 nt, synthetic construct
  source 1..15: mol_type = other RNA
cgggccgggcagcat

SEQ ID NO: 765 - RNA, 15 nt, synthetic construct
  source 1..15: mol_type = other RNA
ctggccgggcagcat

SEQ ID NO: 766 - RNA, 15 nt, synthetic construct
  source 1..15: mol_type = other RNA
ctggccgggcagcat

SEQ ID NO: 767 - RNA, 15 nt, synthetic construct
  source 1..15: mol_type = other RNA
tctggctggcagcat

SEQ ID NO: 768 - RNA, 15 nt, synthetic construct
  source 1..15: mol_type = other RNA
tctggccggcagcat

SEQ ID NO: 769 - RNA, 15 nt, synthetic construct
  source 1..15: mol_type = other RNA
tctggcctgcagcat

SEQ ID NO: 770 - RNA, 15 nt, synthetic construct
  source 1..15: mol_type = other RNA
tctggccggcagcat

SEQ ID NO: 771 - RNA, 15 nt, synthetic construct
  source 1..15: mol_type = other RNA
ctctggccgcagcat

SEQ ID NO: 772 - RNA, 15 nt, synthetic construct
  source 1..15: mol_type = other RNA
tctcggggccagcat

SEQ ID NO: 773 - RNA, 15 nt, synthetic construct
  source 1..15: mol_type = other RNA
ctcggggctcagcat

SEQ ID NO: 774 - RNA, 15 nt, synthetic construct
  source 1..15: mol_type = other RNA
cggggctgccagcat

SEQ ID NO: 775 - RNA, 15 nt, synthetic construct
  source 1..15: mol_type = other RNA
gcgccgtttcagcat

SEQ ID NO: 776 - RNA, 15 nt, synthetic construct
  source 1..15: mol_type = other RNA
gcgccgtttcagcat

SEQ ID NO: 777 - RNA, 15 nt, synthetic construct
  source 1..15: mol_type = other RNA
tgcgccgttcagcat

SEQ ID NO: 778 - RNA, 15 nt, synthetic construct
  source 1..15: mol_type = other RNA
tgtgcgccgcagcat

SEQ ID NO: 779 - RNA, 15 nt, synthetic construct
  source 1..15: mol_type = other RNA
tctcggggccagcat

SEQ ID NO: 780 - RNA, 15 nt, synthetic construct
  source 1..15: mol_type = other RNA
tctcggggccagcat

SEQ ID NO: 781 - RNA, 15 nt, synthetic construct
  source 1..15: mol_type = other RNA
tctcggggccagcat

SEQ ID NO: 782 - RNA, 15 nt, synthetic construct
  source 1..15: mol_type = other RNA
cttggggctcagcat

SEQ ID NO: 783 - RNA, 15 nt, synthetic construct
  source 1..15: mol_type = other RNA
tcccggggccagcat

SEQ ID NO: 784 - RNA, 15 nt, synthetic construct
  source 1..15: mol_type = other RNA
ctcggggctcagcat

SEQ ID NO: 785 - RNA, 15 nt, synthetic construct
  source 1..15: mol_type = other RNA
tctcggggccagcat

SEQ ID NO: 786 - RNA, 15 nt, synthetic construct
  source 1..15: mol_type = other RNA
tcgtggctgcagcat

SEQ ID NO: 787 - RNA, 15 nt, synthetic construct
  source 1..15: mol_type = other RNA
ctcggggctcagcat

SEQ ID NO: 788 - RNA, 15 nt, synthetic construct
  source 1..15: mol_type = other RNA
tcggggctgcagcat

SEQ ID NO: 789 - RNA, 15 nt, synthetic construct
  source 1..15: mol_type = other RNA
tcttggggccagcat

SEQ ID NO: 790 - RNA, 15 nt, synthetic construct
  source 1..15: mol_type = other RNA
ctcggggctcagcat

SEQ ID NO: 791 - RNA, 15 nt, synthetic construct
  source 1..15: mol_type = other RNA
tctcggggccagcat

SEQ ID NO: 792 - RNA, 15 nt, synthetic construct
  source 1..15: mol_type = other RNA
ctcggggctcagcat

SEQ ID NO: 793 - RNA, 15 nt, synthetic construct
  source 1..15: mol_type = other RNA
ctcgggctgcagcat

SEQ ID NO: 794 - RNA, 15 nt, synthetic construct
  source 1..15: mol_type = other RNA
tcccttggtcagcat

SEQ ID NO: 795 - RNA, 15 nt, synthetic construct
  source 1..15: mol_type = other RNA
ggtctcgctcagcat

SEQ ID NO: 796 - RNA, 15 nt, synthetic construct
  source 1..15: mol_type = other RNA
tgtctcgctcagcat

SEQ ID NO: 797 - RNA, 15 nt, synthetic construct
  source 1..15: mol_type = other RNA
ttttgtccccagcat

SEQ ID NO: 798 - RNA, 15 nt, synthetic construct
  source 1..15: mol_type = other RNA
tttgtctcgcagcat

SEQ ID NO: 799 - RNA, 15 nt, synthetic construct
  source 1..15: mol_type = other RNA
tttcctctccagcat

SEQ ID NO: 800 - RNA, 15 nt, synthetic construct
  source 1..15: mol_type = other RNA
tgtcctctccagcat

SEQ ID NO: 801 - RNA, 15 nt, synthetic construct
  source 1..15: mol_type = other RNA
cctctcgtccagcat

SEQ ID NO: 802 - RNA, 15 nt, synthetic construct
  source 1..15: mol_type = other RNA
cttttcttccagcat

SEQ ID NO: 803 - RNA, 15 nt, synthetic construct
  source 1..15: mol_type = other RNA
tctgcgcttcagcat

SEQ ID NO: 804 - RNA, 15 nt, synthetic construct
  source 1..15: mol_type = other RNA
cttttcgtccagcat

SEQ ID NO: 805 - RNA, 15 nt, synthetic construct
  source 1..15: mol_type = other RNA
tcttttcgtcagcat

SEQ ID NO: 806 - RNA, 15 nt, synthetic construct
  source 1..15: mol_type = other RNA
ctctcttcccagcat

SEQ ID NO: 807 - RNA, 15 nt, synthetic construct
  source 1..15: mol_type = other RNA
ttcctctttcagcat

SEQ ID NO: 808 - RNA, 15 nt, synthetic construct
  source 1..15: mol_type = other RNA
ttcttttctcagcat

SEQ ID NO: 809 - RNA, 15 nt, synthetic construct
  source 1..15: mol_type = other RNA
ttctgtgctcagcat

SEQ ID NO: 810 - RNA, 15 nt, synthetic construct
  source 1..15: mol_type = other RNA
cctctcttccagcat

SEQ ID NO: 811 - RNA, 15 nt, synthetic construct
  source 1..15: mol_type = other RNA
ctgtctcctcagcat

SEQ ID NO: 812 - RNA, 15 nt, synthetic construct
  source 1..15: mol_type = other RNA
tgttctccccagcat

SEQ ID NO: 813 - RNA, 15 nt, synthetic construct
  source 1..15: mol_type = other RNA
cttcctgtccagcat

SEQ ID NO: 814 - RNA, 15 nt, synthetic construct
  source 1..15: mol_type = other RNA
tttcctgtccagcat

SEQ ID NO: 815 - RNA, 15 nt, synthetic construct
  source 1..15: mol_type = other RNA
tgtgtgtgtcagcat

SEQ ID NO: 816 - RNA, 15 nt, synthetic construct
  source 1..15: mol_type = other RNA
tgcttcctgcagcat

SEQ ID NO: 817 - RNA, 15 nt, synthetic construct
  source 1..15: mol_type = other RNA
tctggccggcagcat

SEQ ID NO: 818 - RNA, 15 nt, synthetic construct
  source 1..15: mol_type = other RNA
tctcggggccagcat

SEQ ID NO: 819 - RNA, 15 nt, synthetic construct
  source 1..15: mol_type = other RNA
tctggccggcagcat

SEQ ID NO: 820 - RNA, 15 nt, synthetic construct
  source 1..15: mol_type = other RNA
ccgcccgcccagcat

SEQ ID NO: 821 - RNA, 15 nt, synthetic construct
  source 1..15: mol_type = other RNA
gtgttgttccagcat

SEQ ID NO: 822 - RNA, 15 nt, synthetic construct
  source 1..15: mol_type = other RNA
ccttctggccagcat

SEQ ID NO: 823 - RNA, 15 nt, synthetic construct
  source 1..15: mol_type = other RNA
ggcgtcccgcagcat

SEQ ID NO: 824 - RNA, 15 nt, synthetic construct
  source 1..15: mol_type = other RNA
tttctgcggcagcat

SEQ ID NO: 825 - RNA, 15 nt, synthetic construct
  source 1..15: mol_type = other RNA
gcctgccgtcagcat

SEQ ID NO: 826 - RNA, 44 nt, synthetic construct
  source 1..44: mol_type = other RNA
gaaaagctgggttgagggggttttgttttcgacccaactccccc

SEQ ID NO: 827 - RNA, 74 nt, synthetic construct
  source 1..74: mol_type = other RNA
gaaactctggtggaggtccgtagcggtcctgacgtttttgtttgagaccacctccaggcatcgccaggactgca

SEQ ID NO: 828 - RNA, 38 nt, synthetic construct
  source 1..38: mol_type = other RNA
gaattgtgagcggataattttgttaacactcgcctatt

SEQ ID NO: 829 - RNA, 48 nt, synthetic construct
  source 1..48: mol_type = other RNA
gataagggaagtcggcaaaatattttgtattcccttcagccgttttat

SEQ ID NO: 830 - RNA, 66 nt, synthetic construct
  source 1..66: mol_type = other RNA
gcatgagtggttcagtggtagaattctcgccttttggtactcaccaagtcaccatcttaagagcgg

SEQ ID NO: 831 - RNA, 40 nt, synthetic construct
  source 1..40: mol_type = other RNA
ggccagtggcgcaatggattttgcggtcaccgcgttacct

SEQ ID NO: 832 - RNA, 76 nt, synthetic construct
  source 1..76: mol_type = other RNA
ggctgggtcggtcgggctggggcgcgaagcggggccttttgcgacccagccagcccgaccccgcgcttcgccccgg

SEQ ID NO: 833 - RNA, 44 nt, synthetic construct
  source 1..44: mol_type = other RNA
ggtgatgtcggcgatataggttttgcactacagccgctatatcc

SEQ ID NO: 834 - RNA, 46 nt, synthetic construct
  source 1..46: mol_type = other RNA
taacccgtatcgtgagcatcctttttttgggcatagcactcgtagg

SEQ ID NO: 835 - RNA, 70 nt, synthetic construct
  source 1..70: mol_type = other RNA
tacaaccccccactgctaaatttgactggcttcttttttgttggggggtgacgatttaaactgaccgaag

SEQ ID NO: 836 - RNA, 48 nt, synthetic construct
  source 1..48: mol_type = other RNA
tatcccgccgttagccaccatcttttttagggcggcaatcggtggtag

SEQ ID NO: 837 - RNA, 48 nt, synthetic construct
  source 1..48: mol_type = other RNA
tcatgcaactcgtaggacaggttttttgtacgttgagcatcctgtcca

SEQ ID NO: 838 - RNA, 52 nt, synthetic construct
  source 1..52: mol_type = other RNA
tggaagactagtgattctgttgtttttttccttctgatcactaagacaacaa

SEQ ID NO: 839 - RNA, 60 nt, synthetic construct
  source 1..60: mol_type = other RNA
tggtccgaaggtagtgggttatctcaattttttccaggcttccatcacccaatagagtta

SEQ ID NO: 840 - RNA, 46 nt, synthetic construct
  source 1..46: mol_type = other RNA
ttggcggatgagataagattttttttaccgcctactctattctaaa

SEQ ID NO: 841 - RNA, 70 nt, synthetic construct
  source 1..70: mol_type = other RNA
tttccgtagtgtagtggttatcacgttcgcctgtttttaaggcatcacatcaccaatagtgcaagcggac

SEQ ID NO: 842 - RNA, 48 nt, synthetic construct
  source 1..48: mol_type = other RNA
tttcgccacctctgacttgggctttttaagcggtggagactgaacccg

SEQ ID NO: 843 - RNA, 50 nt, synthetic construct
  source 1..50: mol_type = other RNA
actacgatacgggagggcttaccttttagatgctatgccctcccgaatgg

SEQ ID NO: 844 - RNA, 46 nt, synthetic construct
  source 1..46: mol_type = other RNA
aagaacgtcgcgcactgctagttttatcttgcagcgcgtgacgatc

SEQ ID NO: 845 - RNA, 48 nt, synthetic construct
  source 1..48: mol_type = other RNA
catgctcgtgaactggcgtatgttttctacgagcacttgaccgcatac

SEQ ID NO: 846 - RNA, 52 nt, synthetic construct
  source 1..52: mol_type = other RNA
cgtgtggaattctcgggtgccaaattttccacaccttaagagcccacggttt

SEQ ID NO: 847 - RNA, 46 nt, synthetic construct
  source 1..46: mol_type = other RNA
tccgggtagtctcgagaaacatttttggcccatcagagctctttgt

SEQ ID NO: 848 - RNA, 46 nt, synthetic construct
  source 1..46: mol_type = other RNA
tgaacacccggacctggctattttttcttgtgggcctggaccgata

SEQ ID NO: 849 - RNA, 48 nt, synthetic construct
  source 1..48: mol_type = other RNA
aaacgacgagcgtggcaccacgttttattgctgctcgcaccgtggtgc

SEQ ID NO: 850 - RNA, 46 nt, synthetic construct
  source 1..46: mol_type = other RNA
ctttcgcttcgttatctactcttttcaaagcgaagcaatagatgag

SEQ ID NO: 851 - RNA, 38 nt, synthetic construct
  source 1..38: mol_type = other RNA
aaaggaaggaatcgaacttttattccttccttagcttg

SEQ ID NO: 852 - RNA, 66 nt, synthetic construct
  source 1..66: mol_type = other RNA
agcagagtggcgcagcggaagcgtgctaggcttttacgtctcaccgcgtcgccttcgcacgatccg

SEQ ID NO: 853 - RNA, 66 nt, synthetic construct
  source 1..66: mol_type = other RNA
agcagagttgcgcagcggaagcgtgctggacttttacgtctcaacgcgtcgccttcgcacgacctg

SEQ ID NO: 854 - RNA, 68 nt, synthetic construct
  source 1..68: mol_type = other RNA
aggctggtccgatggtagtgggttattagaacttttaccgaccaggctaccatcacccaataatcttg

SEQ ID NO: 855 - RNA, 46 nt, synthetic construct
  source 1..46: mol_type = other RNA
ataaccaggtccgggtgttcattttaattggtccaggcccacaagt

SEQ ID NO: 856 - RNA, 44 nt, synthetic construct
  source 1..44: mol_type = other RNA
agcgtggtggtgccgatggtttttacgcaccaccacggctacca

SEQ ID NO: 857 - RNA, 46 nt, synthetic construct
  source 1..46: mol_type = other RNA
tgaacacccggacccggttattttttcttgtgggcctgggccaata

SEQ ID NO: 858 - RNA, 38 nt, synthetic construct
  source 1..38: mol_type = other RNA
aacagcagttggacatgttttatgtcgtcaacctgtac

SEQ ID NO: 859 - RNA, 72 nt, synthetic construct
  source 1..72: mol_type = other RNA
attggtcgtggctgtagtccgtgcgagaataccattttaaaccagcaccgacatcaggcacgctcttatggt

SEQ ID NO: 860 - RNA, 44 nt, synthetic construct
  source 1..44: mol_type = other RNA
catggaacgggttggcatggttttctaccttgcccaaccgtacc

SEQ ID NO: 861 - RNA, 46 nt, synthetic construct
  source 1..46: mol_type = other RNA
cattcgatggtgtctgggatcttttctaagctaccacagaccctag

SEQ ID NO: 862 - RNA, 64 nt, synthetic construct
  source 1..64: mol_type = other RNA
cattggtggttcagtagtagaattctcgccttttctaaccaccaagtcatcatcttaagagcgg

SEQ ID NO: 863 - RNA, 44 nt, synthetic construct
  source 1..44: mol_type = other RNA
cccgagactcagtgaaattgttttcggctctgagtcactttaac

SEQ ID NO: 864 - RNA, 74 nt, synthetic construct
  source 1..74: mol_type = other RNA
aagaaggaaagaagaaggggaagaaaagaaggggattttatcttcctttcttcttccccttcttttcttcccct

SEQ ID NO: 865 - RNA, 52 nt, synthetic construct
  source 1..52: mol_type = other RNA
aagacgggaggaaagaagggagatttttatctgccctcctttcttccctcta

SEQ ID NO: 866 - RNA, 82 nt, synthetic construct
  source 1..82: mol_type = other RNA
cggcggcggcggcggtggcggcggcggcggcggcgggacttttcccgccgccgccgccaccgccgccgccgccgccgccctg

SEQ ID NO: 867 - RNA, 48 nt, synthetic construct
  source 1..48: mol_type = other RNA
gagaactgaattccatgggtttttttgtcttgacttaaggtacccaaa

SEQ ID NO: 868 - RNA, 44 nt, synthetic construct
  source 1..44: mol_type = other RNA
gaggaggaggaggaggacggttttgtcctcctcctcctcctgcc

SEQ ID NO: 869 - RNA, 44 nt, synthetic construct
  source 1..44: mol_type = other RNA
gaggtagtaggttgcatagtttttgtccatcatccaacgtatca

SEQ ID NO: 870 - RNA, 44 nt, synthetic construct
  source 1..44: mol_type = other RNA
gagtaagacgctgccggaatttttgtcattctgcgacggcctta

SEQ ID NO: 871 - RNA, 50 nt, synthetic construct
  source 1..50: mol_type = other RNA
gatggggaagatcgggctcgccattttgtaccccttctagcccgagcggt

SEQ ID NO: 872 - RNA, 70 nt, synthetic construct
  source 1..70: mol_type = other RNA
gcattggtggttcagtggtagaattctcgcctgttttggtaaccaccaagtcaccatcttaagagcggac

SEQ ID NO: 873 - RNA, 56 nt, synthetic construct
  source 1..56: mol_type = other RNA
gcatttgtggttcagtggtagaattcttttggtaaacaccaagtcaccatcttaag

SEQ ID NO: 874 - RNA, 62 nt, synthetic construct
  source 1..62: mol_type = other RNA
ggctggtccgaaggtagtgagttatctcattttgcgaccaggcttccatcactcaatagagt

SEQ ID NO: 875 - RNA, 52 nt, synthetic construct
  source 1..52: mol_type = other RNA
gtaaacatccttgactggaagcttttttgatttgtaggaactgaccttcgaa

SEQ ID NO: 876 - RNA, 50 nt, synthetic construct
  source 1..50: mol_type = other RNA
tagcttatcagactgacgttgacttttttcgaatagtctgactgcaactg

SEQ ID NO: 877 - RNA, 52 nt, synthetic construct
  source 1..52: mol_type = other RNA
tagcttatcagactgatgttgactttttttcgaatagtctgactacaactga

SEQ ID NO: 878 - RNA, 48 nt, synthetic construct
  source 1..48: mol_type = other RNA
tattgcagttgtcccggcctgtttttttaacgtcaacagggccggaca

SEQ ID NO: 879 - RNA, 46 nt, synthetic construct
  source 1..46: mol_type = other RNA
tgagatgaagcactgtagcaatttttctctacttcgtgacatcgtt

SEQ ID NO: 880 - RNA, 44 nt, synthetic construct
  source 1..44: mol_type = other RNA
tgaggggcagagagcgagattttttctccccgtctctcgctcta

SEQ ID NO: 881 - RNA, 46 nt, synthetic construct
  source 1..46: mol_type = other RNA
tgaggggcagagagcgaggcttttttctccccgtctctcgctccga

SEQ ID NO: 882 - RNA, 50 nt, synthetic construct
  source 1..50: mol_type = other RNA
tgaggggcagagagcgagacctttttttctccccgtctctcgctctggaa

SEQ ID NO: 883 - RNA, 48 nt, synthetic construct
  source 1..48: mol_type = other RNA
tgaggggcagagagcgagaagatttttctccccgtctctcgctcttct

SEQ ID NO: 884 - RNA, 50 nt, synthetic construct
  source 1..50: mol_type = other RNA
tgaggtagtaggttgtgtggtcttttttctccatcatccaacacaccaga

SEQ ID NO: 885 - RNA, 42 nt, synthetic construct
  source 1..42: mol_type = other RNA
tggagtgtgacaatggtgatttttcctcacactgttaccact

SEQ ID NO: 886 - RNA, 48 nt, synthetic construct
  source 1..48: mol_type = other RNA
tgtaaacatccccgactgaaagtttttcatttgtaggggctgactttc

SEQ ID NO: 887 - RNA, 50 nt, synthetic construct
  source 1..50: mol_type = other RNA
tgtaaatatccccgactggaagctttttcatttataggggctgaccttcg

SEQ ID NO: 888 - RNA, 50 nt, synthetic construct
  source 1..50: mol_type = other RNA
tgtaaacatccttgactggaagttttttcatttgtaggaactgaccttca

SEQ ID NO: 889 - RNA, 40 nt, synthetic construct
  source 1..40: mol_type = other RNA
ttgggagcgggcgggcggtttttaccctcgcccgcccgcc

SEQ ID NO: 890 - RNA, 46 nt, synthetic construct
  source 1..46: mol_type = other RNA
tttagtaatggtaatggttcttttttaatcattaccattaccaaga

SEQ ID NO: 891 - RNA, 64 nt, synthetic construct
  source 1..64: mol_type = other RNA
tttccgtagtgtagtggttatcacgttcgctttttaaggcatcacatcaccaatagtgcaagcg

SEQ ID NO: 892 - RNA, 42 nt, synthetic construct
  source 1..42: mol_type = other RNA
tttgttcgttcggctcgcgtttttaacaagcaagccgagcgc

SEQ ID NO: 893 - RNA, 54 nt, synthetic construct
  source 1..54: mol_type = other RNA
acggcggcggcggcggcggcgggggttttagccgccgccgccgccgccgccccc

SEQ ID NO: 894 - RNA, 46 nt, synthetic construct
  source 1..46: mol_type = other RNA
agaggaggaagaggaggaggattttactcctccttctcctcctcct

SEQ ID NO: 895 - RNA, 66 nt, synthetic construct
  source 1..66: mol_type = other RNA
aggctggtccgatggtagtgggttatcagaattttaccgaccaggctaccatcacccaatagtctt

SEQ ID NO: 896 - RNA, 54 nt, synthetic construct
  source 1..54: mol_type = other RNA
agggcggcggcggcggcggcgggggttttacccgccgccgccgccgccgccccc

SEQ ID NO: 897 - RNA, 52 nt, synthetic construct
  source 1..52: mol_type = other RNA
agggcggcggcggcggcggcggggttttacccgccgccgccgccgccgcccc

SEQ ID NO: 898 - RNA, 40 nt, synthetic construct
  source 1..40: mol_type = other RNA
attccggttgttcttctcttttaaaggccaacaagaagag

SEQ ID NO: 899 - RNA, 46 nt, synthetic construct
  source 1..46: mol_type = other RNA
atgaggtagtaggttgtgtggttttaactccatcatccaacacacc

SEQ ID NO: 900 - RNA, 46 nt, synthetic construct
  source 1..46: mol_type = other RNA
aacattcaacgctgtcggtgtttttatgtaagttgcgacagccaca

SEQ ID NO: 901 - RNA, 46 nt, synthetic construct
  source 1..46: mol_type = other RNA
caacaccagtcgatgggctgtttttcttgtggtcagctacccgaca

SEQ ID NO: 902 - RNA, 40 nt, synthetic construct
  source 1..40: mol_type = other RNA
caacggaatcccaaaagcttttcttgccttagggttttcg

SEQ ID NO: 903 - RNA, 44 nt, synthetic construct
  source 1..44: mol_type = other RNA
aaccgggcggaaacaccaaattttatggcccgcctttgtggttt

SEQ ID NO: 904 - RNA, 40 nt, synthetic construct
  source 1..40: mol_type = other RNA
cattcttgcgacccgggtttttctaagaacgctgggccca

SEQ ID NO: 905 - RNA, 70 nt, synthetic construct
  source 1..70: mol_type = other RNA
cccctgtggtctagtggttaggattcggcgctcttttcgggacaccagatcaccaatcctaagccgcgag

SEQ ID NO: 906 - RNA, 44 nt, synthetic construct
  source 1..44: mol_type = other RNA
aaaagctgggttgagagggattttatttcgacccaactctccct

SEQ ID NO: 907 - RNA, 60 nt, synthetic construct
  source 1..60: mol_type = other RNA
ctggtccgaaggtagtgagttatctcaattttcaccaggcttccatcactcaatagagtt

SEQ ID NO: 908 - RNA, 46 nt, synthetic construct
  source 1..46: mol_type = other RNA
aaaagctgggctgagagggcgttttatttcgacccgactctcccgc

SEQ ID NO: 909 - RNA, 44 nt, synthetic construct; identical to SEQ ID NO: 906
  source 1..44: mol_type = other RNA

SEQ ID NO: 910 - RNA, 50 nt, synthetic construct
  source 1..50: mol_type = other RNA
aaaagctgggttgagagggcgagttttatttcgacccaactctcccgctc

SEQ ID NO: 911 - RNA, 48 nt, synthetic construct
  source 1..48: mol_type = other RNA
aagctgccagttgaagaactgcttttatcgacggtcaacttcttgacg

SEQ ID NO: 912 - RNA, 48 nt, synthetic construct
  source 1..48: mol_type = other RNA
agctacattgtctgctgggtttttttacgatgtaacagacgacccaaa

SEQ ID NO: 913 - RNA, 50 nt, synthetic construct
  source 1..50: mol_type = other RNA
agctcggcctgaggcccctcagtttttacgagccggactccggggagtca

SEQ ID NO: 914 - RNA, 70 nt, synthetic construct
  source 1..70: mol_type = other RNA
aggctggtccgatggtagtgggttatcagaactttttaccgaccaggctaccatcacccaatagtcttga

SEQ ID NO: 915 - RNA, 54 nt, synthetic construct; identical to SEQ ID NO: 896
  source 1..54: mol_type = other RNA

SEQ ID NO: 916 - RNA, 50 nt, synthetic construct
  source 1..50: mol_type = other RNA
atattgcacttgtcccggcctgtttttaataacgtgaacagggccggaca

SEQ ID NO: 917 - RNA, 48 nt, synthetic construct
  source 1..48: mol_type = other RNA
atcctgtactgagctgccccgattttaaggacatgactcgacggggct

SEQ ID NO: 918 - RNA, 50 nt, synthetic construct
  source 1..50: mol_type = other RNA
atcctgtactgagctgccccgagttttaaggacatgactcgacggggctc

SEQ ID NO: 919 - RNA, 44 nt, synthetic construct
  source 1..44: mol_type = other RNA
attgcacttgtcccggcctgttttaaacgtgaacagggccggac

SEQ ID NO: 920 - RNA, 46 nt, synthetic construct
  source 1..46: mol_type = other RNA
attgcacttgtcccggcctgtttttaaacgtgaacagggccggaca

SEQ ID NO: 921 - RNA, 46 nt, synthetic construct
  source 1..46: mol_type = other RNA
attgcacttgccccggcctgtttttaaacgtgaacggggccggaca

SEQ ID NO: 922 - RNA, 46 nt, synthetic construct
  source 1..46: mol_type = other RNA
cattgcacttgtcccggcctgttttctaacgtgaacagggccggac

SEQ ID NO: 923 - RNA, 48 nt, synthetic construct
  source 1..48: mol_type = other RNA
cattgcacttgtctcggtctggttttctaacgtgaacagagccagacc

SEQ ID NO: 924 - RNA, 64 nt, synthetic construct
  source 1..64: mol_type = other RNA
cattggtggttcagtggtagaattctcgccttttctaaccaccaagtcaccatcttaagagcgg

SEQ ID NO: 925 - RNA, 58 nt, synthetic construct
  source 1..58: mol_type = other RNA
ccccccgctgctaaatttgactggcttttttcgggggcgacgatttaaactgaccgaa

SEQ ID NO: 926 - RNA, 50 nt, synthetic construct
  source 1..50: mol_type = other RNA
ccctgtactgagctgccccgagattttcggacatgactcgacggggctct

SEQ ID NO: 927 - RNA, 48 nt, synthetic construct
  source 1..48: mol_type = other RNA
cctgtactgagctgccccgagattttcgacatgactcgacggggctct

SEQ ID NO: 928 - RNA, 46 nt, synthetic construct
  source 1..46: mol_type = other RNA
cctgtactgagctgccccgagttttcgacatgactcgacggggctc

SEQ ID NO: 929 - RNA, 44 nt, synthetic construct
  source 1..44: mol_type = other RNA
cgaggaggaagaggaggacgttttcctcctccttctcctcctgc

SEQ ID NO: 930 - RNA, 44 nt, synthetic construct
  source 1..44: mol_type = other RNA
cgaggtagtaggttgtatagttttcctccatcatccaacatatc

SEQ ID NO: 931 - RNA, 44 nt, synthetic construct
  source 1..44: mol_type = other RNA
ctatacgacctgctgcctttttttcatatgctggacgacggaaa

SEQ ID NO: 932 - RNA, 44 nt, synthetic construct
  source 1..44: mol_type = other RNA
ctgtactgagctgccccgagttttcacatgactcgacggggctc

SEQ ID NO: 933 - RNA, 64 nt, synthetic construct
  source 1..64: mol_type = other RNA
gaattggtggttcagtggtagaattctcgcttttgttaaccaccaagtcaccatcttaagagcg

SEQ ID NO: 934 - RNA, 68 nt, synthetic construct
  source 1..68: mol_type = other RNA
gcatcggtggttcagtggtagaattctcgcctttttggtagccaccaagtcaccatcttaagagcgga

SEQ ID NO: 935 - RNA, 68 nt, synthetic construct
  source 1..68: mol_type = other RNA
gcattagtggttcagtggtagaattctcgcctttttggtaatcaccaagtcaccatcttaagagcgga

SEQ ID NO: 936 - RNA, 66 nt, synthetic construct
  source 1..66: mol_type = other RNA
gcattgatggttcagtggtagaattctcgccttttggtaactaccaagtcaccatcttaagagcgg

SEQ ID NO: 937 - RNA, 66 nt, synthetic construct
  source 1..66: mol_type = other RNA
gcattggtagttcagtggtagaattctcgccttttggtaaccatcaagtcaccatcttaagagcgg

SEQ ID NO: 938 - RNA, 70 nt, synthetic construct
  source 1..70: mol_type = other RNA
gcattggtggttcagtggtagaattctcgcccgttttggtaaccaccaagtcaccatcttaagagcgggc

SEQ ID NO: 939 - RNA, 66 nt, synthetic construct
  source 1..66: mol_type = other RNA
gcattggtggttcagtggtagaattctcgtcttttggtaaccaccaagtcaccatcttaagagcag

SEQ ID NO: 940 - RNA, 68 nt, synthetic construct
  source 1..68: mol_type = other RNA
gcattggtggttcagtggtagaattttcgcctttttggtaaccaccaagtcaccatcttaaaagcgga

SEQ ID NO: 941 - RNA, 66 nt, synthetic construct
  source 1..66: mol_type = other RNA
gcattggtggttcagtggtaggattctcgccttttggtaaccaccaagtcaccatcctaagagcgg

SEQ ID NO: 942 - RNA, 66 nt, synthetic construct
  source 1..66: mol_type = other RNA
gcattggtggttcagtggtagagttctcgccttttggtaaccaccaagtcaccatctcaagagcgg

SEQ ID NO: 943 - RNA, 66 nt, synthetic construct
  source 1..66: mol_type = other RNA
gcattggtggttcggtggtagaattctcgccttttggtaaccaccaagccaccatcttaagagcgg

SEQ ID NO: 944 - RNA, 68 nt, synthetic construct
  source 1..68: mol_type = other RNA
gcgccgctggtgtagtggcatcatgcaagattttttggcggcgaccacatcaccgtagtacgttctaa

SEQ ID NO: 945 - RNA, 66 nt, synthetic construct
  source 1..66: mol_type = other RNA
gcgttggtggttcagtggtagaattctcgccttttggcaaccaccaagtcaccatcttaagagcgg

SEQ ID NO: 946 - RNA, 72 nt, synthetic construct
  source 1..72: mol_type = other RNA
ggctggcccgatggtagtgggttatcagaacttattttgcgaccgggctaccatcacccaatagtcttgaat

SEQ ID NO: 947 - RNA, 64 nt, synthetic construct
  source 1..64: mol_type = other RNA
ggctggtccgaaggtagtgagttacctcaattttgcgaccaggcttccatcactcaatggagtt

SEQ ID NO: 948 - RNA, 64 nt, synthetic construct
  source 1..64: mol_type = other RNA
ggctggtccgaaggcagtgagttatctcaattttgcgaccaggcttccgtcactcaatagagtt

SEQ ID NO: 949 - RNA, 70 nt, synthetic construct
  source 1..70: mol_type = other RNA
ggctggtccgatagtagtgggttatcagaacttttttgcgaccaggctatcatcacccaatagtcttgaa

SEQ ID NO: 950 - RNA, 64 nt, synthetic construct
  source 1..64: mol_type = other RNA
ggctggtccgatggtagtgggtcatcagaattttgcgaccaggctaccatcacccagtagtctt

SEQ ID NO: 951 - RNA, 64 nt, synthetic construct
  source 1..64: mol_type = other RNA
ggctggtccgatggtagcgggttatcagaattttgcgaccaggctaccatcgcccaatagtctt

SEQ ID NO: 952 - RNA, 70 nt, synthetic construct
  source 1..70: mol_type = other RNA
ggctggtccgatggtagtgggttgtcagaacttttttgcgaccaggctaccatcacccaacagtcttgaa

SEQ ID NO: 953 - RNA, 66 nt, synthetic construct
  source 1..66: mol_type = other RNA
ggctggtccgatggtagtaggttatcagaacttttgcgaccaggctaccatcatccaatagtcttg

SEQ ID NO: 954 - RNA, 46 nt, synthetic construct
  source 1..46: mol_type = other RNA
ggggaattgcgagcggataacttttgcccttaacgctcgcctattg

SEQ ID NO: 955 - RNA, 46 nt, synthetic construct
  source 1..46: mol_type = other RNA
ggggaattgtgagcggataatttttgcccttaacactcgcctatta

SEQ ID NO: 956 - RNA, 46 nt, synthetic construct
  source 1..46: mol_type = other RNA
ggggaattgtgagcggacaacttttgcccttaacactcgcctgttg

SEQ ID NO: 957 - RNA, 48 nt, synthetic construct
  source 1..48: mol_type = other RNA
taagcggcggggtcggaacaggtttttttcgccgccccagccttgtcc

SEQ ID NO: 958 - RNA, 46 nt, synthetic construct
  source 1..46: mol_type = other RNA
taaggggcagagagcgagacttttttttccccgtctctcgctctga

SEQ ID NO: 959 - RNA, 48 nt, synthetic construct
  source 1..48: mol_type = other RNA
taatgcacttgtcccggcctgttttttttacgtgaacagggccggaca

SEQ ID NO: 960 - RNA, 48 nt, synthetic construct
  source 1..48: mol_type = other RNA
tagcagcacgtaaatatcggcgttttttcgtcgtgcatttatagccgc

SEQ ID NO: 961 - RNA, 48 nt, synthetic construct
  source 1..48: mol_type = other RNA
tagcttatcagactgacgttgattttttcgaatagtctgactgcaact

SEQ ID NO: 962 - RNA, 48 nt, synthetic construct
  source 1..48: mol_type = other RNA
tagcttatcagactgatgttaattttttcgaatagtctgactacaatt

SEQ ID NO: 963 - RNA, 44 nt, synthetic construct
  source 1..44: mol_type = other RNA
tagcttatcagactgatgttttttttcgaatagtctgactacaa

SEQ ID NO: 964 - RNA, 48 nt, synthetic construct
  source 1..48: mol_type = other RNA
tattgcacttgtcacggcctgtttttttaacgtgaacagtgccggaca

SEQ ID NO: 965 - RNA, 48 nt, synthetic construct
  source 1..48: mol_type = other RNA
tattgcacttgccccggcctgtttttttaacgtgaacggggccggaca

SEQ ID NO: 966 - RNA, 46 nt, synthetic construct
  source 1..46: mol_type = other RNA
tattgcacttgtcccggtctgttttttaacgtgaacagggccagac

SEQ ID NO: 967 - RNA, 48 nt, synthetic construct
  source 1..48: mol_type = other RNA
tattgcacttgtcccagcctgtttttttaacgtgaacagggtcggaca

SEQ ID NO: 968 - RNA, 48 nt, synthetic construct
  source 1..48: mol_type = other RNA
tattgcactcgtcccggcctgtttttttaacgtgagcagggccggaca

SEQ ID NO: 969 - RNA, 48 nt, synthetic construct
  source 1..48: mol_type = other RNA
tattgcacttgtcccggccagtttttttaacgtgaacagggccggtca

SEQ ID NO: 970 - RNA, 46 nt, synthetic construct
  source 1..46: mol_type = other RNA
tattgcacttgtcccggcccgttttttaacgtgaacagggccgggc

SEQ ID NO: 971 - RNA, 46 nt, synthetic construct
  source 1..46: mol_type = other RNA
tattgcacttgtcccggcctgttttttaacgtgaacagggccggac

SEQ ID NO: 972 - RNA, 46 nt, synthetic construct
  source 1..46: mol_type = other RNA
tattgcgcttgtcccggcctgttttttaacgcgaacagggccggac

SEQ ID NO: 973 - RNA, 48 nt, synthetic construct
  source 1..48: mol_type = other RNA
tattgcacttgtccctgcctgtttttttaacgtgaacagggacggaca

SEQ ID NO: 974 - RNA, 48 nt, synthetic construct
  source 1..48: mol_type = other RNA
tattgcacttatcccggcctgtttttttaacgtgaatagggccggaca

SEQ ID NO: 975 - RNA, 48 nt, synthetic construct
  source 1..48: mol_type = other RNA
tattgcacttgtccaggcctgtttttttaacgtgaacaggtccggaca

SEQ ID NO: 976 - RNA, 48 nt, synthetic construct
  source 1..48: mol_type = other RNA
tattgctcttgtcccggcctgtttttttaacgagaacagggccggaca

SEQ ID NO: 977 - RNA, 50 nt, synthetic construct
  source 1..50: mol_type = other RNA
tattgcacttgtcccggcctgtgttttttaacgtgaacagggccggacac

SEQ ID NO: 978 - RNA, 50 nt, synthetic construct
  source 1..50: mol_type = other RNA
tcccgtactgagctgccccgagatttttgggcatgactcgacggggctct

SEQ ID NO: 979 - RNA, 48 nt, synthetic construct
  source 1..48: mol_type = other RNA
tcccgtactgagctgccccgagtttttgggcatgactcgacggggctc

SEQ ID NO: 980 - RNA, 44 nt, synthetic construct
  source 1..44: mol_type = other RNA
tcccgtactgagctgccccgtttttgggcatgactcgacggggc

SEQ ID NO: 981 - RNA, 68 nt, synthetic construct
  source 1..68: mol_type = other RNA
tccctggtggtctaatggttaggattcggcgctttttgggaccaccagattaccaatcctaagccgcg

SEQ ID NO: 982 - RNA, 68 nt, synthetic construct
  source 1..68: mol_type = other RNA
tccctggtggtctagtagttaggattcggcgctttttgggaccaccagatcatcaatcctaagccgcg

SEQ ID NO: 983 - RNA, 70 nt, synthetic construct
  source 1..70: mol_type = other RNA
tccctggtgatctagtggttaggattcggcgcttttttgggaccactagatcaccaatcctaagccgcga

SEQ ID NO: 984 - RNA, 74 nt, synthetic construct
  source 1..74: mol_type = other RNA
tccctggtggtctagtggtcaggattcggcgctcttttttgggaccaccagatcaccagtcctaagccgcgaga

SEQ ID NO: 985 - RNA, 50 nt, synthetic construct
  source 1..50: mol_type = other RNA
tcctgcactgagctgccccgagatttttggacgtgactcgacggggctct

SEQ ID NO: 986 - RNA, 50 nt, synthetic construct
  source 1..50: mol_type = other RNA
tcctgtactaagctgccccgagatttttggacatgattcgacggggctct

SEQ ID NO: 987 - RNA, 50 nt, synthetic construct
  source 1..50: mol_type = other RNA
tcctgtaccgagctgccccgagttttttggacatggctcgacggggctca

SEQ ID NO: 988 - RNA, 48 nt, synthetic construct
  source 1..48: mol_type = other RNA
tcctgtactgagctgccccaagtttttggacatgactcgacggggttc

SEQ ID NO: 989 - RNA, 50 nt, synthetic construct
  source 1..50: mol_type = other RNA
tcctgtactgagctgccccgggatttttggacatgactcgacggggccct

SEQ ID NO: 990 - RNA, 48 nt, synthetic construct
  source 1..48: mol_type = other RNA
tcctgtactgagctgccccgtgtttttggacatgactcgacggggcac

SEQ ID NO: 991 - RNA, 50 nt, synthetic construct
  source 1..50: mol_type = other RNA
tcctgtaccgagctgccccgagatttttggacatggctcgacggggctct

SEQ ID NO: 992 - RNA, 46 nt, synthetic construct
  source 1..46: mol_type = other RNA
tcctgtactgagctgccacgatttttggacatgactcgacggtgct

SEQ ID NO: 993 - RNA, 48 nt, synthetic construct
  source 1..48: mol_type = other RNA
tcctgtaccgagctgccccgagtttttggacatggctcgacggggctc

SEQ ID NO: 994 - RNA, 46 nt, synthetic construct
  source 1..46: mol_type = other RNA
tcctgtactgagctgccccgttttttggacatgactcgacggggca

SEQ ID NO: 995 - RNA, 50 nt, synthetic construct
  source 1..50: mol_type = other RNA
tcctgtactgagctgccccaagatttttggacatgactcgacggggttct

SEQ ID NO: 996 - RNA, 48 nt, synthetic construct
  source 1..48: mol_type = other RNA
tcctgtactgagttgccccgagtttttggacatgactcaacggggctc

SEQ ID NO: 997 - RNA, 50 nt, synthetic construct
  source 1..50: mol_type = other RNA
tcctgtactgggctgccccgagttttttggacatgacccgacggggctca

SEQ ID NO: 998 - RNA, 48 nt, synthetic construct
  source 1..48: mol_type = other RNA
tcctgtactgggctgccccgagtttttggacatgacccgacggggctc

SEQ ID NO: 999 - RNA, 46 nt, synthetic construct
  source 1..46: mol_type = other RNA
tcctgtactgagctgcccgagtttttggacatgactcgacgggctc

SEQ ID NO: 1000 - RNA, 46 nt, synthetic construct
  source 1..46: mol_type = other RNA
tgagaactgaattccatgggttttttctcttgacttaaggtaccca

SEQ ID NO: 1001 - RNA, 46 nt, synthetic construct
  source 1..46: mol_type = other RNA
tgaggggcagagagcgagacctttttctccccgtctctcgctctgg

SEQ ID NO: 1002 - RNA, 46 nt, synthetic construct
  source 1..46: mol_type = other RNA
tgagggacagagagcgagacttttttctccctgtctctcgctctga

SEQ ID NO: 1003 - RNA, 52 nt, synthetic construct
  source 1..52: mol_type = other RNA
tgaggggcagagagcgggactttttttttctccccgtctctcgccctgaaaa

SEQ ID NO: 1004 - RNA, 50 nt, synthetic construct
  source 1..50: mol_type = other RNA
tgaggggcagagggcgagacttttttttctccccgtctcccgctctgaaa

SEQ ID NO: 1005 - RNA, 50 nt, synthetic construct
  source 1..50: mol_type = other RNA
tgaggtagtaggtcgtgtggttttttttctccatcatccagcacaccaaa

SEQ ID NO: 1006 - RNA, 50 nt, synthetic construct; identical to SEQ ID NO: 884
  source 1..50: mol_type = other RNA

SEQ ID NO: 1007 - RNA, 44 nt, synthetic construct
  source 1..44: mol_type = other RNA
tgaggtagtaggtcgtgtggtttttctccatcatccagcacacc

SEQ ID NO: 1008 - RNA, 44 nt, synthetic construct
  source 1..44: mol_type = other RNA
tgaggtagtaggttgtatagtttttctccatcatccaacatatc

SEQ ID NO: 1009 - RNA, 46 nt, synthetic construct
  source 1..46: mol_type = other RNA
tgaggtagtagtttgcgctgttttttctccatcatcaaacgcgaca

SEQ ID NO: 1010 - RNA, 44 nt, synthetic construct
  source 1..44: mol_type = other RNA
tgaggtagtaggtcgtatagtttttctccatcatccagcatatc

SEQ ID NO: 1011 - RNA, 46 nt, synthetic construct
  source 1..46: mol_type = other RNA
tgaggtagtaggtcgtatagttttttctccatcatccagcatatca

SEQ ID NO: 1012 - RNA, 42 nt, synthetic construct
  source 1..42: mol_type = other RNA
tgaggtagtaggttgtgtgtttttctccatcatccaacacac

SEQ ID NO: 1013 - RNA, 48 nt, synthetic construct
  source 1..48: mol_type = other RNA
tgaggtagtaggttatgtggtttttttctccatcatccaatacaccaa

SEQ ID NO: 1014 - RNA, 48 nt, synthetic construct
  source 1..48: mol_type = other RNA
tgaggtagtggattgtatagtttttttctccatcacctaacatatcaa

SEQ ID NO: 1015 - RNA, 48 nt, synthetic construct
  source 1..48: mol_type = other RNA
tgaggtagtagtttgcacagtttttttctccatcatcaaacgtgtcaa

SEQ ID NO: 1016 - RNA, 44 nt, synthetic construct
  source 1..44: mol_type = other RNA
tgaggtagtaggttgtgtggtttttctccatcatccaacacacc

SEQ ID NO: 1017 - RNA, 48 nt, synthetic construct
  source 1..48: mol_type = other RNA
tggagtgtgacaatggtgtctgtttttcctcacactgttaccacagac

SEQ ID NO: 1018 - RNA, 46 nt, synthetic construct
  source 1..46: mol_type = other RNA
tgggtttacgttgggagaacttttttcccaaatgcaaccctcttga

SEQ ID NO: 1019 - RNA, 48 nt, synthetic construct
  source 1..48: mol_type = other RNA
tgtaaacatccttgactggaagtttttcatttgtaggaactgaccttc

SEQ ID NO: 1020 - RNA, 48 nt, synthetic construct
  source 1..48: mol_type = other RNA
tgtaaacatccccgactggaaatttttcatttgtaggggctgaccttt

SEQ ID NO: 1021 - RNA, 46 nt, synthetic construct
  source 1..46: mol_type = other RNA
tgtaaacatcctacactctcatttttcatttgtaggatgtgagagt

SEQ ID NO: 1022 - RNA, 52 nt, synthetic construct
  source 1..52: mol_type = other RNA
tgtagacatccttgactggaagcttttttcatctgtaggaactgaccttcga

SEQ ID NO: 1023 - RNA, 48 nt, synthetic construct
  source 1..48: mol_type = other RNA
tgttgcacttgtcccggcctgttttttcaacgtgaacagggccggaca

SEQ ID NO: 1024 - RNA, 50 nt, synthetic construct
  source 1..50: mol_type = other RNA
ttctgtactgagctgccccgagatttttagacatgactcgacggggctct

SEQ ID NO: 1025 - RNA, 44 nt, synthetic construct
  source 1..44: mol_type = other RNA
ttgcacttgtcccggcctgttttttacgtgaacagggccggaca

SEQ ID NO: 1026 - RNA, 40 nt, synthetic construct; identical to SEQ ID NO: 889
  source 1..40: mol_type = other RNA

SEQ ID NO: 1027 - RNA, 40 nt, synthetic construct; identical to SEQ ID NO: 898
  source 1..40: mol_type = other RNA

SEQ ID NO: 1028 - RNA, 48 nt, synthetic construct
  source 1..48: mol_type = other RNA
ttgtagcactggggccagatggtttttacatcgtgaccccggtctacc

SEQ ID NO: 1029 - RNA, 44 nt, synthetic construct
  source 1..44: mol_type = other RNA
agatgagaggacgggacgccttttactactctcctgccctgcgg

SEQ ID NO: 1030 - RNA, 48 nt, synthetic construct
  source 1..48: mol_type = other RNA
tatggaagtgccgccgctgaaattttttaccttcacggcggcgacttt

SEQ ID NO: 1031 - RNA, 48 nt, synthetic construct
  source 1..48: mol_type = other RNA
tattaaatcaatctcggctggcttttttaatttagttagagccgaccg

SEQ ID NO: 1032 - RNA, 20 nt, synthetic construct
  source 1..20: mol_type = other RNA
ccccctcaacccagcttttc

SEQ ID NO: 1033 - RNA, 35 nt, synthetic construct
  source 1..35: mol_type = other RNA
acgtcaggaccgctacggacctccaccagagtttc

SEQ ID NO: 1034 - RNA, 17 nt, synthetic construct
  source 1..17: mol_type = other RNA
ttatccgctcacaattc

SEQ ID NO: 1035 - RNA, 22 nt, synthetic construct
  source 1..22: mol_type = other RNA
tattttgccgacttcccttatc

SEQ ID NO: 1036 - RNA, 31 nt, synthetic construct
  source 1..31: mol_type = other RNA
ggcgagaattctaccactgaaccactcatgc

SEQ ID NO: 1037 - RNA, 18 nt, synthetic construct
  source 1..18: mol_type = other RNA
tccattgcgccactggcc

SEQ ID NO: 1038 - RNA, 36 nt, synthetic construct
  source 1..36: mol_type = other RNA
ggccccgcttcgcgccccagcccgaccgacccagcc

SEQ ID NO: 1039 - RNA, 20 nt, synthetic construct
  source 1..20: mol_type = other RNA
cctatatcgccgacatcacc

SEQ ID NO: 1040 - RNA, 21 nt, synthetic construct
  source 1..21: mol_type = other RNA
ggatgctcacgatacgggtta

SEQ ID NO: 1041 - RNA, 33 nt, synthetic construct
  source 1..33: mol_type = other RNA
gaagccagtcaaatttagcagtggggggttgta

SEQ ID NO: 1042 - RNA, 22 nt, synthetic construct
  source 1..22: mol_type = other RNA
gatggtggctaacggcgggata

SEQ ID NO: 1043 - RNA, 22 nt, synthetic construct
  source 1..22: mol_type = other RNA
acctgtcctacgagttgcatga

SEQ ID NO: 1044 - RNA, 24 nt, synthetic construct
  source 1..24: mol_type = other RNA
aacaacagaatcactagtcttcca

SEQ ID NO: 1045 - RNA, 28 nt, synthetic construct
  source 1..28: mol_type = other RNA
attgagataacccactaccttcggacca

SEQ ID NO: 1046 - RNA, 21 nt, synthetic construct
  source 1..21: mol_type = other RNA
aaatcttatctcatccgccaa

SEQ ID NO: 1047 - RNA, 33 nt, synthetic construct
  source 1..33: mol_type = other RNA
caggcgaacgtgataaccactacactacggaaa

SEQ ID NO: 1048 - RNA, 22 nt, synthetic construct
  source 1..22: mol_type = other RNA
gcccaagtcagaggtggcgaaa

SEQ ID NO: 1049 - RNA, 23 nt, synthetic construct
  source 1..23: mol_type = other RNA
ggtaagccctcccgtatcgtagt

SEQ ID NO: 1050 - RNA, 21 nt, synthetic construct
  source 1..21: mol_type = other RNA
ctagcagtgcgcgacgttctt

SEQ ID NO: 1051 - RNA, 22 nt, synthetic construct
  source 1..22: mol_type = other RNA
catacgccagttcacgagcatg

SEQ ID NO: 1052 - RNA, 24 nt, synthetic construct
  source 1..24: mol_type = other RNA
tttggcacccgagaattccacacg

SEQ ID NO: 1053 - RNA, 21 nt, synthetic construct
  source 1..21: mol_type = other RNA
tgtttctcgagactacccgga

SEQ ID NO: 1054 - RNA, 21 nt, synthetic construct
  source 1..21: mol_type = other RNA
atagccaggtccgggtgttca

SEQ ID NO: 1055 - RNA, 22 nt, synthetic construct
  source 1..22: mol_type = other RNA
cgtggtgccacgctcgtcgttt

SEQ ID NO: 1056 - RNA, 21 nt, synthetic construct
  source 1..21: mol_type = other RNA
gagtagataacgaagcgaaag

SEQ ID NO: 1057 - RNA, 17 nt, synthetic construct
  source 1..17: mol_type = other RNA
gttcgattccttccttt

SEQ ID NO: 1058 - RNA, 31 nt, synthetic construct
  source 1..31: mol_type = other RNA
gcctagcacgcttccgctgcgccactctgct

SEQ ID NO: 1059 - RNA, 31 nt, synthetic construct
  source 1..31: mol_type = other RNA
gtccagcacgcttccgctgcgcaactctgct

SEQ ID NO: 1060 - RNA, 32 nt, synthetic construct
  source 1..32: mol_type = other RNA
gttctaataacccactaccatcggaccagcct

SEQ ID NO: 1061 - RNA, 21 nt, synthetic construct
  source 1..21: mol_type = other RNA
tgaacacccggacctggttat

SEQ ID NO: 1062 - RNA, 20 nt, synthetic construct
  source 1..20: mol_type = other RNA
accatcggcaccaccacgct

SEQ ID NO: 1063 - RNA, 21 nt, synthetic construct
  source 1..21: mol_type = other RNA
ataaccgggtccgggtgttca

SEQ ID NO: 1064 - RNA, 17 nt, synthetic construct
  source 1..17: mol_type = other RNA
catgtccaactgctgtt

SEQ ID NO: 1065 - RNA, 34 nt, synthetic construct
  source 1..34: mol_type = other RNA
tggtattctcgcacggactacagccacgaccaat

SEQ ID NO: 1066 - RNA, 20 nt, synthetic construct
  source 1..20: mol_type = other RNA
ccatgccaacccgttccatg

SEQ ID NO: 1067 - RNA, 21 nt, synthetic construct
  source 1..21: mol_type = other RNA
gatcccagacaccatcgaatg

SEQ ID NO: 1068 - RNA, 30 nt, synthetic construct
  source 1..30: mol_type = other RNA
ggcgagaattctactactgaaccaccaatg

SEQ ID NO: 1069 - RNA, 20 nt, synthetic construct
  source 1..20: mol_type = other RNA
caatttcactgagtctcggg

SEQ ID NO: 1070 - RNA, 35 nt, synthetic construct
  source 1..35: mol_type = other RNA
tccccttcttttcttccccttcttctttccttctt

SEQ ID NO: 1071 - RNA, 24 nt, synthetic construct
  source 1..24: mol_type = other RNA
atctcccttctttcctcccgtctt

SEQ ID NO: 1072 - RNA, 39 nt, synthetic construct
  source 1..39: mol_type = other RNA
gtcccgccgccgccgccgccgccaccgccgccgccgccg

SEQ ID NO: 1073 - RNA, 22 nt, synthetic construct
  source 1..22: mol_type = other RNA
aaacccatggaattcagttctc

SEQ ID NO: 1074 - RNA, 20 nt, synthetic construct
  source 1..20: mol_type = other RNA
ccgtcctcctcctcctcctc

SEQ ID NO: 1075 - RNA, 20 nt, synthetic construct
  source 1..20: mol_type = other RNA
actatgcaacctactacctc

SEQ ID NO: 1076 - RNA, 20 nt, synthetic construct
  source 1..20: mol_type = other RNA
attccggcagcgtcttactc

SEQ ID NO: 1077 - RNA, 23 nt, synthetic construct
  source 1..23: mol_type = other RNA
tggcgagcccgatcttccccatc

SEQ ID NO: 1078 - RNA, 33 nt, synthetic construct
  source 1..33: mol_type = other RNA
caggcgagaattctaccactgaaccaccaatgc

SEQ ID NO: 1079 - RNA, 26 nt, synthetic construct
  source 1..26: mol_type = other RNA
gaattctaccactgaaccacaaatgc

SEQ ID NO: 1080 - RNA, 29 nt, synthetic construct
  source 1..29: mol_type = other RNA
tgagataactcactaccttcggaccagcc

SEQ ID NO: 1081 - RNA, 24 nt, synthetic construct
  source 1..24: mol_type = other RNA
aagcttccagtcaaggatgtttac

SEQ ID NO: 1082 - RNA, 23 nt, synthetic construct
  source 1..23: mol_type = other RNA
gtcaacgtcagtctgataagcta

SEQ ID NO: 1083 - RNA, 24 nt, synthetic construct
  source 1..24: mol_type = other RNA
agtcaacatcagtctgataagcta

SEQ ID NO: 1084 - RNA, 22 nt, synthetic construct
  source 1..22: mol_type = other RNA
acaggccgggacaactgcaata

SEQ ID NO: 1085 - RNA, 21 nt, synthetic construct
  source 1..21: mol_type = other RNA
ttgctacagtgcttcatctca

SEQ ID NO: 1086 - RNA, 20 nt, synthetic construct
  source 1..20: mol_type = other RNA
atctcgctctctgcccctca

SEQ ID NO: 1087 - RNA, 21 nt, synthetic construct
  source 1..21: mol_type = other RNA
agcctcgctctctgcccctca

SEQ ID NO: 1088 - RNA, 23 nt, synthetic construct
  source 1..23: mol_type = other RNA
aaggtctcgctctctgcccctca

SEQ ID NO: 1089 - RNA, 22 nt, synthetic construct
  source 1..22: mol_type = other RNA
tcttctcgctctctgcccctca

SEQ ID NO: 1090 - RNA, 23 nt, synthetic construct
  source 1..23: mol_type = other RNA
agaccacacaacctactacctca

SEQ ID NO: 1091 - RNA, 19 nt, synthetic construct
  source 1..19: mol_type = other RNA
tcaccattgtcacactcca

SEQ ID NO: 1092 - RNA, 22 nt, synthetic construct
  source 1..22: mol_type = other RNA
ctttcagtcggggatgtttaca

SEQ ID NO: 1093 - RNA, 23 nt, synthetic construct
  source 1..23: mol_type = other RNA
gcttccagtcggggatatttaca

SEQ ID NO: 1094 - RNA, 23 nt, synthetic construct
  source 1..23: mol_type = other RNA
acttccagtcaaggatgtttaca

SEQ ID NO: 1095 - RNA, 18 nt, synthetic construct
  source 1..18: mol_type = other RNA
ccgcccgcccgctcccaa

SEQ ID NO: 1096 - RNA, 21 nt, synthetic construct
  source 1..21: mol_type = other RNA
agaaccattaccattactaaa

SEQ ID NO: 1097 - RNA, 30 nt, synthetic construct
  source 1..30: mol_type = other RNA
gcgaacgtgataaccactacactacggaaa

SEQ ID NO: 1098 - RNA, 19 nt, synthetic construct
  source 1..19: mol_type = other RNA
cgcgagccgaacgaacaaa

SEQ ID NO: 1099 - RNA, 25 nt, synthetic construct
  source 1..25: mol_type = other RNA
cccccgccgccgccgccgccgccgt

SEQ ID NO: 1100 - RNA, 21 nt, synthetic construct
  source 1..21: mol_type = other RNA
tcctcctcctcttcctcctct

SEQ ID NO: 1101 - RNA, 31 nt, synthetic construct
  source 1..31: mol_type = other RNA
ttctgataacccactaccatcggaccagcct

SEQ ID NO: 1102 - RNA, 25 nt, synthetic construct
  source 1..25: mol_type = other RNA
cccccgccgccgccgccgccgccct

SEQ ID NO: 1103 - RNA, 24 nt, synthetic construct
  source 1..24: mol_type = other RNA
ccccgccgccgccgccgccgccct

SEQ ID NO: 1104 - RNA, 18 nt, synthetic construct
  source 1..18: mol_type = other RNA
gagaagaacaaccggaat

SEQ ID NO: 1105 - RNA, 21 nt, synthetic construct
  source 1..21: mol_type = other RNA
ccacacaacctactacctcat

SEQ ID NO: 1106 - RNA, 21 nt, synthetic construct
  source 1..21: mol_type = other RNA
acaccgacagcgttgaatgtt

SEQ ID NO: 1107 - RNA, 21 nt, synthetic construct
  source 1..21: mol_type = other RNA
acagcccatcgactggtgttg

SEQ ID NO: 1108 - RNA, 18 nt, synthetic construct
  source 1..18: mol_type = other RNA
gcttttgggattccgttg

SEQ ID NO: 1109 - RNA, 20 nt, synthetic construct
  source 1..20: mol_type = other RNA
tttggtgtttccgcccggtt

SEQ ID NO: 1110 - RNA, 18 nt, synthetic construct
  source 1..18: mol_type = other RNA
acccgggtcgcaagaatg

SEQ ID NO: 1111 - RNA, 33 nt, synthetic construct
  source 1..33: mol_type = other RNA
gagcgccgaatcctaaccactagaccacagggg

SEQ ID NO: 1112 - RNA, 20 nt, synthetic construct
  source 1..20: mol_type = other RNA
tccctctcaacccagctttt

SEQ ID NO: 1113 - RNA, 28 nt, synthetic construct
  source 1..28: mol_type = other RNA
ttgagataactcactaccttcggaccag

SEQ ID NO: 1114 - RNA, 21 nt, synthetic construct
  source 1..21: mol_type = other RNA
cgccctctcagcccagctttt

SEQ ID NO: 1115 - RNA, 20 nt, synthetic construct
  source 1..20: mol_type = other RNA
tccctctcaacccagctttt

SEQ ID NO: 1116 - RNA, 23 nt, synthetic construct
  source 1..23: mol_type = other RNA
ctcgccctctcaacccagctttt

SEQ ID NO: 1117 - RNA, 22 nt, synthetic construct
  source 1..22: mol_type = other RNA
gcagttcttcaactggcagctt

SEQ ID NO: 1118 - RNA, 22 nt, synthetic construct
  source 1..22: mol_type = other RNA
aaacccagcagacaatgtagct

SEQ ID NO: 1119 - RNA, 23 nt, synthetic construct
  source 1..23: mol_type = other RNA
actgaggggcctcaggccgagct

SEQ ID NO: 1120 - RNA, 33 nt, synthetic construct
  source 1..33: mol_type = other RNA
agttctgataacccactaccatcggaccagcct

SEQ ID NO: 1121 - RNA, 25 nt, synthetic construct
  source 1..25: mol_type = other RNA
cccccgccgccgccgccgccgccct

SEQ ID NO: 1122 - RNA, 23 nt, synthetic construct
  source 1..23: mol_type = other RNA
acaggccgggacaagtgcaatat

SEQ ID NO: 1123 - RNA, 22 nt, synthetic construct
  source 1..22: mol_type = other RNA
tcggggcagctcagtacaggat

SEQ ID NO: 1124 - RNA, 23 nt, synthetic construct
  source 1..23: mol_type = other RNA
ctcggggcagctcagtacaggat

SEQ ID NO: 1125 - RNA, 20 nt, synthetic construct
  source 1..20: mol_type = other RNA
caggccgggacaagtgcaat

SEQ ID NO: 1126 - RNA, 21 nt, synthetic construct
  source 1..21: mol_type = other RNA
acaggccgggacaagtgcaat

SEQ ID NO: 1127 - RNA, 21 nt, synthetic construct
  source 1..21: mol_type = other RNA
acaggccggggcaagtgcaat

SEQ ID NO: 1128 - RNA, 21 nt, synthetic construct
  source 1..21: mol_type = other RNA
caggccgggacaagtgcaatg

SEQ ID NO: 1129 - RNA, 22 nt, synthetic construct
  source 1..22: mol_type = other RNA
ccagaccgagacaagtgcaatg

SEQ ID NO: 1130 - RNA, 30 nt, synthetic construct
  source 1..30: mol_type = other RNA
ggcgagaattctaccactgaaccaccaatg

SEQ ID NO: 1131 - RNA, 27 nt, synthetic construct
  source 1..27: mol_type = other RNA
aagccagtcaaatttagcagcgggggg

SEQ ID NO: 1132 - RNA, 23 nt, synthetic construct
  source 1..23: mol_type = other RNA
tctcggggcagctcagtacaggg

SEQ ID NO: 1133 - RNA, 22 nt, synthetic construct
  source 1..22: mol_type = other RNA
tctcggggcagctcagtacagg

SEQ ID NO: 1134 - RNA, 21 nt, synthetic construct
  source 1..21: mol_type = other RNA
ctcggggcagctcagtacagg

SEQ ID NO: 1135 - RNA, 20 nt, synthetic construct
  source 1..20: mol_type = other RNA
cgtcctcctcttcctcctcg

SEQ ID NO: 1136 - RNA, 20 nt, synthetic construct
  source 1..20: mol_type = other RNA
ctatacaacctactacctcg

SEQ ID NO: 1137 - RNA, 20 nt, synthetic construct
  source 1..20: mol_type = other RNA
aaaggcagcaggtcgtatag

SEQ ID NO: 1138 - RNA, 20 nt, synthetic construct
  source 1..20: mol_type = other RNA
ctcggggcagctcagtacag

SEQ ID NO: 1139 - RNA, 30 nt, synthetic construct
  source 1..30: mol_type = other RNA
gcgagaattctaccactgaaccaccaattc

SEQ ID NO: 1140 - RNA, 32 nt, synthetic construct
  source 1..32: mol_type = other RNA
aggcgagaattctaccactgaaccaccgatgc

SEQ ID NO: 1141 - RNA, 32 nt, synthetic construct
  source 1..32: mol_type = other RNA
aggcgagaattctaccactgaaccactaatgc

SEQ ID NO: 1142 - RNA, 31 nt, synthetic construct
  source 1..31: mol_type = other RNA
ggcgagaattctaccactgaaccatcaatgc

SEQ ID NO: 1143 - RNA, 31 nt, synthetic construct
  source 1..31: mol_type = other RNA
ggcgagaattctaccactgaactaccaatgc

SEQ ID NO: 1144 - RNA, 33 nt, synthetic construct
  source 1..33: mol_type = other RNA
cgggcgagaattctaccactgaaccaccaatgc

SEQ ID NO: 1145 - RNA, 31 nt, synthetic construct
  source 1..31: mol_type = other RNA
gacgagaattctaccactgaaccaccaatgc

SEQ ID NO: 1146 - RNA, 32 nt, synthetic construct
  source 1..32: mol_type = other RNA
aggcgaaaattctaccactgaaccaccaatgc

SEQ ID NO: 1147 - RNA, 31 nt, synthetic construct
  source 1..31: mol_type = other RNA
ggcgagaatcctaccactgaaccaccaatgc

SEQ ID NO: 1148 - RNA, 31 nt, synthetic construct
  source 1..31: mol_type = other RNA
ggcgagaactctaccactgaaccaccaatgc

SEQ ID NO: 1149 - RNA, 31 nt, synthetic construct
  source 1..31: mol_type = other RNA
ggcgagaattctaccaccgaaccaccaatgc

SEQ ID NO: 1150 - RNA, 32 nt, synthetic construct
  source 1..32: mol_type = other RNA
aatcttgcatgatgccactacaccagcggcgc

SEQ ID NO: 1151 - RNA, 31 nt, synthetic construct
  source 1..31: mol_type = other RNA
ggcgagaattctaccactgaaccaccaacgc

SEQ ID NO: 1152 - RNA, 34 nt, synthetic construct
  source 1..34: mol_type = other RNA
taagttctgataacccactaccatcgggccagcc

SEQ ID NO: 1153 - RNA, 30 nt, synthetic construct
  source 1..30: mol_type = other RNA
ttgaggtaactcactaccttcggaccagcc

SEQ ID NO: 1154 - RNA, 30 nt, synthetic construct
  source 1..30: mol_type = other RNA
ttgagataactcactgccttcggaccagcc

SEQ ID NO: 1155 - RNA, 33 nt, synthetic construct
  source 1..33: mol_type = other RNA
aagttctgataacccactactatcggaccagcc

SEQ ID NO: 1156 - RNA, 30 nt, synthetic construct
  source 1..30: mol_type = other RNA
ttctgatgacccactaccatcggaccagcc

SEQ ID NO: 1157 - RNA, 30 nt, synthetic construct
  source 1..30: mol_type = other RNA
ttctgataacccgctaccatcggaccagcc

SEQ ID NO: 1158 - RNA, 33 nt, synthetic construct
  source 1..33: mol_type = other RNA
aagttctgacaacccactaccatcggaccagcc

SEQ ID NO: 1159 - RNA, 31 nt, synthetic construct
  source 1..31: mol_type = other RNA
gttctgataacctactaccatcggaccagcc

SEQ ID NO: 1160 - RNA, 21 nt, synthetic construct
  source 1..21: mol_type = other RNA
gttatccgctcgcaattcccc

SEQ ID NO: 1161 - RNA, 21 nt, synthetic construct
  source 1..21: mol_type = other RNA
attatccgctcacaattcccc

SEQ ID NO: 1162 - RNA, 21 nt, synthetic construct
  source 1..21: mol_type = other RNA
gttgtccgctcacaattcccc

SEQ ID NO: 1163 - RNA, 22 nt, synthetic construct
  source 1..22: mol_type = other RNA
cctgttccgaccccgccgctta

SEQ ID NO: 1164 - RNA, 21 nt, synthetic construct
  source 1..21: mol_type = other RNA
agtctcgctctctgcccctta

SEQ ID NO: 1165 - RNA, 22 nt, synthetic construct
  source 1..22: mol_type = other RNA
acaggccgggacaagtgcatta

SEQ ID NO: 1166 - RNA, 22 nt, synthetic construct
  source 1..22: mol_type = other RNA
cgccgatatttacgtgctgcta

SEQ ID NO: 1167 - RNA, 22 nt, synthetic construct
  source 1..22: mol_type = other RNA
tcaacgtcagtctgataagcta

SEQ ID NO: 1168 - RNA, 22 nt, synthetic construct
  source 1..22: mol_type = other RNA
ttaacatcagtctgataagcta

SEQ ID NO: 1169 - RNA, 20 nt, synthetic construct
  source 1..20: mol_type = other RNA
aacatcagtctgataagcta

SEQ ID NO: 1170 - RNA, 22 nt, synthetic construct
  source 1..22: mol_type = other RNA
acaggccgtgacaagtgcaata

SEQ ID NO: 1171 - RNA, 22 nt, synthetic construct
  source 1..22: mol_type = other RNA
acaggccggggcaagtgcaata

SEQ ID NO: 1172 - RNA, 21 nt, synthetic construct
  source 1..21: mol_type = other RNA
cagaccgggacaagtgcaata

SEQ ID NO: 1173 - RNA, 22 nt, synthetic construct
  source 1..22: mol_type = other RNA
acaggctgggacaagtgcaata

SEQ ID NO: 1174 - RNA, 22 nt, synthetic construct
  source 1..22: mol_type = other RNA
acaggccgggacgagtgcaata

SEQ ID NO: 1175 - RNA, 22 nt, synthetic construct
  source 1..22: mol_type = other RNA
actggccgggacaagtgcaata

SEQ ID NO: 1176 - RNA, 21 nt, synthetic construct
  source 1..21: mol_type = other RNA
cgggccgggacaagtgcaata

SEQ ID NO: 1177 - RNA, 21 nt, synthetic construct
  source 1..21: mol_type = other RNA
caggccgggacaagtgcaata

SEQ ID NO: 1178 - RNA, 21 nt, synthetic construct
  source 1..21: mol_type = other RNA
caggccgggacaagcgcaata

SEQ ID NO: 1179 - RNA, 22 nt, synthetic construct
  source 1..22: mol_type = other RNA
acaggcagggacaagtgcaata

SEQ ID NO: 1180 - RNA, 22 nt, synthetic construct
  source 1..22: mol_type = other RNA
acaggccgggataagtgcaata

SEQ ID NO: 1181 - RNA, 22 nt, synthetic construct
  source 1..22: mol_type = other RNA
acaggcctggacaagtgcaata

SEQ ID NO: 1182 - RNA, 22 nt, synthetic construct
  source 1..22: mol_type = other RNA
acaggccgggacaagagcaata

SEQ ID NO: 1183 - RNA, 23 nt, synthetic construct
  source 1..23: mol_type = other RNA
cacaggccgggacaagtgcaata

SEQ ID NO: 1184 - RNA, 23 nt, synthetic construct
  source 1..23: mol_type = other RNA
tctcggggcagctcagtacggga

SEQ ID NO: 1185 - RNA, 22 nt, synthetic construct
  source 1..22: mol_type = other RNA
ctcggggcagctcagtacggga

SEQ ID NO: 1186 - RNA, 20 nt, synthetic construct
  source 1..20: mol_type = other RNA
cggggcagctcagtacggga

SEQ ID NO: 1187 - RNA, 32 nt, synthetic construct
  source 1..32: mol_type = other RNA
gcgccgaatcctaaccattagaccaccaggga

SEQ ID NO: 1188 - RNA, 32 nt, synthetic construct
  source 1..32: mol_type = other RNA
gcgccgaatcctaactactagaccaccaggga

SEQ ID NO: 1189 - RNA, 33 nt, synthetic construct
  source 1..33: mol_type = other RNA
agcgccgaatcctaaccactagatcaccaggga

SEQ ID NO: 1190 - RNA, 35 nt, synthetic construct
  source 1..35: mol_type = other RNA
agagcgccgaatcctgaccactagaccaccaggga

SEQ ID NO: 1191 - RNA, 23 nt, synthetic construct
  source 1..23: mol_type = other RNA
tctcggggcagctcagtgcagga

SEQ ID NO: 1192 - RNA, 23 nt, synthetic construct
  source 1..23: mol_type = other RNA
tctcggggcagcttagtacagga

SEQ ID NO: 1193 - RNA, 23 nt, synthetic construct
  source 1..23: mol_type = other RNA
actcggggcagctcggtacagga

SEQ ID NO: 1194 - RNA, 22 nt, synthetic construct
  source 1..22: mol_type = other RNA
cttggggcagctcagtacagga

SEQ ID NO: 1195 - RNA, 23 nt, synthetic construct
  source 1..23: mol_type = other RNA
tcccggggcagctcagtacagga

SEQ ID NO: 1196 - RNA, 22 nt, synthetic construct
  source 1..22: mol_type = other RNA
cacggggcagctcagtacagga

SEQ ID NO: 1197 - RNA, 23 nt, synthetic construct
  source 1..23: mol_type = other RNA
tctcggggcagctcggtacagga

SEQ ID NO: 1198 - RNA, 21 nt, synthetic construct
  source 1..21: mol_type = other RNA
tcgtggcagctcagtacagga

SEQ ID NO: 1199 - RNA, 22 nt, synthetic construct
  source 1..22: mol_type = other RNA
ctcggggcagctcggtacagga

SEQ ID NO: 1200 - RNA, 21 nt, synthetic construct
  source 1..21: mol_type = other RNA
acggggcagctcagtacagga

SEQ ID NO: 1201 - RNA, 23 nt, synthetic construct
  source 1..23: mol_type = other RNA
tcttggggcagctcagtacagga

SEQ ID NO: 1202 - RNA, 22 nt, synthetic construct
  source 1..22: mol_type = other RNA
ctcggggcaactcagtacagga

SEQ ID NO: 1203 - RNA, 23 nt, synthetic construct
  source 1..23: mol_type = other RNA
actcggggcagcccagtacagga

SEQ ID NO: 1204 - RNA, 22 nt, synthetic construct
  source 1..22: mol_type = other RNA
ctcggggcagcccagtacagga

SEQ ID NO: 1205 - RNA, 21 nt, synthetic construct
  source 1..21: mol_type = other RNA
ctcgggcagctcagtacagga

SEQ ID NO: 1206 - RNA, 21 nt, synthetic construct
  source 1..21: mol_type = other RNA
acccatggaattcagttctca

SEQ ID NO: 1207 - RNA, 21 nt, synthetic construct
  source 1..21: mol_type = other RNA
ggtctcgctctctgcccctca

SEQ ID NO: 1208 - RNA, 21 nt, synthetic construct
  source 1..21: mol_type = other RNA
agtctcgctctctgtccctca

SEQ ID NO: 1209 - RNA, 24 nt, synthetic construct
  source 1..24: mol_type = other RNA
aaaagtcccgctctctgcccctca

SEQ ID NO: 1210 - RNA, 23 nt, synthetic construct
  source 1..23: mol_type = other RNA
aaagtctcgccctctgcccctca

SEQ ID NO: 1211 - RNA, 23 nt, synthetic construct
  source 1..23: mol_type = other RNA
aaaccacacgacctactacctca

SEQ ID NO: 1212 - RNA, 23 nt, synthetic construct
  source 1..23: mol_type = other RNA
agaccacacaacctactacctca

SEQ ID NO: 1213 - RNA, 20 nt, synthetic construct
  source 1..20: mol_type = other RNA
ccacacgacctactacctca

SEQ ID NO: 1214 - RNA, 20 nt, synthetic construct
  source 1..20: mol_type = other RNA
ctatacaacctactacctca

SEQ ID NO: 1215 - RNA, 21 nt, synthetic construct
  source 1..21: mol_type = other RNA
acagcgcaaactactacctca

SEQ ID NO: 1216 - RNA, 20 nt, synthetic construct
  source 1..20: mol_type = other RNA
ctatacgacctactacctca

SEQ ID NO: 1217 - RNA, 21 nt, synthetic construct
  source 1..21: mol_type = other RNA
actatacgacctactacctca

SEQ ID NO: 1218 - RNA, 19 nt, synthetic construct
  source 1..19: mol_type = other RNA
cacacaacctactacctca

SEQ ID NO: 1219 - RNA, 22 nt, synthetic construct
  source 1..22: mol_type = other RNA
aaccacataacctactacctca

SEQ ID NO: 1220 - RNA, 22 nt, synthetic construct
  source 1..22: mol_type = other RNA
aactatacaatccactacctca

SEQ ID NO: 1221 - RNA, 22 nt, synthetic construct
  source 1..22: mol_type = other RNA
aactgtgcaaactactacctca

SEQ ID NO: 1222 - RNA, 20 nt, synthetic construct
  source 1..20: mol_type = other RNA
ccacacaacctactacctca

SEQ ID NO: 1223 - RNA, 22 nt, synthetic construct
  source 1..22: mol_type = other RNA
cagacaccattgtcacactcca

SEQ ID NO: 1224 - RNA, 21 nt, synthetic construct
  source 1..21: mol_type = other RNA
agttctcccaacgtaaaccca

SEQ ID NO: 1225 - RNA, 22 nt, synthetic construct
  source 1..22: mol_type = other RNA
cttccagtcaaggatgtttaca

SEQ ID NO: 1226 - RNA, 22 nt, synthetic construct
  source 1..22: mol_type = other RNA
tttccagtcggggatgtttaca

SEQ ID NO: 1227 - RNA, 21 nt, synthetic construct
  source 1..21: mol_type = other RNA
tgagagtgtaggatgtttaca

SEQ ID NO: 1228 - RNA, 24 nt, synthetic construct
  source 1..24: mol_type = other RNA
agcttccagtcaaggatgtctaca

SEQ ID NO: 1229 - RNA, 22 nt, synthetic construct
  source 1..22: mol_type = other RNA
acaggccgggacaagtgcaaca

SEQ ID NO: 1230 - RNA, 23 nt, synthetic construct
  source 1..23: mol_type = other RNA
tctcggggcagctcagtacagaa

SEQ ID NO: 1231 - RNA, 20 nt, synthetic construct
  source 1..20: mol_type = other RNA
acaggccgggacaagtgcaa

SEQ ID NO: 1232 - RNA, 18 nt, synthetic construct
  source 1..18: mol_type = other RNA
ccgcccgcccgctcccaa

SEQ ID NO: 1233 - RNA, 18 nt, synthetic construct
  source 1..18: mol_type = other RNA
gagaagaacaaccggaat

SEQ ID NO: 1234 - RNA, 22 nt, synthetic construct
  source 1..22: mol_type = other RNA
ccatctggccccagtgctacaa

SEQ ID NO: 1235 - RNA, 20 nt, synthetic construct
  source 1..20: mol_type = other RNA
ggcgtcccgtcctctcatct

SEQ ID NO: 1236 - RNA, 22 nt, synthetic construct
  source 1..22: mol_type = other RNA
tttcagcggcggcacttccata

SEQ ID NO: 1237 - RNA, 22 nt, synthetic construct
  source 1..22: mol_type = other RNA
gccagccgagattgatttaata

SEQ ID NO: 1238 - RNA, 22 nt, synthetic construct
  source 1..22: mol_type = other RNA
tggaattcctcgggtgccaagg

SEQ ID NO: 1239 - RNA, 29 nt, synthetic construct
  source 1..29: mol_type = other RNA
tctttccctacacgacgctcttccgatct